(19) United States
(12) Patent Application Publication
(10) Pub. No.: US 2024/0052382 A1
(43) Pub. Date: Feb. 15, 2024

(54) PROCESS CONTROL FOR 3-HYDROXYPROPIONIC ACID PRODUCTION BY ENGINEERED STRAINS OF ASPERGILLUS NIGER

(71) Applicant: Battelle Memorial Institute, Richland, WA (US)

(72) Inventors: Kyle R. Pomraning, Walden, CO (US); Ziyu Dai, Richland, WA (US); Jon K. Magnuson, Richland, WA (US); Beth A. Hofstad, Richland, WA (US); Jeffrey J. Czajka, Richland, WA (US); Joonhoon Kim, Kirkland, WA (US); Shuang Deng, Richland, WA (US)

(21) Appl. No.: 18/365,091

(22) Filed: Aug. 3, 2023

Related U.S. Application Data
(60) Provisional application No. 63/394,663, filed on Aug. 3, 2022.

(51) Int. Cl. C12P 7/42; C12N 9/88; C12N 9/10; C12N 9/04

(52) U.S. Cl. CPC C12P 7/42; C12N 9/88; C12Y 401/01011; C12N 9/1096; C12Y 206/01018; C12N 9/0006; C12Y 101/01059

(57) ABSTRACT

Provided herein are recombinant Aspergillus niger capable of producing 3-hydroxypropionic acid (3-HP). Also provided are methods of producing 3-hydroxypropionic acid (3-HP) and related kits.

### CROSS REFERENCE TO RELATED APPLICATION

This application claims priority to U.S. Provisional Application No. 63/394,663, filed Aug. 3, 2022, which is incorporated by reference in its entirety.

### ACKNOWLEDGMENT OF GOVERNMENT SUPPORT

This disclosure was made with Government support under Contract DE-AC0576RL01830 awarded by the U.S. Department of Energy. The Government has certain rights in the invention.

### INCORPORATION OF SEQUENCE LISTING

The Sequence Listing is submitted as an XML file named “Sequence.xml” (173,791 bytes), created on Aug. 3, 2023, which is incorporated by reference herein.

### FIELD

This application relates to the biosynthesis of 3-hydroxypropionic acid (3-HP), particularly in Aspergillus niger.

### BACKGROUND

Fuels and chemicals derived from non-fossil sources are needed to lessen human impacts on the environment while providing a healthy and growing economy. 3-hydroxypropionic acid (3-HP) is an important chemical building block that can be used for many products. While biosynthetic pathways have been designed to produce 3-HP from a variety of feedstocks in different microorganisms, low production is typically observed. Thus, there is a need for improved compositions and methods for 3-HP biosynthesis.

### SUMMARY

Provided herein are recombinant Aspergillus niger capable of producing 3-hydroxypropionic acid (3-HP). The recombinant Aspergillus niger include one or more exogenous nucleic acid molecules encoding aspartate 1-decarboxylase (PAND), β-alanine-pyruvate aminotransferase (BAPAT), and 3-hydroxypropionate dehydrogenase (HPDH). In some examples, the copy number of each of PAND, BAPAT, and HPDH is independently about 1 to about 80, for example, about 10 to about 30, or about 25 to about 30. In some implementations, the recombinant Aspergillus niger includes an exogenous nucleic acid molecule encoding pyruvate carboxylase (pyc), aspartate aminotransferase (aat1), and/or monocarboxylate transporter (mct1), resulting in overexpression of pyc, aat1, and/or mct1, respectively, in the recombinant Aspergillus niger. In some implementations, an ald6a, ald6b, ald3, and/or oahA gene is not present or does not produce a functional product in the recombinant Aspergillus niger. In a non-limiting example, the recombinant Aspergillus niger includes one or more exogenous nucleic acid molecules encoding PAND, BAPAT, HPDH, and pyc; and ald6a is not present or does not produce a functional product in the recombinant Aspergillus niger. In some examples, the PAND is from Tribolium castaneum. In some examples, the BAPAT is from Bacillus cereus. In some examples, the HPDH is from Escherichia coli. In some examples, the pyc is from Aspergillus niger. The exogenous nucleic acid molecules can be operably linked to a promoter and/or included on a vector.

Also disclosed are methods of producing 3-HP, including inoculating a media with Aspergillus niger comprising a 3-HP β-alanine pathway (e.g., a recombinant Aspergillus niger disclosed herein) thereby generating a cultured media, and fermenting the cultured media at a temperature of 30° C. to 37° C., under acidic and microaerobic conditions. In a non-limiting example, fermentation is performed at about 34° C., at about pH 2, and with a dissolved oxygen (DO) content of less than 10%. In some implementations, the media is Riscaldati B medium or a modified Riscaldati B medium. The media can further include trace elements, corn steep liquor, peptone, tryptone, soy protein, sodium chloride, yeast extract, sorbitol, and/or phosphorous. The media can further include glucose, maltose, fructose, maltodextrin, and/or a glucose:xylose mixture as a carbon source.

### BRIEF DESCRIPTION OF THE DRAWINGS

FIGS. 1A-1B. FIG. 1A: The effects of different trace elements and YPD on 3-HP production in the A. pseudoterreus transgenic strain Ap3HP6. The strain was inoculated in mRDM medium with various amounts of components and grown at 30° C. and 200 rpm. Each data point is the average of three biological replicates. FIG. 1B: The effects of additional copies of 3HP pathway (3HP::hph: 3HP4070) or 3HP pathway+aat1 over-expression (3HP::hph/aat1: 3HP4071) on 3-HP production in the A. pseudoterreus transgenic strain Ap3HP6 background. The strain was inoculated in mRDM medium and grown at 30° C. and 200 rpm for 7 days. Each data point is the average of three biological replicates.

FIGS. 2A-2D. 3-hydroxypropionic acid production FIG. 2A: in the selected A. niger transgenic strains of An3HP5, An3HP9, and An3HP10; FIG. 2B: in the selected A. niger transgenic strains with over-expression of pyruvate carboxylase (pyc) or cytosolic aspartate aminotransferase (aat) in An3HP9 strain; FIG. 2C: in the A. niger transgenic strains with gene disruption of ald6a, ald6b, ald3, and oahA or over-expression of mct1 gene in the A. niger transgenic strain An3HP9/pyc2 strain; FIG. 2D: in the selected A. niger transgenic strains with additional copies of (3-alanine 3HP pathway. The strains were grown in mRDM medium at 30° C. and 200 rpm for 7 days. The data is the average of three biological replicates.

FIGS. 3A-3D. Multi-omic analyses of 3-HP production in Aspergillus species. FIG. 3A: Shake-flask cultivation of the indicated Aspergillus strains engineered to produce 3-HP. FIG. 3B: Extracellular metabolites detected and quantified by GC-MS at day four. FIG. 3C: Protein and intracellular metabolite levels relative to the un-engineered parent strains at day four. FIG. 3D: Targeted peptide quantification of the heterologous pathway proteins at day four. Multiple boxes for a single protein indicate different targeted peptides. The data represents four biological replicates.

FIGS. 4A-4B. The effects of manganese and nitrogen sources on 3-hydroxypropionic acid production in A. niger transgenic strain An3HP/pyc2 grown at 30° C. and 200 rpm for 7 days. FIG. 4A: the effects of manganese on 3-HP production. FIG. 4B: the effects of different nitrogen sources on 3-HP production. The data is the average of three biological replicates.

FIGS. 5A-5C. Optimization of 3-HP production in RDM with sugars from DDR-EH using A. niger strain An3HP9/pyc2/ald6aA at 200 rpm for 7 days. FIG. 5A: Ability of A. niger to germinate and grow in increasing DDR-EH concentrations from 100 to 250 g/L total sugar. FIG. 5B: 3-HP production in DDR-EH-100 at 30, 34, 37, and 40° C. FIG. 5C: 3-HP production in DDR-EH-100 with limiting (0.2×) and excess (5×) concentrations of phosphate, ammonia, and trace elements. The data is the average of three biological replicates and is corrected for evaporative loss. Asterisks indicate statistically significant differences (p<0.05) from the baseline condition (RDM with 100 g/L total sugars from DDR-EH at 30° C.).

FIG. 6. The impact of nitrogen and trace elements on 3-HP production in RDM with 150 g/L sugars from DDR-EH using A. niger strain An3HP9/pyc2/ald6aA/3HP-6 at 200 rpm and 34° C. for 7 days. The data is the average of three biological replicates and is corrected for evaporative loss. Asterisks indicate statistically significant differences (p<0.05) from the baseline condition (RDM with 150 g/L total sugars from DDR-EH at 34° C.).

FIG. 7. Summary of strain improvements for 3-HP production in A. niger. Yields achieved by over-expression or deletion (indicated by an ‘x’) of genes involved in 3-HP production. All yields achieved are in the 3HP-PYC background except for the β-alanine base and final pathway yields and the PYC yield.

FIGS. 8A-8B. Parent and transgenic strains of A. pseudoterreus and A. niger used herein.

FIG. 9. Copy number estimate of genes in selected strains from whole genome sequencing

FIG. 10. Nutritional conditions for 3-HP production from sugar monomers.

FIGS. 11A-11D. The diagram of the β-alanine 3HP pathway transgene expression cassette with A. pseudoterreus cad1 gene locus targeting and Southern blotting analyses of transgenic A. pseudoterreus. FIG. 11A: The diagram of the β-alanine 3HP pathway (3HP4028, Ap3HP). FIG. 11B: The diagram of the β-alanine 3HP pathway with the pattern of restriction endonuclease BamHI, EcoRV, and HindIII. FIG. 11C: The restriction fragment length polymorphism of BamHI or EcoRV in selected transgenic strains. FIG. 11D: The restriction fragment length polymorphism of HindIII in selected transgenic strains.

FIGS. 12A-12B. Itaconic acid (FIG. 12A) and 3-hydroxypropionic acid (FIG. 12B) production in the selected individual transgenic strains of A. pseudoterreus with over-expression of the β-alanine 3HP pathway transgene expression cassette in the modified RDM medium at 30° C. and 200 rpm for 7 days. The strain 2 and 6 were with the homologous recombination of transgene expression cassette Ap3HP at the cad1 locus (cad1Δ), while strain 4 & 5 with the random integration of the transgene cassette into the chromosomes. All data were average of two biological replates.

FIG. 13. A diagram of a β-alanine 3HP pathway transgene expression cassette with two identical copies of the β-alanine 3HP pathway (3HP4046, 2×3HP). 5′-Cad1, upstream region of cis-aconitate decarboxylase; gpdAp, glyceraldehyde-3-phosphate dehydrogenase promoter from either A. niger or A. nidulans; PAND, T. castaneum aspartate 1-decarboxylase; elf3t, A. niger elongation factor 3 transcriptional terminator; BAPAT, B. cereus β-alanine-pyruvate aminotransferase; eno1p, A. niger enolase promoter; HPDH, E. coli 3-hydroxypropionate dehydrogenase; trpCt, A. nidulans trpC transcriptional terminator; ptrA, pyrithiamine resistance gene of A. oryzae, and 3′-Cad1, downstream region of cis-aconitate decarboxylase.

FIG. 14. A diagram of a β-alanine 3HP pathway transgene expression cassette with E. coli hygromycin B phosphotransferase (hph) marker gene (3HP4070). 5′-C, 326 bp upstream region of cis-aconitate decarboxylase of A. pseudoterreus; gpdAp, glyceraldehyde-3-phosphate dehydrogenase promoter from either A. niger or A. nidulans; PAND, T. castaneum aspartate 1-decarboxylase; elf3t, A. niger elongation factor 3 transcriptional terminator; BAPAT, Bacillus cereus β-alanine-pyruvate aminotransferase; eno1p, A. niger enolase promoter; HPDH, E. coli 3-hydroxypropionate dehydrogenase; trpCt, A. nidulans trpC transcriptional terminator; hph, E. coli hygromycin B phosphotransferase gene marker, and 3′-C, 283 bp upstream region of ptrA gene.

FIG. 15. A diagram of a β-alanine 3HP pathway along with an A. pseudoterreus aat1 transgene overexpression under the control of A. pseudoterreus tef1 gene promoter (3HP4071). 5′-C, 326 bp upstream region of cis-aconitate decarboxylase of A. pseudoterreus; gpdAp, glyceraldehyde-3-phosphate dehydrogenase promoter from either A. niger or A. nidulans; PAND, T. castaneum aspartate 1-decarboxylase; elf3t, A. niger elongation factor 3 transcriptional terminator; BAPAT, B. cereus β-alanine-pyruvate aminotransferase; eno1p, A. niger enolase promoter; HPDH, E. coli 3-hydroxypropionate dehydrogenase; trpCt, A. nidulans trpC transcriptional terminator; hph, E. coli hygromycin B phosphotransferase gene marker, and 3′-C, 283 bp upstream region of ptrA gene; tefip, translation elongation factor 1 (tef1) gene promoter of A. pseudoterreus, and aat1, A. pseudoterreus aspartate aminotransferase gene.

FIGS. 16A-16C. Southern blot analysis confirmed the β-alanine 3HP pathway random integrations into the chromosomes of A. niger in the selected single spore isolates of transgenic strain An3HP5, An3HP9, An3HP10, and An2×3HP1 with multiple copies of chromosomal insertion. FIG. 16A: restriction map of the plasmid DNA fragments containing the β-alanine 3HP pathway used for random integration with restriction endonucleases of BamHI (FIG. 16B, southern blot), or HindIII (FIG. 16C, southern blot). Southern blots show the hybridization pattern of the parent strain (no detection) and single spore isolates of transgenic strain An3HP5, An3HP9, An3HP10, and An2×3HP1. The expected sizes of the hybridizing fragments for the restriction endonuclease digestion were >2.34 kb (BamHI) or >7.43 kb (HindIII). P is probe used for Southern blot analysis.

FIGS. 17A-17E. Diagram of transgene overexpression cassettes of A. niger aspartate aminotransferase (aat1), pyruvate carboxylase (pyc) or their combination, and monocarboxylate transporter (mct1). FIG. 17A: The ble, the bacterial bleomycin resistance gene; tef1p, A. niger tef1 gene promoter; aat1, A. niger aspartate aminotransferase; pgkt, A. niger pgk gene transcriptional terminator (3HP4074); FIG. 17B & FIG. 17C: mbf1P, A. niger mbf1 gene promoter; pyc, A. niger pyc gene without aat1 (3HP4076) & with aat1 (3HP4077); FIG. 17D: the nat1 (Streptomyces noursei nourseothricin N-acetyl transferase optimized for the codon usage of Saccharomyces cerevisiae) selection marker under the control of A. nidulans trpC gene promoter and A. niger trpC gene transcriptional terminator FIG. 17E: the nat selection marker gene; the mct1, A. niger monocarboxylate transporter; the mct1t, A. niger mct1 gene transcriptional terminator.

FIGS. 18A-18C. 3-hydroxypropionic acid production in the selected individual transgenic strains of A. niger with overexpression of cytosolic aspartate aminotransferase (aat, FIG. 18A), pyruvate carboxylase (pyc, FIG. 18B), or aspartate aminotransferase+pyruvate carboxylase (aat-pyc, FIG. 18C) in An3HP9 strain grown in modified RDM medium at 30° C. and 200 rpm for 7 days. All data were average of two biological replates.

FIGS. 19A-19E. A diagram of gene disruption constructs for A. niger oahA, ald6a, ald6b, ald3, and uga2 genes. The hph, E. coli hygromycin B phosphotransferase (hph) gene for selection marker; FIG. 19A: 5′-oahA and 3′-oahA are DNA fragments of upstream and downstream fragments of oahA gene (3HP4102); FIG. 19B: 5′-ald6a and 3′-ald6a are DNA fragments of upstream and downstream of ald6a gene (3HP4103); FIG. 19C: 5′-ald6b and 3′-ald6b are DNA fragments of upstream and downstream of ald6b gene (3HP4104); FIG. 19D: 5′-ald3 and 3′-ald3 are DNA fragments of upstream and downstream of ald3 gene (3HP4108); and FIG. 19E: 5′-uga2 and 3′-uga2 are DNA fragments of upstream and downstream fragments of uga2 gene (3HP4109).

FIGS. 20A-20E. A diagram of the Tet-On/Cre-loxP (3HP4140) system and the new β-alanine 3-HP pathway transgene expression cassette (3HP4145) with loxP-nptII marker gene recycle for A. niger. FIG. 20A: the Tet-On/Cre-loxP system (3HP4140) for marker gene recycle conditionally activated by doxycycline, nat1, S. noursei nourseothricin acetyltransferase (resistance) gene; ubi1S27p, A. niger ubi1S27 promoter; rtTA2A, the reverse tetracycline transactivator; TetO7, tetracycline resistance operon; Pmn, A. nidulans gpdA minimal promoter; Cre, Cre recombinase; ActrpCt, A. carbonarius trpC transcriptional terminator; FIG. 20B: the bacterial neomycin-resistance (nptII) marker gene under the control of A. niger malate dehydrogenase (mdhp) promoter and A. nidulans trpC transcriptional terminator (trpCt) (3HP4134); FIG. 20C: the nptII marker gene cassette with 31 bp loxP fragments fused at 5′- and 3′-end of its marker gene cassette (3HP3136); FIG. 20D: the intermediate transgene expression cassette (3HP4144) contains the ubi4p, A. niger ubi4 gene promoter; PAND, T, castaneum aspartate 1-decarboxylase; elf3t, A. niger elongation factor 3 transcriptional terminator; BAPAT, B. cereus β-alanine-pyruvate aminotransferase; and ubiSp, A. niger ubi1S promoter. FIG. 20E: the final new β-alanine 3-HP pathway (3HP4145) was assembled with the HPDH, K coli 3-hydroxypropionate dehydrogenase under the control of A. niger mbfA promoter (mbfAp) and A. nidulans trpC transcriptional terminator (trpCt).

FIGS. 21A-21B. The effects of culture medium and pH on 3-HP production and spore germination in A. niger strain An3HP9 grown at 30° C. and 200 rpm for 7 days. FIG. 21A: 3-HP and DCW titer and yield and (FIG. 21B) the percentage of spore germination. The data is the average of three biological replicates. Asterisks indicate statistically significant differences (p<0.05) from mRDM, pH 3.4.

FIG. 22. Establishing scale-up parameters in 0.5 L benchtop bioreactors. 3-HP production across a wide range of pH using different bases for pH control (potassium hydroxide (KOH), sodium hydroxide (NaOH) or ammonia (NH3)) at a high aeration rate (2 vvm). 3-HP was produced as a free acid down to ˜pH 2, where the pH drifts to naturally without control. Second, 3-HP production across aeration rates at the best performing pH and base feeding solution (pH 2, NaOH). Highly aerobic conditions were detrimental to production and resulted in by-product secretion, such as citric acid. At low aeration rates, the fungus produces very small amounts of citric acid while maintaining yield. All reactors were inoculated with spores to an initial concentration of 1×10≢spores/mL. Growth occurred at 34° C.

FIGS. 23A-23C. 0.5 L fermentation dynamic profiles and byproduct ranges using optimal pH (2.0, NaOH base addition) and aeration rates (0.2 vvm) at various sugar concentrations. The base medium (control) was RisB with glucose as the sole carbon source (150 g/L, 150 G reactors). The G+X reactors had a 2:1 glucose:xylose ratio (100 g/L glucose, 50 g/L xylose). The 150 G+salt stress reactors added NaCl as an osmolyte to examine production during salt stress. All reactors were inoculated with spores to an initial concentration of 1×10\hat{ }6 spores/mL. Growth occurred at 34° C.

FIG. 24. Composite of 3-HP production rates from multiple bioreactor (0.5 L) runs compared to the average remaining glucose concentration. Across the campaigns, there was a correlation of 3-HP production rates and sugar concentrations, with a notable decrease of production observed at ˜50 g/L. All reactors were inoculated with spores to an initial concentration of 1×10\hat{ }6 spores/mL. Growth occurred at 34° C.

FIG. 25. 3-HP titers under various conditions in shaking flasks (related to FIGS. 26-28C). The control media was RisB medium with glucose at an initial concentration of 150 g/L. In selected conditions, glucose was replaced with a glucose:xylose mixture (2:1 g to g ratio). In other conditions, additives were included in the media at the specified concentrations (2 g/L of Potassium Phosphate (2 g/L P), yeast extract (YE), sodium chloride at various weight percents, lactic acid). In one condition, 5 M NaOH was added at 48 and 96 h to raise the pH to ˜2.5. Cultures were inoculated from spores at a concentration of 1×10\hat{ }6 spores/mL. Growth occurred at 30° C.

FIG. 26. 3-HP rate of production under various conditions in shaking flasks (related to FIG. 25 and FIGS. 27-28C). The control media was RisB medium with glucose at an initial concentration of 150 g/L. In selected conditions, glucose was replaced with a glucose:xylose mixture (2:1 g to g ratio). In other conditions, additives were included in the media at the specified concentrations (2 g/L of Potassium Phosphate (2 g/L P), yeast extract (YE), sodium chloride at various weight percents, lactic acid). In one condition, 5 M NaOH was added at 48 and 96 h to raise the pH to ˜2.5. Cultures were inoculated from spores at a concentration of 1×10\hat{ }6 spores/mL. Growth occurred at 30° C.

FIG. 27. 3-HP yields under various conditions in shaking flasks (related to FIGS. 25, 26, and 28A-28C). The control media was RisB medium with glucose at an initial concentration of 150 g/L. In selected conditions, glucose was replaced with a glucose:xylose mixture (2:1 g to g ratio). In other conditions, additives were included in the media at the specified concentrations (2 g/L of potassium phosphate (2 g/L P), yeast extract (YE), sodium chloride at various weight percents, lactic acid). In one condition, 5 M NaOH was added at 48 and 96 h to raise the pH to ˜2.5. Cultures were inoculated from spores at a concentration of 1×10\hat{ }6 spores/mL. Growth occurred at 30° C.

FIGS. 28A-28C. 3HP titers (FIG. 28A), rates (FIG. 28B), and yields (FIG. 28C) of selected shaking flask cultivations (related to FIGS. 25-27). The control media was RisB medium with glucose at an initial concentration of 150 g/L. In selected conditions, glucose was replaced with a glucose:xylose mixture (2:1 g to g ratio). In other conditions, additives were included in the media at the specified concentrations (2 g/L of potassium phosphate (2 g/L P), yeast extract (YE)). Cultures were inoculated from spores at a concentration of 1×10\hat{ }6 spores/mL. Growth occurred at 30° C.

FIG. 29. 3-HP titers from shaking flask conditions taken at 48 and 96 h (related to FIG. 30). The control medium (RisB+glucose at 100 g/L) was modified by substituting glucose with either maltose, fructose, or maltodextrose (aka maltodextrin). Total sugar concentration was initially at a concentration of 100 g/L. Additives of Peptone, Tryptone, Corn Steep Liquor or Soy Protein were added at a weight percent of 0.2% to media conditions. Ammonia Sulfate (AS) was removed from the based medium of certain conditions with the specified additives. Additional phosphorus (2×P) was also added in select conditions. Growth occurred at 30° C.

FIG. 30. Sugar uptake rate (Carbon mmol consumed per h) from shaking flask conditions taken at 48 and 96 h (related to FIG. 29). The control medium (RisB+glucose) was modified by substituting glucose with either maltose, fructose, or maltodextrose (aka maltodextrin). Total sugar concentration was at an initial concentration of 100 g/L. Additives of Peptone, Tryptone, Corn Steep Liquor or Soy Protein were added at a weight percent of 0.2% to media conditions. Ammonia Sulfate (AS) was removed from the based medium of certain conditions with the specified additives. Additional phosphorus (2×P) was also added in select conditions. Maltodextrin was not measurable via HPLC. Growth occurred at 30° C.

FIGS. 31A-31C. 3-HP production in shaking flask (related to FIGS. 32-24) at day 1 and day 2. The control media is RisB with glucose, 20×Trace elements (20×TE), and 1× Potassium Phosphate (1×-P). The phosphate level was varied and from 1× to 10×. Samples were taken across the shaking flask cultivations. Cultivations occurred at 35° C. and were inoculated at 10% vol/vol from a 10% seed culture. Samples were measure at Day 1 (FIG. 31A), Day 2 (FIG. 31B), and converted sugars on Day 2 (FIG. 31° C.).

FIGS. 32A-32B. 3-HP rates and yields in shaking flask (related to FIGS. 31A-31C and 33A-34). The control media is RisB with Glucose and 20×Trace elements (20×TE) and 1× Potassium Phosphate (1×-P). The phosphate level was varied and from 1× to 10×. Samples were taken across the shaking flask cultivations at Day 2 (FIG. 32A) and Day 3 (FIG. 32B). Cultivations occurred at 35° C. and were inoculated at 10% vol/vol from a 10% seed culture.

FIGS. 33A-33B. 3-HP titers on maltose and maltodextrin (day 2) in shaking flask (related to FIGS. 31A-32B and 34). The control media is RisB with Glucose and 20×Trace elements (20×TE) and 1× Potassium Phosphate (1×-P). The phosphate level was varied and from 1× to 10×. Samples were taken across the shaking flask cultivations for maltose at Day 2 (FIG. 33A) and maltodextrin at Day 2 (FIG. 33B). Cultivations occurred at 35° C. and were inoculated at 10% vol/vol from a 10% seed culture.

FIG. 34. Dry cell weigh measurements from 3-HP production in shaking flask (related to FIGS. 31A-33B). The control media is RisB with Glucose and 20×Trace elements (20×TE) and 1× Potassium Phosphate (1×-P). The phosphate level was varied and from 1× to 10×. Samples were taken across the shaking flask cultivations. Cultivations occurred at 35° C. and were inoculated at 10% vol/vol from a 10% seed culture.

### SEQUENCE LISTING

The nucleic and amino acid sequences listed in the accompanying sequence listing are shown using standard letter abbreviations for nucleotide bases, and single letter code for amino acids, as defined in 37 C.F.R. 1.822. Only one strand of each nucleic acid sequence is shown, but the complementary strand is understood as included by any reference to the displayed strand. In the accompanying sequence listing:

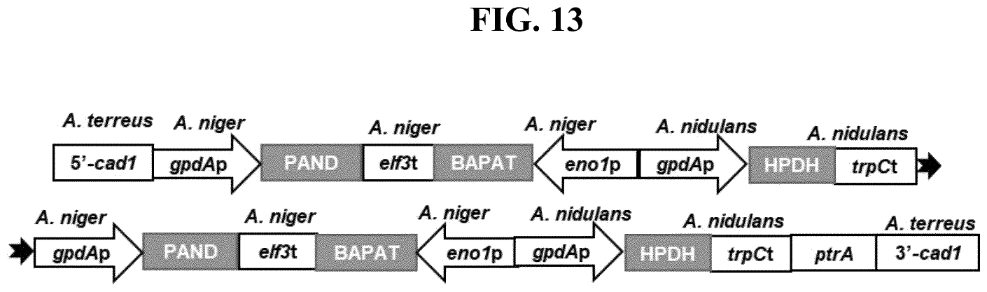

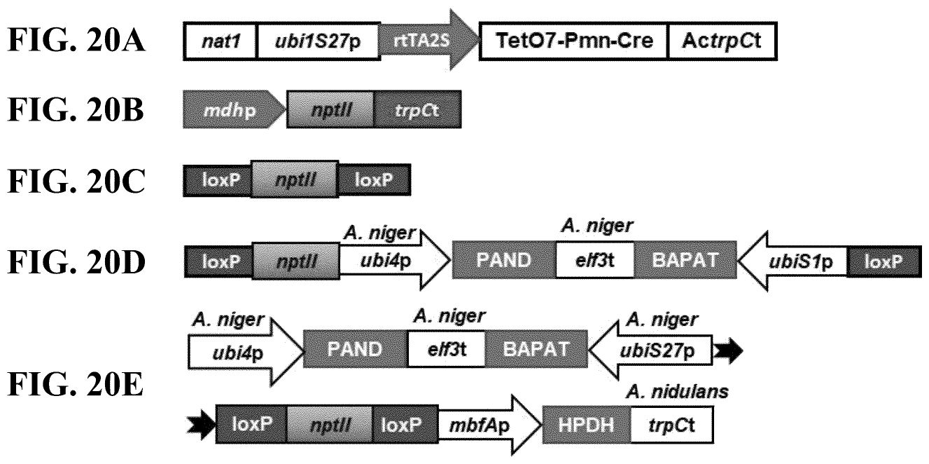

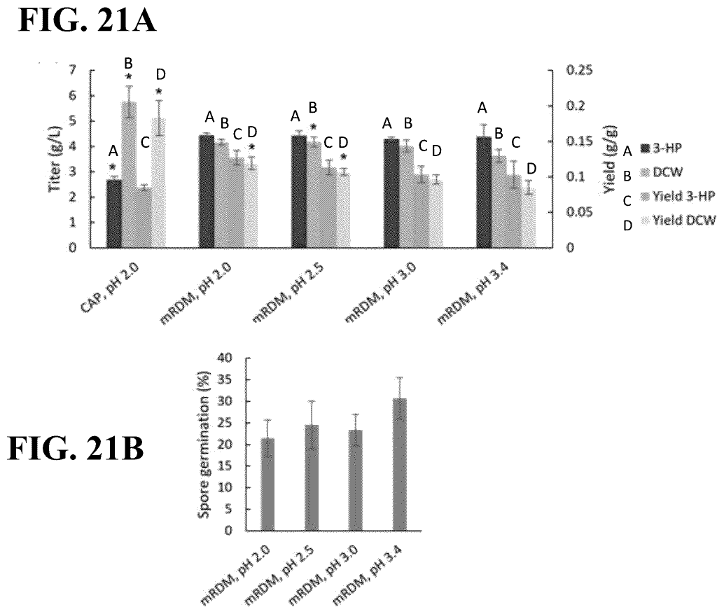

SEQ ID NO: 1 is an exemplary aspartate
1-decarboxylase (PAND) amino acid sequence
(Tribolium castaneum).
MPATGEDQDLVQDLIEEPATFSDAVLSSDEELFHQKCPKP
APIYSPVSKPVSFESLPNRRLHEEFLRSSVDVLLQEAVFE
GTNRKNRVLQWREPEELRRLMDFGVRSAPSTHEELLEVLK
KVVTYSVKTGHPYFVNQLFSAVDPYGLVAQWATDALNPSV
YTYEVSPVFVLMEEVVLREMRAIVGFEGGKGDGIFCPGGS
IANGYAISCARYRFMPDIKKKGLHSLPRLVLFTSEDAHYS
IKKLASFQGIGTDNVYLIRTDARGRMDVSHLVEEIERSLR
EGAAPFMVSATAGTTVIGAFDPIEKIADVCQKYKLWLHVD
AAWGGGALVSAKHRHLLKGIERADSVTWNPHKLLTAPQQC
STLLLRHEGVLAEAHSTNAAYLFQKDKFYDTKYDTGDKHI
QCGRRADVLKFWFMWKAKGTSGLEKHVDKVFENARFFTDC
IKNREGFEMVIAEPEYTNICFWYVPKSLRGRKDEADYKDK
LHKVAPRIKERMMKEGSMMVTYQAQKGHPNFFRIVFQNSG
LDKADMVHLVEEIERLGSDL
SEQ ID NO: 2 is an exemplary β-alanine-
pyruvate aminotransferase (BAPAT) amino
acid sequence (Bacillus cereus).
MELMIVQVTEQTQSLKKTDEKYLWHAMRGAAPSPTNLIIT
KAEGAWVTDIDGNRYLDGMSGLWCVNVGYGRKELARAAFE
QLEEMPYFPLTQSHVPAIKLAEKLNEWLDDEYVIFFSNSG
SEANETAFKIARQYHQQKGDHGRYKFISRYRAYHGNSMGA
LAATGQAQRKYKYEPLGQGFLHVAPPDTYRNPEDVHTLAS
AEEIDRVMTWELSQTVAGVIMEPIITGGGILMPPDGYMEK
VKEICEKHGALLICDEVICGFGRTGKPFGFMNYGVKPDII
TMAKGITSAYLPLSATAVRREVYEAFVGSDDYDRFRHVNT
FGGNPAACALALKNLEIMENEKLIERSKELGERLLYELED
VKEHPNVGDVRGKGLLLGIELVEDKQTKEPASIEKMNKVI
NACKEKGLIIGKNGDTVAGYNNILOLAPPLSITEEDFTFI
VKTMKECLAQL
SEQ ID NO: 3 is an exemplary
3-hydroxypropionate dehydrogenase
(HPDH) amino acid sequence (E. coli).
MIVLVTGATAGFGECITRRFIQQGHKVIATGRRQERLQEL
KDELGDNLYIAQLDVRNRAAIEEMLASLPAEWCNIDILVN
NAGLALGMEPAHKASVEDWETMIDTNNKGLVYMTRAVLPG
MVERNHGHIINIGSTAGSWPYAGGNVYGATKAFVRQFSLN
LRTDLHGTAVRVTDIEPGLVGGTEFSNVRFKGDDGKAEKT
YQNTVALTPEDVSEAVWWVSTLPAHVNINTLEMMPVTQSY
AGLNVHRQ
SEQ ID NO: 4 is an exemplary nucleic acid
sequence encoding a 3-HP β-alanine pathway
expression cassette (including aspartate
1-decarboxylase, β-alanine-pyruvate
aminotransferase, and 3-hydroxypropionate
dehydrogenase; see, FIG. 13).
GGTTGTAGCAGCGTAAACACATGGATAGTTAAATAATCGG
ATGTACACCCACTGTTGGAAATGACGGGGGCCTACAACAC
GAGATTATCTGATCCAATTTCTGTTCGTTGGCATTCTATC
ATTCGCAGCGAAAATTGTCCTATTAAATTGACCATGACCA
AACAATCTGCGGACAGCAACGCAAAGTCAGGAGTTACGTC
CGAAATATGTCATTGGGCATCCAACCTGGCCACTGACGAC
ATCCCTTCGGACGTATTAGAAAGAGCAAAATACCTTATTC
TCGACGGTATTGCATGTGCCTGGGTTGGTGCAAGAGTGCC
TTGGTCAGAGAAGTATGTTCAGGCAACGATGAGCTTTGAG
CCGCCGGGGGCCTGCAGGGTGATTGGATATGGACAGGTAA
ATTTTATTCACTCTAGACGGTCCACAAAGTATACTGACGA
TCCTTCGTATAGAAACTGGGGCCTGTTGCAGCAGCCATGA
CCAATTCCGCTTTCATACAGGCTACGGAGCTTGACGACTA
CCACAGCGAAGCCCCCCTACACTCTGCAAGCATTGTCCTT
CCTGCGGTCTTTGCAGCAAGTGAGGTCTTAGCCGAGCAGG
GCAAAACAATTTCCGGTATAGATGTTATTCTAGCCGCCAT
TGTGGGGTTTGAATCTGGCCCACGGATCGGCAAAGCAATC
TACGGATCGGACCTCTTGAACAACGGCTGGCATTGTGGAG
CTGTGTATGGCGCTCCAGCCGGTGCGCTGGCCACAGGAAA
GCTCCTCGGTCTAACTCCAGACTCCATGGAAGATGCTCTC
GGAATTGCGTGCACGCAAGCCTGTGGTTTAATGTCGGCGC
AATACGGAGGCATGGTAAAGCGTGTGCAACACGGATTCGC
AGCGCGTAATGGTCTTCTTGGGGGACTGTTGGCCCATGGT
GGGTACGAGGCAATGAAAGGTGTCCTGGAGAGATCTTACG
GCGGTTTCCTCAAGATGTTCACCAAGGCTACTATGAAAGA
CCGCGATGGGCCGATAGTAGTAGTTACTTCCATTACATCA
TCTCATCCGCCCGGTTCCTCGCCTCCGCGGCAGTCTACGG
GTAGGATCGTAGCAAAAACCCGGGGGATAGACCCGTCGTC
CCGAGCTGGAGTTCCGTATAACCTAGGTAGAAGGTATCAA
TTGAACCCGAACAACTGGCAAAACATTCTCGAGATCGTAG
GAGTGAGTACCCGGCGTGATGGAGGGGGAGCACGCTCATT
GGTCCGTACGGCAGCTGCCGAGGGGGAGCAGGAGATCCAA
ATATCGTGAGTCTCCTGCTTTGCCCGGTGTATGAAACCGG
AAAGGACTGCTGGGGAACTGGGGAGCGGCGCAAGCCGGGA
ATCCCAGCTGACAATTGACCCATCCTCATGCCGTGGCAGA
GCTTGAGGTAGCTTTTGCCCCGTCTGTCTCCCCGGTGTGC
GCATTCGACTGGGCGCGGCATCTGTGCCTCCTCCAGGAGC
GGAGGACCCAGTAGTAAGTAGGCCTGACCTGGTCGTTGCG
TCAGTCCAGAGGTTCCCTCCCCTACCCTTTTTCTACTTCC
CCTCCCCCGCCGCTCAACTTTTCTTTCCCTTTTACTTTCT
CTCTCTCTTCCTCTTCATCCATCCTCTCTTCATCACTTCC
CTCTTCCCTTCATCCAATTCATCTTCCAAGTGAGTCTTCC
TCCCCATCTGTCCCTCCATCTTTCCCATCATCATCTCCCT
TCCCAGCTCCTCCCCTCCTCTCGTCTCCTCACGAAGCTTG
ACTAACCATTACCCCGCCACATAGACACATCTAAACAATG
CCCGCCACCGGCGAGGACCAGGACCTGGTGCAGGACCTGA
TCGAGGAACCCGCCACCTTCTCCGACGCCGTCCTGTCCTC
CGACGAGGAACTGTTCCACCAGAAGTGCCCCAAGCCGGCT
CCGATCTACAGCCCCGTCAGCAAGCCCGTCAGCTTCGAGT
CCCTGCCGAACCGCCGCCTGCACGAAGAGTTCCTCCGCTC
CTCCGTCGACGTCCTGCTGCAAGAGGCCGTGTTCGAGGGC
ACCAACCGCAAGAACCGCGTCCTGCAGTGGCGCGAGCCCG
AAGAACTGCGCCGCCTGATGGACTTCGGCGTCCGCAGCGC
CCCGTCCACGCATGAGGAACTGCTCGAGGTCCTGAAGAAG
GTCGTCACCTACTCCGTCAAGACCGGCCATCCGTACTTCG
TCAACCAGCTGTTCTCCGCCGTCGATCCCTACGGCCTGGT
CGCCCAGTGGGCCACCGACGCGCTGAACCCCTCCGTCTAC
ACCTACGAGGTCAGCCCCGTGTTCGTCCTGATGGAAGAGG
TCGTCCTGCGCGAGATGCGCGCCATCGTCGGCTTCGAAGG
CGGCAAAGGCGACGGCATCTTCTGCCCTGGCGGCTCGATC
GCCAACGGCTACGCCATCAGCTGCGCCCGCTACCGCTTCA
TGCCCGACATCAAGAAGAAGGGCCTGCACTCCCTGCCGCG
CCTGGTCCTGTTCACCTCCGAGGACGCCCACTACTCGATC
AAGAAGCTGGCCTCGTTCCAAGGCATCGGCACCGACAACG
TCTACCTGATCCGCACCGACGCTCGCGGTCGCATGGACGT
CAGCCACCTGGTCGAAGAGATCGAGCGCTCCCTCCGCGAG
GGCGCTGCCCCGTTCATGGTCAGCGCCACCGCCGGCACCA
CCGTCATCGGCGCCTTCGATCCCATCGAGAAGATCGCCGA
CGTCTGCCAGAAGTACAAGCTCTGGCTGCACGTCGACGCC
GCCTGGGGCGGAGGCGCTCTGGTGTCCGCCAAGCACCGCC
ATCTGCTGAAGGGCATCGAGCGCGCCGACTCCGTCACCTG
GAATCCCCACAAGCTGCTGACCGCTCCGCAGCAGTGCAGC
ACCCTGCTGCTGCGCCACGAGGGCGTCCTGGCCGAGGCGC
ACTCCACCAACGCCGCCTACCTGTTCCAGAAGGACAAGTT
CTACGACACCAAGTACGACACCGGCGACAAGCACATCCAG
TGCGGCCGTCGCGCCGACGTGCTGAAGTTCTGGTTCATGT
GGAAGGCCAAGGGCACCTCCGGCCTCGAGAAGCACGTGGA
CAAGGTGTTCGAGAACGCCCGCTTCTTCACCGACTGCATC
AAGAACCGTGAGGGCTTCGAGATGGTGATCGCCGAGCCTG
AGTACACCAACATCTGTTTCTGGTACGTCCCCAAGAGCCT
GCGCGGACGCAAGGACGAGGCCGACTACAAGGACAAGCTG
CACAAGGTCGCCCCTCGCATCAAAGAACGCATGATGAAGG
AAGGCTCCATGATGGTCACCTACCAGGCGCAGAAGGGCCA
TCCGAATTTCTTCCGCATCGTCTTTCAGAACTCCGGCCTG
GACAAGGCCGACATGGTCCATCTGGTCGAGGAAATCGAAC
GCCTGGGCTCCGACCTCTGATGGGTTGGATGACGATGACT
TCATGTGATTTTGTTATTTAGAATATTTTATATTTCCTTT
TCTTCTTCTCACCACCGATCCCCTTAACACTCTTGCTTCA
TTTGCTTCAGATTTCTCGGTTTCTTCTTTTTTCTTCTCCC
CAGTTATCCACTATATCTTTGCTAGACCGGCCTGCGCCCT
GGCATGCATCATAAAATCATGTCCGTTGGTCATCATCTGT
TTTGTATATCCGTCATATAAAGTATTCTTTTATTCCCTCC
CCCCTCGGTCGTCTTTCGCTGTCCCGCTTCCTACCTCCGG
TTTATAGAGCATGGTTCATCTCTTCCGTACATTTCCGTTG
GTACTAGCATTTATGTCTTCAGCTAGTATAGAAGCTGCCG
CAGTTGTTCGCTTACTACCTGCCTAAGTCCTTAACTTTTT
AAAGTGTTTAACCTATACGTAGTGTTAAACGAGTACTGGG
AGGTGGTGAGGTAGAAAATGTTCTGCACGGGCAGTGGGTA
TTTGGTAGTGTGTAAGGCGGTTATTTATCAGGCTGACGCT
AAAGACTTCTATGGGAGCAGTATGGGATCGCGGCTCATAG
AAGTACACAAAATCTAAGAGTCGTTTGATAATTAATTGAT
TCCCGGCAGGGTCTTCTTGGGATTGAGAGAACTGGTTACT
TTGATTTGAGATATTGTAAAGCTTAAGGCTCTTAACACGT
ACGAGCGAAACAGCAGGGGGGAAATCGGGAAAAGGGGCGT
GGGGTGAATAAAAAAGTTGAAATAAGACACTGTATCTTGC
TGGGGGTGAATAAAGAGAGAATAAAAGAGAGGTAAATTCC
ACTCAGCCCCTTTTCTTCGCTCTCCAAACATCAAACTCCG
CCGGCCGACCCACAGGATCCCGAACAAGTGGAAGATATGT
GCCGGTCCAGACCCTTCGCACAGCTAAAAGCAGACCTTCA
TAAGCGTTTCCGGGTAGTATTCGCACACCTGAACTGGCAC
GTCGGGGACACAACTGTTTTTGATACACAAGAACACACAC
CACCCATCTAGGACTCAGAGCTGGGCCAGACATTCCTTCA
TAGTCTTGACGATGAAGGTGAAGTCCTCTTCGGTGATGGA
CAGCGGAGGGGCGAGCTGCAGGATGTTGTTGTAGCCGGCC
ACGGTGTCGCCGTTCTTGCCGATGATCAGACCCTTCTCTT
TGCAGGCGTTGATGACCTTGTTCATCTTTTCGATGGAGGC
GGGCTCTTTGGTCTGCTTATCCTCGACGAGTTCGATACCC
AGCAGGAGGCCCTTGCCGCGGACGTCCCCGACGTTGGGGT
GCTCTTTGACGTCCTCCAACTCGTACAGCAGGCGCTCGCC
CAGTTCTTTGGACCGCTCGATGAGCTTCTCGTTTTCCATG
ATCTCGAGGTTCTTCAGGGCCAGCGCGCAGGCGGCAGGGT
TGCCGCCGAAGGTGTTGACATGGCGGAAGCGGTCGTAGTC
GTCGGAGCCGACGAAGGCCTCGTAGACCTCGCGGCGGACC
GCGGTGGCAGACAGCGGCAGGTAGGCCGAGGTGATACCCT
TGGCCATGGTGATAATGTCGGGCTTGACGCCGTAGTTCAT
GAAGCCGAAGGGCTTGCCGGTGCGACCGAAGCCGCAGATG
ACCTCGTCGCAGATCAGCAGGGCGCCGTGCTTTTCGCAGA
TCTCTTTGACCTTTTCCATGTAGCCGTCCGGCGGCATCAG
GATGCCACCACCGGTGATGATGGGTTCCATGATGACGCCG
GCGACGGTCTGGGACAGCTCCCAGGTCATGACGCGGTCGA
TTTCTTCGGCGGAGGCCAGGGTGTGCACGTCCTCGGGGTT
GCGATAGGTGTCCGGAGGGGCCACGTGCAGGAAGCCCTGA
CCGAGGGGCTCGTACTTGTACTTGCGCTGGGCCTGACCGG
TCGCGGCCAGGGCACCCATGGAGTTGCCGTGGTAGGCGCG
GTAGCGAGAGATGAACTTGTAGCGGCCGTGGTCACCCTTC
TGCTGGTGGTACTGGCGGGCGATCTTGAAGGCGGTTTCGT
TGGCCTCCGAGCCGGAGTTGGAGAAGAAGATGACGTACTC
GTCGTCCAGCCACTCGTTCAGCTTCTCGGCCAGCTTGATG
GCGGGGACGTGCGACTGCGTCAGCGGGAAGTACGGCATCT
CTTCCAGCTGCTCGAAGGCAGCGCGAGCCAGCTCTTTGCG
GCCGTAGCCGACGTTGACGCACCACAGGCCGGACATGCCG
TCCAGGTAGCGGTTGCCGTCGATGTCGGTGACCCACGCGC
CTTCGGCCTTGGTGATGATCAGGTTGGTCGGACTCGGAGC
GGCACCGCGCATGGCGTGCCACAGGTACTTCTCGTCGGTT
TTCTTCAGGCTCTGGGTCTGCTCGGTGACCTGGACGATCA
TCAGTTCCATGTTGATGGACTGGAGGGGGATGAGTTATGG
ATCAGTGAAACTGGGAGAAAACAAAGATGGCAAAGGGAGA
ACATGGCCCAGATATAGGAAAAAACGGAGGAGGCAAAAAT
GTAAGCGCTCCGGACTTGCTGTTTCGGTGTGCACTAGCAG
CAGCGGGGGGGAAGGTGGTGAGTGTTCACCGAGGACCCAA
AAAGAATGAGCGGATGGCGGATGAGTGACGGAGAAGGGAA
GGACGGGGGGGGAATTAGAGGTGGAGAGGTCCGATCCATC
AAATAGACCAGGCTCGGCACAGCCAAGTTTCCCAAATGAT
CAACTAATCAATGGGACTTGGTGCTAAATCCGGAGATGCC
AGATCATTGATAGACAGACAGGATGGAGTGATGGCATATA
GACAGGAGGATGGATGGATGGATAGATGGAGGGGTCAAGC
ACAACATGGTGGGATGATGGCGGGGTCATGACTAGCAGCT
AAGAGGAAGAAGAGGAGGATGAAATGGACAGAGAAAGATG
GGAGGGGTGATAAAATGAGTATATGGGACAAGTCATACTT
ACAGGACCTTGAAGATGGTGGTTGTACTATCTAAGAAAGG
CTTTTTTTGAGAGTACTCTTAACACAAGAGGAGGAGGGAG
GAGGGGGAAGTAGTAGATAAATAATAAACACGACCACAGA
CTTGCTACAGGCTACTTCTTGTAAGCTCGAGTTTCTGTAC
AGTGACCGGTGACTCTTTCTGGCATGCGGAGAGACGGACG
GACGCAGAGAGAAGGGCTGAGTAATAAGCCACTGGCCAGA
CAGCTCTGGCGGCTCTGAGGTGCAGTGGATGATTATTAAT
CCGGGACCGGCCGCCCCTCCGCCCCGAAGTGGAAAGGCTG
GTGTGCCCCTCGTTGACCAAGAATCTATTGCATCATCGGA
GAATATGGAGCTTCATCGAATCACCGGCAGTAAGCGAAGG
AGAATGTGAAGCCAGGGGTGTATAGCCGTCGGCGAAATAG
CATGCCATTAACCTAGGTACAGAAGTCCAATTGCTTCCGA
TCTGGTAAAAGATTCACGAGATAGTACCTTCTCCGAAGTA
GGTAGAGCGAGTACCCGGCGCGTAAGCTCCCTAATTGGCC
CATCCGGCATCTGTAGGGCGTCCAAATATCGTGCCTCTCC
TGCTTTGCCCGGTGTATGAAACCGGAAAGGCCGCTCAGGA
GCTGGCCAGCGGCGCAGACCGGGAACACAAGCTGGCAGTC
GACCCATCCGGTGCTCTGCACTCGACCTGCTGAGGTCCCT
CAGTCCCTGGTAGGCAGCTTTGCCCCGTCTGTCCGCCCGG
TGTGTCGGCGGGGTTGACAAGGTCGTTGCGTCAGTCCAAC
ATTTGTTGCCATATTTTCCTGCTCTCCCCACCAGCTGCTC
TTTTCTTTTCTCTTTCTTTTCCCATCTTCAGTATATTCAT
CTTCCCATCCAAGAACCTTTATTTCCCCTAAGTAAGTACT
TTGCTACATCCATACTCCATCCTTCCCATCCCTTATTCCT
TTGAACCTTTCAGTTCGAGCTTTCCCACTTCATCGCAGCT
TGACTAACAGCTACCCCGCTTGAGCAGACATCACCATGAT
CGTGCTGGTCACGGGCGCGACCGCCGGTTTCGGCGAGTGC
ATCACCCGCCGCTTCATCCAGCAGGGCCACAAGGTGATCG
CTACCGGACGCCGCCAAGAGCGCCTCCAAGAGCTGAAGGA
TGAGCTGGGCGACAACCTGTACATTGCCCAGCTGGACGTG
CGCAACCGGGCTGCCATCGAAGAAATGCTCGCCTCGCTGC
CCGCCGAGTGGTGCAACATCGACATCCTGGTCAACAACGC
CGGTCTGGCCCTCGGCATGGAACCGGCGCACAAGGCCAGC
GTCGAGGACTGGGAAACCATGATCGACACCAACAACAAGG
GACTCGTCTACATGACCCGCGCTGTGCTGCCCGGCATGGT
CGAGCGCAACCACGGCCACATCATCAACATCGGCTCCACC
GCTGGCAGCTGGCCCTACGCTGGCGGCAACGTCTATGGCG
CGACCAAGGCGTTCGTCCGCCAGTTCTCCCTGAACCTGCG
CACCGACCTGCACGGCACCGCCGTCCGCGTGACCGACATT
GAGCCCGGTCTGGTCGGCGGCACCGAGTTCAGCAACGTCC
GCTTCAAGGGCGACGACGGCAAGGCCGAGAAAACCTACCA
GAACACCGTCGCTCTGACCCCTGAGGATGTCAGCGAGGCC
GTCTGGTGGGTCAGCACTCTGCCCGCGCACGTCAACATCA
ACACCCTCGAGATGATGCCCGTCACGCAGTCCTACGCCGG
CCTGAACGTCCACCGCCAATAGGACCGATGGCTGTGTAGA
AGTACTCGCCGATAGTGGAAACCGACGCCCCAGCACTCGT
CCGAGGGCAAAGGAATAGAGTAGATGCCGACCGGGATCCA
CTTAACGTTACTGAAATCATCAAACAGCTTGACGAATCTG
GATATAAGATCGTTGGTGTCGATGTCAGCTCCGGAGTTGA
GACAAATGGTGTTCAGGATCTCGATAAGATACGTTCATTT
GTCCAAGCAGCAAAGAGTGCCTTCTAGTGATTTAATAGCT
CCATGTCAACAAGAATAAAACGCGTTTCGGGTTTACCTCT
TCCAGATACAGCTCATCTGCAATGCATTAATGCATTGGAC
CTCGCAACCCTAGTACGCCCTTCAGGCTCCGGCGAAGCAG
AAGAATAGCTTAGCAGAGTCTATTTTCATTTTCGGGAGAC
GAGATCAAGCAGATCAACGGTCGTCAAGAGACCTACGAGA
CTGAGGAATCCGCTCTCTGACAGACGGGCAATTGATTACG
GGATCCCATTGGTAACGAAATGTAAAAGCTAGGAGATCGT
CCGCCGATGTCAGGATGATTTCACTTGTTTCTTGTCCGGC
TCACCGGTCAAAGCTAAAGAGGAGCAAAAGGAACGGATAG
AATCGGGTGCCGCTGATCTATACGGTATAGTGCCCTTATC
ACGTTGACTCAACCCATGCTATTTAACTCAACCCCTCCTT
CTGAACCCCACCATCTTCTTCCTTTTCCTCTCATCCCACA
CAATTCTCTATCTCAGATTTGAATTCCAAAAGTCCTCGGA
CGAAACTGAACAAGTCTTCCTCCCTTCGATAAACCTTTGG
TGATTGGAATAACTGACCATCTTCTATAGTTCCCAAACCA
ACCGACAATGTAAATACACTCCTCGATTAGCCCTCTAGAG
GGCATACGATGGAAGTCATGGAATACTTTTGGCTGGACTC
TCACAATGATCAAGGTATCTTAGGTAACGTCTTTGGCGTG
GGCCGGTGTTCGTTCCCAGTCATCGATGCATTCACATGCC
CTCCCTAAGCTGGGCCCTAGACTCTAGGATCCTAGTCTAG
AAGGACATGGCATCGATGGACTGGGTTCGTTCTGAGATTA
TACGGCTAAAACTTGATCTGGATAATACCAGCGAAAAGGG
TCATGCCTTCTCTCGTTCTTCCTGTTGATGGAATGGCTAA
CAGATGATAGTCATTGCAACTTGAAACATGTCTCCTCCAG
CTGCCATCTACGAACCCACTGTGGCCGCTACCGGCCTCAA
GGGTAAGGTCGTGGTTTCTGAGACCGTCCCCGTTGAGGGA
GCTTCTCAGACCAAGCTGTTGGACCATTTCGGTGGCAAGT
GGGACGAGTTCAAGTTCGCCCCTATCCGCGAAAGCCAGGT
CTCTCGTGCCATGACCAGACGTTACTTTGAGGACCTGGAC
AAGTACGCTGAAAGTGACGTTGTCATTGTTGGTGCTGGTT
CCTGCGGTCTGAGCACTGCGTACGTCTTGGCCAAGGCTCG
TCCGGACCTGAAGATTGCTATCGTCGAGGCCAGCGTCTCT
CCTGGTCAGTAGTCCATGATGGATTGCCTTGCACTCAGCT
TTCCGGAACTAACGTGCAATAGGTGGCGGTGCCTGGTTGG
GTGGCCAACTCTTTTCTGCTATGGTCATGCGCCGTCCCGC
GGAAGTCTTCCTGAACGAGCTGGGTGTTCCTTACGAAGAG
GACGCAAACCCCAACTACGTTGTCGTCAAGCACGCCTCCC
TGTTTACCTCGACACTCATGTCGAAGGTTCTCTCCTTCCC
CAATGTCAAGCTCTTCAATGCTACCGCTGTTGAGGACTTG
ATCACCCGTCCGACCGAGAACGGCAACCCCCAGATTGCTG
GTGTTGTCGTCAACTGGACGCTGGTCACCCTTCACCACGA
TGATCACTCCTGCATGGACCCCAACACTATCAACGCTCCT
GTCATCATCAGTACCACTGGTCACGATGGGCCATTCGGCG
CCTTCTGTGCGAAGCGCTTGGTGTCCATGGGCAGCGTCGA
CAAGCTAGGTGGCATGCGTGGTCTCGACATGAGCTCGGCC
GAGGATGCCATCGTCAAGAACACCCGCGAGGTTACTAAGG
GCTTGATAATCGGCGGTATGGAGCTGTCTGAAATTGATGG
CTTTAACCGCATGGGCCCTACCTTCGGTGCCATGGTTCTC
AGTGGTGTCAAGGCTGCCGAGGAGGCATTGAAGGTGTTCG
ACGAGCGTCAGCGCGAGTGTGCTGAGTAAATGACTCACTA
CCCGAATGGGTTCAGTGCATGAACCGGATTTGTCTTACGG
TCTTTGACGATAGGGGAATGATGATTATGTGATAGTTCTG
AGATTTGAATGAACTCGTTAGCTCGTAATCCACATGCATA
TGTAAATGGCTGTGTCCCGTATGTAACGGTGGGGCATTCT
AGAATAATTATGTGTAACAAGAAAGACAGTATAATACAAA
CAAAGATGCAAGAGCGGCTCCTCAGCAACATTCGCCATGT
TCATGTACAGCTTTCAACGGCCTCGAACAGTCACTGTGGA
TGGATACCAGAGGAGAGACCCATCAGTTCAATCGCAGGGC
AGATGAGTGTCGCATACATTCTCGCCGTCCAGCTGGTCGA
CCAGCAATGTCTTTTGTCCCAGTTTTCTGAGTTTGATGAC
AACCTGGAGAGGCCAGAAGTTTGGGATCTGGCCAGGAAGG
TTACTTCATCTCAAAGCGAAGAGTTTGATCAAGACGGCAA
CTGTCTCAGTGCGGGTCGCGTGAGGATTGAGTTCAACGAT
GGTTCTTCTATTACGGAAAGTGTCGAGAAGCCTCTTGGTG
TCAAAGAGCCCATGCCAAACGAACGGATTCTCCACAAATA
CCGAACCCTTGCTGGTAGCGTGACGGACGAATCCCGGGTG
AAAGAGATTGAGGATCTTGTCCTCGGCCTGGACAGGCTCA
CCGACATTAGCCCATTGCTGGAGCTGCTGAATTGCCCCGT
GAAATCGCCACTGGTATAAATGGGAAGCGATATGGAAACA
TTTCATGTCACGGGCACAAATTCTAGGTCATATCGTACCT
GGATGGTGAAACCACCAGCGGTTTAGCAGATAGAAGATAG
ACTCCTTCTGCTCTGCGTTGCGTCTTGAATTTAGTTCGTT
CACTGGCTTAAGAACTTAGAATGCAATACAGTCTCTCTTA
TTTCTTATTAAAATCACGTATTCCCACATTCGGCGACTGG
AGGATACGAAAGCAGTGTTGGTGGTGCTCCCCGTAATGGA
TATGATTTTGCTGACTGGACTATTCTATGACCATTCCCTC
CAACGGAGATCCTTTCTCGACACTTTAGATGTTGACGCTG
TCTGGAGGAACTACTTTTGCGCTGCAAAGACTATGAGCAG
TGGAGCTG
SEQ ID NO: 5 is an exemplary pyruvate
carboxylase (pyc) amino acid sequence from
A. niger.
MAAPRQPEEAVDDTEFIDDHHDQHRDSVHTRLRANSAIMQ
FQKILVANRGEIPIRIFRTAHELSLQTVAVYSHEDRLSMH
RQKADEAYMIGKRGQYTPVGAYLAIDEIVKIALEHGVHLI
HPGYGFLSENAEFARKVEQSGMVFVGPTPQTIESLGDKVS
ARQLAIRCDVPVVPGTPGPVERYEEVKAFTDTYGFPIIIK
AAFGGGGRGMRVVRDQAELRDSFERATSEARSAFGNGTVF
VERFLDRPKHIEVQLLGDNHGNVVHLFERDCSVQRRHQKV
VEIAPAKDLPADVRDRILADAVKLAKSVNYRNAGTAEFLV
DQQNRYYFIEINPRIQVEHTITEEITGIDIVAAQIQIAAG
ATLEQLGLTQDRISTRGFAIQCRITTEDPSKGFSPDTGKI
EVYRSAGGNGVRLDGGNGFAGAIITPHYDSMLVKCTCRGS
TYEIARRKVVRALVEFRIRGVKTNIPFLTSLLSHPVFVDG
TCWTTFIDDTPELFALVGSQNRAQKLLAYLGDVAVNGSSI
KGQIGEPKLKGDIIKPVLHDAAGKPLDVSVPATKGWKQIL
DSEGPEAFARAVRANKGCLIMDTTWRDAHQSLLATRVRTI
DLLNIAHETSHALANAYSLECWGGATFDVAMRFLYEDPWD
RLRKLRKAVPNIPFQMLLRGANGVAYSSLPDNAIYHFCKQ
AKKCGVDIFRVFDALNDVDQLEVGIKAVHAAEGVVEATIC
YSGDMLNPSKKYNLPYYLDLVDKVVQFKPHVLGIKDMAGV
LKPQAARLLIGSIRERYPDLPIHVHTHDSAGTGVASMIAC
AQAGADAVDAATDSLSGMTSQPSIGAILASLEGTEHDPGL
NSAQVRALDTYWAQLRLLYSPFEAGLTGPDPEVYEHEIPG
GQLTNLIFQASQLGLGQQWAETKKAYESANDLLGDVVKVT
PTSKVVGDLAQFMVSNKLTAEDVIARAGELDFPGSVLEFL
EGLMGQPYGGFPEPLRSRALRDRRKLDKRPGLYLEPLDLA
KIKSQIRENYGAATEYDVASYAMYPKVFEDYKKFVAKFGD
LSVLPTRYFLAKPEIGEEFHVELEKGKVLILKLLAIGPLS
EQTGQREVFYEVNGEVRQVSVDDKKASVENTARPKAELGD
SSQVGAPMSGVVVEIRVHDGLEVKKGDPIAVLSAMKMEMV
ISAPHSGKVSSLLVKEGDSVDGQDLVCKIVKA
SEQ ID NO: 6 is an exemplary nucleic acid
sequence encoding ald6a from A. niger.
ATGGCCGCCCGTCGGTCTATTGTCCGTTCCATCCCCGCTC
TCCGCGCCGCCTCTCCCCGCACGGCCGTGGCCACCTCCAG
ATCTGTAGCTGCAGCTTCGGCCTCGAGGCGCCTTTCTACT
GTTATAATGCCCTCTTCTTCTTCGACAACTTCTTCTGCAA
CATCTAAGCTCTCCTCATCCTCACCTGCCACCATGTCAGC
AACACGTCGACTCCACGCTACTGCCCAGCAGTTCACCCCC
GCAACTACCTCCGCGGCTACTACCGCCACCGAGTACCCGA
CGGACCATAGCCCCATCGCCAACCCCATCGACACCGCCAA
TTTCCTGGACAACCAATTTGTGGCTTCTAAGGCTACCACC
TGGATCGATCTGCACGATCCTGCTACCAACAACCTCGTTA
CTCGGGTGCCGCAAAGCACCGATGAGGAACTCCGTGCCGC
CGTCGAGTCGGCCGAGAAGGCCTTCCCAGCCTGGCGCGCA
ACCAGCGTTATTGCCAAGCAGCAGATCATGTTCAAGTTCG
TGAGCTTGATTCGTGCCAACTGGGACCGACTTGCTGCATC
CATCACTCTCGAGCAGGGCAAGACCTTTGCCGATGCCAAG
GGCGATGTTCTCCGCGGTCTTCAAGTTGCCGAAACAGCAT
GTGGTATTACCACCCAGCTAACGGGCGAGGTGCTCGAAGT
GGCCAAGGATATGGAGACTAGGAGCTACAGAGAGCCCTTG
GGTGTGGTTGCTGCCATTTGTCCTTTCAGTAAGTGATTCT
GCATCCGTCCTGCAAGCTAGGTGACATTGTCCGACGCTAA
CCGCACCCCCTCGCCTTCTCGGCAGACTTCCCCGCAATGA
TTCCCCTGTGGTGCATCCCCATCGCTACCGTGACGGGCAA
CTGTCTCATCCTCAAGCCATCTGAGCGGGACCCCGGAGCT
GCAATGATCCTGGCCGAGCTGGCCCGGGAAGCAGGCTTCC
CCCCCGGTGTCATCAACATTGTCCACGGCTCCGCCAAGAC
AGTCGATTTCATCCTCGACGAACCCGCCATCAAGGCCATC
AGCTTCGTCGGCAGCAACCGCGCCGGAGAGTACATCTACA
CCCGCGGCTCTGCCAACGGCAAGCGTGTCCAGGCCAATCT
GGGCGCCAAGAACCATGCCGCCGTAGTCCCAGACTGCAAC
AAGAACCAGACCCTGAACGCCCTCGTCGGAGCGGCCTTCG
GTGCTGCCGGCCAACGCTGCATGGCCCTCAGTACGGTCGT
CATGGTCGGCGAGACTCAGGACTGGCTCCCCGAGATGGCC
GAACGGGCCAAGGCCCTGAACGTCAACGGAGGCTTCGAAG
AAGGCGCTGACCTCGGCCCCGTCATCAGCCCCGAAAGCAA
GAAGCGCATCGAAGACCTGATCGCCAGCGCCGAAGAAGAG
GGTGCGACCATCCTCCTCGACGGCAGAGGCTACAAGCCAG
AGAAGTACCCCAACGGCAACTTTGTAAGCTTCCACCCAAC
CCCCTTCCCCGTCTCCTATCCAGCTACATCTTCTCTCTAA
CCAGCACCAAAACCACAGGTTGGCCCCACCATCATCACCA
ACGTAACCCCCGAAATGAAATGCTACAAGGAAGAAATCTT
CGGCCCCGTCCTCGTCTGCTTGTCCGTGCCCACACTAGAC
GACGCCATTGAGCTCATCAACAAGAACGAGTACGGAAACG
GAGCTGCCATCTTCACTCGCTCCGGTTCCACGGCCTCTCG
CTTCCAGAAGGACATCGAGGCCGGCCAGGTCGGTGTTAAC
GTGCCCATTCCTGTGCCGTTGCCCATGTTCTCTTTCACCG
GTAACAAGAAGAGTATTGCCGGCGGTGGCGCTAATACTTT
CTATGGTAAGCCCGGGTTGCAATTTTACACGCAGCAGAAG
ACGGTGACTAGTTTGTGGAGGAGTGAGGATGCTGTTAGCA
CTAAGGCTCATACGGTTATGCCGACTCATTCGTGA
SEQ ID NO: 7 is an exemplary nucleic acid
sequence encoding ald6b from A. niger.
ATGTCAAGATCGCAGAATTGGACGAATGCTTTGGATCCGG
TATTTGGTTTGATCAACCTCAGATTATTGCGATTAGTGCT
AAAGTGAAGGACAGGTTTCACAACGTCTACTTTGCAGGGT
CCCGGGGAGTACGCTTCAGGAAGTAAAGCGGGCTGTTGAT
GCTGCTGAAGATGCCCAGCCTGGATGGGCTGCTCTGGGGT
TCCAGGTGAGGCGTGAACATCTGTTGCGGCTGGTGGATGT
ATTGAGACAGATGTCTCCTGAGATTGTAAGCTGCCGGGAC
AGCTGACAGGCAAAGGAGTGGAACTGACGATCGTAGGTGA
CCTGCCTGTCTCGAGAAGTTGGAAAGACATTAGCCGATGC
AGACGCGGAGGTTTTCCGTGGTCTGGACTGCATACATGCC
GCATGTTCGATCGGGCCTGAAATGGCTGGCATGTTTTTAG
GAGGCGATGCAACATTGCTGCAAACTTTCTACGAGCCAGT
TGTGAGTCTAGATGCCGGTTAACTACAGATATGCTACTAA
CAGACCGTGGCAGGGCGTCTGTGTCTCAATCACTCCATTT
AGCTTTCCCTTCATGATTCCCCTGTGGTCGCTTCCGTACG
CGCTTATCACTGGCAACACCGTCATCCTGAAACCATCCGA
GAAAACACCAACTACGTCTTCGTTATTAGCGCAGGCATTC
ATCAAGACTGGCTTCCCTCCCGGGGTCTTCAATGTGCTCC
ACGGCGGCCCATCCACAGTGCAGATGCTTGTGACCCAGCC
GACGGTTCAAGCGGTGAGTTTCGTTGGGTCCGAGTCAGCA
GCTAGGCAGGTGCATGACCTGGCAAGGGCCGCAGGGAAGC
GAATACAAGCCGAGTGTGGTGGTAAGAATCATGGGGTTGT
CTTGGAGGATGCTAATATGTCCTCGACGCTTTTTGCGATC
GCTGGAAGTGCCTTTGGGGCCGCCGGTCAGCGTTGTATGG
CCTTGAGCGTAGCGGTGTTTGTAGGTGCGACGAGAGACTG
GATACCTAGGCTGGTGGAGCTGGCACAGTCGATGGTAGTA
GGATGTGGTGGTGATCAGGAATCTAAGATTGGGCCGTTGA
TTGACAAAACAGCCAAAGACAAAGTCAGCGAGATGATCCA
GCGAGCAGTTGAGGAGAGAGCAACCGTTCTTTTGGACGGT
CGAGATATTGAGGTCCCCGGCTATCCGGATGGCAACTTCA
TGGGCCCAACTATTCTCGGGGATGTGCAGACTTACATGGA
ATGCTATCAGGCGGAAATATTTGGGCCTGTGCTCATCTGC
ATGGAGGTGGACACTCTTGAGGAAGCGATCGATCTGATAA
GCCAAAATAAGTGTCAGTCTCCCACAACCCGGGCTTGAAG
GGGATGGATTGCTGACCTGATCGCAGATGGTAACGGATGC
TCTATTTTCACCACCAGCGGCAAGCATGCAAATACGTTCC
AACGCTGTGTCAATGTTGGTCAGATTGGTGTCAACATCCC
GTTGATCGGTATGGCGGTCCCCATTTCCGTAGCGCAACCA
ACCCCTGACAGTACAACAGCGCCGTATGGAACCGCGGTCC
GAACGAGCAACAAAGATTCTTTCCTGGGTGGTGAGTGCCC
AGACATCTCCGGTGGAAGGAAAGCTGACCTCTGCAGATCG
ACATTCCCCTGGGAAGACATATTGGCCATTTTTCACAACA
ACTAAAACGGTCTCATCTCGATGGGATCAGTGA
SEQ ID NO: 8 is an exemplary nucleic acid
sequence encoding ald3 from A. niger.
ATGCCCTTCTCCTCCTTCCCCGCTCCCTTTCGCTGTCTTT
CCTTCTCCTCTCTGCGGCTGCGTGGTGTGTCATTCTCCCG
TCTGCCAGTTGTTCCTTTCTCCCCTCATCGTCATCACTTC
CATAGCTCGTCCATCACAATGAGCGATCTGTCCGTGCAGC
TGACAGCCCCGAATGGGCGCACATACGCCCAGCCGGTTGG
TTTGTTCATCAACAATGAATTTGTCGCTTCCAAGTCCGGC
GAGAAGTTCGCCACCATCAACCCTTCGTACGTGACCTCCC
AATCCTAGTATCTTTCTTGGATCTGCTGCTGACCAGTGCT
AGAAATGAAGCGGAAATCACCTCGGTCTATGCTGCGGGTG
AAGAAGACGTGGATATTGCTGTGAAGGCTGCCCGGAAAGC
CCTCAACCACCCGTCATGGAAGTTGTTGCCTCCGACTGAA
CGGGGCACCCTGATGTTGAAGCTGGCCGACTTGATCGAGC
AGCACAAGGAGACGCTTGCCACCATTGAAACGTGGGACAA
TGGTAGGGAGCCTCGCATTGTCCTCAAAGGCGATCACCAT
GTACTGATTCTCTCCAGGCAAGCCATACTCAGTTTCGCTG
AACGATGACCTGGGAGAGGTTGTTGGCTGCCTCCGGTACT
ATGCTGGATACGCCGACAAGGTCCATGGTCAGACTATTAG
CACCACCCCGGCCAAGTTTGCCTATACTCTTCGTCAGCCA
ATTGGTGTCGTGGGTCAGATTATCCCCTGGAACTTCCCTC
TCGCGATGGCTGCCTGGAAGTTGGGTCCTGCCTTGGCCTG
CGGAAACACCGTGGTTCTGAAACCGGCTGAGCAGACTCCG
CTCAGTGTTCTCTATCTTGCCAATCTCATCAAGGAAGCCG
GTTTCCCGCCAGGTGTTGTCAACATCCTGAACGGCTTTGG
ACGCGTAGCTGGAAGTGCGCTCGTGACCCACCCCGGGGTG
GACAAGGTTGCCTTCACAGGTTCGACGTTGACTGGCCGCG
AAATTATGAAGTTGGCTGCAGGCACCCTGAAGAACATTAC
GCTCGAGACAGGTGGCAAATCTCCTCTGGTCGTCTTCAGT
GATGCCGATATCGACCAGGCCGCTAAGTGGGCGCATGCGG
GTATCATGTACAACCAAGGACAGGTTTGCACCGCCACCTC
TCGTATCCTGGTGCACGAGTCCGTCTATGACAAGTTCGTG
GCGCTCTTCAAGGAAGCCGTGGCCAACACCAGCAAGGTGG
GCGATCCCTTTGCCGACGACACCTTCCAGGGTCCCCAGGT
CACCAAGGCCCAATACGACCGCGTGCTCTCCTATATCGAA
GCTGGAAAGTCCGAAGGAGCGACCCTGGTGGCCGGTGGCG
AGCCGTTCAAGAACGTCGGCGACGGCAAGGGTTTCTTTAT
CGCGCCCACCATCTTCACCAACGTCAAGGATAACATGCGG
ATCTACCGGGAGGAGGTTTTCGGACCATTTGTCGTGATTT
CCAGCTTCTCAGAGGAGGATGAGGCTGTGAGACGGGCCAA
CGATACGACCTATGGACTGGGCGCTGCCCTGTTCACCAAG
GACATTGAGCGTGCACACCGGGTTGCGTCGGAGATCGAAG
CGGGCATGGTCTGGATCAACAGCAGCAACGACAGCGATAT
CCGTGTGCCTTTCGGGGGTGTGAAGCAGAGCGGAATTGGG
CGCGAACTTGGCGAGGCCGGTCTGGAGGCCTACTCACAGA
TCAAAGCGGTCCATGTCAACTTGGGAACGAAGCTGTAG
SEQ ID NO: 9 is an exemplary nucleic acid
sequence encoding oahA from A. niger.
ATGAAAGTTGATACCCCCGATTCTGCTTCcaccatcagca
tgaccaacactatcaccatcaccGTAGAGCAGGACGGTAT
CTATGAGATCAACGGTGCCCGTCAAGAGCCCGTGGTCAAC
CTGAACATGGTCACCGGTGCGAGCAAACTGCGCAAGCAGC
TTCGCGAGACCAATGAGTTGCTCGTGTGTCCTGGTGTGTA
CGACGGTCTGTCCGCCCGTATTGCCATCAACCTGGGCTTC
AAGGGCATGTACATGGTATGTTGGATTCCTCACACTACCT
TTCCCCACAGTCAACACTTCTCCGCTTCCGCGATGGAGAA
AAAAGATCATACTAACGGAAGGGTCAGACCGGCGCCGGTA
CTACCGCGTCTAGACTGGGCATGGCCGATCTGGGTCTAGC
CCACATCTACGACATGAAGACCAACGCGGAGATGATCGCA
AACCTGGACCCCTACGGTCCTCCCCTGATCGCAGACATGG
ACACTGGCTACGGAGGTGAGAATCCCCCATCTCCACTGTC
TGCCAAGACATAATGATCTACCCGCGCCAAAAAGCAAAAC
GGCAATATAGACCCAGTTCCCCACTAACACAAAAAAAAAA
CAAAAATAGGCCCCCTGATGGTCGCCCGTTCCGTTCAACA
ATACATCCAAGCCGGAGTCGCGGGATTCCACATCGAAGAT
CAGATCCAAAACAAGCGATGCGGACACCTGGCAGGCAAGC
GCGTCGTCACCATGGACGAATACTTGACTCGCATCCGCGC
CGCCAAGCTCACCAAGGACCGCCTCCGCAGCGACATCGTG
CTGATTGCCCGCACCGACGCCCTCCAGCAGCACGGCTACG
ACGAGTGCATTCGCCGCCTTAAGGCCGCCCGCGATCTTGG
CGCCGATGTTGGTCTCCTCGAGGGCTTCACCAGTAAGGAG
ATGGCGAGGCGGTGTGTCCAGGACCTTGCGCCTTGGCCGC
TTCTTCTCAACATGGTGGAGAACGGTGCTGGGCCGGTTAT
TTCCGTCGATGAGGCTAGGGAAATGGGCTTCCGCATTATG
ATCTTCTCGTTCGCTTGCATTACTCCTGCCTATATGGGGA
TTACGGCTGCTCTGGAGAGGCTCAAGAAGGATGGTGTGGT
TGGGTTGCCCGAGGGGATGGGGCCGAAGAAGCTGTTTGAG
GTTTGCGGATTGATGGACTCGGTGAGGGTTGATACCGAGG
CTGGTGGAGATGGGTTTGCTAATGGTGTTTAA
SEQ ID NOS: 10-128 and 143 are
oligonucleotide primer sequences.
SEQ ID NO: 129 is an exemplary nucleic acid
sequence encoding mctl from A. niger.
ATGCATACGACCGAGAAGATACCCAGCTTCCTATCTCGCC
GGAAACCCAGCAATGATTCAGAACACGTACCCCCACCACC
CGATGGAGGAGTCCAAGCCTGGACCCAAGTCGTCTGCATG
CACTTCGTATTCTTCAACACCTGGGGGATCACCACTAGCT
TCTCCGTCTTTGAGCAATTATACACCAAAACCCTACCACA
GTCAGCATCCTCTATCTCCTGGATCGGCAGCGTCCAGGTA
TCTCTCCTGTTCTTCCTGAGCGCCCTTGCCGGACGCGCAA
CAGACGCCGGCTTCTTCAGAGTCATGTACCCACTCGGGGT
CCTCCTCCAGCTCGTGGGAATCTTCATGCTCTCTCTGTGC
AAGACCTACTGGCAAATCTTCCTCGCTCAGGCGGTATGCA
TGGGTCTCGGCAATGGACTTACCTTCAGCCCCGGACTTTC
AGTCATGTCCGCCTACTTCATGAAAAACCGAGCCTTTGCA
GTGGGCTTGGCTGCGTCTGGTGCAGCAACAGGGGGTTTGA
TATACCCCGTACTCATCAACCAATTGCTCTATAATCACCA
GATAGGCTTCGCATGGACAATACGCGCAGCAGGACTTCTC
ATGCTAATTACCCATATCTTTGGCCTGGTTTTCTTCAGAC
CCCGTCTTCCACCCCGGACCACCGGCCCTCTCATCGAACT
CGGCGCATTTACCGAGCCGCCTTTCGTCTTCTTCACCTTA
TGCTACTTCTTCACCTTCTGGGGCCTGTACTTCGCCTGGT
TCTATCTTGGCACCTTTGCCCGAGACCGTCTCGGCATCGC
GGATACACAGAACCTGCTCCTTGTGTTGAATGGAGTCGGC
ATCGTCGGACGTATCGGGCCGAGTATAATTGGTGATCGAT
GGACGGGGCGATTAAACATCTTGATCCCCATCACTCTTTC
ATGTGCGATTCTGATGTTTTGCTGGATAGCCGTCAGTACT
GTTGCAGGGCTGTATGCGTTTGTTGTTGTGTATGGGCTCG
TGGGTGGCGCCGCGCAATCGGTGATCCCTGCGACGGCGAC
GACCATGACGCCGGATATCAATCGGACGGGTACAAGGCTG
GGAATGATCATGAGTATTGTGGGATTTGCTACGCTGACTG
GACCGGCTATTGAAGGGGCGTTGATCGCTACTGACGGGGG
GAGGTATGTGGCGGCTCAGATTTTTGCGGGTGTGAGTATT
CTGCTGGGCGTGTGTGCGGTTGCGGCGGCGCGGGTGGCTA
AGGCTGGGTGGGGGTTGGATGTGAAGGTGTGA
SEQ ID NO: 130 is an exemplary nucleic acid
encoding aspartate 1-decarboxylase
(PAND) (codon optimized for Aspergillus).
ATGCCCGCCACCGGCGAGGACCAGGACCTGGTGCAGGACC
TGATCGAGGAACCCGCCACCTTCTCCGACGCCGTCCTGTC
CTCCGACGAGGAACTGTTCCACCAGAAGTGCCCCAAGCCG
GCTCCGATCTACAGCCCCGTCAGCAAGCCCGTCAGCTTCG
AGTCCCTGCCGAACCGCCGCCTGCACGAAGAGTTCCTCCG
CTCCTCCGTCGACGTCCTGCTGCAAGAGGCCGTGTTCGAG
GGCACCAACCGCAAGAACCGCGTCCTGCAGTGGCGCGAGC
CCGAAGAACTGCGCCGCCTGATGGACTTCGGCGTCCGCAG
CGCCCCGTCCACGCATGAGGAACTGCTCGAGGTCCTGAAG
AAGGTCGTCACCTACTCCGTCAAGACCGGCCATCCGTACT
TCGTCAACCAGCTGTTCTCCGCCGTCGATCCCTACGGCCT
GGTCGCCCAGTGGGCCACCGACGCGCTGAACCCCTCCGTC
TACACCTACGAGGTCAGCCCCGTGTTCGTCCTGATGGAAG
AGGTCGTCCTGCGCGAGATGCGCGCCATCGTCGGCTTCGA
AGGCGGCAAAGGCGACGGCATCTTCTGCCCTGGCGGCTCG
ATCGCCAACGGCTACGCCATCAGCTGCGCCCGCTACCGCT
TCATGCCCGACATCAAGAAGAAGGGCCTGCACTCCCTGCC
GCGCCTGGTCCTGTTCACCTCCGAGGACGCCCACTACTCG
ATCAAGAAGCTGGCCTCGTTCCAAGGCATCGGCACCGACA
ACGTCTACCTGATCCGCACCGACGCTCGCGGTCGCATGGA
CGTCAGCCACCTGGTCGAAGAGATCGAGCGCTCCCTCCGC
GAGGGCGCTGCCCCGTTCATGGTCAGCGCCACCGCCGGCA
CCACCGTCATCGGCGCCTTCGATCCCATCGAGAAGATCGC
CGACGTCTGCCAGAAGTACAAGCTCTGGCTGCACGTCGAC
GCCGCCTGGGGCGGAGGCGCTCTGGTGTCCGCCAAGCACC
GCCATCTGCTGAAGGGCATCGAGCGCGCCGACTCCGTCAC
CTGGAATCCCCACAAGCTGCTGACCGCTCCGCAGCAGTGC
AGCACCCTGCTGCTGCGCCACGAGGGCGTCCTGGCCGAGG
CGCACTCCACCAACGCCGCCTACCTGTTCCAGAAGGACAA
GTTCTACGACACCAAGTACGACACCGGCGACAAGCACATC
CAGTGCGGCCGTCGCGCCGACGTGCTGAAGTTCTGGTTCA
TGTGGAAGGCCAAGGGCACCTCCGGCCTCGAGAAGCACGT
GGACAAGGTGTTCGAGAACGCCCGCTTCTTCACCGACTGC
ATCAAGAACCGTGAGGGCTTCGAGATGGTGATCGCCGAGC
CTGAGTACACCAACATCTGTTTCTGGTACGTCCCCAAGAG
CCTGCGCGGACGCAAGGACGAGGCCGACTACAAGGACAAG
CTGCACAAGGTCGCCCCTCGCATCAAAGAACGCATGATGA
AGGAAGGCTCCATGATGGTCACCTACCAGGCGCAGAAGGG
CCATCCGAATTTCTTCCGCATCGTCTTTCAGAACTCCGGC
CTGGACAAGGCCGACATGGTCCATCTGGTCGAGGAAATCG
AACGCCTGGGCTCCGACCTCTGA
SEQ ID NO: 131 is an exemplary nucleic acid
encoding β-alanine-pyruvate aminotransferase
(BAPAT) (codon optimized for Aspergillus).
ATGGAACTGATGATCGTCCAGGTCACCGAGCAGACCCAGA
GCCTGAAGAAAACCGACGAGAAGTACCTGTGGCACGCCAT
GCGCGGTGCCGCTCCGAGTCCGACCAACCTGATCATCACC
AAGGCCGAAGGCGCGTGGGTCACCGACATCGACGGCAACC
GCTACCTGGACGGCATGTCCGGCCTGTGGTGCGTCAACGT
CGGCTACGGCCGCAAAGAGCTGGCTCGCGCTGCCTTCGAG
CAGCTGGAAGAGATGCCGTACTTCCCGCTGACGCAGTCGC
ACGTCCCCGCCATCAAGCTGGCCGAGAAGCTGAACGAGTG
GCTGGACGACGAGTACGTCATCTTCTTCTCCAACTCCGGC
TCGGAGGCCAACGAAACCGCCTTCAAGATCGCCCGCCAGT
ACCACCAGCAGAAGGGTGACCACGGCCGCTACAAGTTCAT
CTCTCGCTACCGCGCCTACCACGGCAACTCCATGGGTGCC
CTGGCCGCGACCGGTCAGGCCCAGCGCAAGTACAAGTACG
AGCCCCTCGGTCAGGGCTTCCTGCACGTGGCCCCTCCGGA
CACCTATCGCAACCCCGAGGACGTGCACACCCTGGCCTCC
GCCGAAGAAATCGACCGCGTCATGACCTGGGAGCTGTCCC
AGACCGTCGCCGGCGTCATCATGGAACCCATCATCACCGG
TGGTGGCATCCTGATGCCGCCGGACGGCTACATGGAAAAG
GTCAAAGAGATCTGCGAAAAGCACGGCGCCCTGCTGATCT
GCGACGAGGTCATCTGCGGCTTCGGTCGCACCGGCAAGCC
CTTCGGCTTCATGAACTACGGCGTCAAGCCCGACATTATC
ACCATGGCCAAGGGTATCACCTCGGCCTACCTGCCGCTGT
CTGCCACCGCGGTCCGCCGCGAGGTCTACGAGGCCTTCGT
CGGCTCCGACGACTACGACCGCTTCCGCCATGTCAACACC
TTCGGCGGCAACCCTGCCGCCTGCGCGCTGGCCCTGAAGA
ACCTCGAGATCATGGAAAACGAGAAGCTCATCGAGCGGTC
CAAAGAACTGGGCGAGCGCCTGCTGTACGAGTTGGAGGAC
GTCAAAGAGCACCCCAACGTCGGGGACGTCCGCGGCAAGG
GCCTCCTGCTGGGTATCGAACTCGTCGAGGATAAGCAGAC
CAAAGAGCCCGCCTCCATCGAAAAGATGAACAAGGTCATC
AACGCCTGCAAAGAGAAGGGTCTGATCATCGGCAAGAACG
GCGACACCGTGGCCGGCTACAACAACATCCTGCAGCTCGC
CCCTCCGCTGTCCATCACCGAAGAGGACTTCACCTTCATC
GTCAAGACTATGAAGGAATGTCTGGCCCAGCTCTGA
SEQ ID NO: 132 is an exemplary nucleic acid
sequence encoding 3-hydroxypropionate
dehydrogenase (HPDH) (codon optimized
for Aspergillus).
ATGATCGTGCTGGTCACGGGCGCGACCGCCGGTTTCGGCG
AGTGCATCACCCGCCGCTTCATCCAGCAGGGCCACAAGGT
GATCGCTACCGGACGCCGCCAAGAGCGCCTCCAAGAGCTG
AAGGATGAGCTGGGCGACAACCTGTACATTGCCCAGCTGG
ACGTGCGCAACCGGGCTGCCATCGAAGAAATGCTCGCCTC
GCTGCCCGCCGAGTGGTGCAACATCGACATCCTGGTCAAC
AACGCCGGTCTGGCCCTCGGCATGGAACCGGCGCACAAGG
CCAGCGTCGAGGACTGGGAAACCATGATCGACACCAACAA
CAAGGGACTCGTCTACATGACCCGCGCTGTGCTGCCCGGC
ATGGTCGAGCGCAACCACGGCCACATCATCAACATCGGCT
CCACCGCTGGCAGCTGGCCCTACGCTGGCGGCAACGTCTA
TGGCGCGACCAAGGCGTTCGTCCGCCAGTTCTCCCTGAAC
CTGCGCACCGACCTGCACGGCACCGCCGTCCGCGTGACCG
ACATTGAGCCCGGTCTGGTCGGCGGCACCGAGTTCAGCAA
CGTCCGCTTCAAGGGCGACGACGGCAAGGCCGAGAAAACC
TACCAGAACACCGTCGCTCTGACCCCTGAGGATGTCAGCG
AGGCCGTCTGGTGGGTCAGCACTCTGCCCGCGCACGTCAA
CATCAACACCCTCGAGATGATGCCCGTCACGCAGTCCTAC
GCCGGCCTGAACGTCCACCGCCAATAG
SEQ ID NO: 133 is an exemplary nucleic acid
encoding pyruvate carboxylase (pyc) from
A. niger.
ATGGCTGCTCCCCGCCAGCCCGAGGAGGCGGTCGATGACA
CCGAGTTCATCGATGACCACCATGACCAGCACCGGGACTC
TGTCCACACCCGTCTGCGTGCCAATTCGGCTATCATGCAG
TTCCAAAAGATCCTTGTTGCCAACCGTGGTGAAATCCCCA
TTCGTATCTTCCGGACGGCTCACGAGCTGTCCCTGCAGAC
CGTCGCCGTCTACTCCCATGAGGACCGTCTCTCCATGCAC
CGTCAGAAGGCCGACGAGGCCTACATGATTGGCAAGCGCG
GTCAATATACACCGGTTGGGGCCTACTTGGCCATTGACGA
GATCGTCAAGATTGCTCTGGAGCATGGTGTACACCTGATC
CACCCGGGTTACGGTTTCCTGTCCGAGAACGCCGAGTTTG
CCCGCAAGGTGGAGCAGTCCGGCATGGTTTTCGTCGGCCC
TACCCCCCAGACCATCGAGAGCCTCGGTGACAAGGTCTCC
GCCCGTCAGCTGGCTATCCGCTGCGACGTGCCCGTCGTGC
CCGGTACCCCGGGCCCTGTCGAGCGCTACGAGGAGGTCAA
GGCCTTCACCGACACCTACGGCTTCCCCATCATCATCAAG
GCCGCCTTTGGTGGTGGTGGTCGTGGTATGCGTGTCGTTC
GCGACCAGGCCGAACTGCGTGACTCCTTCGAGCGTGCCAC
CTCCGAGGCCCGCTCTGCCTTTGGCAACGGCACCGTCTTC
GTCGAGCGCTTCCTCGACCGCCCCAAGCACATCGAAGTCC
AGCTGCTGGGTGACAACCACGGCAACGTCGTCCACCTGTT
CGAGCGTGACTGCTCCGTCCAGCGTCGCCACCAGAAGGTC
GTTGAAATTGCCCCGGCCAAGGACCTGCCTGCCGATGTCC
GTGACCGCATCCTGGCTGATGCTGTCAAGCTGGCCAAGTC
GGTCAACTACCGCAACGCCGGTACTGCAGAGTTCCTGGTT
GACCAGCAGAACCGTTACTACTTCATTGAGATCAACCCCC
GTATCCAGGTCGAGCACACTATCACCGAAGAGATCACGGG
TATCGATATCGTTGCTGCTCAGATCCAGATTGCGGCCGGT
GCTACCCTGGAACAGCTGGGTCTGACCCAGGACCGCATCT
CCACTCGTGGATTCGCCATTCAGTGCCGTATCACCACCGA
GGACCCCTCCAAGGGCTTCTCCCCCGACACCGGTAAGATC
GAGGTCTACCGCTCCGCCGGTGGTAACGGTGTCCGTCTGG
ATGGTGGAAACGGTTTCGCCGGTGCCATCATCACCCCTCA
CTACGACTCCATGTTGGTCAAGTGTACCTGCCGTGGTTCC
ACCTATGAGATCGCTCGCCGCAAGGTCGTTCGTGCTTTGG
TCGAGTTCCGTATCCGTGGTGTCAAGACCAACATTCCTTT
CCTCACCTCTCTTCTGAGTCACCCTGTGTTCGTGGATGGT
ACCTGCTGGACCACGTTCATTGATGACACTCCCGAGCTGT
TCGCCCTTGTCGGCAGTCAGAACCGTGCCCAGAAGCTGCT
GGCTTACCTGGGTGATGTGGCTGTCAACGGCAGCAGCATC
AAGGGCCAGATCGGCGAGCCCAAGCTCAAGGGCGATATCA
TCAAGCCCGTTCTGCATGACGCTGCCGGCAAGCCCCTCGA
CGTCTCTGTCCCCGCCACCAAGGGATGGAAGCAGATCCTG
GACAGTGAGGGTCCCGAGGCTTTTGCCCGCGCTGTGCGTG
CCAACAAGGGCTGCTTGATCATGGATACTACCTGGCGTGA
TGCCCACCAGTCGCTGCTGGCCACTCGTGTGCGTACCATT
GACCTCCTGAACATCGCCCACGAGACGAGCCACGCCCTCG
CCAACGCCTACAGTTTGGAATGCTGGGGTGGTGCCACCTT
CGATGTCGCCATGCGCTTCCTGTACGAGGACCCCTGGGAC
CGTCTTCGCAAGCTGCGCAAGGCCGTTCCCAACATCCCCT
TCCAGATGTTGCTCCGTGGTGCCAACGGTGTTGCTTACTC
CTCCCTCCCTGACAACGCCATCTACCACTTCTGTAAGCAG
GCCAAGAAGTGCGGTGTCGACATCTTCCGTGTCTTCGATG
CTCTCAACGACGTTGACCAGCTCGAGGTCGGTATCAAGGC
TGTCCACGCTGCCGAGGGTGTTGTTGAGGCTACTATTTGC
TACAGTGGTGATATGCTCAACCCCAGCAAGAAGTACAACC
TGCCTTACTACCTCGACCTTGTTGATAAGGTGGTCCAATT
CAAGCCCCACGTCTTGGGTATCAAGGACATGGCTGGTGTG
CTGAAGCCCCAGGCTGCTCGTCTGCTGATCGGTTCCATCC
GCGAGCGCTACCCCGACCTCCCCATCCACGTGCACACCCA
CGATTCTGCTGGTACCGGTGTGGCTTCCATGATTGCTTGC
GCTCAGGCCGGTGCTGATGCCGTTGATGCTGCCACGGACA
GCCTTTCCGGTATGACCTCCCAGCCCAGCATTGGTGCTAT
CCTCGCTTCCCTCGAGGGAACTGAGCACGACCCCGGCCTC
AACTCGGCCCAGGTTCGCGCCCTTGACACCTACTGGGCGC
AGCTGCGTCTCCTCTACTCGCCCTTCGAGGCAGGTCTGAC
TGGTCCCGACCCCGAGGTTTACGAGCATGAGATCCCTGGT
GGTCAATTGACCAACCTGATCTTCCAGGCCAGCCAGCTTG
GTCTGGGCCAGCAGTGGGCGGAGACGAAGAAGGCTTACGA
GTCTGCCAACGATCTTTTGGGCGATGTCGTCAAGGTCACC
CCCACTTCCAAGGTGGTCGGTGATTTGGCTCAGTTCATGG
TCTCCAACAAGTTGACTGCTGAAGATGTGATTGCTCGCGC
CGGCGAGCTTGACTTCCCCGGTTCCGTTCTGGAGTTCCTC
GAGGGTCTCATGGGCCAGCCCTACGGTGGATTCCCCGAGC
CTCTGCGCTCTCGCGCTCTGCGTGACCGTCGCAAGCTCGA
CAAGCGCCCTGGTCTGTACCTGGAGCCCCTTGACCTGGCC
AAGATCAAGAGCCAGATCCGGGAGAACTATGGCGCAGCTA
CCGAGTACGATGTGGCCAGCTATGCCATGTACCCCAAGGT
CTTCGAGGATTACAAGAAGTTTGTCGCCAAGTTCGGTGAT
TTGTCCGTCCTGCCCACGCGTTACTTCTTGGCCAAGCCCG
AGATCGGCGAGGAATTCCACGTCGAGCTGGAGAAGGGTAA
GGTGCTGATCCTGAAGTTGTTGGCCATTGGTCCCCTCTCC
GAGCAGACCGGCCAGCGTGAGGTCTTCTACGAAGTCAACG
GTGAGGTTCGCCAGGTTAGTGTGGACGACAAGAAGGCGTC
CGTCGAGAACACCGCCCGCCCCAAGGCCGAGCTGGGTGAC
AGCAGCCAGGTTGGTGCTCCTATGAGCGGTGTGGTTGTCG
AGATCCGCGTCCACGACGGCCTGGAAGTCAAGAAGGGTGA
CCCCATTGCCGTCCTGAGCGCCATGAAGATGGAAATGGTT
ATCTCTGCTCCCCACAGTGGCAAGGTCTCCAGCTTGCTGG
TCAAGGAAGGTGACTCGGTGGATGGCCAGGATCTTGTCTG
CAAGATCGTCAAGGCCTAG
SEQ ID NO: 134 is an exemplary ubi4p
sequence from A. niger.
CTTCGGAGTAGCAACGAGTATTTTCACCGGGAGTTTCAAC
GGGTTCTATTTCAGGAACACGGCTGCGGTCTGGATTGGGT
CGGGCTGAGATACCGACTGGTGGCGTCAGTGGCGGGTACG
GACGGAGTCGTCCTGGCCGCTCGTAGACACTCCCCCGGAC
TGATATCAGGCCCCGGCAACTGGCTTCGTCTCACTCCAGG
GCATCAGGAGTGCCTACCACATGGGTTCAGGCTTTGCCCC
GTCGTCTAAGTTTGCAGGACAAAATTTTCGTATGCGTTAC
CACTCTTTCCTTTCAGCAACCATTCCGTAGTGAAAACCCA
ATAATAGGTGGCTGCCGTGGGAGCCTGAGTCAACCCAACC
AGAACCTTTCTAGTAGATTCTCCCCCAAGCGCTTCAGCAA
CGAAGCGTATTGGAGAACCAAATGACGCAGACCAAGCGGA
TTCCGGTGCAATAGCCGGATGGCAAGGGAATCCCCCAGGA
GGTGCCAGAAGCGTCGCCCGAAAGGTACTTCGTCTGACAG
GCTAACACCGCTCGGGCTAAGGTCCCTGCTGCTCTTTTCC
CTTTATTGCGACTTAACCTCTAAGCCATTCCCTTGCATCA
CGTTATCTCACTGACCGACCTCTGACTAAGGCGCTTCGCC
TCCGCCGCCTCCCCTCATTCACCTCCTCTCCTGACTACTT
AAGCCTTCTCTTCCTTCCTCTCACTACCAACCCTCCTTCA
TCCCTCATACCTCTCATCCTACCACTCACCTTTCGCGCAT
CGCCATCTGCGATCCTCCCCACAACACTCCACCTAGATAC
ATACACCATTAACTGCGCTTCTACAAC
SEQ ID NO: 135 is an exemplary ubi1S27p
sequence from A. niger.
CATTATCGATCTATCCACATTGCCTCTTATAACTGACGGG
AGGAGGCGACCTGTGGCCTGTGAGGGTTTGAAACATTATT
TCACCCTTCCTCTGCAATAGCCGATTTAATAATTGACGCC
TATCCCGCATGCTATTCAATCAATAATTAGCAACAGCGAG
CCGAGGGATGAGAGGGCGGCAAATGATGACGATGACGAAG
TTGACGTCGATGCTGAGAGGCTGGATTGACCACTTCGTCT
AGTTGGTTTTAGGCCTAGTCGTACTTTCTGCCTGGCTTTT
CCGGGCCGTGCCTATGCCTGGCGATGCAGGCACCAAACTG
ACGTATCCAAGAAACCGTTAAAGTCAGCAAATCCCGTGAC
CTCAGCCGCAGACCATCACATGACTGAGGACCCGGAGGGC
TGATTATCCGAGTGTCCACCCGCACTACGCCTGACTCGGT
AATCTGGACACCACGTGACCGCACAACCGATTTTAACGAA
GCCCACCACCACCGAGGATATTTTCCTACCACGATCGCCG
AAGGTTTAGGAAGACAAGTCTTGTCGAGGTGAGTTGATGA
CCCTATACATTTACTATGTGACAAGGAAAAGGATTCAATA
TTATTTGGAACATCTCATCTTCATTTCTTTCCACTTATCC
CAGAAAATACCTCCTGCGCACACTTTAGCGCGCGAGATAC
CAAAATTCCGGGGGAGTAATATGTTGAACAAGAAACGAAG
ATGAGAGCTGCGACGCAGATAATATTGAACTTTATCCTTG
CTCACGCACCAGTATCAAGTAAAACAATGAACTAACATCC
TTTTTCCAACCCAGACCTTCATCAAG
SEQ ID NO: 136 is an exemplary mbfAp
sequence from A. niger.
GGACATGCTGGAAGGGATTTTCTGGCTCAATACCACGTCT
GTATTTGACCCTTTCCAGACAGTTAATCCGCTGCAGGAGG
GCGAACTGTAGCTCCTCGTTCTCCTTGTAGCGCTTGATCC
AGTCTTTTTGGATGTTGCACTTGCTTGGCCTATGCTTCTC
ATATAATCTTGCCCTGTCATAGAGACGACGTCTGAGATTG
TAGCGTTCGTCTTTGATCACCCGGAGCCAGATAGGCCTGA
GTATATCTGACATTAGATCAAAGGGTCTGTGGATAGTCTC
CTTCAGCATCAGCGACGCATGTGACTCGCATGTCGGAGAG
AGCTTGTGGGTGGTCATCTTTGATGGCGTCCTCTGCTTTC
CCTTGATTTTCGTTGATTGTTTTTCGAAAGTTAAGTCTGG
AAGTCAAGAGAATCCTTCTGCCAGACATTATATTTACGTA
TACTGACGTAGTAGAAACAGCGTCAGGATGAGGACATGGT
GTGTGCTGGACCACGGAATCATAGTTCATCAGTATATTGG
GTTGGACAAATAACGCTGAGCATGTATATGTCTTTACACA
CTATAAAAGCCAGCGAACGCCAATAAAATAGGGCATATTG
ATGTGAAAATATGACACCAGTTAAAAGCAGTGTATTGATT
TTATCTCTCTTCACCTCGGACCTATACTACCGTATACAAG
ACTCAACTTACTTCCAGATATAGTAATATACACCCTATGG
ACGAACCAGCACAATAATTACAGCCAAACAACACCACCCA
AATGGCATATTCCTAATCAGCACTAAGCACAAATACCACT
GTCATCACAGCATAATCAATAAGAATCCCAGACAACCGAC
TCACTCTGACTCACCTTACACAAACCCCCAAGCAAAGCGC
AGCCCAGAACCTCAGCCAACAATCGGGCAACGTACGGGGA
AAGATTGGCCGATCCATGATGTCAGCAGCCCTAACCCAAA
GCGGACTAGCGCATACCGCCCCTCTGACTCCGCCATCCCA
GGGCTCGAGAAGCTTCCGTGGCGTCGATATAAATTCAGCG
GGCCTTGAACATCCCTCCTTACGACACACCTCACGCGATC
GATTTTGACACTCACACACCGCCACCCTCACATCCTCCAC
CCACACCACACCCCTTAATCAACCCACCATCACCGCTAGA
ACGTCTATCTCATCACCGACTTCTCATCCATCTTCAAA
SEQ ID NO: 137 is an exemplary mct1 amino
acid sequence from A. niger.
MHTTEKIPSFLSRRKPSNDSEHVPPPPDGGVQAWTQVVCM
HFVFFNTWGITTSFSVFEQLYTKTLPQSASSISWIGSVQV
SLLFFLSALAGRATDAGFFRVMYPLGVLLQLVGIFMLSLC
KTYWQIFLAQAVCMGLGNGLTFSPGLSVMSAYFMKNRAFA
VGLAASGAATGGLIYPVLINQLLYNHQIGFAWTIRAAGLL
MLITHIFGLVFFRPRLPPRTTGPLIELGAFTEPPFVFFTL
CYFFTFWGLYFAWFYLGTFARDRLGIADTQNLLLVLNGVG
IVGRIGPSIIGDRWTGRLNILIPITLSCAILMFCWIAVST
VAGLYAFVVVYGLVGGAAQSVIPATATTMTPDINRTGTRL
GMIMSIVGFATLTGPAIEGALIATDGGRYVAAQIFAGVSI
LLGVCAVAAARVAKAGWGLDVKV
SEQ ID NO: 138 is an exemplary aat1 amino
acid sequence from A. niger.
MGSTTPSVFSTAVVPAAPEDALFGLAQAFRQDSSDKKVDL
VIGAYRDDNAKPWILPVVKKADELVRNDPALNHEYLPIKG
LADYTTAAQKLIIGADSPAIRENRVCTFQTISGTGAVHLG
ALFLSKFHPSNPKPTVYLSNPTWANHNQIFTNVNLSLANY
PYFDPQTKGLNFSGMLSALRDAPTGSIILLHVCAHNPTGV
DLTQSQWKDVAVVMRERNHFPFFDCAYQGFASGDLIRDSW
AVRYFVEQGFELCVAQSFAKNFGLYGQRTGAFHFVSAPGA
EASQANAHVASQLAILQRSEISNPPAYGARIASRVLNDEG
LFKEWEEDLKTMSGRIAEMRQGLRERLEKKGTPGTWNHIT
DQIGMFSFTGLTESQVKVLKEKWHVYMTKNGRISMAGLNT
HNLDYFAEAVDSVVRETS
SEQ ID NO: 139 is an exemplary cDNA
encoding aat1 from A. niger.
ATGTCGCCCCTTTCCTCCTCTTCTTCTTCTCCATCTTCTT
CTCTCTCCCTCACTTCTTCTTCATCCTCTCCATCCGCCTC
CTCCTCCATCGCTTCTACTTCTACTTCTACCGCTCGTTCC
CGTCTCGCTTCTCTCTCCTCCCACATCATGGGCTCTACCA
CCCCCTCCGTCTTTTCCACCGCCGTCGTGCCCGCCGCGCC
AGAAGATGCTCTCTTCGGTCTGGCTCAGGCGTTTCGCCAG
GATTCCTCCGACAAGAAGGTCGATCTAGTCATTGGCGCTT
ATCGCGACGACAATGCCAAACCCTGGATCCTGCCCGTGGT
CAAGAAGGCTGACGAGCTCGTTCGCAATGACCCCGCCCTC
AATCACGAATACCTCCCCATCAAAGGTCTCGCCGACTACA
CCACTGCCGCCCAGAAATTGATCATTGGCGCTGATAGCCC
TGCCATTCGCGAGAACCGCGTATGCACCTTCCAAACCATC
TCCGGCACCGGTGCCGTGCACCTGGGCGCTCTCTTCCTCT
CCAAATTCCACCCCTCCAACCCTAAACCCACCGTCTACCT
CTCCAACCCAACCTGGGCCAACCACAACCAAATCTTCACC
AACGTCAACCTCTCCCTCGCCAACTACCCCTACTTCGACC
CGCAAACCAAAGGCCTCAACTTCTCCGGCATGCTCTCCGC
CCTGCGCGATGCTCCCACCGGCTCCATCATCCTCCTGCAC
GTCTGCGCCCACAACCCCACCGGTGTCGATCTGACCCAGT
CCCAATGGAAGGACGTCGCCGTCGTCATGCGCGAGCGCAA
CCATTTCCCCTTCTTTGACTGCGCCTACCAGGGTTTCGCC
TCTGGTGATCTCATTCGCGACTCCTGGGCCGTGCGCTACT
TCGTCGAGCAGGGCTTCGAGCTGTGCGTGGCCCAATCCTT
CGCTAAGAACTTTGGTCTGTATGGCCAGCGCACGGGCGCA
TTCCATTTCGTTTCCGCACCCGGTGCGGAGGCCAGCCAGG
CTAATGCGCATGTCGCGTCGCAGCTGGCTATTCTGCAGCG
CAGTGAGATCAGTAACCCGCCGGCGTATGGCGCGCGTATC
GCTAGCCGGGTGTTGAATGATGAGGGACTGTTCAAGGAGT
GGGAGGAGGATCTGAAGACGATGAGTGGTCGCATTGCGGA
GATGCGCCAGGGCCTGAGGGAGCGCTTGGAGAAGAAGGGC
ACGCCGGGCACTTGGAACCACATTACGGATCAGATTGGCA
TGTTCAGTTTCACGGGTTTGACCGAGTCGCAAGTTAAGGT
GTTGAAGGAGAAGTGGCATGTTTACATGACCAAGAACGGC
CGCATCTCCATGGCGGGTCTAAACACACATAACCTGGACT
ACTTCGCCGAAGCAGTGGACAGCGTAGTTCGGGAGACTTC
ATAA
SEQ ID NO: 140 is an exemplary gpdA
promoter (gpdAp) sequence from A. niger.
CTACTATGAAAGACCGCGATGGGCCGATAGTAGTAGTTAC
TTCCATTACATCATCTCATCCGCCCGGTTCCTCGCCTCCG
CGGCAGTCTACGGGTAGGA
TCGTAGCAAAAACCCGGGGGATAGACCCGTCGTCCCGAGC
TGGAGTTCCGTATAACCTAGGTAGAAGGTATCAATTGAAC
CCGAACAACTGGCAAAACATTCTCGAGATCGTAGGAGTGA
GTACCCGGCGTGATGGAGGGGGAGCACGCTCATTGGTCCG
TACGGCAGCTGCCGAGGGGGAGCAGGAGATCCAAATATCG
TGAGTCTCCTGCTTTGCCCGGTGTATGAAACCGGAAAGGA
CTGCTGGGGAACTGGGGAGCGGCGCAAGCCGGGAATCCCA
GCTGACAATTGACCCATCCTCATGCCGTGGCAGAGCTTGA
GGTAGCTTTTGCCCCGTCTGTCTCCCCGGTGTGCGCATTC
GACTGGGCGCGGCATCTGTGCCTCCTCCAGGAGCGGAGGA
CCCAGTAGTAAGTAGGCCTGACCTGGTCGTTGCGTCAGTC
CAGAGGTTCCCTCCCCTACCCTTTTTCTACTTCCCCTCCC
CCGCCGCTCAACTTTTCTTTCCCTTTTACTTTCTCTCTCT
CTTCCTCTTCATCCATCCTCTCTTCATCACTTCCCTCTTC
CCTTCATCCAATTCATCTTCCAAGTGAGTCTTCCTCCCCA
TCTGTCCCTCCATCTTTCCCATCATCATCTCCCTTCCCAG
CTCCTCCCCTCCTCTCGTCTCCTCACGAAGCTTGACTAAC
CATTACCCCGCCACATAGACACATCTAAACA
SEQ ID NO: 141 is an exemplary eno1 promoter
(eno1p) sequence from A. niger.
CTCGAGCTTACAAGAAGTAGCCTGTAGCAAGTCTGTGGTC
GTGTTTATTATTTATCTACTACTTCCCCCTCCTCCCTCCT
CCTCTTGTGTTAAGAGTACTCTCAAAAAAAGCCTTTCTTA
GATAGTACAACCACCATCTTCAAGGTCCTGTAAGTATGAC
TTGTCCCATATACTCATTTTATCACCCCTCCCATCTTTCT
CTGTCCATTTCATCCTCCTCTTCTTCCTCTTAGCTGCTAG
TCATGACCCCGCCATCATCCCACCATGTTGTGCTTGACCC
CtccatctatccatccatccatccTCCTGTCTATATGCCA
TCACTCCATCCTGTCTGTCTATCAATGATCTGGCATCTCC
GGATTTAGCACCAAGTCCCATTGATTAGTTGATCATTTGG
GAAACTTGGCTGTGCCGAGCCTGGTCTATTTGATGGATCG
GACCTCTCCACCTCTAATTCCCCCCCCGTCCTTCCCTTCT
CCGTCACTCATCCGCCATCCGCTCATTCTTTTTGGGTCCT
CGGTGAACACTCACCACCTTCCCCCCCGCTGCTGCTAGTG
CACACCGAAACAGCAAGTCCGGAGCGCTTACATTTTTGCC
TCCTCCGTTTTTTCCTATATCTGGGCCATGTTCTCCCTTT
GCCATCTTTGTTTTCTCCCAGTTTCACTGATCCATAACTC
ATCCCCCTCCAGTCCATCAACATG
SEQ ID NO: 142 is an exemplary nucleic acid
sequence encoding the construct of FIG. 20E.
GTTTAAACTTCGGAGTAGCAACGAGTATTTTCACCGGGAG
TTTCAACGGGTTCTATTTCAGGAACACGGCTGCGGTCTGG
ATTGGGTCGGGCTGAGATACCGACTGGTGGCGTCAGTGGC
GGGTACGGACGGAGTCGTCCTGGCCGCTCGTAGACACTCC
CCCGGACTGATATCAGGCCCCGGCAACTGGCTTCGTCTCA
CTCCAGGGCATCAGGAGTGCCTACCACATGGGTTCAGGCT
TTGCCCCGTCGTCTAAGTTTGCAGGACAAAATTTTCGTAT
GCGTTACCACTCTTTCCTTTCAGCAACCATTCCGTAGTGA
AAACCCAATAATAGGTGGCTGCCGTGGGAGCCTGAGTCAA
CCCAACCAGAACCTTTCTAGTAGATTCTCCCCCAAGCGCT
TCAGCAACGAAGCGTATTGGAGAACCAAATGACGCAGACC
AAGCGGATTCCGGTGCAATAGCCGGATGGCAAGGGAATCC
CCCAGGAGGTGCCAGAAGCGTCGCCCGAAAGGTACTTCGT
CTGACAGGCTAACACCGCTCGGGCTAAGGTCCCTGCTGCT
CTTTTCCCTTTATTGCGACTTAACCTCTAAGCCATTCCCT
TGCATCACGTTATCTCACTGACCGACCTCTGACTAAGGCG
CTTCGCCTCCGCCGCCTCCCCTCATTCACCTCCTCTCCTG
ACTACTTAAGCCTTCTCTTCCTTCCTCTCACTACCAACCC
TCCTTCATCCCTCATACCTCTCATCCTACCACTCACCTTT
CGCGCATCGCCATCTGCGATCCTCCCCACAACACTCCACC
TAGATACATACACCATTAACTGCGCTTCTAAACAATGCCC
GCCACCGGCGAGGACCAGGACCTGGTGCAGGACCTGATCG
AGGAACCCGCCACCTTCTCCGACGCCGTCCTGTCCTCCGA
CGAGGAACTGTTCCACCAGAAGTGCCCCAAGCCGGCTCCG
ATCTACAGCCCCGTCAGCAAGCCCGTCAGCTTCGAGTCCC
TGCCGAACCGCCGCCTGCACGAAGAGTTCCTCCGCTCCTC
CGTCGACGTCCTGCTGCAAGAGGCCGTGTTCGAGGGCACC
AACCGCAAGAACCGCGTCCTGCAGTGGCGCGAGCCCGAAG
AACTGCGCCGCCTGATGGACTTCGGCGTCCGCAGCGCCCC
GTCCACGCATGAGGAACTGCTCGAGGTCCTGAAGAAGGTC
GTCACCTACTCCGTCAAGACCGGCCATCCGTACTTCGTCA
ACCAGCTGTTCTCCGCCGTCGATCCCTACGGCCTGGTCGC
CCAGTGGGCCACCGACGCGCTGAACCCCTCCGTCTACACC
TACGAGGTCAGCCCCGTGTTCGTCCTGATGGAAGAGGTCG
TCCTGCGCGAGATGCGCGCCATCGTCGGCTTCGAAGGCGG
CAAAGGCGACGGCATCTTCTGCCCTGGCGGCTCGATCGCC
AACGGCTACGCCATCAGCTGCGCCCGCTACCGCTTCATGC
CCGACATCAAGAAGAAGGGCCTGCACTCCCTGCCGCGCCT
GGTCCTGTTCACCTCCGAGGACGCCCACTACTCGATCAAG
AAGCTGGCCTCGTTCCAAGGCATCGGCACCGACAACGTCT
ACCTGATCCGCACCGACGCTCGCGGTCGCATGGACGTCAG
CCACCTGGTCGAAGAGATCGAGCGCTCCCTCCGCGAGGGC
GCTGCCCCGTTCATGGTCAGCGCCACCGCCGGCACCACCG
TCATCGGCGCCTTCGATCCCATCGAGAAGATCGCCGACGT
CTGCCAGAAGTACAAGCTCTGGCTGCACGTCGACGCCGCC
TGGGGCGGAGGCGCTCTGGTGTCCGCCAAGCACCGCCATC
TGCTGAAGGGCATCGAGCGCGCCGACTCCGTCACCTGGAA
TCCCCACAAGCTGCTGACCGCTCCGCAGCAGTGCAGCACC
CTGCTGCTGCGCCACGAGGGCGTCCTGGCCGAGGCGCACT
CCACCAACGCCGCCTACCTGTTCCAGAAGGACAAGTTCTA
CGACACCAAGTACGACACCGGCGACAAGCACATCCAGTGC
GGCCGTCGCGCCGACGTGCTGAAGTTCTGGTTCATGTGGA
AGGCCAAGGGCACCTCCGGCCTCGAGAAGCACGTGGACAA
GGTGTTCGAGAACGCCCGCTTCTTCACCGACTGCATCAAG
AACCGTGAGGGCTTCGAGATGGTGATCGCCGAGCCTGAGT
ACACCAACATCTGTTTCTGGTACGTCCCCAAGAGCCTGCG
CGGACGCAAGGACGAGGCCGACTACAAGGACAAGCTGCAC
AAGGTCGCCCCTCGCATCAAAGAACGCATGATGAAGGAAG
GCTCCATGATGGTCACCTACCAGGCGCAGAAGGGCCATCC
GAATTTCTTCCGCATCGTCTTTCAGAACTCCGGCCTGGAC
AAGGCCGACATGGTCCATCTGGTCGAGGAAATCGAACGCC
TGGGCTCCGACCTCTGATGGGTTGGATGACGATGACTTCA
TGTGATTTTGTTATTTAGAATATTTTATATTTCCTTTTCT
TCTTCTCACCACCGATCCCCTTAACACTCTTGCTTCATTT
GCTTCAGATTTCTCGGTTTCTTCTTTTTTCTTCTCCCCAG
TTATCCACTATATCTTTGCTAGACCGGCCTGCGCCCTGGC
ATGCATCATAAAATCATGTCCGTTGGTCATCATCTGTTTT
GTATATCCGTCATATAAAGTATTCTTTTATTCCCTCCCCC
CTCGGTCGTCTTTCGCTGTCCCGCTTCCTACCTCCGGTTT
ATAGAGCATGGTTCATCTCTTCCGTACATTTCCGTTGGTA
CTAGCATTTATGTCTTCAGCTAGTATAGAAGCTGCCGCAG
TTGTTCGCTTACTACCTGCCTAAGTCCTTAACTTTTTAAA
GTGTTTAACCTATACGTAGTGTTAAACGAGTACTGGGAGG
TGGTGAGGTAGAAAATGTTCTGCACGGGCAGTGGGTATTT
GGTAGTGTGTAAGGCGGTTATTTATCAGGCTGACGCTAAA
GACTTCTATGGGAGCAGTATGGGATCGCGGCTCATAGAAG
TACACAAAATCTAAGAGTCGTTTGATAATTAATTGATTCC
CGGCAGGGTCTTCTTGGGATTGAGAGAACTGGTTACTTTG
ATTTGAGATATTGTAAAGCTTAAGGCTCTTAACACGTACG
AGCGAAACAGCAGGGGGGAAATCGGGAAAAGGGGCGTGGG
GTGAATAAAAAAGTTGAAATAAGACACTGTATCTTGCTGG
GGGTGAATAAAGAGAGAATAAAAGAGAGGTAAATTCCACT
CAGCCCCTTTTCTTCGCTCTCCAAACATCAAACTCCGCCG
GCCGACCCACAGGATCCCGAACAAGTGGAAGATATGTGCC
GGTCCAGACCCTTCGCACAGCTAAAAGCAGACCTTCATAA
GCGTTTCCGGGTAGTATTCGCACACCTGAACTGGCACGTC
GGGGACACAACTGTTTTTGATACACAAGAACACACACCAC
CCATCTAGGACTCAGAGCTGGGCCAGACATTCCTTCATAG
TCTTGACGATGAAGGTGAAGTCCTCTTCGGTGATGGACAG
CGGAGGGGCGAGCTGCAGGATGTTGTTGTAGCCGGCCACG
GTGTCGCCGTTCTTGCCGATGATCAGACCCTTCTCTTTGC
AGGCGTTGATGACCTTGTTCATCTTTTCGATGGAGGCGGG
CTCTTTGGTCTGCTTATCCTCGACGAGTTCGATACCCAGC
AGGAGGCCCTTGCCGCGGACGTCCCCGACGTTGGGGTGCT
CTTTGACGTCCTCCAACTCGTACAGCAGGCGCTCGCCCAG
TTCTTTGGACCGCTCGATGAGCTTCTCGTTTTCCATGATC
TCGAGGTTCTTCAGGGCCAGCGCGCAGGCGGCAGGGTTGC
CGCCGAAGGTGTTGACATGGCGGAAGCGGTCGTAGTCGTC
GGAGCCGACGAAGGCCTCGTAGACCTCGCGGCGGACCGCG
GTGGCAGACAGCGGCAGGTAGGCCGAGGTGATACCCTTGG
CCATGGTGATAATGTCGGGCTTGACGCCGTAGTTCATGAA
GCCGAAGGGCTTGCCGGTGCGACCGAAGCCGCAGATGACC
TCGTCGCAGATCAGCAGGGCGCCGTGCTTTTCGCAGATCT
CTTTGACCTTTTCCATGTAGCCGTCCGGCGGCATCAGGAT
GCCACCACCGGTGATGATGGGTTCCATGATGACGCCGGCG
ACGGTCTGGGACAGCTCCCAGGTCATGACGCGGTCGATTT
CTTCGGCGGAGGCCAGGGTGTGCACGTCCTCGGGGTTGCG
ATAGGTGTCCGGAGGGGCCACGTGCAGGAAGCCCTGACCG
AGGGGCTCGTACTTGTACTTGCGCTGGGCCTGACCGGTCG
CGGCCAGGGCACCCATGGAGTTGCCGTGGTAGGCGCGGTA
GCGAGAGATGAACTTGTAGCGGCCGTGGTCACCCTTCTGC
TGGTGGTACTGGCGGGCGATCTTGAAGGCGGTTTCGTTGG
CCTCCGAGCCGGAGTTGGAGAAGAAGATGACGTACTCGTC
GTCCAGCCACTCGTTCAGCTTCTCGGCCAGCTTGATGGCG
GGGACGTGCGACTGCGTCAGCGGGAAGTACGGCATCTCTT
CCAGCTGCTCGAAGGCAGCGCGAGCCAGCTCTTTGCGGCC
GTAGCCGACGTTGACGCACCACAGGCCGGACATGCCGTCC
AGGTAGCGGTTGCCGTCGATGTCGGTGACCCACGCGCCTT
CGGCCTTGGTGATGATCAGGTTGGTCGGACTCGGAGCGGC
ACCGCGCATGGCGTGCCACAGGTACTTCTCGTCGGTTTTC
TTCAGGCTCTGGGTCTGCTCGGTGACCTGGACGATCATCA
GTTCCATCTTGATGAAGGTCTGGGTTGGAAAAAGGATGTT
AGTTCATTGTTTTACTTGATACTGGTGCGTGAGCAAGGAT
AAAGTTCAATATTATCTGCGTCGCAGCTCTCATCTTCGTT
TCTTGTTCAACATATTACTCCCCCGGAATTTTGGTATCTC
GCGCGCTAAAGTGTGCGCAGGAGGTATTTTCTGGGATAAG
TGGAAAGAAATGAAGATGAGATGTTCCAAATAATATTGAA
TCCTTTTCCTTGTCACATAGTAAATGTATAGGGTCATCAA
CTCACCTCGACAAGACTTGTCTTCCTAAACCTTCGGCGAT
CGTGGTAGGAAAATATCCTCGGTGGTGGTGGGCTTCGTTA
AAATCGGTTGTGCGGTCACGTGGTGTCCAGATTACCGAGT
CAGGCGTAGTGCGGGTGGACACTCGGATAATCAGCCCTCC
GGGTCCTCAGTCATGTGATGGTCTGCGGCTGAGGTCACGG
GATTTGCTGACTTTAACGGTTTCTTGGATACGTCAGTTTG
GTGCCTGCATCGCCAGGCATAGGCACGGCCCGGAAAAGCC
AGGCAGAAAGTACGACTAGGCCTAAAACCAACTAGACGAA
GTGGTCAATCCAGCCTCTCAGCATCGACGTCAACTTCGTC
ATCGTCATCATTTGCCGCCCTCTCATCCCTCGGCTCGCTG
TTGCTAATTATTGATTGAATAGCATGCGGGATAGGCGTCA
ATTATTAAATCGGCTATTGCAGAGGAAGGGTGAAATAATG
TTTCAAACCCTCACAGGCCACAGGTCGCCTCCTCCCGTCA
GTTATAAGAGGCAATGTGGATAGATCGATAATGGAGATAG
GGTGGTTGTCTGGAGGTCGACGGTATCGATAATAACTTCG
TATAGCATACATTATACGAAGTTATGAACGACTCCAGAAG
TGACTAAGCAACCGTAAACCGGAAGCAACAATCCGGCTCT
CATGGCAAAGATGGCATGTCCGGATTGCGGAGGAAAGATT
TGGCGTGTGCCTGACGGTTTTGTTCCACAAGGACCCTGTA
AACTTGGTGATGGTAATGAGGTGCTCTGGCCTTGATTGAG
CAAAAGTCAGCTCGCGGGGACTACGTGAGTTGATGGTGGA
GGATCATGTACTAAATAGAGCATACCTAGGGGATGGTGAA
CGGACGTCCAGGGTTCCGTAGAATTGTAACAACTCTACTC
GACTTGCTGCCCACTGGGGTATGTAAATACATGCCTTTTC
AAAGTTGCCGAGTGTACTAAATGGAGTGTATGTTTGGCCA
ATGTCATTTGGGAGGGGCCGGGAACCCTTTGGGACACCCG
CGGATGGGCTGGATCTCGGTCACTGGAGACATAGCCTAGT
GGTCTAGTAGTCTAGCAGCAGGACAGCTTCATGGTTAACC
CCACATGTGATCCTCCGTCCGGTTTCCGTAGTATATAATG
GACCTGAAGCTTCTCCCTCTCTCGATGCGGTTGAAACATA
AATTTGCTCATAACGTATGTCCATCTAACCCTTTCATTGT
TTCTTGACCACTGTCTCCTTTCTGGAAAAAAAAAGACTCT
GGTAAGTATGGGCCTAGGATCGAGCTACTGATCCCCCAAT
CCTTCCTAATCCCTCTGCCACTTGCCCCGACCTGTTCACC
GTGACCGTACCACCACCACCCCGCCCCCTCCTCAACTAAA
CCCCTCCCTTCCCCTCCCCCCTGATGTTTTCTCTTTTCTT
TAATCTGACTGTCACCTCCCTCTCTACTTCTTTTCCCTCA
ATTTTCTTCTCCTCTTCTTACTCTCTGTCATCTGTTTCGA
TACCCATACTAATCTCGCGTGCTTTTTCTTCACCTTCGTG
TATAGATTTGTCACAATCCCAAATTTCACCATGATTGAAC
AAGATGGATTGCACGCAGGTTCTCCGGCCGCTTGGGTGGA
GAGGCTATTCGGCTATGACTGGGCACAACAGACAATCGGC
TGCTCTGATGCCGCCGTGTTCCGGCTGTCAGCGCAGGGGC
GCCCGGTTCTTTTTGTCAAGACCGACCTGTCCGGTGCCCT
GAATGAACTGCAGGACGAGGCAGCGCGGCTATCGTGGCTG
GCCACGACGGGCGTTCCTTGCGCAGCTGTGCTCGACGTTG
TCACTGAAGCGGGAAGGGACTGGCTGCTATTGGGCGAAGT
GCCGGGGCAGGATCTCCTGTCATCTCACCTTGCTCCTGCC
GAGAAAGTATCCATCATGGCTGATGCAATGCGGCGGCTGC
ATACGCTTGATCCGGCTACCTGCCCATTCGACCACCAAGC
GAAACATCGCATCGAGCGAGCACGTACTCGGATGGAAGCC
GGTCTTGTCGATCAGGATGATCTGGACGAAGAGCATCAGG
GGCTCGCGCCAGCCGAACTGTTCGCCAGGCTCAAGGCGCG
CATGCCCGACGGCGATGATCTCGTCGTGACCCATGGCGAT
GCCTGCTTGCCGAATATCATGGTGGAAAATGGCCGCTTTT
CTGGATTCATCGACTGTGGCCGGCTGGGTGTGGCGGACCG
CTATCAGGACATAGCGTTGGCTACCCGTGATATTGCTGAA
GAGCTTGGCGGCGAATGGGCTGACCGCTTCCTCGTGCTTT
ACGGTATCGCCGCTCCCGATTCGCAGCGCATCGCCTTCTA
TCGCCTTCTTGACGAGTTCTTCTGAGCGGGACTCTGGGGT
TCGAAATGACCGACCAAGCGACGATCCACTTAACGTTACT
GAAATCATCAAACAGCTTGACGAATCTGGATATAAGATCG
TTGGTGTCGATGTCAGCTCCGGAGTTGAGACAAATGGTGT
TCAGGATCTCGATAAGATACGTTCATTTGTCCAAGCAGCA
AAGAGTGCCTTCTAGTGATTTAATAGCTCCATGTCAACAA
GAATAAAACGCGTTTCGGGTTTACCTCTTCCAGATACAGC
TCATCTGCAATGCATTAATGCATTGGACCTCGCAACCCTA
GTACGCCCTTCAGGCTCCGGCGAAGCAGAAGAATAGCTTA
GCAGAGTCATAACTTCGTATAGCATACATTATACGAAGTT
ATTGCAGCCCGGGACATGCTGGAAGGGATTTTCTGGCTCA
ATACCACGTCTGTATTTGACCCTTTCCAGACAGTTAATCC
GCTGCAGGAGGGCGAACTGTAGCTCCTCGTTCTCCTTGTA
GCGCTTGATCCAGTCTTTTTGGATGTTGCACTTGCTTGGC
CTATGCTTCTCATATAATCTTGCCCTGTCATAGAGACGAC
GTCTGAGATTGTAGCGTTCGTCTTTGATCACCCGGAGCCA
GATAGGCCTGAGTATATCTGACATTAGATCAAAGGGTCTG
TGGATAGTCTCCTTCAGCATCAGCGACGCATGTGACTCGC
ATGTCGGAGAGAGCTTGTGGGTGGTCATCTTTGATGGCGT
CCTCTGCTTTCCCTTGATTTTCGTTGATTGTTTTTCGAAA
GTTAAGTCTGGAAGTCAAGAGAATCCTTCTGCCAGACATT
ATATTTACGTATACTGACGTAGTAGAAACAGCGTCAGGAT
GAGGACATGGTGTGTGCTGGACCACGGAATCATAGTTCAT
CAGTATATTGGGTTGGACAAATAACGCTGAGCATGTATAT
GTCTTTACACACTATAAAAGCCAGCGAACGCCAATAAAAT
AGGGCATATTGATGTGAAAATATGACACCAGTTAAAAGCA
GTGTATTGATTTTATCTCTCTTCACCTCGGACCTATACTA
CCGTATACAAGACTCAACTTACTTCCAGATATAGTAATAT
ACACCCTATGGACGAACCAGCACAATAATTACAGCCAAAC
AACACCACCCAAATGGCATATTCCTAATCAGCACTAAGCA
CAAATACCACTGTCATCACAGCATAATCAATAAGAATCCC
AGACAACCGACTCACTCTGACTCACCTTACACAAACCCCC
AAGCAAAGCGCAGCCCAGAACCTCAGCCAACAATCGGGCA
ACGTACGGGGAAAGATTGGCCGATCCATGATGTCAGCAGC
CCTAACCCAAAGCGGACTAGCGCATACCGCCCCTCTGACT
CCGCCATCCCAGGGCTCGAGAAGCTTCCGTGGCGTCGATA
TAAATTCAGCGGGCCTTGAACATCCCTCCTTACGACACAC
CTCACGCGATCGATTTTGACACTCACACACCGCCACCCTC
ACATCCTCCACCCACACCACACCCCTTAATCAACCCACCA
TCACCGCTAGAACGTCTATCTCATCACCGACTTCTCATCC
ATCTTCAAAATGATCGTGCTGGTCACGGGCGCGACCGCCG
GTTTCGGCGAGTGCATCACCCGCCGCTTCATCCAGCAGGG
CCACAAGGTGATCGCTACCGGACGCCGCCAAGAGCGCCTC
CAAGAGCTGAAGGATGAGCTGGGCGACAACCTGTACATTG
CCCAGCTGGACGTGCGCAACCGGGCTGCCATCGAAGAAAT
GCTCGCCTCGCTGCCCGCCGAGTGGTGCAACATCGACATC
CTGGTCAACAACGCCGGTCTGGCCCTCGGCATGGAACCGG
CGCACAAGGCCAGCGTCGAGGACTGGGAAACCATGATCGA
CACCAACAACAAGGGACTCGTCTACATGACCCGCGCTGTG
CTGCCCGGCATGGTCGAGCGCAACCACGGCCACATCATCA
ACATCGGCTCCACCGCTGGCAGCTGGCCCTACGCTGGCGG
CAACGTCTATGGCGCGACCAAGGCGTTCGTCCGCCAGTTC
TCCCTGAACCTGCGCACCGACCTGCACGGCACCGCCGTCC
GCGTGACCGACATTGAGCCCGGTCTGGTCGGCGGCACCGA
GTTCAGCAACGTCCGCTTCAAGGGCGACGACGGCAAGGCC
GAGAAAACCTACCAGAACACCGTCGCTCTGACCCCTGAGG
ATGTCAGCGAGGCCGTCTGGTGGGTCAGCACTCTGCCCGC
GCACGTCAACATCAACACCCTCGAGATGATGCCCGTCACG
CAGTCCTACGCCGGCCTGAACGTCCACCGCCAATAGGACC
GATGGCTGTGTAGAAGTACTCGCCGATAGTGGAAACCGAC
GCCCCAGCACTCGTCCGAGGGCAAAGGAATAGAGTAGATG
CCGACCGGGATCCACTTAACGTTACTGAAATCATCAAACA
GCTTGACGAATCTGGATATAAGATCGTTGGTGTCGATGTC
AGCTCCGGAGTTGAGACAAATGGTGTTCAGGATCTCGATA
AGATACGTTCATTTGTCCAAGCAGCAAAGAGTGCCTTCTA
GTGATTTAATAGCTCCATGTCAACAAGAATAAAACGCGTT
TCGGGTTTACCTCTTCCAGATACAGCTCATCTGCAATGCA
TTAATGCATTGGACCTCGCAACCCTAGTACGCCCTTCAGG
CTCCGGCGAAGCAGAAGAATAGCTTAGCAGAGTCTATTTT
CATTTTCGGGAGACGAGATCAAGCAGATCAACGGTCGTCA
AGAGACCTACGAGACTGAGGAATCCGCTCTCTGACAGACG
GGCAATTGATTACGGGATCCCATTGGTTTAAAC

### DETAILED DESCRIPTION

### I. Abbreviations

3-HP: 3-hydroxypropionic acid
aat1: aspartate aminotransferase
ald3: malonate semialdehyde dehydrogenase 3
ald6: malonate semialdehyde dehydrogenase 6
BAPAT: β-alanine-pyruvate aminotransferase
cad1: cis-aconitate decarboxylase
CAP: citric acid production medium
DDR-EH: disk refining and enzymatic hydrolysis
CM: complete medium
DBTL: design-build-test-learn
elf3: elongation factor 3
eno1: alpha-enolase
gpdA: glyceraldehyde-3-phosphate dehydrogenase
HPDH (3-HPDH): 3-hydroxypropionate dehydrogenase
hph: hygromycin B phosphotransferase marker gene
KOH: potassium hydroxide
mct1: monocarboxylate transporter
MM: minimal medium
mRDM: modified production medium B
nat1: nourseothricin N-acetyl transferase
NaOH: sodium hydroxide
NH₃: ammonia
nptII: bacterial neomycin-resistance (nptII) marker gene
oahA: oxaloacetate hydrolase
PAND: aspartate 1-decarboxylase
PDA: potato dextrose agar
pyc: pyruvate carboxylase
RDM: production medium B
TE: trace elements
trpC: tryptophan C gene
uga2: succinate semialdehyde dehydrogenase

### II. Summary of Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “a gene” includes singular or plural genes and can be considered equivalent to the phrase “at least one gene.” As used herein, the term “comprises” means “includes.” Unless context clearly indicates otherwise, “about” refers to plus or minus 5% of a reference value. For example, “about” 100 refers to 95 to 105. It is further to be understood that any and all base sizes or amino acid sizes, and all molecular weight or molecular mass values, given for nucleic acids or polypeptides are approximate, and are provided for descriptive purposes, unless otherwise indicated. The sequences associated with the GenBank Accession numbers provided herein are incorporated herein by reference in their entireties (specifically the sequences available on Aug. 3, 2023).

Although many methods and materials similar or equivalent to those described herein can be used, particular suitable methods and materials are described herein. The materials, methods, and examples are illustrative only and not intended to be limiting. In case of conflict, the present specification, including explanations of terms, will control. To facilitate review of the disclosure, the following explanations of terms are provided:

3-HP β-alanine pathway: An artificial pathway for the production of 3-HP in fungi that includes aspartate decarboxylase (PAND), β-alanine pyruvate transaminase (BAPAT), and 3-HP dehydrogenase (HPDH) (see, e.g., Borodina et al. “Establishing a synthetic pathway for high-level production of 3-hydroxypropionic acid in Saccharomyces cerevisiae via β-alanine,” Metab Eng. 27:57-64, 2015). In some examples, the PAND gene is from Tribolium castaneum. In some examples, the BAPAT gene is from Bacillus cereus. In some examples, the HPDH is from Escherichia coli. In some examples, one or more genes of the 3-HP β-alanine pathway are codon optimized for expression in a fungus, for example, in an Aspergillus species (e.g., Aspergillus pseudoterreus).

3-hydroxypropionic acid (3-HP or 3HP): A 3-hydroxy monocarboxylic acid that is propionic acid in which one of the hydrogens attached to the terminal carbon is replaced by a hydroxy group (see, PubChem CID 68152). It can be used in the production of chemicals, such as acrylic acid, malonic acid, acrylamide, 1,3-propanediol, and biodegradable polymers.

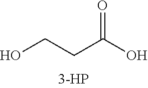

3-hydroxypropionate dehydrogenase (HPDH): An enzyme that catalyzes the reduction of 3-oxopropanoate to 3-hydroxypropanoate:

3-oxopropanoate+NADH+H⁺⇄3-hydroxypropanoate+NAD⁺

Expression of HPDH in a recombinant Aspergillus niger also expressing BAPAT and PAND (and in some examples also having a genetically inactivated ald6a gene and/or over-expressed pyc gene) results in a recombinant A. niger capable of producing 3-HP at higher titers.

An exemplary HPDH amino acid sequence is provided in SEQ ID NO: 3, however, additional HPDH sequences are also publicly available. For example, GenBank® Accession No: WP_000636571 discloses a HPDH protein sequence; GenBank® Accession Nos. FR729477.2 (nt 1005136 . . . 1005885) and CBY27203.1 disclose exemplary Yersinia enterocolitica subsp. palearctica Y11 HPDH nucleic acid and protein sequences, respectively; and GenBank® Accession Nos: CP004083.1 (complement(1399227 . . . 1399973)) and AJQ99264.1 disclose exemplary Enterobacteriaceae bacterium bta3-1 HPDH nucleic acid and protein sequences, respectively. In some examples, a nucleic acid sequence encoding HPDH is codon optimized for expression in Aspergillus (see, e.g., SEQ ID NO: 132). A practitioner will appreciate that HPDH sequences include variant sequences (such as allelic variants or homologs) that retain HPDH activity when expressed in Aspergillus niger.

Aspartate 1-decarboxylase (PAND): An enzyme that catalyzes the formation of β-alanine from aspartic acid in the following chemical reaction:

L-aspartate+H⁺⇄β-alanine+CO₂

Expression of PAND in a recombinant Aspergillus niger also expressing BAPAT and HPDH (and in some examples also having a genetically inactivated ald6a gene and/or over-expressed pyc gene) results in a recombinant A. niger capable of producing 3-HP in high titers.

An exemplary PAND amino acid sequence is provided in SEQ ID NO: 1, however, additional PAND sequences are also publicly available. For example, GenBank® Accession Nos: NM_001102585.1 and NP_001096055.1 disclose Tribolium castaneum PAND nucleic acid and protein sequences, respectively; GenBank® Accession Nos. CP002745.1 (complement(4249351 . . . 4249824)) and AEK63458.1 disclose exemplary Collimonas fungivorans Ter331 PAND nucleic acid and protein sequences, respectively; and GenBank® Accession Nos: CP029034.1 (nt 1201611 . . . 1201994) and AWE15802.1 disclose exemplary Bacillus velezensis PAND nucleic acid and protein sequences, respectively. In some examples, a nucleic acid sequence encoding PAND is codon optimized for expression in Aspergillus (see, e.g., SEQ ID NO: 130). A practitioner will appreciate that PAND sequences include variant sequences (such as allelic variants or homologs) that retain PAND activity when expressed in Aspergillus niger.

β-alanine-pyruvate aminotransferase (BAPAT): An enzyme that catalyzes the following chemical reaction:

L-alanine+3-oxopropanoate⇄β-alanine+pyruvate

Expression of BAPAT in a recombinant Aspergillus niger also expressing PAND and HPDH (and in some examples also having a genetically inactivated ald6a gene and/or over-expressed pyc gene) results in a recombinant A. niger capable of producing 3-HP in high titers.

An exemplary BAPAT amino acid sequence is provided in SEQ ID NO: 2, however additional BAPAT sequences are also publicly available. For example, GenBank® Accession Nos: ACMS01000158.1 (complement(10606 . . . 11961)) and EEL86940.1 disclose Bacillus cereus AH1272 BAPAT nucleic acid and protein sequences, respectively; GenBank® Accession Nos. DF820429.1 (complement (241627 . . . 242967)) and GAK28710.1 disclose exemplary Serratia liquefaciens FK01 BAPAT nucleic acid and protein sequences, respectively; and GenBank Accession Nos: LGUJ01000001.1 complement (92812 . . . 94140) and KOY12524.1 disclose exemplary Bradyrhizobium diazoefficiens BAPAT nucleic acid and protein sequences, respectively. In some examples, a nucleic acid sequence encoding BAPAT is codon optimized for expression in Aspergillus (see, e.g., SEQ ID NO: 131). A practitioner will appreciate that BAPAT sequences include variant sequences (such as allelic variants or homologs) that retain BAPAT activity when expressed in Aspergillus niger.

Control: A reference standard. A control can be a positive or negative control. In some examples, the control is a historical control or standard reference value or range of values (such as a previously tested control sample or a group of samples that represent baseline or normal values). In some examples, the control is wild-type (unmodified) A. niger. In some examples, the control is A. niger expressing the 3-HP β-alanine pathway (absent further modifications, such as an exogenous nucleic acid encoding pyc and/or mct1).

A difference between a test sample and a control can be an increase or conversely a decrease. In some examples, the difference is statistically significant. In some examples, a difference is an increase relative to a control, for example, an increase of at least 5%, at least 10%, at least 20%, at least 30%, at least 40%, at least 50%, at least 60%, at least 70%, at least 80%, at least 90%, at least 100%, at least 150%, at least 200%, at least 250%, at least 300%, at least 350%, at least 400%, or at least 500%. In other examples, a difference is a decrease relative to a control, for example, a decrease of at least 5%, at least 10%, at least 20%, at least 30%, at least 40%, at least 50%, at least 60%, at least 70%, at least 80%, at least 90%, at least 95%, at least 98%, or at least 99%.

Copy number: The number of copies of a particular gene in a particular genotype, for example, the number of copies of a gene (e.g., PAND, BAPAT, HPDH, pyc, and/or mct1) in a recombinant A. niger strain.

Detectable: Capable of having an existence or presence ascertained.

Exogenous: Originating from a different source. For example, a nucleic acid molecule that is exogenous to an organism (e.g., Aspergillus niger) is a nucleic acid that originated from a source other than the organism itself (e.g., a synthetic nucleic acid introduced into the Aspergillus niger). A cell including an exogenous nucleic acid is not, or excludes, any naturally occurring cell or organism. In some examples, the PAND, BAPAT, and/or HPDH nucleic acid or protein does not naturally occur in Aspergillus niger, and therefore are exogenous to Aspergillus niger. For example, PAND, BAPAT, and HPDH nucleic acid molecules introduced into an Aspergillus niger can be from another organism, such as a bacterial PAND, BAPAT, and HPDH sequence.

Expression cassette: A nucleic acid fragment designed for expression of a particular gene (or genes) in a host cell (e.g., A. niger). Expression cassettes can be included in a vector. An expression cassette can include regulatory elements, such as promoters and/or terminators.

Genetic inactivation or down-regulation: When used in reference to the expression of a nucleic acid molecule, such as a gene, gene inactivation refers to any process which results in a decrease in production of a gene product. A gene product can be RNA (such as mRNA, rRNA, tRNA, and structural RNA) or protein. Therefore, gene down-regulation or deactivation includes processes that decrease transcription of a gene or translation of mRNA. A mutation, such as a substitution, partial or complete deletion, insertion, or other variation, can be introduced into a gene sequence to significantly reduce (and in some cases eliminate) production of a gene product or renders the gene product substantially or completely non-functional.

For example, a genetic inactivation of an endogenous ald6a, ald6b, ald3, and/or oahA gene in Aspergillus niger results in the Aspergillus having non-functional or non-detectable ald6a, ald6b, ald3, and/or oahA protein, respectively. In some examples, genetic inactivation of an endogenous gene in Aspergillus niger (including a recombinant Aspergillus niger disclosed herein) increases production of 3-HP by the Aspergillus niger. Genetic inactivation is also referred to herein as a “functional deletion.”

Increase or decrease: A positive (increase) or negative (decrease) difference relative to a reference value, such as a control. The difference can be a qualitative or quantitative. In some examples, the difference is statistically significant (e.g., P-Value less than 0.05 or 0.01). In some examples, the difference is an increase relative to a control of at least 5%, such as at least 10%, at least 20%, at least 30%, at least 40%, at least 50%, at least 60%, at least 70%, at least 80%, at least 90%, at least 100%, at least 150%, at least 200%, at least 250%, at least 300%, at least 350%, at least 400%, at least 500%, or greater than 500%. In some examples, the difference is a decrease relative to a control of at least 5%, such as at least 10%, at least 20%, at least 30%, at least 40%, at least 50%, at least 60%, at least 70%, at least 80%, at least 90%, at least 95%, at least 98%, or 100%.

Isolated or purified: An “isolated” or “purified” biological component (such as a nucleic acid, protein, or cell) is one that has been substantially separated from other biological components in the environment in which the component occurs, e.g., separated from other chromosomal and extra-chromosomal DNA and RNA, proteins and/or cells. Nucleic acids and proteins that have been “isolated” include nucleic acids and proteins purified by standard purification methods. The term also embraces nucleic acids and proteins prepared by recombinant expression in a host cell as well as chemically synthesized nucleic acids. An “isolated” microorganism (such as a recombinant Aspergillus niger) has been substantially separated or purified away from microorganisms of different types, strains, or species. Microorganisms can be isolated by a variety of techniques, such as serial culturing and/or using selection markers conferring resistance to certain chemicals, such as antibiotics.

Absolute purity or isolation is not required, it is intended as a relative term. Thus, for example, a purified/isolated protein, nucleic acid, or cell preparation is one in which the protein, nucleic acid, or cell is more enriched than the protein, nucleic acid, or cell is in its initial environment. In one example, a preparation is purified/isolated such that the protein, nucleic acid, or cell represents at least 50% of the total content of the preparation, such as at least 60%, at least 70%, at least 80%, at least 90%, at least 95%, at least 98%, or at least 99% pure. In some examples, an isolated, recombinant Aspergillus niger strain is at least 90% (for example, at least 95%, as at least 98%, at least 99%, or at least 99.99%) pure.

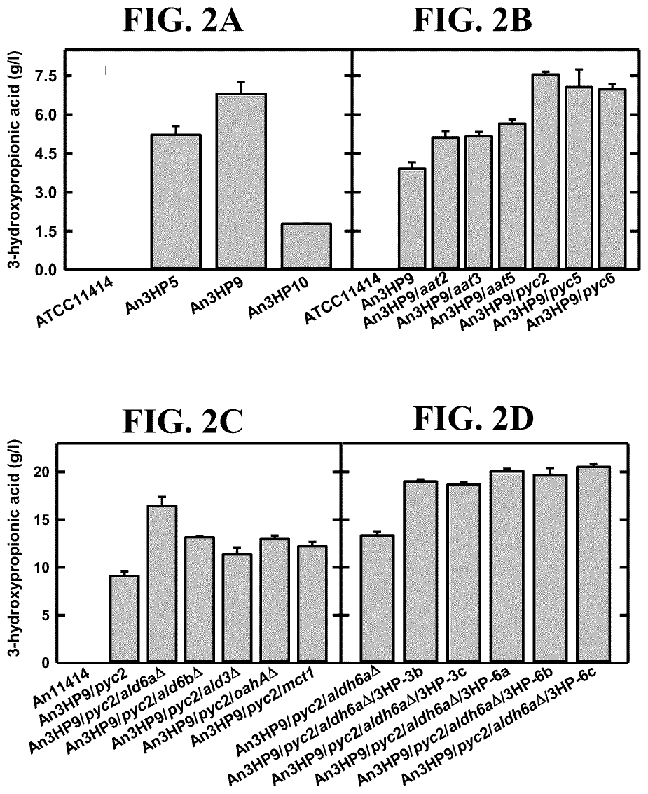

Malonate semialdehyde dehydrogenase (ald): An enzyme identified as potentially involved in metabolism of the 3-HP pathway (see, e.g., Pomraning et al., Front Bioeng Biotechnol 9:603832, 2021). Multiple malonate semialdehyde dehydrogenase homologs were identified in A. niger, including ald6a (Anald6a), ald6b (Anald6b), and ald3 (Anald3). The results disclosed herein show that while Anald6a is likely the major malonate semialdehyde dehydrogenase, all three (Anald6a, Anald6b, and Anald3) contribute to directing flux (precursor) away from 3-HP in Aspergillus niger (see, e.g., FIG. 2C). An exemplary genomic nucleic acid sequence of A. niger ald6a is provided as SEQ ID NO: 6. An exemplary genomic nucleic acid sequence of A. niger ald6b is provided as SEQ ID NO: 7. An exemplary genomic nucleic acid sequence of A. niger ald3 is provided as SEQ ID NO: 8.

Monocarboxylate transporter 1 (mct1): A monocarboxylate transporter and putative 3-HP transporter. An exemplary nucleic acid sequence encoding mct1 from A. niger is provided as SEQ ID NO: 129. An exemplary amino acid sequence of mct1 from A. niger is provided as SEQ ID NO: 137. A practitioner will appreciate that functional mct1 sequences include variant sequences (such as allelic variants or homologs) that retain mct1 activity when expressed in Aspergillus niger.

Microaerobic or oxygen limited condition: When oxygen demand of an organism is greater than the oxygen transfer rate from the gaseous to aqueous phase. In some examples, a dissolved oxygen content near zero (0-10% DO) in a reactor (e.g., an aerated reactor) is a microaerobic (oxygen limited) condition.

Mutation: A change in a nucleic acid sequence (such as a gene sequence) or amino acid sequence, for example as compared to a reference sequence (e.g., a wild-type or native sequence). In particular examples, a mutation is introduced into an endogenous ald6a, ald6b, ald3, and/or oahA gene in a recombinant Aspergillus niger, thereby rendering the gene(s) non-functional. Mutations can be introduced, for example using standard molecular biology methods. In particular examples, a mutation includes one or more nucleotide substitutions, deletions, insertions, or combinations thereof.

Oxaloacetate hydrolase (oahA): An enzyme involved in the production of oxalate in fungi. An exemplary nucleic acid sequence encoding oahA from A. niger is provided as SEQ ID NO: 9.

Operably linked: A nucleic acid sequence is “operably linked” when it is placed in a functional relationship with a second nucleic acid sequence. For instance, a promoter is operably linked to a coding sequence if the promoter affects the transcription or expression of the coding sequence. Generally, operably linked DNA sequences are contiguous and, where necessary to join two protein-coding regions, are in the same reading frame.

Overexpressed: An organism can be modified to overexpress a gene or gene product. An overexpressed gene or gene product is one that has increased expression/accumulation relative to the expression/accumulation of the gene or gene product in the organism prior to overexpression of the gene. For example, overexpression of pyc in Aspergillus niger results in increased expression/accumulation of pyc in the Aspergillus niger relative to the expression/accumulation of pyc prior to overexpression.

Promoter: A nucleic acid control sequence that directs transcription of a nucleic acid. A promoter includes necessary nucleic acid sequences near the start site of transcription. A promoter also optionally includes distal enhancer or repressor elements. A “constitutive promoter” is a promoter that is continuously active and is not subject to regulation by external signals or molecules. Constitutive promoters are often used to overexpress a nucleic acid in a host. In contrast, the activity of an “inducible promoter” is regulated by an external signal or molecule (for example, a transcription factor). Native and non-native promoters (i.e., endogenous and exogenous) can be used to drive expression of a gene, such as PAND, BAPAT, HPDH, pyc, and/or MCTL.

Exemplary promoters that can be used include but are not limited to: SV40 promoter, the CMV enhancer-promoter, the CMV enhancer/β-actin promoter, trpC promoter (trpCp), translation elongation factor 1 gene promoter (tef1p), mbfA promoter (mbfAp), ubi4 gene promoter (ubi4p), ubi1S27 promoter (ubi1S27p or ubiS27p), glyceraldehyde-3-phosphate dehydrogenase promoter (gpdAp), and enolase promoter (eno1p). In some examples, the promoter is an Aspergillus niger promoter (e.g., Aspergillus niger gpdAp, eno1p, teflp, ubi4p, ubi1S27p or mbfAp). Both constitutive and inducible promoters can be used in the fungi and methods provided herein (see e.g., Bitter et al., Methods in Enzymology 153:516-544, 1987). Also included are promoter elements which are sufficient to render promoter-dependent gene expression controllable for cell-type specific, tissue-specific, or inducible by external signals or agents; such elements may be located in the 5′ or 3′ regions of the gene. Promoters produced by recombinant DNA or synthetic techniques can also be used to provide for transcription of the nucleic acid sequences.

Pyruvate carboxylase (pyc): An enzyme that catalyzes the conversion of pyruvate to oxaloacetate. Typically, yeast have two pyruvate carboxylase genes, pyc1 and pyc2, however, Aspergillus niger only has one pyruvate carboxylase gene, which is referred to as “pyc.” Reference to “pyc2” as used herein a strain name (e.g., An3HP9/pyc2/ald6aD/3HP-6) refers to a particular transgenic strain expressing A. niger pyc (pyc strain 2). An exemplary amino acid sequence of Aspergillus niger pyc is provided as SEQ ID NO: 5. An exemplary nucleic acid sequence encoding Aspergillus niger pyc is provided as SEQ ID NO: 133. A practitioner will appreciate that functional pyc sequences include variant sequences (such as allelic variants or homologs) that retain pyc activity when expressed in Aspergillus niger.

Recombinant: A nucleic acid or protein that has a sequence made by an artificial combination of two otherwise separated segments of sequence (e.g., a “chimeric” sequence). This artificial combination can be accomplished by chemical synthesis or by manipulation of isolated segments of nucleic acids, for example, by standard molecular biology techniques (e.g., cloning; see, Sambrook et al. (ed.), Molecular Cloning: A Laboratory Manual, 3d ed., vol. 1-3, Cold Spring Harbor Laboratory Press, Cold Spring Harbor, N Y, 2001). A recombinant or transformed organism or cell, such as a recombinant Aspergillus niger, is one that includes at least one exogenous nucleic acid molecule, such as an exogenous nucleic acid molecule encoding one or more of PAND, BAPAT, HPDH, pyc, and/or MCT1.

Regulatory element: A term that includes promoters, enhancers, internal ribosomal entry sites (IRES), and other expression control elements (e.g., transcription termination signals, such as polyadenylation signals and poly-U sequences). Regulatory elements include those that direct constitutive expression of a nucleotide sequence in many types of host cells and those that direct expression of the nucleotide sequence only in certain host cells (e.g., tissue-specific regulatory sequences). Regulatory elements may also direct expression in a temporal-dependent manner, such as in a cell-cycle dependent or developmental stage-dependent manner, which may or may not also be tissue or cell-type specific.

Sequence identity/similarity: The degree of similarity between amino acid or nucleic acid sequences. Sequence identity is frequently measured in terms of percentage identity (or percent identity); the higher the percentage, the more similar the two sequences are. Homologs of a polypeptide (or nucleotide sequence) will possess a relatively high degree of sequence identity when aligned using standard methods.

Methods of alignment of sequences for comparison have been described. The NCBI Basic Local Alignment Search Tool (BLAST) tool is often used and is available from several sources, including the National Center for Biotechnology Information (blast.ncbi.nlm.nih.gov/Blast.cgi). Various types of BLAST are available, for example, blastp, blastn, blastx, tblastn and tblastx. BLASTN is used to compare nucleic acid sequences, while BLASTP is used to compare amino acid sequences. To compare two nucleic acid sequences, the options can be set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to −1; -r is set to 2; and all other options are left at their default setting. For example, the following command can be used to generate an output file containing a comparison between two sequences: C:\B12seq -i c:\seq1.txt -j c:\seq2.txt -p blastn -o c:\output.txt -q -1 -r 2. In some examples, percent sequence identity is determined by using BLAST with default parameters. Further description of how to determine sequence identity using various blast programs is available on the NCBI website and other resources.

Other various programs and alignment algorithms are also described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (e.g., 100 consecutive nucleotides or amino acid residues from a sequence set forth in an identified sequence), followed by multiplying the resulting value by 100. For example, a nucleic acid sequence that has 1166 matches when aligned with a test sequence having 1554 nucleotides is 75.0 percent identical to the test sequence (i.e., 1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (i.e., 15÷20*100=75).

For comparisons of amino acid sequences of greater than about 30 amino acids, the Blast 2 sequences function is employed using the default BLOSUM62 matrix set to default parameters, (gap existence cost of 11, and a per residue gap cost of 1). Homologs are typically characterized by possession of at least 70% sequence identity counted over the full-length alignment with an amino acid sequence using the NCBI Basic Blast 2.0, gapped blastp with databases such as the nr or swissprot database. Queries searched with the blastn program are filtered with DUST (Hancock and Armstrong, 1994, Comput. Appl. Biosci. 10:67-70). Other programs use SEG. In addition, a manual alignment can be performed. Proteins with even greater similarity will show increasing percentage identities when assessed by this method, such as at least 75%, 80%, 85%, 90%, 95%, or 99% sequence identity.

Nucleic acid sequences that do not show a high degree of identity may nevertheless encode identical or similar (conserved) amino acid sequences, due to the degeneracy of the genetic code. Changes in a nucleic acid sequence can be made using this degeneracy to produce multiple nucleic acid molecules that all encode substantially the same protein. Such homologous nucleic acid sequences can, for example, possess at least 60%, 70%, 80%, 90%, 95%, 98%, or 99% sequence identity determined by this method.

Sequence identity ranges are provided for guidance only; it is possible that strongly significant homologs could be obtained that fall outside the ranges provided. Thus, a variant PAND, BAPAT, HPDH, or other protein or nucleic acid molecule disclosed herein can have at least 80%, at least 85%, at least 90%, at least 91%, at least 92%, at least 93%, at least 94%, at least 95%, at least 96%, at least 97%, at least 98% or at least 99% sequence identity to the SEQ ID NOs: and/or GenBank® Accession Nos. provided herein.

Transformed: A cell, such as a fungal cell, into which a nucleic acid molecule has been introduced, for example by molecular biology methods. As used herein, the term transformation encompasses all techniques by which a nucleic acid molecule might be introduced into such a cell, including, but not limited to chemical methods (e.g., calcium-polyethylene glycol transfection), physical methods (e.g., electroporation, microinjection, particle bombardment), fusion (e.g., liposomes), receptor-mediated endocytosis (e.g., DNA-protein complexes, viral envelope/capsid-DNA complexes), by biological infection by recombinant Agrobacterium tumefaciens or viruses, such as recombinant viruses, etc. In one example, a protoplast transformation provided herein is used, such as in Example 1.

Variant: A nucleic acid or protein that varies in sequence, but retains a desired function. A degenerate variant refers to a nucleic acid sequence variant due to degeneracy (redundancy) of the genetic code. Thus, while the nucleic acid sequence of degenerate variants differ, they encode the same amino acid sequence.

Vector: A nucleic acid molecule that can be introduced into a host cell (for example, by transformation), thereby producing a transformed host cell. Recombinant DNA vectors are vectors having recombinant DNA. A vector may include nucleic acid sequences that permit it to replicate in the host cell, such as an origin of replication. A vector can include a promoter, and/or selectable marker genes, and other genetic elements. A vector can include one or more genes (e.g., PAND, BAPAT, HPDH, pyc, and/or MCT1) and/or a sequence used to genetically inactivate a gene (e.g., ald6a, ald6b, ald3, and/or oahA). A vector can transduce, transform or infect a cell, thereby causing the cell to express nucleic acids and/or proteins. A vector optionally includes materials to aid in achieving entry of the nucleic acid into the cell, such as a viral particle, liposome, protein coating or the like. In one example, a vector is a plasmid, such as a plasmid exogenous to the cell or organism into which it is introduced.

### II. Overview

The biosynthesis of 3-HP as a secreted monomer or an intracellular polymer has been examined in a variety of microorganisms and from various feedstocks. Production of monomeric 3-HP as a precursor for commodity chemical production has been proposed and developed primarily using sugars or glycerol as feedstocks, both of which have the potential to offer economic and green-house gas emission benefits compared to petroleum derived feedstocks (Bhagwat et al., ACS Sustain Chem Eng 9(49):16659-69, 2021). Production of 3-HP from glycerol as a feedstock using bacterial hosts has made substantial gains toward economic viability (Chen et al., PloS one 12(9):e0185734, 2017; Wu et al., PloS one 14(9):e0222143, 2019), however, high-yield production from sugar feedstocks has been more challenging (Ji et al., Front Microbiol 9:2185, 2018) with only modest yields achieved in hosts that require expensive nutrient supplements and are limited to production of 3-hydroxypropionate near neutral pH production conditions rather than 3-hydroxypropionic acid. Therefore, engineering the β-alanine pathway in the filamentous fungal hosts A. pseudoterreus and A. niger is an attractive option. The production can be scaled for industrial production of organic acids at acidic pH (<2.0) and has the capability of converting mixed sugar feedstocks with minimal nutrients and without pH neutralization requirement. After several DBTL (design-build-test-learn) cycles and culture optimizations, the 3-HP production in the best A. niger transgenic strain reached 36.0 g/l with the DDR-EH derived sugars from corn stover.

In the first design-build-test-learn (DBTL) cycle, the β-alanine pathway was functionally expressed in the filamentous fungus A. pseudoterreus and genes related to 3-HP degradation enzymes (Apald6, Apald3, Apuga2) and monocarboxylate transport across the plasma membrane (Apmct1) were identified via transcriptomic and proteomic analyses (Pomraning et al., Front Bioeng Biotechnol 9:603832, 2021). Next, the process was improved by the transfer of the β-alanine pathway into the A. niger, citric acid production strain (Perlman et al., Arch Biochem Biophys 11:123-9, 1946). The same transgene expression cassette used in A. pseudoterreus was randomly chromosomally integrated into A. niger and conferred a higher initial titer of 3-HP. Analysis of both species revealed that increased 3-HP yield correlates to copy number and expression level of the β-alanine pathway, which may explain the higher initial titers in the A. niger strains. Production of other organic acids, such as citric acid was below the detection limit in A. niger allowing more carbon to be routed toward 3-HP production.

The effects of increasing the intracellular pool of precursor metabolites such as oxaloacetate and aspartic acid on 3-HP production was first evaluated by increasing the expression of aat1, or pyc in the An3HP9 transgenic strain with about 30 to 90% improvements in 3-HP titer in the selected transgenic strains. Elimination of carbon fluxes competing with β-alanine pathway metabolic intermediates oxaloacetate, malonic semialdehyde, and glutamate/α-ketoglutarate was also examined in transgenic strain An3HP9/pyc2. All exhibited positive effects on 3-HP production and 83% improvement in titer of 3-HP production was observed with the disruption of ald6a gene. Previously significant improvements in flux toward 3-HP in A. pseudoterreus were observed after disruption of Apald6 (Pomraning et al., Front Bioeng Biotechnol 9:603832, 2021) that are consistent with results from the homolog in Candida albicans (Otzen et al., J Biol Chem 289(12):8151-69, 2014). This highlights that while ald6a may be the major contributor to the pathway competition, A. niger encodes at least three enzymes capable of metabolizing the 3-HP precursor malonic semialdehyde that may need to be simultaneously disrupted to maximize yield of 3-HP.

To test for the possibility of reactions limiting the final steps in conversion of pyruvate to 3-HP, the expression level of the heterologous enzymes was increased in the β-alanine pathway in the An3HP9/pyc2/ald6aA strain by increasing the chromosomally integrated copy-number of the pathway from 12 to 27. MCT1, a putative 3-HP transporter (Pomraning et al., Front Bioeng Biotechnol 9:603832, 2021), was overexpressed to test whether export may be limiting. In both cases, increased expression improved the yield of 3-HP suggesting that expression level of enzymes for 3-HP production is still limiting in the engineered strains and highlighting the need for novel metabolic engineering tools in Aspergillus species capable of increasing expression level of enzymes orders of magnitude beyond what is currently possible.

In addition to modifications of pathway enzymes in the transgenic strains, culture conditions play a role in directing metabolic flux toward 3-HP. The initial pH in A. niger cultures for citric acid production is 2.0 and manganese is limited to 10 ppb (Dai et al., Appl Environ Microbiol 70(4):2474-85, 2004). Here, an initial pH ranges from 3.4 to 2 did not significantly affect 3-HP yield in the An3HP9 strain though the spore germination frequency is higher at pH 3.4. 3-HP is produced in CAP medium which supports overflow metabolism, but to a lesser extent than in the mRDM medium. Therefore, the effects of the mRDM medium on 3-HP production was further optimized by alternation of individual components. Maintenance of manganese at a very low concentration (˜10 ppb) is required to support high-yield production of citric acid in A. niger (Dai et al., Appl Environ Microbiol 70(4):2474-85, 2004; Karaffa et al., Microorganisms 9(6):1267, 2021) as well as itaconic acid in A. pseudoterreus (Karaffa et al., Appl Microbiol Biotechnol 99(19):7937-44, 2015). In this study, however, 100-fold higher concentrations of manganese in the culture medium support 3-HP production highlighting the difference in metabolism between production of 3-HP and the organic acids typically produced by these organisms. Tolerance, or even a requirement for higher concentrations of trace elements for 3-HP production avoids some of typical challenges with organic acid production by Aspergilli whereby micro-nutrients leaching from metal fermentation vessels or present as contaminants in feedstock sugars negatively impact yield (Karaffa et al., Microorganisms 9(6):1267, 2021).

A component of economically viable 3-HP production is utilization of inexpensive feedstocks (Werpy et al., National Renewable Energy Lab., Golden, CO (US), 2004). The 3-HP production on a DDR-EH derived sugars from corn stover was evaluated and optimized with the higher 3-HP production transgenic strains. Raw DDR-EH requires supplementation macro-nutrients (nitrogen and phosphate) to support growth and 3-HP production by A. niger, but that the trace elements added to RDM can be eliminated as an additive to reduce cost. The optimal temperature for 3-HP production was around 34° C., which is consistent with optimal temperatures for enzyme activities of PAND (50° C.) of T. castaneum (Wang et al., J Ind Microbiol Biotechnol 47(6-7):465-74, 2020), BAPAT (35° C.) of B. cereus (Nakano et al., J Biochem 81(5):1375-81, 1977), and HPDH (37° C.) of E. coli K12-TG1 (Guyot et al., MicrobiologyOpen 3(1):52-63, 2014). Increasing the concentration of (NH₄)₂SO₄ to the optimal level in RDM supported more growth but reduced the yield of 3-HP in DDR-EH indicating a lower concentration of nitrogen can be used to further reduce production costs.

Engineering efforts to produce 3-HP in fungi have demonstrated modest yields from pure glucose and xylose but typically supplement with vitamins (Kildegaard et al., Microb Cell Fact 15(1):1-13, 2016; Borodina et al., Metab Eng 27:57-64, 2015; Takayama et al., Microb Cell Factories 17(1):1-11, 2018; Kildegaard et al., Metab Eng Commun 2:132-6, 2015). Efforts have been made to produce 3-HP in the yeast S. cerevisiae in acidic conditions below the pKa of 3-HP, however yield was nearly half that achieved at pH 5 (Borodina et al., Metab Eng 27:57-64, 2015). Engineering efforts to produce 3-HP from mixed sugars in bacteria (E. coli and C. glutamicum) have also been successful but typically require growth at neutral pH and supplementation with vitamins and a complex nutrient source such as yeast extract or corn-steep liquor (Chen et al., Metab Eng 39:151-8, 2017; Heo et al., Bioresource Technol 285:121320, 2019; Lee et al., Bioresour Technol 299:122600, 2020; Jung et al., Bioresour Technol 198:709-16, 2015). Production of 3-HP using A. niger alleviates many of the economic issues presented by model yeast and bacterial hosts by eliminating the need to supplement with costly vitamins and complex nutrients and allowing for production as a free acid amenable to low-cost purification strategies.

Results from a DBTL cycle comparing multiple species and strains in conjunction with optimization of cultivation conditions dramatically increased the yield of 3-HP to 48% of the no-growth theoretical yield from a corn-stover feedstock. This work establishes Aspergillus species as a platform for commercial production of renewable 3-HP as a precursor for a variety of fossil-derived chemicals including 1,3-propanediol, acrylic acid, methyl acrylate, acrylamide, and acrylonitrile. In addition, fermentation conditions were investigated, and it was determined that fermentation at 34° C. under acidic pH (pH 2) and microaerobic conditions provided optimal 3-HP production. It was unexpected that low oxygen availability would be ideal as this is not the case for established organic acid fermentations with Aspergillus species. In addition, high production at very acidic pH is also unexpected as this condition is typically toxic for most organisms.

### III. Recombinant Aspergillus niger

Disclosed herein are recombinant Aspergillus niger including a 3-HP β-alanine pathway, which are capable of producing 3-hydroxypropionic acid (3-HP). The recombinant Aspergillus niger includes one or more exogenous nucleic acid molecules encoding aspartate 1-decarboxylase (PAND), β-alanine-pyruvate aminotransferase (BAPAT), and 3-hydroxypropionate dehydrogenase (HPDH). In some examples, one exogenous nucleic acid molecule encodes all of PAND, BAPT, and HPDH. In some examples, PAND, BAPT, and HPDH are encoded on separate exogenous nucleic acid molecules.

The copy number of each of PAND, BAPT, and HPDH in the recombinant Aspergillus niger is independently about 1 to about 80. Thus, in a non-limiting example, the copy number of PAND can be about 26, the copy number of BAPT can be about 29, and the copy number of HPDH can be about 25. In some examples, the copy number of PAND, BAPT, and HPDH are the same, or about the same, for example, all of PAND, BAPT, and HPDH each have a copy number of about 30. In some examples, the copy number of each of PAND, BAPT, and/or HPDH independently is about 5 to about 80, for example, about 5 to about 80, about 10 to about 80, about 15 to about 80, about 20 to about 80, about 25 to about 80, about 30 to about 80, about 35 to about 80, about 40 to about 80, about 45 to about 80, about 50 to about 80, about 60 to about 80, about 70 to about 80, about 5 to about 60, about 10 to about 60, about 15 to about 60, about 20 to about 60, about 25 to about 60, about 30 to about 60, about 35 to about 60, about 40 to about 60, about 45 to about 60, about 50 to about 60, about 5 to about 50, about 10 to about 50, about 15 to about 50, about 20 to about 50, about 25 to about 50, 30 to about 50, about 35 to about 50, about 40 to about 50, about 45 to about 50, about 5 to about 45, about 10 to about 45, about 15 to about 45, about 20 to about 45, about 25 to about 45, about 30 to about 45, about 35 to about 45, about 40 to about 45, about 5 to about 40, about 10 to about 40, about 15 to about 40, about 20 to about 40, about 25 to about 40, about 30 to about 40, about 35 to about 40, about 5 to about 35, about 10 to about 35, about 15 to about 35, about 20 to about 35, about 25 to about 35, about 30 to about 35, about 5 to about 30, about 10 to about 30, about 15 to about 30, about 20 to about 30, about 25 to about 30, about 5 to about 25, about 10 to about 25, about 15 to about 25, about 20 to about 25, about 5 to about 20, about 10 to about 20, about 15 to about 20, or about 12 to about 27. In some examples, the copy number is about 12 to about 27. In some examples, the copy number is about 25 to about 29.

In some examples, the copy number of each of PAND, BAPT, and/or HPDH independently is at least 5, for example, at least 7, at least 10, at least 12, at least 15, at least 20, at least 25, at least 27, at least 30, at least 35, at least 40, at least 45, at least 50, or more. In a non-limiting example, the copy number of each of PAND, BAPT, and HPDH is at least 12. In some examples, the copy number is about 5, about 10, about 12, about 15, about 20, about 25, about 26, about 27, about 29, about 30, about 35, about 40, about 45, or about 50. In a non-limiting example, the copy number for each of PAND, BAPT, and HPDH is about 27.

The PAND, BAPT, and/or HPDH encoded by the one or more exogenous nucleic acid molecules can be prokaryotic or eukaryotic in origin. In some examples, the PAND, BAPT, and/or HPDH nucleic acid molecules are from an animal (e.g., insect) or bacteria. In a non-limiting example, the PAND coding sequence is from Tribolium castaneum, the BAPAT coding sequence is from Bacillus cereus, and/or the HPDH coding sequence is from Escherichia coli. In a further non-limiting example, the PAND is from Tribolium castaneum, the BAPAT is from Bacillus cereus, and the HPDH is from Escherichia coli. In some examples, the one or more exogenous nucleic acids encoding PAND, BAPT, and/or HPDH are codon optimized for expression in a fungi, for example, in Aspergillus. In some examples, the PAND, BAPT, and HPDH coding sequences are optimized for expression in an Aspergillus species.

In some examples, the PAND amino acid sequence comprises at least 80% sequence identity to SEQ ID NO: 1, for example, at least 85%, at least 90%, at least 95%, at least 98%, at least 99%, or 100% sequence identity to SEQ ID NO: 1. In a non-limiting example, the PAND amino acid sequence comprises at least 95% sequence identity to SEQ ID NO: 1. In a further non-limiting example, the PAND amino acid sequence comprises at least 98% sequence identity to SEQ ID NO: 1. In some examples, the PAND amino acid sequence comprises or consists of SEQ ID NO: 1.

In some examples, the exogenous nucleic acid molecule encoding PAND comprises at least 80% sequence identity to SEQ ID NO: 130, for example, at least 85%, at least 90%, at least 95%, at least 98%, at least 99%, or 100% sequence identity to SEQ ID NO: 130. In a non-limiting example, the exogenous nucleic acid molecule encoding PAND comprises at least 95% sequence identity to SEQ ID NO: 130. In a further non-limiting example, the exogenous nucleic acid molecule encoding PAND comprises at least 98% sequence identity to SEQ ID NO: 130. In some examples, the exogenous nucleic acid molecule encoding PAND comprises or consists of SEQ ID NO: 130, or a degenerate variant thereof.

In some examples, the BAPAT amino acid sequence comprises at least 80% sequence identity to SEQ ID NO: 2, for example, at least 85%, at least 90%, at least 95%, at least 98%, at least 99%, or 100% sequence identity to SEQ ID NO: 2. In a non-limiting example, the BAPAT amino acid sequence comprises at least 95% sequence identity to SEQ ID NO: 2. In a further non-limiting example, the BAPAT amino acid sequence comprises at least 98% sequence identity to SEQ ID NO: 2. In some examples, the BAPAT amino acid sequence comprises or consists of SEQ ID NO: 2.

In some examples, the exogenous nucleic acid molecule encoding BAPAT comprises at least 80% sequence identity to SEQ ID NO: 131, for example, at least 85%, at least 90%, at least 95%, at least 98%, at least 99%, or 100% sequence identity to SEQ ID NO: 131. In a non-limiting example, the exogenous nucleic acid molecule encoding BAPAT comprises at least 95% sequence identity to SEQ ID NO: 131. In a further non-limiting example, the exogenous nucleic acid molecule encoding BAPAT comprises at least 98% sequence identity to SEQ ID NO: 131. In some examples, the exogenous nucleic acid molecule encoding BAPAT comprises or consists of SEQ ID NO: 131, or a degenerate variant thereof.

In some examples, the HPDH amino acid sequence comprises at least 80% sequence identity to SEQ ID NO: 3, for example, at least 85%, at least 90%, at least 95%, at least 98%, at least 99%, or 100% sequence identity to SEQ ID NO: 3. In a non-limiting example, the HPDH amino acid sequence comprises at least 95% sequence identity to SEQ ID NO: 3. In a further non-limiting example, the HPDH amino acid sequence comprises at least 98% sequence identity to SEQ ID NO: 3. In some examples, the HPDH amino acid sequence comprises or consists of SEQ ID NO: 3.

In some examples, the exogenous nucleic acid molecule encoding HPDH comprises at least 80% sequence identity to SEQ ID NO: 132, for example, at least 85%, at least 90%, at least 95%, at least 98%, at least 99%, or 100% sequence identity to SEQ ID NO: 132. In a non-limiting example, the exogenous nucleic acid molecule encoding HPDH comprises at least 95% sequence identity to SEQ ID NO: 132. In a further non-limiting example, the exogenous nucleic acid molecule encoding HPDH comprises at least 98% sequence identity to SEQ ID NO: 132. In some examples, the exogenous nucleic acid molecule encoding HPDH comprises or consists of SEQ ID NO: 132, or a degenerate variant thereof.

The PAND, BAPAT, and HPDH can all be encoded on a single exogenous nucleic acid molecule. In some examples, a single exogenous nucleic acid molecule encoding PAND, BAPAT, and HPDH comprises at least 80% sequence identity to SEQ ID NO: 4, for example, at least 85%, at least 90%, at least 95%, at least 98%, at least 99%, or 100% sequence identity to SEQ ID NO: 4. In a non-limiting example, the single exogenous nucleic acid molecule comprises at least 95% sequence identity to SEQ ID NO: 4. In a further non-limiting example, the exogenous nucleic acid molecule comprises at least 98% sequence identity to SEQ ID NO: 4. In some examples, the single exogenous nucleic acid molecule comprises or consists of SEQ ID NO: 4, or a degenerate variant thereof. In some examples, a single exogenous nucleic acid molecule encoding PAND, BAPAT, and HPDH comprises at least 80% sequence identity to SEQ ID NO: 142, for example, at least 85%, at least 90%, at least 95%, at least 98%, at least 99%, or 100% sequence identity to SEQ ID NO: 142. In a non-limiting example, the single exogenous nucleic acid molecule comprises at least 95% sequence identity to SEQ ID NO: 142. In a further non-limiting example, the exogenous nucleic acid molecule comprises at least 98% sequence identity to SEQ ID NO: 142. In some examples, the single exogenous nucleic acid molecule comprises or consists of SEQ ID NO: 142, or a degenerate variant thereof.

In some examples, the recombinant Aspergillus niger includes a nucleic acid molecule having at least 90% sequence identity to SEQ ID NO: 4, and a nucleic acid molecule having at least 90% sequence identity to SEQ ID NO: 142. In some examples, the recombinant Aspergillus niger includes a nucleic acid molecule having at least 95% sequence identity to SEQ ID NO: 4, and a nucleic acid molecule having at least 95% sequence identity to SEQ ID NO: 142. In some examples, the recombinant Aspergillus niger includes a nucleic acid molecule comprising SEQ ID NO: 4, and a nucleic acid molecule comprising SEQ ID NO: 142. In some examples, the recombinant Aspergillus niger includes at least 10 copies (e.g., at least 11, at least 12, at least 13, at least 14, at least 15, at least 16, etc.) of a nucleic acid molecule comprising SEQ ID NO: 4, and at least 10 copies (e.g., at least 11, at least 12, at least 13, at least 14, at least 15, at least 16, etc.) of a nucleic acid molecule comprising SEQ ID NO: 142. In some examples, the recombinant Aspergillus niger includes about 10 to about 15 copies of a nucleic acid molecule comprising SEQ ID NO: 4, and about 10 to about 15 copies of a nucleic acid molecule comprising SEQ ID NO: 142. In a non-limiting example, the recombinant Aspergillus niger includes about 12 copies of a nucleic acid molecule comprising SEQ ID NO: 4, and about 14 copies of a nucleic acid molecule comprising SEQ ID NO: 142.

In some examples, the PAND, BAPAT, and HPDH are encoded by two or more separate exogenous nucleic acid molecules, such as 2 or 3 separate exogenous nucleic acid molecules.

The recombinant Aspergillus niger can include additional genetic modifications, such as an inactivated endogenous gene (a gene native to Aspergillus niger), or include additional exogenous nucleic acid molecules (e.g., encoding additional genes). An inactivated gene includes gene deletions or other genetic modifications that prevent or decrease the production of a functional gene product (e.g., a genetic modification that results in an early stop codon, or a modification that disrupts a binding or active site of an enzyme). A 100% decrease is not required. In some examples, an inactivated gene reduces expression of its corresponding protein by at least 50%, at least 75%, at least 90%, at least 95%, at least 99%, at least 99.9% or even 99.99%. Genes can be inactivated by known methods, such as the cre/lox system, or a genome editing system (e.g., clustered regularly interspaced short palindromic repeats)/Cas (CRISPR/Cas) systems, transcription activator like effector nuclease (TALEN) systems, or zinc finger nuclease (ZFN) systems). In some examples, an endogenous ald6a, ald6b, ald3, and/or oahA gene is inactivated in the recombinant Aspergillus niger. Exemplary genomic sequences of Aspergillus niger ald6a, ald6b, ald3, and oahA are provided herein. An inactivated gene can result in a gene that is not present or does not produce a functional product (e.g., a protein encoded by the gene). In some examples, an endogenous ald6a, ald6b, ald3, and/or oahA gene is not present or does not produce a functional product in the recombinant Aspergillus niger. An Aspergillus niger having an inactivated gene can be identified using known methods, for example, PCR or a nucleic acid hybridization technique (e.g., Northern and Southern analysis) can be used to confirm that a recombinant A. niger includes a genetically inactivated gene. In one example, real-time reverse transcription PCR (qRT-PCR) is used to detect and quantify messenger RNA (mRNA), such as mRNA of ald6a, ald6b, ald3, and/or oahA, to determine whether ald6a, ald6b, ald3, and/or oahA is inactivated. Known biochemical techniques can be used to detect a decrease in functional protein products (e.g., detect whether a protein product (e.g., enzyme) is functional or non-functional).

In some examples, inactivating an endogenous ald6a, ald6b, ald3, and/or oahA gene in the recombinant Aspergillus niger increases 3-HP production, for example, by at least 10%, at least 20%, at least 30%, at least 40%, at least 50%, at least 60%, at least 70%, at least 80%, at least 90%, at least 100%, at least 150%, at least 200%, at least 250%, at least 300%, at least 400%, at least 500%, or more relative to a control. In some examples, inactivating an endogenous ald6a, ald6b, ald3, and/or oahA gene in the recombinant Aspergillus niger increases 3-HP production by 10% to 500%, for example, by 10% to 400%, 10% to 300%, 10% to 200%, 10% to 100%, 10% to 90%, 10% to 80%, 10% to 70%, 10% to 60%, 10% to 50%, 10% to 40%, 10% to 30%, 10% to 25%, 20% to 400%, 20% to 300%, 20% to 200%, 20% to 100%, 20% to 90%, 20% to 80%, 20% to 70%, 20% to 60%, 20% to 50%, 20% to 40%, 20% to 30%, 20% to 25%, 30% to 400%, 30% to 300%, 30% to 200%, 30% to 100%, 30% to 90%, 30% to 80%, 30% to 70%, 30% to 60%, 30% to 50%, 30% to 40%, 40% to 400%, 40% to 300%, 40% to 200%, 40% to 100%, 40% to 90%, 40% to 80%, 40% to 70%, 40% to 60%, or 40% to 50% relative to a control. In a non-limiting example, inactivating an endogenous ald6a gene in the recombinant Aspergillus niger increases 3-HP production by about 40 to 50% relative to the recombinant Aspergillus niger with an unmodified (WT) ald6a gene.

In some examples, the recombinant Aspergillus niger further includes one or more exogenous nucleic acid molecules encoding a pyruvate carboxylase (e.g., pyc), aspartate aminotransferase (e.g., awl), and/or monocarboxylate transporter (e.g., mct1). In some examples, an exogenous nucleic acid encoding pyc, aat1, and/or mct1 is a recombinant nucleic acid molecule. In some examples, an exogenous nucleic acid encoding pyc, aat1, and/or mct1 results in increased expression (overexpression) of pyc, aat1, and/or mct1, respectively, in the recombinant Aspergillus niger. Genes can be overexpressed, for example, by operably linking a nucleic acid encoding the gene to a constitutive promoter. In particular examples, ald6a is not present or does not produce a functional product and pyc and/or aat1 is overexpressed in the recombinant Aspergillus niger.

In some examples, further including one or more exogenous nucleic acid molecules encoding a pyruvate carboxylase (e.g., pyc), aspartate aminotransferase (e.g., aat1), or monocarboxylate transporter (e.g., mct1) in the recombinant Aspergillus niger increases 3-HP production, for example, by at least 10%, at least 20%, at least 30%, at least 40%, at least 50%, at least 60%, at least 70%, at least 80%, at least 90%, at least 100%, at least 150%, at least 200%, at least 250%, at least 300%, at least 400%, at least 500%, or more relative to a control. In some examples, further including one or more exogenous nucleic acid molecules encoding a pyruvate carboxylase (e.g., pyc), aspartate aminotransferase (e.g., aat1), or monocarboxylate transporter (e.g., mct1) in the recombinant Aspergillus niger increases 3-HP production by 10% to 500%, for example, by 10% to 400%, 10% to 300%, 10% to 200%, 10% to 100%, 10% to 90%, 10% to 80%, 10% to 70%, 10% to 60%, 10% to 50%, 10% to 40%, 10% to 30%, 10% to 25%, 20% to 400%, 20% to 300%, 20% to 200%, 20% to 100%, 20% to 90%, 20% to 80%, 20% to 70%, 20% to 60%, 20% to 50%, 20% to 40%, 20% to 30%, 20% to 25%, 30% to 400%, 30% to 300%, 30% to 200%, 30% to 100%, 30% to 90%, 30% to 80%, 30% to 70%, 30% to 60%, 30% to 50%, 30% to 40%, 40% to 400%, 40% to 300%, 40% to 200%, 40% to 100%, 40% to 90%, 40% to 80%, 40% to 70%, 40% to 60%, 40% to 50%, 50% to 400%, 50% to 300%, 50% to 200%, 50% to 100%, 50% to 90%, 50% to 80%, 50% to 70%, 50% to 60%, 75% to 400%, 75% to 300%, 75% to 200%, 75% to 100%, 75% to 90%, 75% to 80%, 80% to 200%, or 80% to 100% relative to a control. In a non-limiting example, further including an exogenous nucleic acid molecule encoding pyc increases 3-HP production by 80% to 90% relative to the recombinant Aspergillus niger without the exogenous pyc. In a non-limiting example, further including an exogenous nucleic acid molecule encoding mct1 increases 3-HP production by 30% to 40% relative to the recombinant Aspergillus niger without the exogenous mct1. In some examples, pyc, aat1, and/or mct1 are Aspergillus niger pyc, aat1, or mct1, respectively.

In some examples, the pyc amino acid sequence has at least 80% sequence identity to SEQ ID NO: 5, for example, at least 85%, at least 90%, at least 95%, at least 98%, at least 99%, or 100% sequence identity to SEQ ID NO: 5. In a non-limiting example, the pyc amino acid sequence has at least 95% sequence identity to SEQ ID NO: 5. In a further non-limiting example, the pyc amino acid sequence has at least 98% sequence identity to SEQ ID NO: 5. In some examples, the pyc amino acid sequence includes or consists of SEQ ID NO: 5.

In some examples, the exogenous nucleic acid molecule encoding pyc has at least 80% sequence identity to SEQ ID NO: 133, for example, at least 85%, at least 90%, at least 95%, at least 98%, at least 99%, or 100% sequence identity to SEQ ID NO: 133. In a non-limiting example, the exogenous nucleic acid molecule encoding pyc has at least 95% sequence identity to SEQ ID NO: 133. In a further non-limiting example, the exogenous nucleic acid molecule encoding pyc has at least 98% sequence identity to SEQ ID NO: 133. In some examples, the exogenous nucleic acid molecule encoding pyc includes or consists of SEQ ID NO: 133, or a degenerate variant thereof.

In some examples, the mct1 amino acid sequence has at least 80% sequence identity to SEQ ID NO: 137, for example, at least 85%, at least 90%, at least 95%, at least 98%, at least 99%, or 100% sequence identity to SEQ ID NO: 137. In a non-limiting example, the mct1 amino acid sequence has at least 95% sequence identity to SEQ ID NO: 137. In a further non-limiting example, the mct1 amino acid sequence has at least 98% sequence identity to SEQ ID NO: 137. In some examples, the mct1 amino acid sequence includes or consists of SEQ ID NO: 137.

In some examples, the exogenous nucleic acid molecule encoding mct1 has at least 80% sequence identity to SEQ ID NO: 129, for example, at least 85%, at least 90%, at least 95%, at least 98%, at least 99%, or 100% sequence identity to SEQ ID NO: 129. In a non-limiting example, the exogenous nucleic acid molecule encoding mct1 has at least 95% sequence identity to SEQ ID NO: 129. In a further non-limiting example, the exogenous nucleic acid molecule encoding mct1 has at least 98% sequence identity to SEQ ID NO: 129. In some examples, the exogenous nucleic acid molecule encoding mct1 includes or consists of SEQ ID NO: 129, or a degenerate variant thereof.

In some examples, the aat1 amino acid sequence has at least 80% sequence identity to SEQ ID NO: 138, for example, at least 85%, at least 90%, at least 95%, at least 98%, at least 99%, or 100% sequence identity to SEQ ID NO: 138. In a non-limiting example, the aat1 amino acid sequence has at least 95% sequence identity to SEQ ID NO: 138. In a further non-limiting example, the aat1 amino acid sequence has at least 98% sequence identity to SEQ ID NO: 138. In some examples, the aat1 amino acid sequence includes or consists of SEQ ID NO: 138.

In some examples, the exogenous nucleic acid molecule encoding aat1 has at least 80% sequence identity to SEQ ID NO: 139, for example, at least 85%, at least 90%, at least 95%, at least 98%, at least 99%, or 100% sequence identity to SEQ ID NO: 139. In a non-limiting example, the exogenous nucleic acid molecule encoding aat1 has at least 95% sequence identity to SEQ ID NO: 139. In a further non-limiting example, the exogenous nucleic acid molecule encoding aat1 has at least 98% sequence identity to SEQ ID NO: 139. In some examples, the exogenous nucleic acid molecule encoding aat1 includes or consists of SEQ ID NO: 139, or a degenerate variant thereof.

In some examples, an exogenous nucleic acid molecule encoding pyc, aat1, and/or mct1 is operably linked to a promoter that is not naturally operably-linked to pyc, aat1, or mct1, respectively, in A. niger.

The copy number of an exogenous nucleic acid molecule encoding pyruvate carboxylase (e.g., pyc), aspartate aminotransferase (e.g., aat1), and/or monocarboxylate transporter (e.g., mct1) in the recombinant Aspergillus niger can be about 1 to about 80, for example, about 5 to about 80, about 10 to about 80, about 15 to about 80, about 20 to about 80, about 25 to about 80, about 30 to about 80, about 35 to about 80, about 40 to about 80, about 45 to about 80, about 50 to about 80, about 60 to about 80, about 70 to about 80, about 5 to about 60, about 10 to about 60, about 15 to about 60, about 20 to about 60, about 25 to about 60, about 30 to about 60, about 35 to about 60, about 40 to about 60, about 45 to about 60, about 50 to about 60, about 5 to about 50, about 10 to about 50, about 15 to about 50, about 20 to about 50, about 25 to about 50, 30 to about 50, about 35 to about 50, about 40 to about 50, about 45 to about 50, about 5 to about 45, about 10 to about 45, about 15 to about 45, about 20 to about 45, about 25 to about 45, about 30 to about 45, about 35 to about 45, about 40 to about 45, about 5 to about 40, about 10 to about 40, about 15 to about 40, about 20 to about 40, about 25 to about 40, about 30 to about 40, about 35 to about 40, about 5 to about 35, about 10 to about 35, about 15 to about 35, about 20 to about 35, about 25 to about 35, about 30 to about 35, about 5 to about 30, about 10 to about 30, about 15 to about 30, about 20 to about 30, about 25 to about 30, about 5 to about 25, about 10 to about 25, about 15 to about 25, about 20 to about 25, about 5 to about 20, about 10 to about 20, about 15 to about 20, about 12 to about 27, about 1 to about 3, about 1 to about 5, or about 1 to about 10. In a non-limiting example, the copy number of the exogenous pyc is about 5 to about 10 in the recombinant Aspergillus niger. In another non-limiting example, the copy number of the exogenous aat1 is about 1 to about 5 in the recombinant Aspergillus niger.

In some examples, the copy number of an exogenous nucleic acid molecule encoding pyc, aat1, and/or mct1 is at least 1, for example, at least 2, at least 3, at least 4, at least 5, at least 6, at least 7, at least 8, at least 9, at least 10, at least 12, at least 15, at least 20, at least 25, at least 27, at least 30, at least 35, at least 40, at least 45, at least 50, or more in the recombinant A. niger. In a non-limiting example, the copy number of an exogenous nucleic acid molecule encoding pyc or mct1 is at least 8 in the recombinant A. niger. In a non-limiting example, the copy number of an exogenous nucleic acid molecule encoding aat1 is at least 1 in the recombinant A. niger. In some examples, the copy number is about 1, about 2, about 3, about 5, about 8, about 10, about 12, about 15, about 20, about 25, about 26, about 27, about 29, about 30, about 35, about 40, about 45, or about 50 in the recombinant A. niger. In a non-limiting example, the copy number of pyc is about 8 in the recombinant A. niger. In a non-limiting example, the copy number of aat1 is about 1 in the recombinant A. niger.

Any of the exogenous nucleic acids disclosed herein (e.g., exogenous nucleic acids encoding PAND, BAPT, HPDH, pyc, aat1, and/or MCT1) can be operably linked to a promoter. Constitutive, inducible, cell/tissue specific, or development specific promoters can be used. Promoters produced by recombinant DNA or synthetic techniques can also be used to provide for transcription of the nucleic acid sequences. Exemplary promoters that can be used include, but are not limited to the SV40 promoter, the CMV enhancer-promoter, CMV enhancer/β-actin promoter, trpC promoter (trpCp), translation elongation factor 1 gene promoter (tef1p), mbfA promoter (mbfAp), ubi4 gene promoter (ubi4p), ubi1S27 promoter (ubi1S27p, ubiS27p, or ubiS1p), glyceraldehyde-3-phosphate dehydrogenase promoter (gpdAp), enolase promoter (eno1p), and malate dehydrogenase 9 (mdhp). In some examples, an exogenous nucleic acid disclosed herein is operably linked to a promoter exogenous to Aspergillus niger. In some examples, an exogenous nucleic acid disclosed herein is operably linked to a promoter endogenous to Aspergillus niger (e.g., Aspergillus niger gpdAp, eno1p, mdhp, tefip, ubi4p, ubi1S27p, or mbf1p). In a non-limiting example, a nucleic acid molecule encoding PAND is operably linked to a ubi4p or gpdAp promoter, a nucleic acid molecule encoding BAPAT is operably linked to an ubi1S27p or eno1p promoter, and/or a nucleic acid molecule encoding HPDH is operably linked to a mbfAp or gpdAp promoter. In some examples, the eno1p, ubi4p, ubi1S27p, and/or mbfAp promoters are from Aspergillus niger. In some examples, the gpdAp promoter is from A. niger or A. nidulans.

Any of the exogenous nucleic acids disclosed herein can be part of a vector, such as a plasmid or viral vector. In some examples, expression of the exogenous nucleic acid molecules is driven by one or more promoters, such as a constitutive or inducible promoter, or a bi-directional promoter. In some examples, the promoter used to drive expression of PAND, BAPAT, and HPDH is a native promoter (e.g., native to the PAND, BAPAT, and HPDH gene being expressed). In other examples, the promoter used to drive expression of PAND, BAPAT, and HPDH is a non-native promoter (e.g., exogenous to the PAND, BAPAT, and HPDH gene being expressed).

Methods of making the recombinant Aspergillus niger disclosed herein are also included in the disclosure. The method includes introducing one or more exogenous nucleic acid molecules encoding PAND, BAPAT, and HPDH, or a vector including the exogenous nucleic acid molecules, into the Aspergillus niger, thereby producing the recombinant Aspergillus niger. In a non-limiting example, Aspergillus niger is transformed with a vector including a PAND, BAPAT, and HPDH gene. Multiple copies of PAND, BAPAT, and/or HPDH can be introduced into the Aspergillus niger, such as 5, 10, 12, 15, 20, 25, 26, 27, 28, 29, 30, 40, 50 or more PAND, BAPAT, and/or HPDH sequences. In some examples, about 12 to about 30 copies of each of PAND, BAPAT, and HPDH are introduced into the Aspergillus niger. The transformed Aspergillus niger cells (the recombinant Aspergillus niger) produce 3-HP. In one example, the cre-lox system is used for site specific recombination of DNA (for example see Steiger et al., Appl. Environ. Microbiol. 77(1):114, 2011). Using recombination techniques, a targeted gene of interest (e.g., ald6a, ald6b, ald3, and/or oahA) can be deleted in the Aspergillus niger genome and replaced with another nucleic acid sequence (e.g., one or more copies of an exogenous nucleic acid encoding PAND, BAPAT, and/or HPDH) flanked by lox sites. Transient expression (by electroporation of a suicide plasmid containing the cre gene under control of a synthetic promoter that functions in Aspergillus) of the cre recombinase results in efficient elimination of the lox flanked marker. This process produces Aspergillus niger containing the desired insertion mutation and one copy of the lox sequence.

The recombinant Aspergillus niger is useful for producing 3-HP. For example, 3-HP can be produced by culturing the recombinant Aspergillus niger under conditions that permit the production of 3-HP. Suitable conditions are described herein in the following section.

### IV. Methods of Producing 3-HP

Also disclosed are methods of producing 3-HP, which include culturing an Aspergillus niger including a 3-HP β-alanine pathway (e.g., the recombinant Aspergillus niger disclosed herein). The method can include a first step of inoculating a media (e.g., liquid media) with Aspergillus niger comprising a 3-HP β-alanine pathway (e.g., the recombinant Aspergillus niger disclosed herein), thereby generating a cultured media. In some examples, 1×10³ to 1×10¹⁰ spores/mL of the Aspergillus niger are used to inoculate the media, for example, 1×10³ to 1×10⁹, 1×10³ to 1×10⁸, 1×10³ to 1×10⁷, 1×10³ to 1×10⁶, 1×10³ to 1×10⁵, 1×10³ to 1×10⁴, 1×10⁴ to 1×10⁹, 1×10⁴ to 1×10⁸, 1×10⁴ to 1×10⁷, 1×10⁴ to 1×10⁶, 1×10⁴ to 1×10⁵, 1×10⁵ to 1×10⁹, 1×10⁵ to 1×10⁸, 1×10⁵ to 1×10⁷, 1×10⁵ to 1×10⁶, 1×10⁶ to 1×10⁹, 1×10⁶ to 1×10⁸, or 1×10⁶ to 1×10⁷ spores/mL. In a specific, non-limiting example, about 1×10⁵ to about 1×10⁷ spores/mL of the Aspergillus niger are used to inoculate the media. In some examples, about 1×10³, about 1×10⁴, about 1×10⁵, about 1×10⁶, about 1×10⁷, or about 1×10⁸ spores/mL of the Aspergillus niger are used to inoculate the media. In a non-limiting example, about 1×10⁶ spores/mL of the Aspergillus niger are used to inoculate the media.

In some examples, a seed culture is used to inoculate the media. A seed culture is a culture grown for the purpose of inoculating a media for fermentation. A seed culture can be prepared using known methods about 6 to 48 hours (e.g., 15 to 18 hours) prior to the inoculation step. The media can be inoculated with about 1% to about 50% volume per volume (v/v) of the seed culture of the Aspergillus niger (e.g., a recombinant Aspergillus niger disclosed herein), for example, 1% to 40%, 1% to 30%, 1% to 25%, 1% to 20%, 1% to 15%, 1% to 10%, 1% to 5%, 5% to 50%, 5% to 40%, 5% to 30%, 5% to 25%, 5% to 20%, 5% to 15%, 5% to 10%, 8% to 50%, 8% to 40%, 8% to 30%, 8% to 25%, 8% to 20%, 8% to 15%, 8% to 12%, 8% to 10%, 10% to 50%, 10% to 40%, 10% to 30%, 10% to 25%, 10% to 20%, 10% to 15%, 15% to 50%, 15% to 40%, 15% to 30%, 15% to 25%, 15% to 20%, 20% to 50%, 20% to 40%, 20% to 30%, 20% to 25%, 25% to 50%, 25% to 40%, or 25% to 30% (v/v) of the seed culture of the Aspergillus niger. In a non-limiting example, the media is inoculated with about 5% to about 20% (vol/vol) of the seed culture of Aspergillus niger. In some examples, the media is inoculated with about 1%, about 5%, about 8%, about 10%, about 12%, about 15%, about 20%, about 25%, about 30%, about 35%, about 40%, about 45%, or about 50% (v/v) of the seed culture of the Aspergillus niger. In a non-limiting example, the media is inoculated with about 10% (vol/vol) of the seed culture of the Aspergillus niger.

The method includes fermenting the cultured media at a temperature of about 20° C. to about 40° C., for example, 20° C. to 37° C., 20° C. to 35° C., 20° C. to 30° C., 20° C. to 27° C., 20° C. to 25° C., 25° C. to 40° C., 25° C. to 37° C., 25° C. to 35° C., 25° C. to 30° C., 25° C. to 27° C., 27° C. to 40° C., 27° C. to 37° C., 27° C. to 35° C., 27° C. to 30° C., 30° C. to 40° C., 30° C. to 37° C., 30° C. to 35° C., 32° C. to 40° C., 32° C. to 37° C., 32° C. to 35° C., 33° C. to 40° C., 33° C. to 37° C., 33° C. to 35° C., 35° C. to 40° C., or 35° C. to 37° C. In a non-limiting example, the cultured media is fermented at a temperature of about 33° C. to about 35° C. In some examples, the cultured media is fermented at a temperature of about 25° C., about 27° C., about 30° C., about 32° C., about 34° C., about 36° C., about 37° C., or about 40° C. In a non-limiting example, the cultured media is fermented at a temperature of about 34° C.

The culture is fermented under acidic conditions. Acidic conditions include a pH of about 1 to about 6, for example, 1 to 6, 1 to 5, 1 to 4, 1 to 3, 1 to 2, 2 to 6, 2 to 5, 2 to 4, 2 to 3, 3 to 6, 3 to 5, 3 to 4, 4 to 6, or 4 to 5, during fermentation. In some examples, the pH is about 1 to about 4 during fermentation. In some examples, the pH is 6 or lower during fermentation, for example, a pH of 5, a pH of 4, a pH of 3, a pH of 2, or a pH of 1. In some examples, the pH is lower than 3 during fermentation. In a non-limiting example, the pH is about 2 during fermentation.

The culture is fermented under microaerobic conditions. A microaerobic condition occurs when oxygen demand of an organism is greater than the oxygen transfer rate from the gaseous to aqueous phase. In some examples, the microaerobic condition is a dissolved oxygen content of about 0 to about 15% (DO) during fermentation, for example, about 1% to about 15%, about 1% to about 10%, about 1% to about 8%, about 1% to about 7%, about 1% to about 6%, about 1% to about 5%, about 1% to about 4%, about 1% to about 3%, about 1% to about 2%, about 2% to about 15%, about 2% to about 10%, about 2% to about 9%, about 2% to about 8%, about 2% to about 7%, about 2% to about 6%, about 2% to about 5%, about 2% to about 4%, about 2% to about 3%, about 4% to about 15%, about 4% to about 10%, about 4% to about 9%, about 4% to about 8%, about 4% to about 7%, about 4% to about 6%, about 4% to about 5%, about 5% to about 15%, about 5% to about 10%, about 5% to about 9%, about 5% to about 8%, about 5% to about 7%, about 5% to about 6%, about 6% to about 15%, about 6% to about 10%, about 6% to about 9%, about 6% to about 8%, about 6% to about 7%, about 8% to about 15%, about 8% to about 10%, or about 8% to about 9% (DO) during fermentation. In some examples, the dissolved oxygen content is less than about 15%, for example, less than 10%, less than 8%, less than 6%, less than 5%, less than 4%, less than 3%, less than 2%, or less than 1% (DO) during fermentation. In some examples, the dissolved oxygen content is about 1%, about 2%, about 3%, about 4%, about 5%, about 6%, about 7%, about 8%, about 9%, about 10%, about 12%, or about 15% (DO) during fermentation. In a specific, non-limiting example, the dissolved oxygen content is about 0% to 10% (DO) during fermentation.

The media can be any suitable liquid media, such as Riscaldati B medium (RisB) (100 g Glucose, 0.11 g KH₂PO₄, 2.36 g (NH₄)₂SO₄, 2.08 g MgSO4*7H₂O, 0.074 g NaCl, 0.13 g CaCl₂)*2H₂O, 1 ml of 1000× trace elements in 1000 ml DI water, adjust pH to 3.4 with H₂SO₄, 1000× trace elements contains 1.3 g/L ZnSO₄*7H₂O, 5.5 g/L FeSO₄*7H₂O, 0.2 g/L CuSO₄*5H₂O, 0.7 g/L MnCl₂*4H₂O)) or modified Riscaldati B medium (RisB) with 20× trace elements. In some examples, the media is an acidic media, such as a media having an initial pH (pH of the media prior to inoculation) of less than 4, such as less than 3.5, for example about pH 2 to 4, 3 to 4, 3.5 to 4, 3.3 to 3.5, for example pH 2, 2.5, 2.8, 2.9, 3, 3.1, 3.2, 3.3, 3.4, 3.5, 3.6, 3.7, 3.8, 3.9 or 4. In some examples, the initial pH of the acidic media is about 3 to about 4.

The media can further include trace elements (TE), corn steep liquor, peptone, tryptone, soy protein, sodium chloride, yeast extract, sorbitol, and/or phosphorous. In biology, trace elements function primarily as catalysts in enzyme systems, for example, some metallic ions, such as iron and copper, participate in oxidation-reduction reactions in energy metabolism. Exemplary trace elements include iron, zinc, nickel, copper, molybdenum, cobalt, chromium, cadmium, selenium, and manganese. In some examples, the trace elements include copper, manganese, iron, and zinc (e.g., 4 mg/l CuSO₄·5H₂O; 110 mg/l FeSO₄·7H₂O; 14 mg/l MnCl₂·4H₂O; and 26 mg/l ZnSO₄·7H₂O). The media can include glucose, maltose, fructose, maltodextrin, and/or a glucose:xylose mixture as a carbon source. In some examples, the media is supplemented with nitrogen, manganese, and/or phosphate.

In some examples, the methods include aerating the culture, for example, by rotation (e.g., 100 to 300 rpm, such as about 150 rpm or about 200 rpm) or other aeration mechanism. In some examples, the Aspergillus niger (e.g., a recombinant Aspergillus niger disclosed herein) are grown in culture containers (such as baffled flasks) or in a bioreactor. The cultured media can be aerated before and/or during fermentation. In some examples, fermentation is performed at atmospheric pressure.

In some examples, the methods include purifying or isolating the 3-HP after fermentation. In some examples, the 3-HP is isolated at least 2 days, at least 3 days, at least 5 days, at least 7 days, at least 8 days or at least 10 days after the inoculation step, such as 2, 3, 4, 5, 6, 7, 8, 9, 10, 11, 12, 13, 14, 15, 16, 17, 18, 19 or 20 days after the inoculation step. In some examples, the 3-HP is purified or isolated after 4 days of fermentation. In some examples, the 3-HP is purified or isolated after 7 days of fermentation.

In some examples, the Aspergillus niger (e.g., a recombinant Aspergillus niger disclosed herein) produces about 0.1 g/l 3-HP to about 50 g/l 3-HP, for example, 0.1 g/l to 40 g/l, 0.1 g/l to 30 g/1, 0.1 g/l to 25 g/1, 0.1 g/l to 20 g/1, 0.1 g/l to 15 g/l, 0.1 g/l to 10 g/l, 0.1 g/l to 5 g/l, 0.1 g/l to 5 g/l, 0.1 g/l to 4.5 g/l, 0.1 g/l to 4 g/l, 0.1 g/l to 3.5 g/l, 0.1 g/l to 3 g/1, 0.1 g/l to 2.5 g/1, 0.1 g/l to 2 g/l, 0.1 g/l to 1.5 g/l, 0.1 g/l to 1 g/l, 0.1 g/l to 0.5 g/l, 0.5 g/l to 40 g/1, 0.5 g/l to 30 g/1, 0.5 g/l to 25 g/1, 0.5 g/l to 20 g/1, 0.5 g/l to 15 g/1, 0.5 g/l to 10 g/l, 0.5 g/l to 5 g/l, 0.5 g/l to 4.5 g/l, 0.5 g/l to 4 g/l, 0.5 g/l to 3.5 g/l, 0.5 g/l to 3 g/l, 0.5 g/l to 2.5 g/l, 0.5 g/l to 2 g/1, 0.5 g/l to 1.5 g/1, 0.5 g/l to 1 g/1, 1 g/l to 40 g/l, 1 g/l to 30 g/1, 1 g/l to 25 g/1, 1 g/l to 20 g/1, 1 g/l to 15 g/1, 1 g/l to 10 g/l, 1 g/l to 5 g/l, 1 g/l to 5 g/l, 1 g/l to 4.5 g/l, 1 g/l to 4 g/l, 1 g/l to 3.5 g/l, 1 g/l to 3 g/1, 1 g/l to 2.5 g/l, 1 g/l to 2 g/l, 1 g/l to 1.5 g/l, 1.5 g/l to 40 g/1, 1.5 g/l to 30 g/1, 1.5 g/l to 25 g/1, 1.5 g/l to 20 g/1, 1.5 g/l to 15 g/1, 1.5 g/l to 10 g/l, 1.5 g/l to 5 g/1, 1.5 g/l to 4.5 g/l, 1.5 g/l to 4 g/1, 1.5 g/l to 3.5 g/1, 1.5 g/l to 3 g/1, 1.5 g/l to 2.5 g/l, 1.5 g/l to 2 g/1, 5 g/l to 50 g/1, 5 g/l to 40 g/1, 5 g/l to 30 g/1, 5 g/l to 25 g/1, 5 g/l to 20 g/1, 5 g/l to 15 g/1, 5 g/l to 10 g/1, 10 g/l to 50 g/1, 10 g/l to 40 g/1, 10 g/l to 30 g/1, 10 g/l to 25 g/1, 10 g/l to 20 g/1, 10 g/l to 15 g/1, 15 g/l to 50 g/1, 15 g/l to 40 g/1, 15 g/l to 30 g/1, 15 g/l to 25 g/1, 15 g/l to 20 g/1, 20 g/l to 50 g/1, 20 g/l to 40 g/1, 20 g/l to 30 g/1, 20 g/l to 25 g/1, 25 g/l to 50 g/1, 25 g/l to 40 g/1, 25 g/l to 30 g/1, 30 g/l to 50 g/1, 30 g/l to 40 g/1, or 30 g/l to 35 g/l 3-HP after about 3, 4, 5, 6, 7, 8, 9, or 10 days of fermentation. In a non-limiting example, 20 g/l to 40 g/l 3-HP is produced after 7 days of fermentation. In a further non-limiting example, 30 g/l to 40 g/l 3-HP is produced after 7 days of fermentation.

In specific examples, the Aspergillus niger (e.g., a recombinant Aspergillus niger disclosed herein) produces at least 0.1 g/l of 3-HP after at least 2 days of fermentation, for example at least 0.2 g/l, at least 0.25 g/l, at least 0.3 g/l, at least 0.4 g/l, at least 0.5 g/l, at least 0.6 g/l, at least 0.7 g/l, at least 0.8 g/l, at least 0.9 g/l, at least 1.1 g/l, at least 1.2 g/l, at least 1.5 g/l, at least 1.6 g/l, at least 1.8 g/l, at least 2.0 g/l, at least 2.5 g/l, at least 3.0 g/l, at least 3.5 g/l, at least 4.0 g/l, at least 5.0 g/l, at least 10 g/l, at least 15 g/l, at least 20 g/l, at least 30 g/l, at least 40 g/l, at least 50 g/l, or more, after at least 3 days, at least 4 days, at least 5 days, at least 6 days, at least 7 days, at least 8 days, or at least 10 days of fermentation.

In some examples, the method further includes purifying or isolating the 3-HP made by the Aspergillus niger (e.g., a recombinant Aspergillus niger disclosed herein). Any suitable known method can be used to isolate the 3-HP. For example, separation techniques (such as filtration) can be used to remove the fungal biomass from the culture medium, and isolation procedures (e.g., filtration, distillation, precipitation, electrodialysis, and ion-exchange procedures) can be used to obtain the 3-HP from the broth (such as a fungi-free broth). The 3-HP can be isolated from the culture medium after the 3-HP production phase has been terminated.

In some examples, the generated 3-HP is used to make other chemicals, such as acrylic acid, malonic acid, methyl acrylate, ethyl 3-hydroxypropionic acid, propiolactone, acrylonitrile 1,3-propanediol, and acrylamide. For example, acrylic acid can be made by dehydration, malonic acid by oxidation, esters by esterification reactions with alcohols, and reduction to 1,3 propanediol. In one example, such chemicals are made ex vivo. In some examples, the recombinant A. niger disclosed herein can be further modified to express the enzymes needed to generate these other chemicals. Thus, further provided are recombinant A. niger that can make 3-HP downstream products (such as acrylic acid, malonic acid, methyl acrylate, ethyl 3-hydroxypropionic acid, propiolactone, acrylonitrile 1,3-propanediol, and acrylamide), as well as methods of making such products.

### V. Kits

Also provided are kits for performing a method disclosed herein. The kits can include a recombinant Aspergillus niger disclosed herein, a media, carbon source, nutrient additive, antifoam, antibiotic, filter, exclusion column, or combinations thereof. In some examples, the Aspergillus niger is freeze dried or lyophilized.

The media can be a media for culturing (including fermenting), storing, or growing the fungus. Exemplary mediums include solid medium (such as those containing agar, for example complete medium (CM) or minimal medium (MM)) and liquid media (such as a fermentation broth, such as CM, MM, Riscaldati B medium (RisB), or CAP medium).

Carbon sources include any chemical or ingredient the Aspergillus niger can metabolize and use as a source of carbon. Exemplary carbon sources include, but are not limited to, glucose, maltose, fructose, maltodextrin, and/or a glucose:xylose mixture. Nutrient additives facilitate Aspergillus niger growth and/or 3-HP production. Exemplary nutrient additives include, but are not limited to, trace elements, corn steep liquor, peptone, tryptone, soy protein, sodium chloride, yeast extract, sorbitol, and/or phosphorous. An antifoam is a chemical that reduces or prevents the formation of foam during culturing, such as during fermentation. Many antifoams are known and available, and a suitable antifoam (e.g., CLEROL FBA975) can be selected by a practitioner. Antibiotics are compounds that prevent the growth of unwanted organisms. Exemplary antibiotics include, but are not limited to, neomycin, bleomycin, and tetracycline.

### VI. Examples

Fuel and chemical products obtained from petroleum refineries have been essential in daily life for more than a century. However, petroleum products are non-renewable and their production and use has contributed to widespread anthropogenic impacts on the earth's atmosphere, lands, and oceans. This has prompted the investigation of alternative routes to produce fuels and chemicals at low carbon intensity from renewable feedstocks.

3-hydroxypropionic acid (3-HP) is a potential bioderived platform chemical that can be converted into various commercial use chemicals, such as acrylic acid, malonic acid, 1,3-propanediol, and acrylamide, as well as direct use for production of biodegradable polymers. Chemical synthesis routes have been explored for 3-HP production, but high costs and adverse environmental impacts have limited chemical synthesis of 3-HP as a bulk chemical (Jiang et al., Appl Microbiol Biotechnol 82(6):995-1003, 2009). However, biological fermentation is a potential route for 3-HP production from renewable feedstocks and has been actively investigated for more than a decade (Kumar et al., 31(6):945-61, 2013; Matsakas et al., Ferment 4(1):13, 2018). Naturally, there exist several 3-HP production processes, such as C02 assimilation in Chloroflexus aurantiacus (Holo, Arch Microbiol. 151(3):252-6, 1989) or cyanobacterium Synechocystis sp. (Lan et al., Metab Eng 31:163-70, 2015; Wang et al., Metab Eng 34:60-70, 2016), glycerol oxidation in Lactobacillus sp. (Talarico et al., Appl Environ Microbiol 56(4):943-8, 1990), acrylic acid degradation in Byssochlamys sp. (Takamizawa et al., Appl Microbiol Biotechnol 40(2):196-200, 1993) or Rhodococcus erythropolis (Lee et al., J Microbiol Biotechnol 19(5):474-81, 2009) and uracil catabolism in Saccharomyces kluyveri (Andersen et al., J mol biol 380(4):656-66, 2008) or E. coli K-12 (Loh et al., Proc Natl Acad Sci USA. 103(13):5114-9, 2006). However, the efficiency of 3-HP production in native microorganisms is very low. Therefore, genetic engineering of non-native hosts with novel synthetic 3-HP production pathways is needed.

Glycerol oxidation through a coenzyme A-independent pathway was initially detailed by Bieble et al. for 1,3-propanediol production in Clostridia and Enterobacteriaceae and the NAD⁺-dependent aldehyde dehydrogenase that can convert 1,3-propanediol to 3-HP in Klebsiella pneumoniae by Raj et al. (Biebl et al., Appl Microbiol Biotechnol 52(3):289-97, 1999; Raj et al., Biotechnol Bioprocess Eng 15(1):131-8, 2010). E. coli or K. pneumoniae was genetically engineered and optimized for 3-HP production by combination of glycerol reduction and 3-HP production, which led to 70 to 80 g/l 3-HP production titer in fed-batch fermentations with glycerol as a carbon source (Li et al., Sci Rep 6(1):26932, 2016; Chu et al., Biotechnol Bioeng 112(2):356-64, 2015).

Biosynthetic routes to 3-HP via malonyl-CoA and β-alanine have been demonstrated in E. coli (Jessen et al., WO/2008/027742, 2008; Rathnasingh et al., J Biotechnol 157(4):633-40, 2012), and further explored or optimized in various microorganisms such as E. coli, cyanobacteria, and Saccharomyces cerevisiae. For example, the malonyl-CoA pathway was introduced into E. coli for conversion of glucose to 3-HP, which resulted in 10 g/l 3-HP production in 36 hrs (Cheng et al., Bioresour Technol 200:897-904, 2016). Similar results were observed for the same pathway after integration into the chromosomes of S. cerevisiae or Schizosaccharoyrnces poinbe (Kildegaard et al., Microb Cell Fact 15(1):1-13, 2016; Suyama et al., J Biosci Bioeng 124(4):392-9, 2017).

The β-alanine 3-HP pathway consisting of Ti?olm castaneum aspartate 1-decarboxylase (PAND), Bacillus cereus β-alanine-pyruvate aminotransferase (BAPAT) and Escherichia coli 3-hydroxypropionate dehydrogenase (HPDH) was constructed and examined in S. cerevisiae, and resulted in a production titer of 13.4 g/l 3-HP in controlled fed-batch fermentation (Borodina et al., Metab Eng 27:57-64, 2015). Recently, it was demonstrated that the β-alanine 3-HP pathway was functional in the acidophilic filamentous fungus Aspergillus pseudoterreus (Pomraning et al., Front Bioeng Biotechnol 9:603832, 2021).

Filamentous fungi such as Aspergillus species are used industrially for organic acid production because of their ability to grow at very low pH (<2.0) and produce secreted metabolites in nutrient-limited growth conditions, which eliminates the requirement of medium pH neutralization. A. pseudoterreus can secrete more than 80 g/l of itaconic acid in culture medium and Aspergillus niger can grow in more than 20% glucose or sucrose and convert more than 90% of the feedstock to citric acid (Dai et al., Appl Environ Microbiol 70(4):2474-85, 2004; Karaffa et al., Appl Microbiol Biotechnol 61(3):189-96, 2003). However, it was previously unknown whether A. niger is a suitable host for 3-HP production. Here, A. niger is confirmed as a host for 3-HP production using the β-alanine 3-HP pathway.

### Example 1: Materials and Methods

### Strains and Media

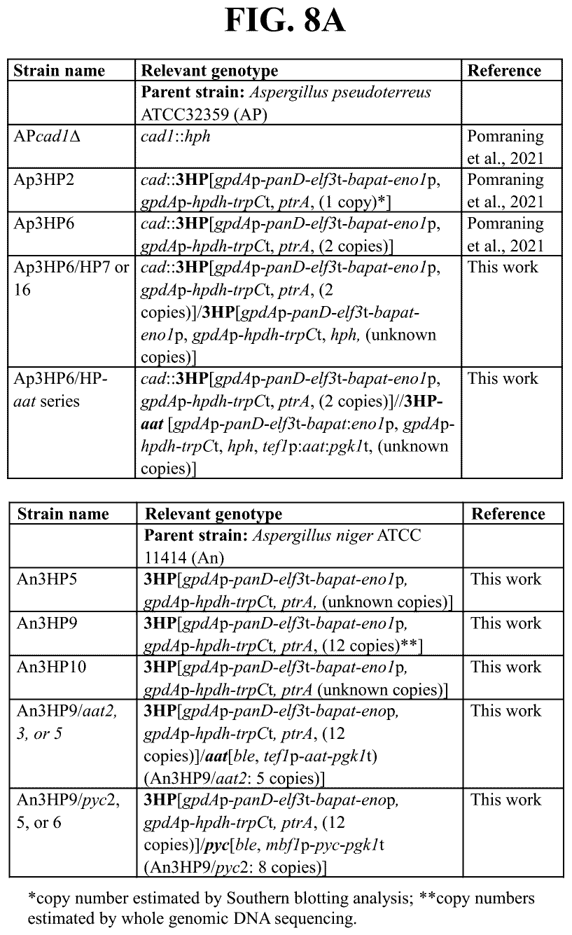

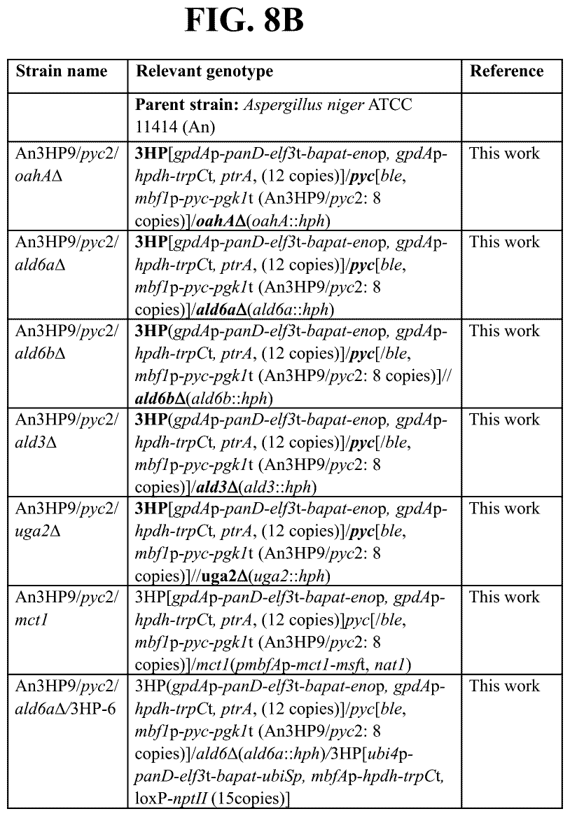

The Escherichia coli strain Top10 was used for routine plasmid DNA preparation. A. pseudoterreus (ATCC 32359) and A. niger (ATCC 11414) from the American Type Culture Collection (Rockville, MD, USA), were grown on complete medium (CM) or potato dextrose agar (PDA) plates at 30° C. for culture maintenance and spore preparation. About 1×10⁴ to 1×10⁵ spores were inoculated on CM agar (petri dish) plates and incubated for four days at 30° C. Spores were harvested by washing with 5 to 10 ml sterile 0.4% TWEEN 80 (polyoxyethylenesorbitan monooleate) and pelleted by centrifugation at 2500 g for 5 min. The spores were re-suspended in sterile 0.4% TWEEN 80 and enumerated with a hemocytometer. Aliquots of the resulting spore suspension (about 10⁸˜10⁹ spores/ml) were used to inoculate different agar-plates or liquid cultures. The preparation of PDA, CM and minimal medium (MM) followed the description of Bennett and Lasure (Bennett and Lasure. More Gene Manipulations in Fungi. San Diego: Academic Press p. 441-58, 1991). All strains used in this study are shown in FIGS. 8A-8B.

Preparation of Transgene Expression Constructs for Gene Over-Expression or Gene Disruption in A. pseudoterreus and A. niger.

The β-alanine pathway transgene expression cassette with pyrithiamine resistance gene (ptrA) of Aspergillus oryzae as a selection marker was described previously (Pomraning et al., Front Bioeng Biotechnol 9:603832, 2021). In this study, all transgene expression cassettes were prepared with Gibson assembly master mix (NEB, Ipswich, MA, USA) and the DNA fragments isolated by PCR with PHUSION high-fidelity DNA polymerase (Thermo Fisher Scientific, Walrham, MA).

### Culture Methods

Pyrex 125 ml or 250 ml glass Erlenmeyer flasks were prepared by filling with 5% CONTRAD 70 (Decon Labs, Inc. King of Prussia, PA, USA) and soaking overnight to remove any potential residues on the inside surface of flasks prior to general dishwashing. Silicon sponge closures were used for all flask cultures. The biomass of transgenic clones and parent strain for genomic DNA isolation were prepared from 2 mL stationary CM cultures with proper antibiotics and grown in 13×100 mm glass culture-tubes for 24 to 36 hrs at 30° C. The biomass formed on the surface of the liquid culture medium was collected, frozen immediately in liquid nitrogen and dried in the VirTis benchtop manifold freeze dryer (SP Scientific, Gardiner, NY, USA). For 3-HP production, 35 ml of citric acid production medium was prepared by following previous descriptions (Dai et al., Appl Environ Microbiol 70(4):2474-85, 2004). Production medium B (RDM) (Riscaldati et al., J Biotechnol 83(3):219-30, 2000) or modified production medium B (mRDM) (Pomraning Front Bioeng Biotechnol 9:603832, 2021; Dai et al., Appl Environ Microbiol 70(4):2474-85, 2004) that contains 20×TE (trace elements: 4 mg/l CuSO₄·5H₂O; 110 mg/l FeSO₄·7H₂O; 14 mg/l MnCl₂·4H₂O; and 26 mg/l ZnSO₄·7H₂O) was also used. The ferment sugars liberated from corn stover by deacetylated and disk refined process (DDR), in which the biomass was deacetylated with dilute alkaline at low temperature first, then mechanically refined in an industrial size disk refiner, and finally enzymatically hydrolyzed (DDR-EH; Batch 1-19-05, 20190829, (Chen et al., Biotechnol Biofuels 8:173, 2015)), which was obtained from the Pilot Plant at National Renewable Energy Laboratory (Golden, CO, USA).

Chemical-Mediated Protoplast Transformation of A. niger

The protoplast preparation and chemical-mediated transformation followed the method described by Dai et al. (Fungal Genet Biol 61:120-32, 2013) for A. niger. Briefly, the 14.4 kb plasmid DNA of the β-alanine pathway transgene expression construct was linearized by restriction enzyme EcoRV and concentrated down to about 1 mg/ml with Microcon-30 kDa centrifugal filter unit (MilliporeSigma, Burlington, MA). Ten microliters of the linearized plasmid DNA were used for protoplast transformation in A. niger. For transgene overexpression of A. niger aat1, pyc, the aat1-pyc, or mct1 gene in A. niger, about 3 to 5 mg of linearized plasmid DNAs by proper restriction enzymes were used for protoplast transformation. For the gene deletion construct of ald6a, ald6b, oah1, or uga2 gene homolog, about 1 mg of linearized plasmid DNAs by restriction enzyme PmeI was used for protoplast transformation in A. niger. Usually, about 5 to 12 transformed clones were picked randomly for evaluation of 3-HP production and the effects of selected genes on 3-HP production. The chemical-mediated protoplast transformation of A. pseudoterreus followed the previous description (Deng et al., Appl Microbiol Biotechnol 104(9):3981-92, 2020).

### Total Genomic DNA Isolation for PCR, Southern Blotting Analysis, and Short-Read Whole Genomic DNA Sequencing.

Total genomic DNA was isolated from A. niger or A. pseudoterreus cells using a cetyltrimethylammonium bromide (CTAB) extraction method with some modifications. Briefly, 50 to 100 mg of lyophilized biomass and two 3.5 mm diameter glass beads were transferred into a 2 mL polypropylene micro-vial, where biomass was pulverized into fine power with a Mini-Beadbeater-8 (Bio Spec Products Inc., Bartlesville, OK) for 50 seconds. The disrupted cells in microcentrifuge tubes were re-suspended with 800 to 900 μl of CTAB solution and incubated at 60° C. for 30-45 min and inverted occasionally. The genomic DNA in the supernatant of the cell extracts was extracted with 300 ml of phenol/chloroform solution and precipitated with 1 volume of 2-propanol. The genomic DNA was resuspended with 200 ml of 50TE (50 mM Tris-HCl, pH8.0 and 10 mM EDTA, pH8.0) and 25 μg of RNase and incubated for 30-45 min at 50° C. After RNase treatment, the genomic DNA was extracted twice with 125 ml of phenol/chloroform solution and once with chloroform. The genomic DNA in the supernatants was precipitated with 1 M NaCl and 2 volume of 95% ethanol for 15 min at room temperature and centrifugation at 10,000×g for 8 min. Finally, the genomic DNA pellet was washed with 70% ethanol and was resuspended in 10 mM Tris-HCl (pH 8.0) buffer at 50° C. for 15 to 20 min and the concentration was determined with a QUBIT fluorometer (Thermo Fisher Scientific, Waltham, MA, USA). Fifty to seventy ng of total genomic DNA were used for PCR analyses.

For Southern blotting analyses of heterologous expression of β-alanine pathway in either A. niger or A. pseudoterreus, 1 mg of total genomics DNA was digested with the restriction endonuclease BamHI, EcoRV, or HindIII. The genomic DNA fragments were separated in 1% agarose gel electrophoretically and transferred onto the Hybond™-N⁺ nylon membrane (GE Healthcare Bio-Sciences, Pittsburgh, PA, USA) with alkaline capillary transfer method. The 1.0 kb 3′-end of genomic DNA fragments of A. pseudoterreus cad1 gene was used for preparation of the biotin-labeled probe. The genomic DNA in the Hybond™-N⁺ nylon membrane was hybridized with the biotin-labeled probe overnight at 60° C. in the Problot™ Hybridization Oven (Labnet International, Edison, NJ, USA). The genomic DNA on the hybridized membrane was visualized with North2South™ chemiluminescent detection kit (Pierce Protein Research Products, Rockford, IL, USA) in Analytik jena UVP ChemStudio (Analytik Jena US, Upland, CA, USA).

The short-read whole genomic DNA sequencing was carried out by Azenta Life Sciences (South Plainfield, NJ, USA). The integration copy number was estimated by fold-increase of reads mapped to the expression construct versus background single copy regions of the genome. The sequenced short-reads were mapped to the reference genome sequence of A. niger ATCC 1015 (mycocosm.jgi.doe.gov/Aspni7/Aspni7.home.html) augmented with the overexpressed gene sequence using BWA-MEM (Li H et al., arXiv preprint 13033997, 2013). The mapped reads were sorted using SAMtools (Li et al., Bioinformatics 25(16):2078-9, 2009) and duplicate reads were marked using Picard Toolkit (github.com/broadinstitute/picard #citing) to produce BAM files for copy number estimation. The copy numbers of β-alanine pathway genes and engineered native genes were estimated using CNVnator (Abyzov et al., Genome Res 21(6):974-84, 2011). The mapped reads were counted using bin sizes of 100, 200, and 1,000 bp, and the read depth signal was partitioned into segments for each bin size. The average and standard deviation of read depth signal were evaluated for bin sizes of 100 and 200 bp, and copy number genotype was estimated based on the normalized read depth using the bin size of 100 bp.

### Metabolites Analysis by HPLC

The extracellular metabolites were quantified by HPLC. Twenty-five microliters of the samples filtered with 0.2 mm syringe filters were analyzed for 45 min using an AMINEX HPX-87H ion exclusion column with a 1 mM H₂SO₄ flow of 0.6 ml/ml. The temperature of the column was 60° C. The refractive index at 45° C. and the UV absorption at 210 nm were measured.

### Sample Preparation for Metabolomics and Proteomics Analyses

Briefly, the culture supernatants or biomass (cell pellet) for A. niger or A. pseudoterreus were harvested at day 4. For quantification of extracellular metabolites diluted spent medium samples (by a 1/8 factor) were dried, prepared, and analyzed as described previously (Pomraning et al., Front Bioeng Biotechnol 9:603832, 2021). The cell pellets were extracted using the MPLex protocol (Nakayasu et al., MSystems 1(3):e00043-16, 2016) and extracts were analyzed using GC-MS as explained previously in detail (Kim et al., Front Microbiol 6:209, 2015). The protein interlayer pellet was digested and prepared for global proteomics analysis and targeted proteomics analysis, the latter using heavy labeled peptides. Instrument acquisition and data analysis was done as described in a previous publication (Pomraning et al., Front Bioeng Biotechnol 9:603832, 2021). Global proteomics data were generated using a Q Exactive Plus™ mass spectrometer (Thermo Fisher Scientific, Waltham, MA, USA) in data-dependent acquisition mode.

### Bioreactor Operations and Shaking Flasks

Transgenic strains were routinely cultured in Riscaldati 13 medium (RisB) that contained 20×TE (trace elements: 4 mg/L CuSO4·5H2O; 110 mg/L FeSO4·7H2O; 14 mg/L MnCl2·4H2O; and 26 mg/L ZnSO4·7H2O). Glucose, maltose, fructose, maltodextrin, or glucose:xylose mixtures (2:1 g/g ratio) were used as the main carbon sources. The medium was modified with various additives (i.e., Corn steep liquor, peptone, tryptone, soy protein, sodium chloride, yeast extract, sorbitol) at the specified concentrations. The base medium was also modified in specific tests to contain additional phosphorus ranging from 1-10× the normal RisB phosphorus concentration, as specified within the figures. Shaking flasks were inoculated with either spores at a concentration of approximately 1×10\hat{ }6 spores/mL or with 10% vol/vol of a seed culture. The seed culture media contained 100 mL/1 L of corn steep liquor, 3 mL/1 L of CLEROL FBA975 antifoam, 1 g/L NaH2PO4·H₂O, 2.04 g/L MgSO4.7H₂O, 100 g/L maltose, 10 g/L glucose, 50 g/L fructose and was inoculated using spores a concentration of approximately 1×10\hat{ }6 spores/mL and grown overnight (15-18 h) at 34 C. Before inoculation, the seed culture was washed with deionized water and resuspended in a RisB salt solution containing 1×phosphate. Shaking flasks production cultures occurred at 30 and 35 C. Bioreactor cultivations were performed in an Infors AG Sixfors Fermenter system with 675 mL vessels in RisB salt mediums containing the specified sugars and additives. A working volume of 450 mL was used through fermentations. The bioreactors were inoculated using the spore procedure specified above and allowed to germinate for approximately six hours before aeration was initiated.

The extracellular metabolites were quantified by HPLC. Twenty-five microliters of the samples filtered with 0.2 μm syringe filters were analyzed for 45 min using an AMINEX HPX-87H ion exclusion column with a 1 mM H2SO4 flow of 0.6 mL/mL. The temperature of the column was 60° C. The refractive index at 45° C. and the UV absorption at 210 nm were measured. For dry cell weight determination, 5-10 mLs of culture was pelleted in a pre-weighed conical tube (5 min at 4,000 g), the cells were washed once with water, and frozen at −20 C. The frozen pellet was then lyophilized for 24-36 h.

Detailed Description of Transgene Vector Construction for Gene Overexpression or Disruption in Aspergillus pseudoterreus or Aspergillus niger.

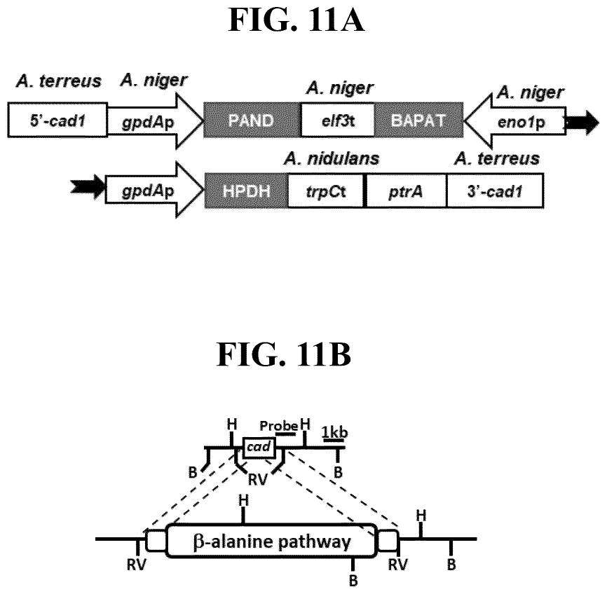

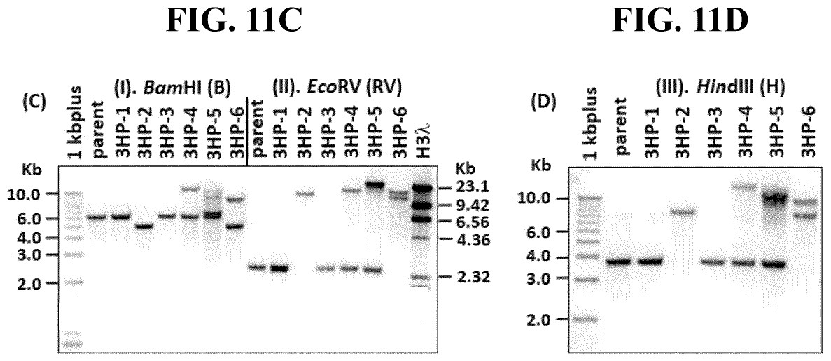

All transgene over-expression or gene disruption constructs were prepared by Gibson assembly method (Ipswich, MA) with DNA fragments isolated by PCR from either related plasmid or genomic DNAs of Aspergillus carbonarius, A. niger, Aspergillus oryzae, or A. pseudoterreus with proper oligo pairs and PHUSION high-fidelity DNA polymerase (Thermo Fisher Scientific, Waltham, MA, USA 02451). The first β-alanine pathway transgene expression cassette with pyrithiamine resistance gene (ptrA; Genbank: AF217503) of A. oryzae as a selection marker shown in FIG. 11A was prepared as follows: the first 987 bp DNA fragment of cad1 (Genbank: UDM55691.1, 5′-cad1) gene upstream region containing 535 bp coding sequence of N-terminus was isolated by PCR with oligo pair 1969cad1/1670cad1 (Table 1); the second 813 bp DNA fragment of A. niger gpdA (Genbank: XP_025477631, gpdAp) promoter with oligo pair 1971gpdA1/1972gpdA2; the third 1623 bp DNA fragment of aspartate 1-decarboxylase (Tribolium castaneum panD, PAND) with 1973pan1/1974pan2 and the synthetic plasmid DNA of codon usage optimization for A. pseudoterreus as a template; the forth 1040 bp DNA fragment of bi-direction transcriptional terminator of A. niger elf3-multifunctional chaperone (elf3, Genbank: CAK43351.1, elf3t) with oligo pair 1975ter1/1976ter2; the fifth 1350 bp reverse complement DNA fragment of b-alanine-pyruvate aminotransferase (Bacillus cereus bapat, BAPAT) with oligo pair 1977bap1/1978bap2 and the synthetic plasmid DNA of codon usage optimization for A. pseudoterreus as a template; and final 704 bp complement DNA fragment of A. niger enolase (Genbank: AM270411.1, eno1p) promoter with oligo pair 1979eno1/1980eno2-HpaI. All 6 DNA fragments were assembled together into the pBSK(−) vector linearized with restriction endonuclease HindIII and PstI to form new intermediate vector 3HP4025. The 885 bp DNA fragment of Aspergillus nidulans gpdA promoter was prepared by PCR with oligo pair 1981gpdA1/1982gpdA2 and pAN7-1 plasmid DNA (Genbank: Z32698.1, gpdAp) as a template; the 744 bp DNA fragment of 3-hydroxypropionate dehydrogenase (Escherichia coli hpdh, HPDH) with oligo pair 1983hpd1/1984hpd2 and the synthetic plasmid DNA of codon usage optimization for A. pseudoterreus as a template; the 473 bp DNA fragment of A. nidulans trpC (trpCt) transcriptional terminator with oligo pair 1985trp1/1986trp2 and pAN7-1 plasmid DNA as a template; the 2005 bp DNA fragment of A. oryzae ptrA gene with oligo pair 1987ptrA1/1988ptrA2 (ptrA); and 908 bp DNA fragment of A. pseudoterreus cad1 gene downstream region containing 519 bp coding sequence of its C-terminus and transcriptional terminator with oligo pair 1989cad3/1990cad4 (3′-cad1). Above 5 DNA fragments were assembled into the intermediate vector 3HP4025 linearized with the restriction endonuclease HpaI, which was introduced into the 3HP4025 with oligo 1980eno2-HpaI, to form the entire b-alanine pathway transgene expression cassette 3HP4028. The entire transgene expression construct was verified by Sanger DNA sequencing. The second 3-alanine pathway transgene expression cassette was to construct two identical copies of the β-alanine pathway, where the plasmid 3HP4028 was linearized by restriction endonuclease EcoRI and assembled with the entire fragment of the β-alanine pathway, which was isolated by PCR with oligo pair 2085HP2F/2086HP2R and 3HP4028 as a DNA template to form the plasmid 3HP4046 (FIG. 13).

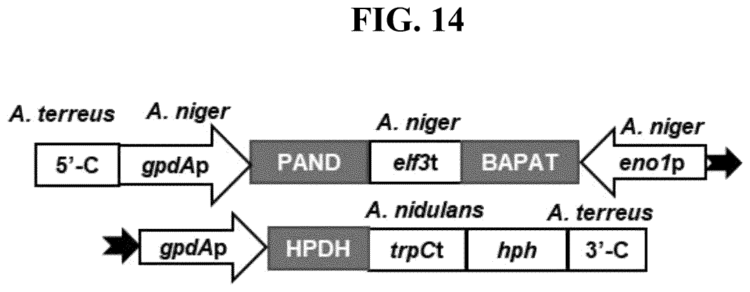

The third β-alanine pathway transgene expression cassette 3HP4070 with E. coli hygromycin B phosphotransferase (hph) marker gene (FIG. 14) was prepared with the plasmid 3HP4028 and pCB1003 (fgsc.net/fgn41/carroll.html) as the DNA templates for PCR as follows: the first 2759 bp DNA fragment containing 326 bp of 5′-end cad1 coding sequence of A. pseudoterreus, 810 bp of A. niger gpdA promoter, and 1623 bp DNA fragment of PAND with oligo pair 2201/2202; the second 3091 bp DNA fragment containing the 1040 bp DNA fragment of bi-direction transcriptional terminator of A. niger elf3, the 1350 bp reverse complement DNA fragment of bapat, and 701 bp reverse complement DNA fragment of A. niger eno1 promoter with oligo pair 2203/2204. These two fragments were assembled into the pBSK(−) vector linearized with restriction endonuclease HindIII and PstI to form new intermediate vector 3HP4069. The 1360 bp DNA fragment of hph marker gene was prepared by PCR with oligo pair 2213hphF/2214hphR and plasmid DNA pCB1003 as a template. The 2385 bp DNA fragment containing 885 bp DNA fragment of A. nidulans gpdA promoter, 744 bp DNA fragment of hpdh, 473 bp DNA fragment of A. nidulans trpC transcriptional terminator, and 283 bp upstream region of A. oryzae ptrA gene was isolated with oligo pair 2215hpdF/2216hpdR. These two fragments were further assembled into the 3HP4069 linearized with restriction endonuclease XbaI to form the 3HP4070 transgene expression vector. The entire transgene expression construct was verified by Sanger DNA sequencing.

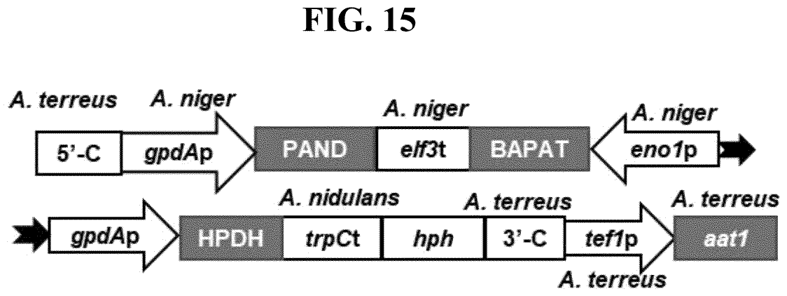

The fourth β-alanine pathway transgene expression cassette with an additional A. pseudoterreus aspartate 1-aminotransferase gene (Apaat1, jgi|Aspte1|7965|ATET_04402; FIG. 15) was prepared by PCR with oligo pairs of PAptefF/PAptefR for 956 bp A. pseudoterreus tef1 gene (Aptef1, jgi|Aspte1|9546|ATET_05983) promoter and the ApaatF/Apaat for 1898 bp A. pseudoterreus aspartate aminotransferase entire coding region and 269 bp its transcriptional terminator. The fragments were assembled into 3HP4070 linearized with restriction endonuclease XbaI to form transgene expression cassette 3HP4071. The entire transgene expression construct was verified by Sanger DNA sequencing.

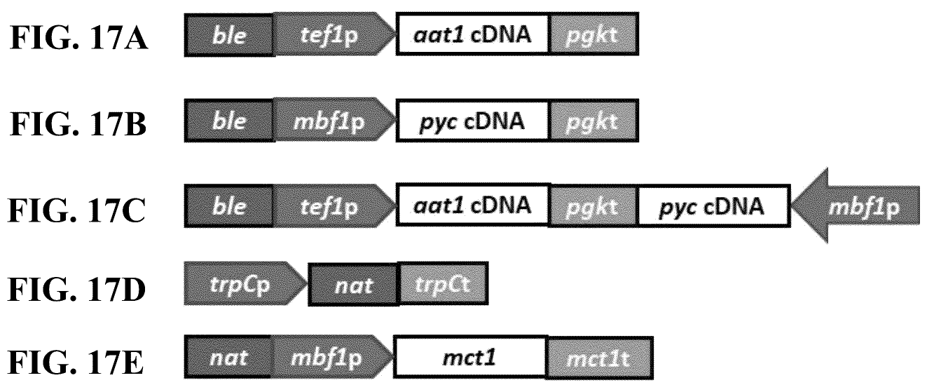

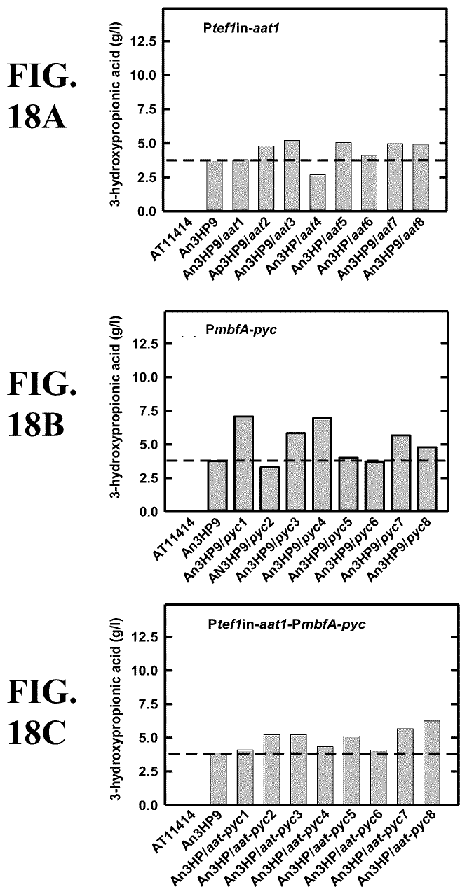

The PCR fragments for A. niger aspartate aminotransferase gene (aat1, GenBank: EHA22111) over-expression construct 3HP4074 (FIG. 17A, ble, tef1P:aat:pgk1T) were prepared with the oligo pair bleF1/bleR1 and plasmid DNA pAN8-1 (GenBank: Z32751.1) for 1550 bp DNA fragment of the bacterial bleomycin resistance (ble) marker gene, the oligo pair PAntef1F/PAntef1R for 870 bp DNA fragment of translation elongation factor 1 (tef1, Genbank: CBJ23536.1) promoter of A. niger, the oligo pair AnaatF/AnaatR for the 1401 bp aat1 cDNA of A. niger, and the oligo pair TAnpgkF1/TAnpgkR1 for 772 bp DNA fragment of the transcriptional terminator of phosphoglerate kinase (pgk, jgi|aspin7|1147902) gene of A. niger. All PCR fragments were assembled together into the pBSK(−) linearized by HindIII and PstI restriction enzymes with Gibson assembly master mix (NEB, Ipswich, MA, USA). Similarly, the over-expression transgene construct for pyruvate carboxylase were prepared with the oligo pairs of bleF1/bleR2 (ble) for 1550 bp DNA fragment of the ble gene, TAnpgkF2/TAnpgkR1 (Tpgk) for 772 bp DNA fragment of the transcriptional terminator of pgk, AnpycF/AnpycR for 3579 bp cDNA fragment of A. niger pyruvate carboxylase cDNA (pyc; jgi|Aspni7|1031996), and PAnmbf1F/PAnmflR for 1499 bp DNA fragment of A. niger mbf1 gene (jgi|Aspni7|1145066) promoter (FIG. 17B, 3HP4076) or their combination, where pyc overexpression cassette was incorporated into the downstream of aat1 overexpression cassette right after the TpgkF2/TpgkR1 (pgkt) DNA fragment (FIG. 17C, 3HP4077). The entire transgene expression construct was verified by Sanger DNA sequencing.

Prior to building the transgene overexpression construct for A. niger monocarboxylate transporter (mct1, jgi|Aspni7|1163060), the new selection marker nat1 (Streptomyces noursei nourseothricin N-acetyl transferase optimized for the codon usage of Saccharomyces cerevisiae) gene under the control of A. nidulans trpC promoter and A. niger trpC transcriptional terminator cassette was prepared. The DNA fragment for A. nidulans trpC promoter and A. niger trpC transcriptional terminator were isolated with the oligo pair 2555trpF1/2556trpR1 and 2559trpF2/2560trpR2 with the pCSN44 plasmid (GenBank: LT726870.1) as a template for trpC promoter and with A. niger genomic DNA as a template for A. niger trpC transcriptional terminator, respectively. The nat1 coding region was obtained by PCR with oligo pair 2557nat1F/2558nat1R and the plasmid DNA CHCp9 (Calvey et al., Curr Genet 60(3):223-30, 2014) as a template. All three DNA fragments were assembled to form vector (FIG. 17D, 3HP4114). The mct1 overexpression construct was prepared with the oligo pairs of nat1F/nat1R for 1250 bp DNA fragment of nat1 marker gene with 3HP4114 as a template, PAnmbf1/PAnmbf1R for 1351 bp DNA fragment of A. niger mbf1 gene promoter and AnmctF/AnmctR for 1544 bp DNA fragment of A. niger mct gene coding sequence and its transcriptional terminator, which were assembled into pBSK(−) linearized with HindIII/PstI restriction endonucleases to form 3HP4126 (FIG. 17E). The entire transgene expression construct was verified by Sanger DNA sequencing.

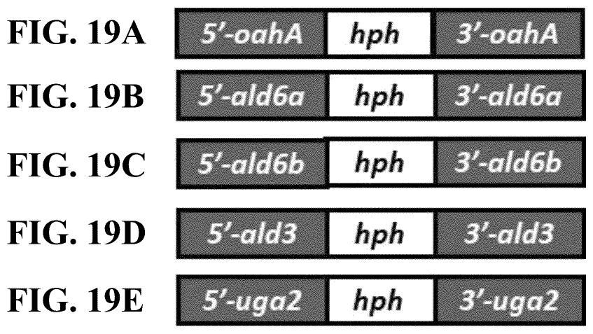

Gene disruption constructs for oahA (jgi/Aspni7/1145269), ald6a (jgi/Aspni7/1182225), ald6b (jgi/Aspni7/201822), ald3 (jgi/Aspni7/1126238), and uga2 (jgi/Aspni7/57046) from A. niger were prepared by Gibson assembly of related DNA fragments isolated via PCR with the following oligo pairs: 5AnoahAF/5AnoahAR, hphF1/hphR1, and 3AnoahAF/3AnoahAR for oahA gene (FIG. 19A, 3HP4102); 5Anald6aF/5Anald6haR, hphF2/hphR2, and 3Anald6aF/3Anald6aR for ald6a gene (FIG. 19B, 3HP4103); 5Anald6bF/5Anald6bR, hphF3/hphR3, and 3Anald6bF/3Anald6bR for ald6b gene (FIG. 19C, 3HP4104); 5Anald3F/5Anald3R, hphF4/hphR4, and 3Anald3F/3Anald3R for ald3 gene (supplementary FIG. 19D, 3HP4108); 5Anuga2F/5Anuga2R, hphF5/hphR5, and 3uga2F/3uga2R for uga2 gene (FIG. 19E; 3HP4109).

For further genetic modification in the transgenic strain, the marker recycling strategy was applied to overcome the limitation of selection marker genes in A. niger. The Tet-On/Cre-loxP system (Jiang et al., Front Microbiol 7:485, 2016; Meyer et al., Appl Environ Microbiol. 77(9):2975-83, 2011) was incorporated into the new transgene expression or gene disruption. The Tet-On/Cre-loxP transgene expression construct was prepared with the oligo pairs of nat1F/nat1R for 1250 bp DNA fragment of entire nat1 marker gene with 3HP4114 as a template, ubilF/ubilR, rtTAF/rtTAR, tetO7F/tetO7R, and trpCF/trpCR for 848 bp DNA fragment of A. niger ubi1S27 gene (jgi|Aspni7|1135631) promoter, 1036 bp DNA fragment of reverse tetracycline transactivator (rtTA2S), 1550 bp DNA fragment of tetracycline resistance operon/gpdA mini promoter of A. nidulans/Cre recombinase (tetO7-Pmn-Cre), in which the rtTA2S and tetO7-Pmn-Cre were synthesized with codon usage optimization for A. niger, and 554 bp DNA fragment of A. carbonarius trpC transcriptional terminator, respectively. All DNA fragments were assembled into the pBSK(−) linearized with HindIII/PstI restriction endonucleases to form transgene expression vector 3HP4140 (FIG. 20A). The entire transgene expression construct was verified by Sanger DNA sequencing.

The nptII [the neomycin phosphotransferase II gene, (Beck et al., Gene 19(3):327-36, 1982)] marker gene for A. niger was constructed with the DNA fragments isolated by PCR with oligo pair 2662mdhFp/2663mdhRp for 965 bp DNA fragment of A. niger mdh (jgi|Aspni7|1143375) gene promoter and oligo pair 2664npt2F/2665npt2R for 1138 bp DNA fragment of nptII gene coding region (GenBank: AF485783.1, 795 bp) and A. nidulans trpC transcriptional terminator (GenBank: U24705.1, 343 bp). The DNA fragments were assembled into the pBSK(−) linearized with HindIII/PstI restriction endonucleases to form plasmid vector 3HP4134 (FIG. 20B). To introduce the 31 bp loxP fragments into the nptII marker gene, the oligo pair loxPnpt2F/loxPnpt2R was used to isolate the nptII marker gene from 3HP4134 plasmid DNA and assembled into the pBSK(−) linearized with HindIII/PstI restriction endonucleases to form plasmid vector 3HP4136 (FIG. 20C).

To construct the fifth b-alanine 3HP pathway transgene expression cassette with loxP-nptW as a selection marker and a new set of A. niger promoters, the 3HP4028 plasmid DNA was used for a DNA template for isolation of panD, bapat, hpdh, elf3t and trpCt with the following oligo pairs: 2728pu4F/2729pu4R for 819 bp DNA fragment of A. niger ubi4 (jgi|Aspni7|1146681) gene promoter, 2730panF/2731panR for 2198 bp DNA fragment of panD gene and a part of elf3t, 2732bapatF/2733bapatR for 1823 bp DNA fragment of bapat gene and a part of elf3t, and 847 bp DNA fragment of 2734pus1F/2735pus1R forA. niger ubiS27 gene promoter in reverse complementation. The first 4 DNA fragments were assembled into the 3HP4136 vector linearized with restriction endonuclease XhoI to form plasmid 3HP4144 (FIG. 20D). The oligo pair 2736pmbF/273pmbR for 1199 bp DNA fragment of A. niger mbfA promoter, and 2738hpdhF/2739hpdhR for 1257 bp DNA fragment of hpdh gene and A. nidulans trpC transcriptional terminator. These two fragments were assembled into the plasmid 3HP4144 linearized with restriction endonuclease XbaI to the final transgene expression construct 3HP4145 shown in FIG. 20E. The entire transgene expression construct was verified by Sanger DNA sequencing.

TABLE 1
Oligos used for transgene vector constructions
of selected gene over-expressions or
disruptions.

| | SEQ ID NO. | |
|---|---|---|
| | | β-alanine pathway transgene expression cassette with |
| | | ptrA marker gene (plasmid vector 3HP4028) |
| 1969cad1 | SEQ ID NO: 10 | ccctcgaggtcgacggtatcgataGATATCGGTTGTAGCAGCGT |
| | | AAACAC |
| 1970cad2 | SEQ ID NO: 11 | tctttcatagtagCCTTGGTGAACATCTTGAGG |
| 1971gpdA1 | SEQ ID NO: 12 | atgttcaccaaggCTACTATGAAAGACCGCGATG |
| 1972gpdA2 | SEQ ID NO: 13 | cgccggtggcgggCATTGTTTAGATGTGTCTATGTG |
| 1973pan1 | SEQ ID NO: 14 | catctaaacaatgCCCGCCACCGGCGAGGACCA |
| 1974pan2 | SEQ ID NO: 15 | atccaacccatcaGAGGTCGGAGCCCAGGCGTTCG |
| 1975ter1 | SEQ ID NO: 16 | gggctccgacctcTGATGGGTTGGATGACGATG |
| 1976ter2 | SEQ ID NO: 17 | tctggcccagctcTGAGTCCTAGATGGGTGGTG |
| 1977bap1 | SEQ ID NO: 18 | catctaggactcaGAGCTGGGCCAGACATTCCTTC |
| 1978bap2 | SEQ ID NO: 19 | gtccatcaacatgGAACTGATGATCGTCCAGGTCAC |
| 1979eno1 | SEQ ID NO: 20 | cgatcatcagttcCATGTTGATGGACTGGAGGG |
| 1980eno2- | SEQ ID NO: 21 | gaactagtggatcccccgggctgcGttaaCTCGAGCTTACAAGAA |
| HpaI | | GTAGCC |
| | | GGCTACTTCTTGTAAGCTCGAGttaaCgcagcccgggggatc |
| | | cactagttc |
| 1981tdhA1 | SEQ ID NO: 143 | acaggctacttcttgtaagctcgagttTCTGTACAGTGACCGGTGA |
| | | C |
| 1982tdhA2 | SEQ ID NO: 22 | tgaccagcacgatCATGGTGATGTCTGCTCAAG |
| | | CTTGAGCAGACATCACCATGatcgtgctggtca |
| 1983hpd1 | SEQ ID NO: 23 | agacatcaccatgATCGTGCTGGTCACGGGCGC |
| 1984hpd2 | SEQ ID NO: 24 | gccatcggtcctaTTGGCGGTGGACGTTCAGGC |
| | | GCCTGAACGTCCACCGCCAAtaggaccgatggc |
| 1985trp1 | SEQ ID NO: 25 | cgtccaccgccaaTAGGACCGATGGCTGTGTAG |
| 1986trp2 | SEQ ID NO: 26 | cccgtctgtcagaGAGCGGATTCCTCAGTCTCG |
| | | CGAGACTGAGGAATCCGCTCtctgacagacggg |
| 1987ptrA1 | SEQ ID NO: 27 | gaggaatccgctcTCTGACAGACGGGCAATTGATTAC |
| 1988ptrA2 | SEQ ID NO: 28 | gaatgttgctgagGAGCCGCTCTTGCATCTTTG |
| | | CAAAGATGCAAGAGCGGCTCctcagcaacattc |
| 1989cad3 | SEQ ID NO: 29 | gcaagagcggctcCTCAGCAACATTCGCCATGTTC |
| 1990cad4 | SEQ ID NO: 30 | actaaagggaacaaaagctggagctCAGCTCCACTGCTCATAGT |
| | | CTTTG |
| | | Two copies of β-alanine pathway transgene expression |
| | | cassette with ptrA marker gene (plasmid vector |
| | | 3HP4046, 2x3HP) |
| 2085HP2F | SEQ ID NO: 31 | acacaattctctatctcagatttgCTACTATGAAAGACCGCGATG |
| | | GGC |
| 2086HP2R | SEQ ID NO: 32 | ttcagtttcgtccgaggacttttggAATTCAAATCTGAGATAGAGA |
| | | ATTG |
| | | β-alanine pathway transgene expression cassette with |
| | | hph marker gene (plasmid vector 3HP4070) |
| 2201 | SEQ ID NO: 33 | cgaggtcgacggtatcgataACGGATCGGCAAAGCAATCTAC |
| | | G |
| 2202 | SEQ ID NO: 34 | atccaacccaTCAGAGGTCGGAGCCCAGGC |
| 2203 | SEQ ID NO: 35 | cgacctctgaTGGGTTGGATGACGATGACTTC |
| 2212 | SEQ ID NO: 36 | agtggatcccccgggctgcaCTCGAGCTTACAAGAAGTAGCC |
| | | TG |
| 2213hphF | SEQ ID NO: 37 | gcccgggggatccactagttGCTGGAGCTAGTGGAGGTCAAC |
| 2214hphR | SEQ ID NO: 38 | ctgtacagagCGGTCGGCATCTACTCTATTC |
| 2215hpdF | SEQ ID NO: 39 | atgccgaccgCTCTGTACAGTGACCGGTGAC |
| 2216hpdR- | SEQ ID NO: 40 | agggaacaaaagctggagcttctAGAGAATTGTGTGGGATGAG |
| XbaI | | |
| | | β-alanine pathway transgene expression cassette with |
| | | hph marker gene and aat1 (plasmid vector 3HP4071) |
| PAptefF | SEQ ID NO: 41 | ctctcatcccacacaattctGAGCATCATCCCATGATAGC |
| PAptefR | SEQ ID NO: 42 | aaaggggcgaACTGTTGTAGAAGATATCCGTTAG |
| ApaatF | SEQ ID NO: 43 | ctacaacagtTCGCCCCTTTCCTCCTCTTC |
| ApaatR | SEQ ID NO: 44 | gggaacaaaagctggagcttctagaCGAGCAATACGGAAGCGAA |
| | | TATC |
| | | Over-expression of A. niger aat (plasmid vector |
| | | 3HP4074) |
| bleF1 | SEQ ID NO: 45 | ctcgaggtcgacggtatcgataGtttaaaCTGAGGTGCAGTGGATG |
| | | ATTATTAATC |
| bleR1 | SEQ ID NO: 46 | gaggtcaacgTTGATCTGCTTGATCTCGTC |
| PAntef1F | SEQ ID NO: 47 | agcagatcaaCGTTGACCTCACAGGGATTTC |
| PAntef1R | SEQ ID NO: 48 | aaaggggcgaCTTACTGTTGTAGAAGATATCCGTTAG |
| Anaat1F | SEQ ID NO: 49 | caacagtaagTCGCCCCTTTCCTCCTCTTC |
| Anaat 1R | SEQ ID NO: 50 | gacagggcagTTATGAAGTCTCCCGAACTACGC |
| TAnpgk1F | SEQ ID NO: 51 | gacttcataaCTGCCCTGTCGAGTAAGTAAATTTG |
| TAnpgk1R | SEQ ID NO: 52 | actagtggatcccccgggctgcaagcttACTACAGAGAGGAGCTG |
| | | AAG |
| | | Over-expression of A. niger pyc (plasmid vector |
| | | 3HP4076) |
| bleR2 | SEQ ID NO: 53 | atggccactgTTGATCTGCTTGATCTCGTCTC |
| 2271TAnpg | SEQ ID NO: 54 | caaggcctagCTGCCCTGTCGAGTAAGTAAATTTG |
| kF2 | | |
| AnpycF | SEQ ID NO: 55 | TCttcagctcctctctgtagtaCTAGGCCTTGACGATCTTGCAG |
| | | ACAAGATCCTG |
| AnpycR | SEQ ID NO: 56 | cttcaaaatgGCTGCTCCCCGCCAGCCCGA |
| PAnmbf1F | SEQ ID NO: 57 | ggggagcagcCATTTTGAAGATGGATGAGAAGTC |
| PAnmbf1R | SEQ ID NO: 58 | CTccaccgcggtggcggccgctGTTTAAACAGTGGCCATGAA |
| | | ATCCAATC |
| | | Nat1 selection marker gene construct (plasmid vector |
| | | 3HP4114) |
| 2555trpF1 | SEQ ID NO: 59 | cgaggtcgacggtatcgataCAGAAGATGATATTGAAGGAGC |
| 2556trpR1 | SEQ ID NO: 60 | aagtagtcatTTGGATGCTTGGGTAGAATAG |
| 2557nat1F | SEQ ID NO: 61 | aagcatccaaATGACTACTTTGGATGACACTG |
| 2558nat1R | SEQ ID NO: 62 | aaagctagagTTATGGACATGGCATGGACATG |
| 2559trpF2 | SEQ ID NO: 63 | atgtccataaCTCTAGCTTTGTATTGTCTTTAAATTTAC |
| 2560trpR2 | SEQ ID NO: 64 | agtggatcccccgggctgcaGTAAGTAGAAAGCTTTGGGG |
| | | mct1 gene overexpression (plasmid vector 3HP4126) |
| nat1F | SEQ ID NO: 65 | cgaggtcgacggtatcgataACAGAAGATGATATTGAAGGAG |
| | | C |
| nat1R | SEQ ID NO: 66 | aacgaagaggCAGTAAGTAGAAAGCTTTGGG |
| PAnmbf1F | SEQ ID NO: 67 | ctacttactgCCTCTTCGTTTCTGTGATGC |
| PAnmbf1R | SEQ ID NO: 68 | cggtcgtatgCATTTTGAAGATGGATGAGAAGTC |
| AnmctF | SEQ ID NO: 69 | cttcaaaatgCATACGACCGAGAAGATACC |
| AnmctR | SEQ ID NO: 70 | agtggatcccccgggctgcaGGTTCTCTCCTTGCAGCAAAG |
| | | oahA gene deletion (plasmid vector 3HP4102) |
| 5AnoahAF | SEQ ID NO: 71 | gaggtcgacggtatcgataagcttCTCAGCTGGGTGAAGAACAA |
| | | C |
| 5AnoahAR | SEQ ID NO: 72 | tagctccagcGTGATAGTGTTGGTCATGCTG |
| hphF1 | SEQ ID NO: 73 | acactatcacGCTGGAGCTAGTGGAGGTCAAC |
| hphR1 | SEQ ID NO: 74 | cgttagtatgCGGTCGGCATCTACTCTATTCC |
| 3AnoahAF | SEQ ID NO: 75 | atgccgaccgCATACTAACGGAAGGGTCAG |
| 3AnoahAR | SEQ ID NO: 76 | agtggatcccccgggctgcaGTACCACGCAAGCTTCGATATG |
| | | aldoa gene deletion (plasmid vector 3HP4103) |
| 5Anald6aF | SEQ ID NO: 77 | gaggtcgacggtatcgatatctaGAGTAAACTGGTGCAGCTATC |
| 5Anald6aR | SEQ ID NO: 78 | tagctccagcAGGTGAGGATGAGGAGAGCTTAG |
| hphF2 | SEQ ID NO: 79 | atcctcacctGCTGGAGCTAGTGGAGGTCAAC |
| hphR2 | SEQ ID NO: 80 | gactgtcttgCGGTCGGCATCTACTCTATTCC |
| 3Anald6aF | SEQ ID NO: 81 | atgccgaccgCAAGACAGTCGATTTCATCCTC |
| 3Anald6aR | SEQ ID NO: 82 | agtggatcccccgggctgcaCACTTGTCCAGACCGAGGTAC |
| | | ald6b gene deletion (plasmid vector 3HP4104) |
| 5Anald6bF | SEQ ID NO: 83 | gaggtcgacggtatcgataGAGGATATGATCTCTTCAACTAT |
| | | AC |
| 5Anald6bR | SEQ ID NO: 84 | tagctccagcTCTCAGGAGACATCTGTCTC |
| hphF3 | SEQ ID NO: 85 | tctcctgagaGCTGGAGCTAGTGGAGGTCAAC |
| hphR3 | SEQ ID NO: 86 | ctaccatcgaCGGTCGGCATCTACTCTATTCC |
| 3Anald6bF | SEQ ID NO: 87 | atgccgaccgTCGATGGTAGTAGGATGTGG |
| 3Anald6bR | SEQ ID NO: 88 | agtggatcccccgggctgcaCAGAGAAGGTGCAGATTGTG |
| | | aldh3 gene deletion (plasmid vector 3HP4108) |
| 5Anald3F | SEQ ID NO: 89 | cgaggtcgacggtatcgataGCTGGCGGTAGAAGGATTTTC |
| 5Anald3R | SEQ ID NO: 90 | tagctccagcAATTGGCTGACGAAGAGTATAG |
| hphF4 | SEQ ID NO: 91 | tcagccaattGCTGGAGCTAGTGGAGGTC |
| hphR4 | SEQ ID NO: 92 | actgaagacgCGGTCGGCATCTACTCTATTC |
| 3Anald3F | SEQ ID NO: 93 | atgccgaccgCGTCTTCAGTGATGCCGATATC |
| 3Anald3R | SEQ ID NO: 94 | agtggatcccccgggctgcaCGTTTGGGCTTTGTCCTTTAG |
| | | uga2 gene deletion (plasmid vector 3HP4109) |
| 5Anuga2F | SEQ ID NO: 95 | cgaggtcgacggtatcgataCCAACTGGCGCTGTATAGATC |
| 5Anuga2R | SEQ ID NO: 96 | tagctccagcGGTCTGAAAGTAGTCCTGTG |
| hphF5 | SEQ ID NO: 97 | ctttcagaccGCTGGAGCTAGTGGAGGTC |
| hphR5 | SEQ ID NO: 98 | acgaggattgCGGTCGGCATCTACTCTATTC |
| 5Anuga2F | SEQ ID NO: 99 | atgccgaccgCAATCCTCGTCGACCATCAAAAAG |
| 5Anuga2R | SEQ ID NO: 100 | agtggatcccccgggctgcaAGAGGGTATGAAGGAGGAG |
| | | rtTA2S-Tet07-Cre system (plasmid vector 3HP4140) |
| nat1F | SEQ ID NO: 101 | aggtcgacggtatcgatatcACAGAAGATGATATTGAAGGAG |
| | | C |
| nat1R | SEQ ID NO: 102 | gatagggtggttgtctggatCAGTAAGTAGAAAGCTTTGGG |
| ubi1F | SEQ ID NO: 103 | cgaggtcgacggtatcgataTCCAGACAACCACCCTATCTC |
| ubi1R | SEQ ID NO: 104 | gtctggacatCTTGATGAAGGTCTGGGTTG |
| rtTAF | SEQ ID NO: 105 | cttcatcaagATGTCCAGACTCGATAAGTC |
| rtTAR | SEQ ID NO: 106 | cgtgatacgcTCCATGATTCATGACGTATATTC |
| tetO7F | SEQ ID NO: 107 | gaatcatggaGCGTATCACGAGGCCCTTTC |
| tetO7R | SEQ ID NO: 108 | gccgaagaccGATCCTCAATCACCATCCTCC |
| trpCF | SEQ ID NO: 109 | attgaggatcGGTCTTCGGCTATAGTTCATTTTTATC |
| trpCR | SEQ ID NO: 110 | agtggatcccccgggctgcaGTTGCGATCAGGTGTGTAATTG |
| | | mdhP-neo marker gene (plasmid vector 3HP4134) |
| 2662mdhFp | SEQ ID NO: 111 | tcatcctcagGAACGACTCCAGAAGTGACTAAG |
| 2663mdhRp | SEQ ID NO: 112 | gttcaatcatGGTGAAATTTGGGATTGTGAC |
| 2664npt2F | SEQ ID NO: 113 | aaatttcaccATGATTGAACAAGATGGATTGC |
| 2665npt2R | SEQ ID NO: 114 | agtggatcccccgggctgcaGACTCTGCTAAGCTATTCTTC |
| | | LoxP-nptII vector (plasmid vector 3HP4136) |
| loxPnpt2F | SEQ ID NO: 115 | ggtcgacggtatcgataATAACTTCGTATAGCATACATTATA |
| | | CGAAGTTATGAACGACTCCAGAAGTGAC |
| loxPnpt2R | SEQ ID NO: 116 | atcccccgggctgcaataacttcgtataatgtatgctatacgaagtt |
| | | atGACTCTGCTAAGCTATTCTTC |
| | | New b-alanine 3-HP pathway |
| | | Intermediate vector (plasmid vector 3HP4144) |
| 2728pu4F | SEQ ID NO: 117 | attgggtaccgggcccccccgtttaaaCTTCGGAGTAGCAACGAG |
| | | TATTTTC |
| 2729pu4R | SEQ ID NO: 118 | attgtttagaAGCGCAGTTAATGGTGTATG |
| 2730panF | SEQ ID NO: 119 | taactgcgctTCTAAACAATGCCCGCCACC |
| 2731panR | SEQ ID NO: 120 | TCCCATACTGCTCCCATAGAAG |
| 2732bapatF | SEQ ID NO: 121 | TCTATGGGAGCAGTATGGGATC |
| 2733bapatR | SEQ ID NO: 122 | cttcatcaagATGGAACTGATGATCGTCCAG |
| 2734pus1F | SEQ ID NO: 123 | tcagttccatCTTGATGAAGGTCTGGGTTG |
| 2735pus1R | SEQ ID NO: 124 | tattatcgataccgtcgaccTCCAGACAACCACCCTATCTC |
| | | Entire new b-alanine 3-HP pathway vector (plasmid |
| | | vector 3HP4145) |
| 2736pmbF | SEQ ID NO: 125 | atacgaagttattgcagcccGGGACATGCTGGAAGGGATTTTC |
| 2737pmbR | SEQ ID NO: 126 | gcacgatcatTTTGAAGATGGATGAGAAGTCGG |
| 2738hpdhF | SEQ ID NO: 127 | catcttcaaaATGATCGTGCTGGTCACGGG |
| 2739hpdhR | SEQ ID NO: 128 | ctagaactagtggatcccccgtttaaaCCAATGGGATCCCGTAATC |
| | | AATTG |

### Example 2: Evaluation of 3-HP Production in Aspergillus pseudoterreus

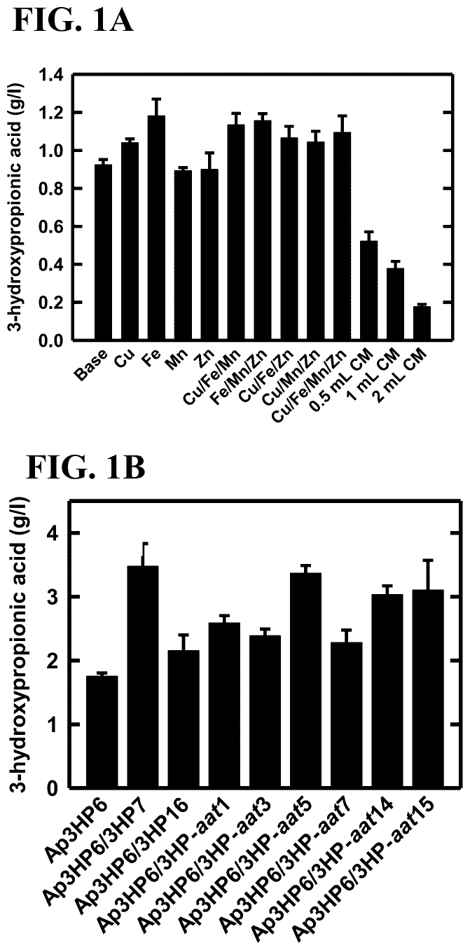

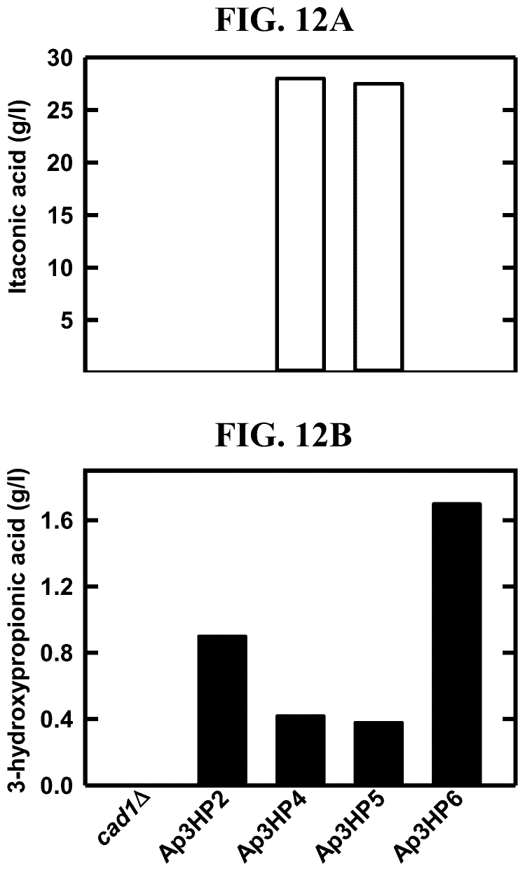

A synthetic β-alanine pathway for 3-HP production using Tribolium castaneum aspartate 1-decarboxylase (PAND), Bacillus cereus β-alanine-pyruvate aminotransferase (BAPAT) and Escherichia coli 3-hydroxypropionate dehydrogenase (HPDH) has been established and demonstrated in Saccharomyces cerevisiae (Borodina et al., Metab Eng 27:57-64, 2015) and Aspergillus pseudoterreus (Pomraning et al., Front Bioeng Biotechnol 9:603832, 2021). The effect of supplementation with trace elements (TE) and complex nutrients on 3-HP production in transgenic strain A. pseudoterreus Ap3HP6 that contains two copies of the β-alanine pathway (FIGS. 11A-11D; FIGS. 12A-12B) was studied, since the production medium B (RDM) was originally optimized for itaconic acid production (Riscaldati et al., J Biotechnol 83(3):219-30, 2000). Individual or combinations of TE were added to the base culture medium at up to 20-fold the original concentration. FIG. 1A shows that both Cu and Fe enhance 3-HP production in modified production medium B (mRDM). However, no synergetic effects were observed when combinations of Cu, Fe, Mn, and Zn were added. In addition, supplementation with small amounts of nutrient rich medium (0.5, 1, or 2 ml YPD) substantially reduced 3-HP production.

The expression of the β-alanine pathway for 3-HP production was limited by overexpressing additional copies of the pathway was then tested. A transgene expression cassette containing one (3HP) or one with the aat over-expression (3HP-aat) of the β-alanine pathway (FIG. 14; FIG. 15) was randomly integrated into the chromosome of transgenic strain Ap3HP6. 3-HP titer in selected transgenic strains was increased up to 3.4 g/l, about twice the concentration of the parent strain (FIG. 1B). In addition to 3-HP, significant amounts of other organic acids such as aconitic acid and citric acid were produced by A. pseudoterreus (Pomraning et al., Front Bioeng Biotechnol 9:603832, 2021) suggesting it may not be an ideal acidophilic filamentous fungus for 3-HP production. Therefore, A. niger, an industrial species used for citric acid production, was examined as a host for 3-HP production.

### Example 3: Evaluation of 3-HP Production in A. niger

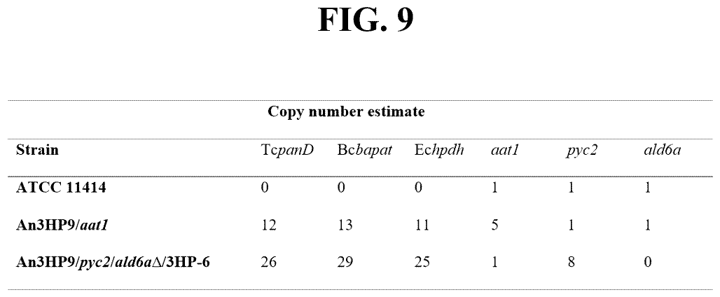

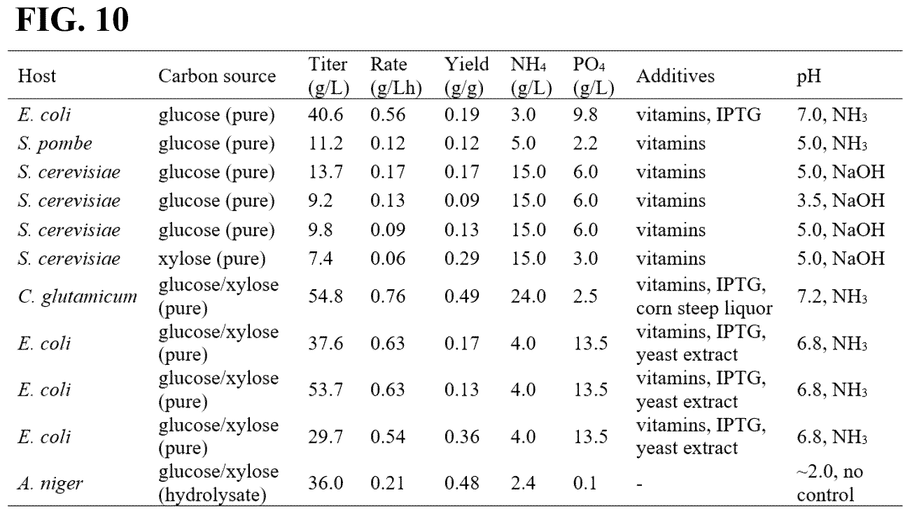

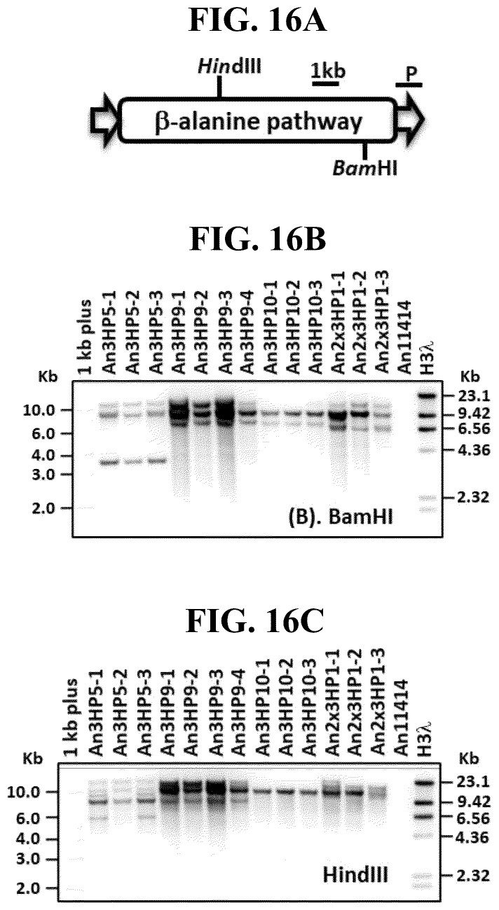

The same linearized transgene expression cassette (FIG. 11A) used in A. pseudoterreus was randomly integrated into the chromosomes of A. niger. Three transgenic A. niger strains, An3HP5, An3HP9, and An3HP10 were selected for evaluation of 3-HP production in mRDM medium. The results in FIG. 2A show that transgenic strain An3HP9 produced the highest 3-HP titer, reaching 6.8 g/l in mRDM, a 200% increase over the highest titers produced by A. pseudoterreus. Prior to further genetic engineering in the An3HP9 strain, the copy number was estimated by Southern blotting analysis. The results in FIGS. 16A-16C show that strain An3HP9 contains more than one copy of the β-alanine pathway for 3-HP production. The actual copies of insertion in An3HP9 were estimated as twelve by short-read whole genomic DNA sequencing (FIG. 9).

### Example 4: Multi-Omics Analysis of 3-HP Production in Aspergillus Species

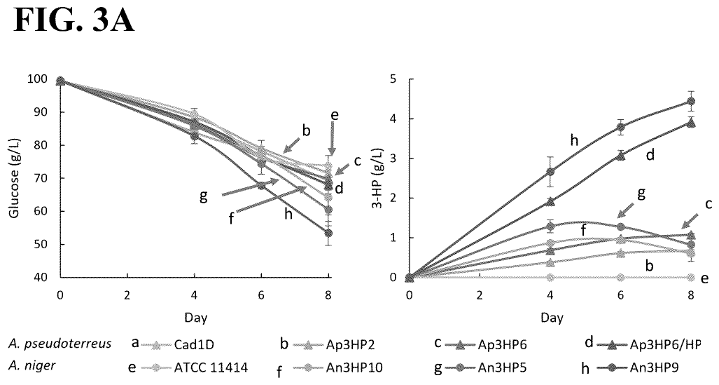

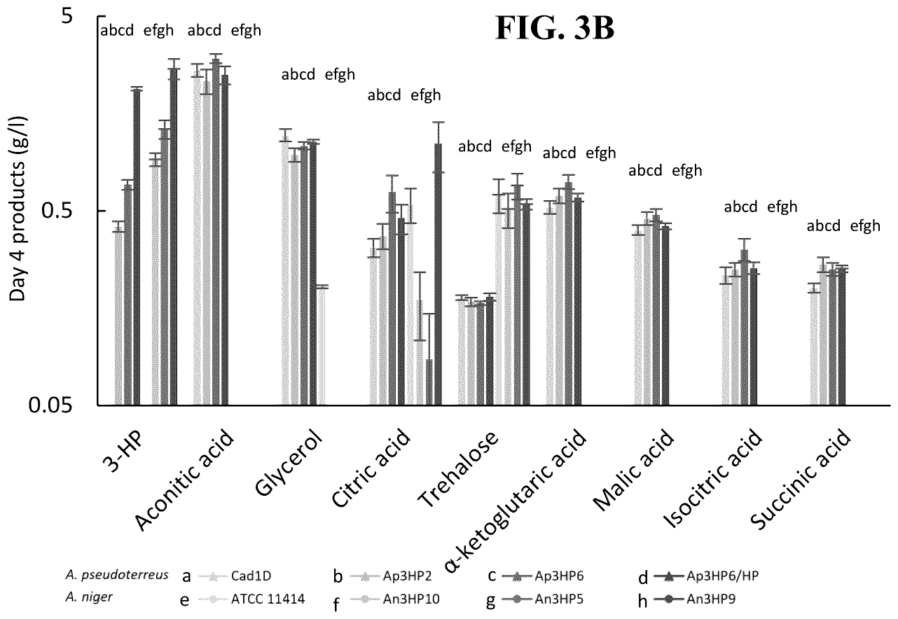

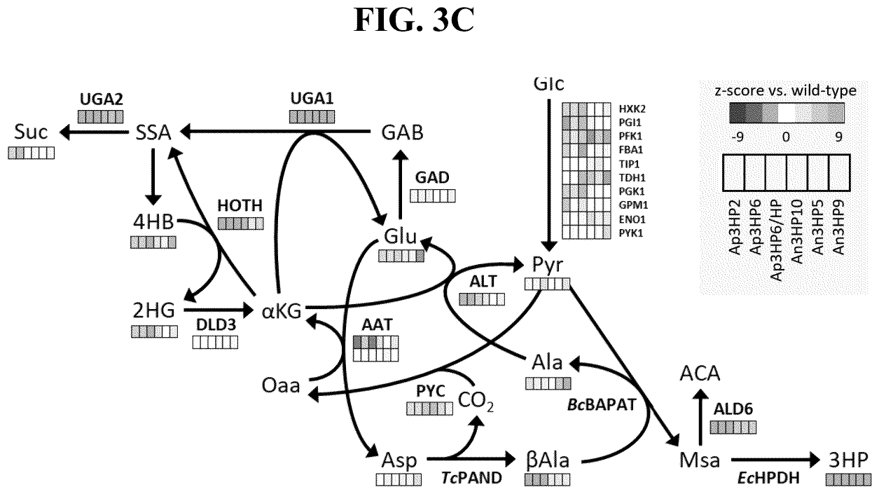

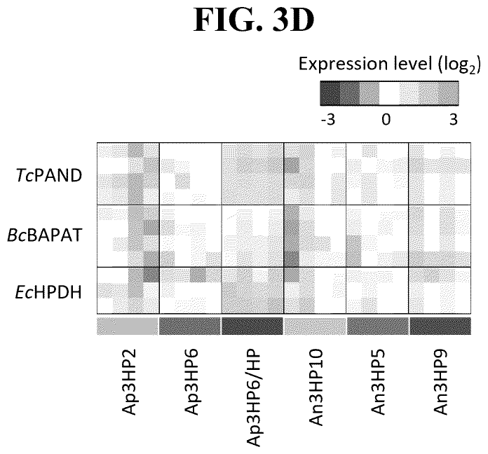

In both A. pseudoterreus and A. niger, transformants of the same β-alanine pathway for 3-HP production were isolated that produce a broad range of 3-HP titers. The transformants of both species were compared during time-course cultivation in shake-flasks in mRDM (FIG. 3A). In some cases, 3-HP titer declined later in the cultivation consistent with previous observations of 3-HP degradation catalyzed by the methylmalonate semialdehyde dehydrogenase ald6 (Pomraning et al., Front Bioeng Biotechnol 9:603832, 2021). Therefore, the biomass and supernatant samples were collected at day four, prior to decrease in titer, to assess the impacts of 3-HP production on metabolism by global and targeted proteomics, and intra- and extracellular metabolomics. While the range of 3-HP yields is comparable for the transformants obtained from the two Aspergillus species, the spectrum of co-products is dissimilar. Aside from 3-HP, from the panel of metabolites quantified, only trehalose and citric acid were detected in the A. niger fermentation broth. In contrast, A. pseudoterreus produced a wide variety of contaminating co-products that include glycerol and most of the tricarboxylic acid (TCA)-cycle derived organic acids (FIG. 3B). Targeted peptides designed for the heterologous enzymes in the β-alanine pathway were used to compare expression level between the species (FIG. 3C) and confirmed that the pathway is expressed at a higher level in strains that produce more 3-HP.

Global proteomics and metabolomics were used to assess the impact of increasing 3-HP production on metabolism. Metabolomics revealed that in both species intracellular β-alanine, 3-HP, and 4-hydroxybutyrate were significantly increased in all transformed strains. Proteomic analysis identified ALD6 as significantly up-regulated in all engineered strains indicating degradation via the pathway intermediate malonate semialdehyde is a conserved aspect of 3-HP metabolism in Aspergillus species. Proteins involved in the GABA shunt (UGA1 and UGA2) and a hydroxyacid-oxoacid transhydrogenase involved in conversion of α-ketoglutarate to succinate semialdehyde and mobilization of 4-hydroxybutyrate (Kardon et al., FEBS lett 580(9):2347-50, 2006) are also responsive to 3-HP production and up-regulated in all engineered strains (FIG. 3D).

Most enzymes along the ideal path from glucose to 3-HP do not exhibit a consistent response to the presence of the heterologous 3-HP production pathway. However, in the highest producing A. niger strains alanine, glutamate and aspartate are depleted suggesting nitrogen pools may be stressed in this host, while in A. pseudoterreus glutamate and alanine are accumulated and both alanine transaminase and aspartate aminotransferase are downregulated.

### Example 5: Augmenting Metabolic Flux to Precursor Molecules Required for 3-HP Production in A. niger

When directly compared, A. niger produced 3-HP with fewer co-products contaminants than A. pseudoterreus. Therefore, improvement of 3-HP yield from glucose in A. niger was focused on. Carbon efficient flux toward 3-HP relies on the precursor metabolites oxaloacetate and aspartate. Omics analysis indicated that nitrogen pools that support flux through these metabolites may be strained and that expression of pyruvate carboxylase, a key step for carbon efficiency, may be limited in some strains. Flux toward oxaloacetate and aspartate was examined for their contribution to 3-HP production in A. niger by overexpression of pyruvate carboxylase (pyc) and aspartate aminotransferase (aat1). cDNA of aat1 or pyc under the control of tef1 promoter (teflp-aat, supplementary FIG. 17A), mbfA (mbfAp-pyc, FIG. 17B) promoter or the combination of aat and pyc (tef1p-aat-pgkt-pyc-mbfAp, FIG. 17C) was integrated into transgenic strain An3HP9. Transformants were evaluated for 3-HP production (FIGS. 18A-18C) and strains with improved titer were purified (FIG. 2B). Overexpression of aat increased titer of 3-HP up to 43%, while pyc overexpression increased titer by as much 93% compared to the original transgenic An3HP9 strain. This indicates that flux toward beach-head metabolites for 3-HP is limiting in An3HP9, however, no synergistic effects were observed in transgenic strains overexpressing both aat and pyc together (FIG. 18C). The copy number of aat or pyc in transgenic strains An3HP9/aat5 and An3HP9/pyc2 (FIG. 9) were estimated as five and eight by short-read whole genomic DNA sequencing, respectively.

### Example 6: Effects of Genes Involved in Metabolism of b-Alanine Pathway Intermediates and 3-HP Transport in A. niger

Recently, genes potentially involved in metabolism of 3-HP pathway intermediates via multi-omics studies in A. pseudoterreus were identified (Pomraning et al., Front Bioeng Biotechnol 9:603832, 2021). Many of these genes are also present in A. niger. In this study, the A. niger homologs of malonate semialdehyde dehydrogenase (jgi|Asppseute1|414254 [Apald6] and jgi|Asppseute11497789 [Apald3]), succinate semialdehyde dehydrogenase (jgi|Asppseute11447301 [Apuga2]), a putative 3-HP transporter identified from A. pseudoterreus (jgi|Asppseute1|474223[Apmct1]), and A. niger oxaloacetate hydrolase (AnoahA) were examined for their effect on 3-HP production in the transgenic An3HP9/pyc2 strain (FIG. 17E; FIGS. 19A-19E). Single gene homologs were identified for all the targets except Apald6, where two homologs were identified in A. niger (Anald6a and Anald6b). The results in FIG. 2C show that disruption of Anald6a in strain 3HP/pyc2 increased 3-HP titer by 83% to 16.5 g/l, while disruption of Anald6b and Anald3 increased titer by 45% and 26% respectively suggesting that, while Anald6a is likely the major malonate semialdehyde dehydrogenase, all three contribute to directing flux away from 3-HP. In contrast, when Anuga2 was deleted, 3-HP production decreased by 37%. In this strain growth and sugar conversion rate was decreased and the specific yield of 3-HP increased. This suggests that yield of 3-HP from sugars may be improved by limiting flux through the GABA shunt, but that deletion of the pathway entirely is overly detrimental to growth. Disruption of oxaloacetate hydrolase (AnoahA) increased the titer of 3-HP by 45% without impacting growth suggesting that flux toward oxalic acid, a product secreted by A. niger (Kobayashi et al., J Ind Microbiol Biotechnol 41(5):749-56, 2014), represents a substantial loss in yield of 3-HP. A monocarboxylate transporter (Anmct1, FIG. 17E), where the homolog in A. pseudoterreus responds to the presence of intracellular 3-HP (Pomraning et al., Front Bioeng Biotechnol 9:603832, 2021) was over-expressed, resulting in a 35% improvement in 3-HP suggesting that transport across the plasma membrane may limit 3-HP production.

### Example 7: Effects of Additional Copies of β-Alanine Pathway on 3-HP Production in A. niger

The initial set of A. niger and A. pseudoterreus strains expressing various levels of the 3-HP production pathway demonstrated that higher expression level of the β-alanine pathway genes increased the yield of 3-HP (FIG. 3). To determine whether flux through the β-alanine pathway was still a limiting factor for the yield, expression was increased by randomly integrating a new transgene expression cassette (FIG. 20E) into the highest producing An3HP9/pyc2/ald6aD strain. Transformants with higher 3-HP titer than the parents were identified and the best performing single-spore isolate produced 20.5 g/l 3-HP, a 53% improvement over the parent strain (FIG. 2D).

Short-read whole genome sequencing was performed on selected strains at critical points in the construction lineage to estimate copy number of the randomly integrated plasmids (FIG. 9). Approximately 12 copies of the β-alanine pathway were randomly integrated into the A. niger chromosomes during construction of strain An3HP9 and that seven additional copies of pyc were integrated into the genome in strain An3HP9/pyc2. In the highest producing strain (An3HP9/pyc2/ald6aA/3HP-6) deletion of ald6a was confirmed and an additional 15 copies of the β-alanine pathway were inserted into the genome bringing the total copy-number of the β-alanine pathway to approximately 27 copies.

### Example 8: Optimization of Culture Conditions for 3-HP Production

Culture conditions for citric acid by A. niger have been optimized and are dependent on pH, as well as carbon, nitrogen, and manganese concentration (Dai et al., Appl Environ Microbiol 70(4):2474-85, 2004). In this study, the effects of pH, manganese, and nitrogen on 3-HP production was examined with initially genetically engineered strain An3HP9 or 3HP9/pyc2. The effects of citric acid production (CAP) versus mRDM medium were examined in the first A. niger transgenic strain An3HP9 by growing them in either CAP medium or mRDM with the pH ranging from 2.0 to 3.4 since pH 2.0 is optimal pH for CAP. 3-HP titer in CAP medium was 2.7 g/L, significantly lower than in mRDM across the pH spectrum. (FIG. 11A). In mRDM, the spore germination rate increased with increasing pH up to 30.7% at pH 3.4 (FIG. 11B) and coincided with increased consumption of glucose resulting in an insignificantly lower yield of 3-HP.

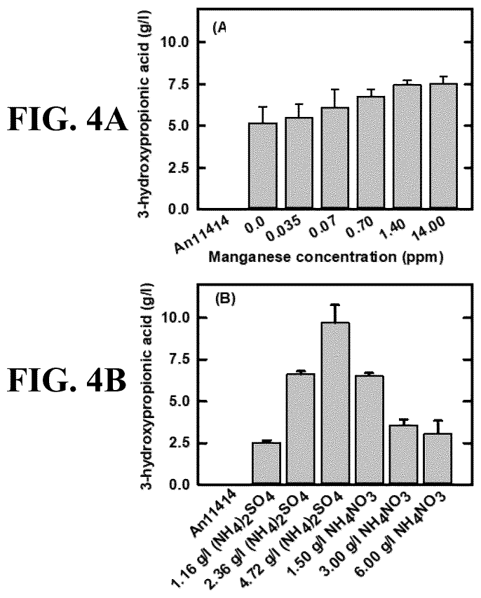

To further increase 3-HP production, effects of manganese on 3-HP production were evaluated in the transgenic strain An3HP9/pyc2 strain, which contains 8 copies of pyc transgene overexpression in An3HP9 with 90% improvement in 3-HP production. When the strain was grown with 0-0.035 ppm manganese, 3-HP titer reached 5.5 g/l but when manganese was increased to 0.07, 0.7, 1.4, 14.0 ppm manganese, the 3-HP titer increased to 6.1, 6.7, 7.4, and 7.5 g/l, respectively suggesting that the required manganese level to support 3-HP metabolism is around 1.4 ppm, about 140-fold higher than that required for citric acid production in A. niger (FIG. 4A). At all manganese concentrations the yield of 3-HP was not significantly different while the yield of biomass trended downward with increasing manganese suggesting the increased titer is due to differences in the rate of 3-HP production. The effect of nitrogen concentration and source, in the form of (NH₄)₂SO₄ or NH₄NO₃, on 3-HP production in the An3HP9/pyc2 strain was also examined (FIG. 4B). 3-HP production increased from 2.5 to 9.7 g/l when the strain was grown in mRDM with 1.16, 2.36, or 4.72 g/l (NH₄)₂SO₄, respectively. Increasing nitrogen increased the sugar conversion rate, but overall yield was still significantly higher at the greatest (NH₄)₂SO₄ concentration (p<0.01). The β-alanine pathway to produce 3-HP is dependent on balanced flux through three transaminases [AAT, alanine transaminase (ALT), and BAPAT] and excess nitrogen may be required to support efficient flux through these reactions. When the strain was grown in mRDM with increasing concentrations of NH₄NO₃, 3-HP production decreased suggesting the impact of NO₃ as a nitrogen source may be detrimental to 3-HP production. The optimal nitrogen source for batch 3-HP production in shake-flasks was 4.72 g/l of (NH₄)₂SO₄, which corresponds to a C/N ratio of 40.

### Example 9: Production of 3-HP from Lignocellulosic Feedstock Derived Sugars

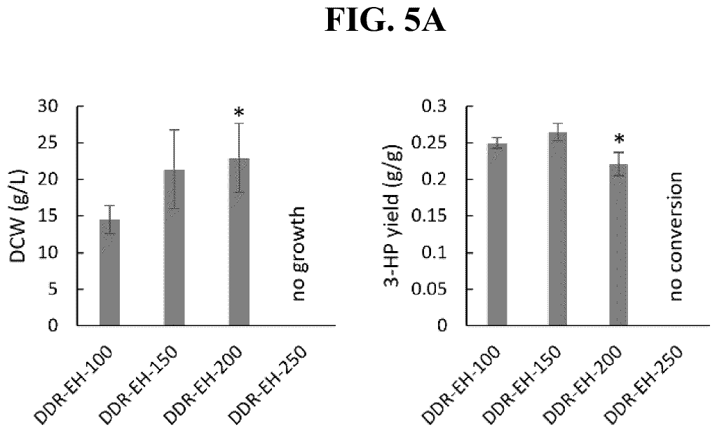

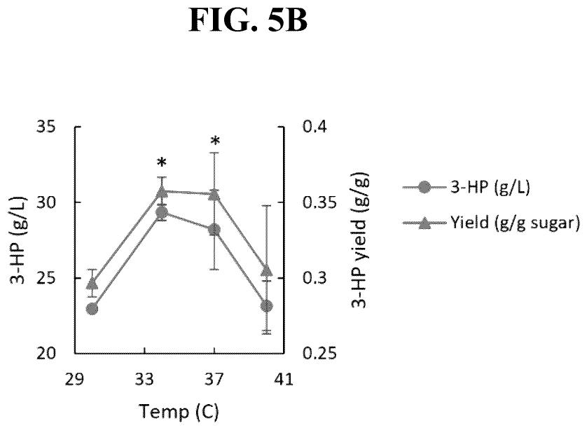

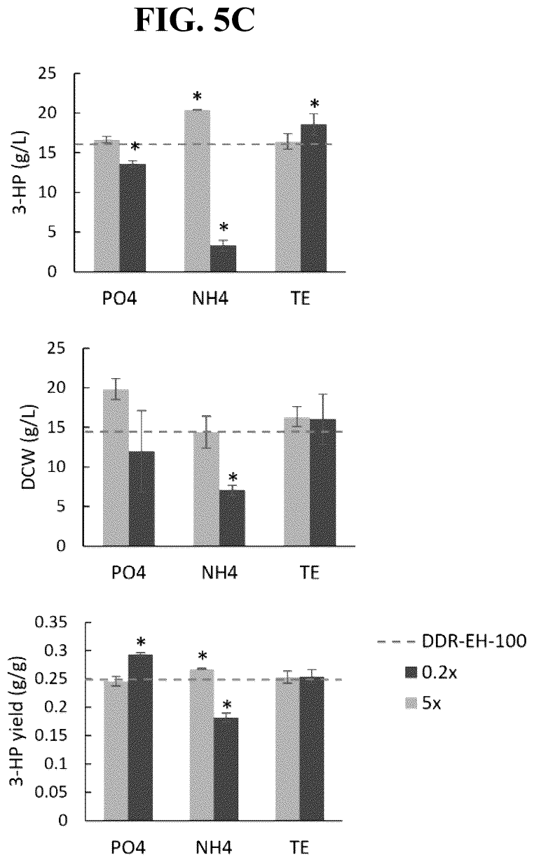

In addition to purified glucose as a carbon source for 3-HP production, sugars derived from lignocellulosic biomass as a feedstock were considered. Corn stover was subjected to dilute alkali deacetylation prior to disk refining and enzymatic hydrolysis (DDR-EH) to release glucose and xylose monomers (Chen et al., Biotechnol Biofuels 7(1):1-12, 2014; Chen et al., Energy Environ Sci 9(4):1237-45, 2016). The conversion of sugars produced by the DDR-EH process using both An3HP9/pyc2/ald6aD and An3HP9/pyc2/ald6aD/3HP-6 strains with the highest 3-HP production titers was investigated. Initially the 3-HP production of An3HP9/pyc-2/ald6aD strain in RDM to provide essential micro and macro-nutrients was tested with increasing concentrations of sugars from the DDR-EH process to identify limits on conversion due to toxicity. A. niger was able to germinate and grow in up to 200 g/L total sugars from the DDR-EH process (FIG. 5A) with a significantly lower yield of 3-HP at the highest sugar concentration where growth was maximized. Next, the impact of temperature on conversion in DDR-EH was studied and it was found that 34° C. to 37° C. significantly improved both 3-HP yield and titer with a maximum of 29.4 g/L 3-HP and 0.35 C-mol 3-HP C-mol⁻¹ sugars (FIG. 5B). Sugars produced using the DDR-EH process contain a wide variety of characterized and unknown metabolites. To determine whether phosphate, nitrogen, and trace elements (TE) required for growth and production are present at necessary levels in DDR-EH, their concentration were modified up and down by 5-fold to represent limiting and excess concentrations (FIG. 5C). Reducing phosphate or nitrogen concentration reduced biomass production and 3-HP titer suggesting DDR-EH needs to be supplemented with both macronutrients to maximize 3-HP production. However, reduction of TE significantly improved 3-HP titer (p<0.05) while excess TE had no impact on growth or productivity suggesting TE in DDR-EH may be present at necessary concentrations without supplementation. When excess nitrogen was supplied, 3-HP titer and yield were both significantly improved without increasing biomass suggesting that supply of precursors for the multiple transaminases of the β-alanine pathway may not be optimized and flux is aided by the presence of excess nitrogen.

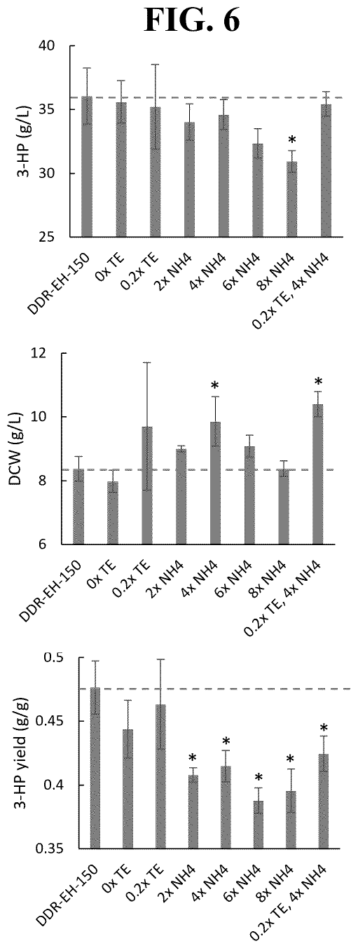

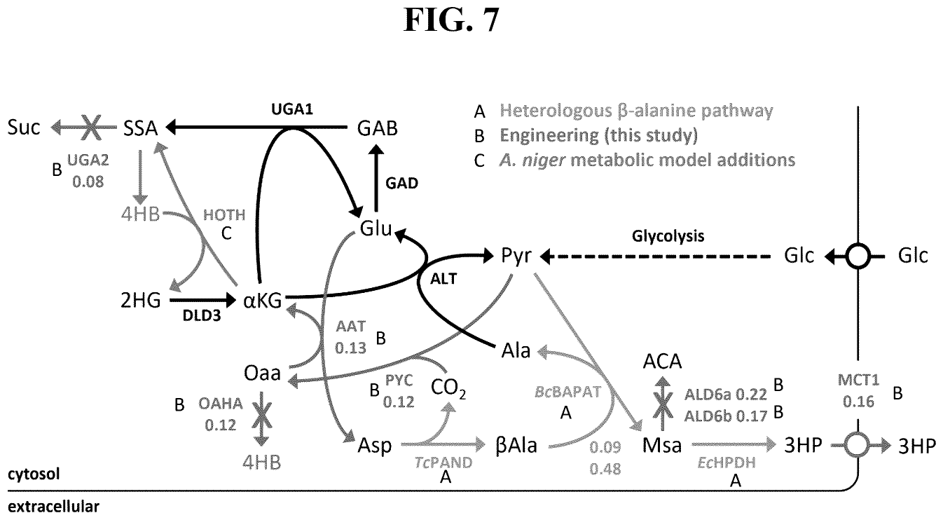

Some of the potential improvements in cultivation conditions to increase yield, rate, or cost for production of 3-HP from DDR-EH derived sugars were combined and tested with the highest yield An3HP9/pyc2/ald6aA/3HP-6 strain. The standard concentration of sugars was increased to 150 g/L (97.5 g/L glucose and 52.5 g/L xylose) and the temperature increased to 34° C. prior to retesting the impacts of nitrogen and trace elements (FIG. 6). With the combined improvements in the standard RDM with 1× TE and 1× N [2.36 g/l (NH₄)₂SO₄], 36.0 g/l 3-HP was produced, and the yield was improved to 0.48 C-mol 3-HP C-mol⁻¹ sugars. Reducing or eliminating the addition of TE from the standard RDM did not have a significant impact on growth or 3-HP production. Increasing the concentration of nitrogen tended to increase the amount of biomass produced and, in all cases, significantly decreased the yield of 3-HP.

### Example 10: Bioreactor Operations

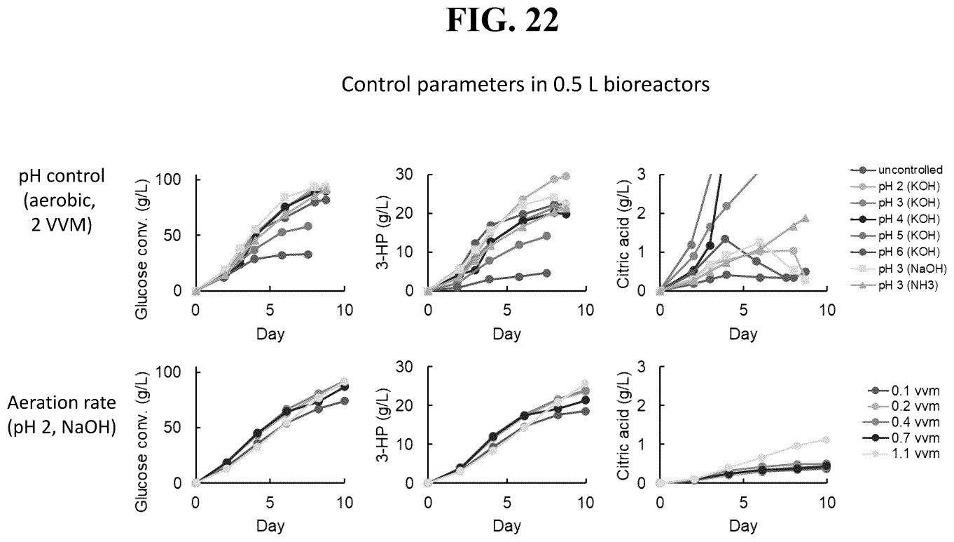

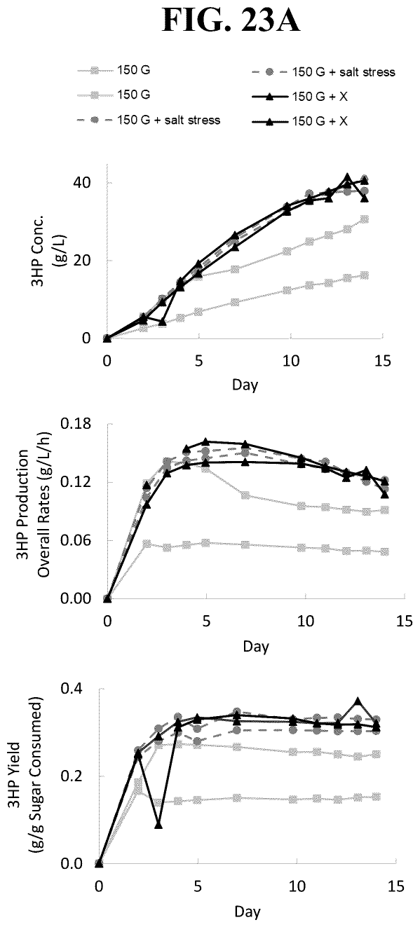

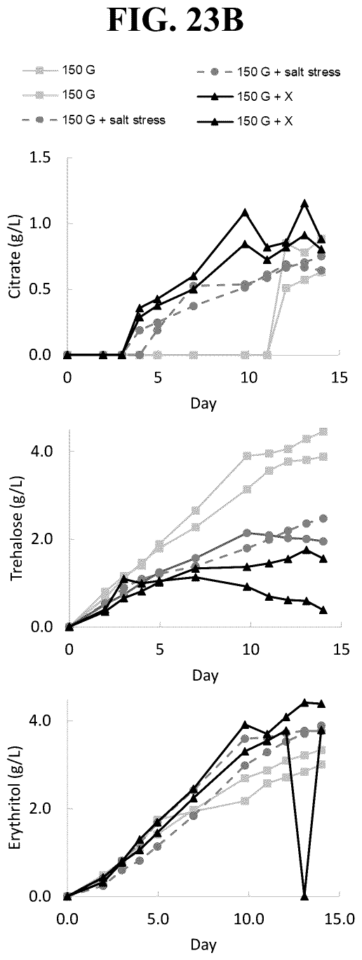

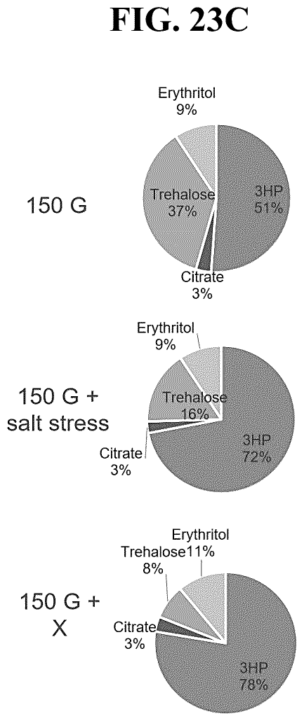

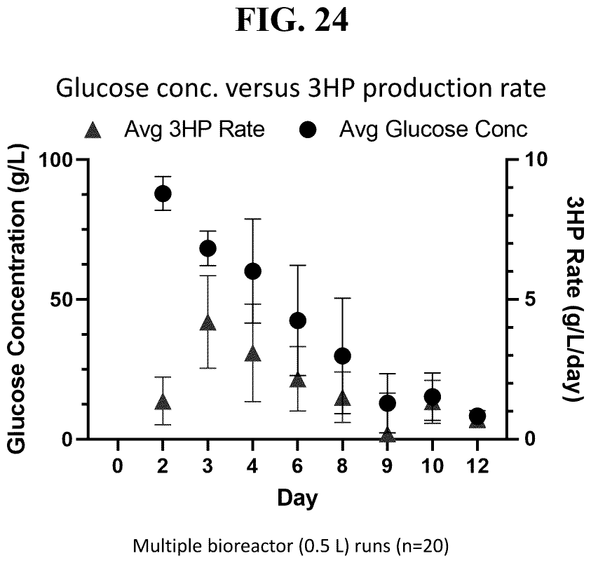

A 3-HP producing A. niger strain capable of secreting high levels of 3-HP (˜35 g/L achieved) in shaking flasks cultivations was established herein. This strain (An3HP9/pyc2/ald6aA/3HP-6) was used to establish the bioreactor production parameter space in 0.5 L benchtop fermenters. A range of maintained pH's was examined and it was determined that 3-HP could be produced as a free acid down to around pH 2 (FIG. 22) The utilization of sodium hydroxide for pH control led to better 3HP production. NaOH addition lead to more osmotic stressed conditions compared to addition of KOH, an unexpected result. Varied aeration rates in the bioreactors using the optimal pH conditions revealed that highly aerobic conditions were detrimental to production and resulted in increased by-product secretion (FIG. 22). Another round of 0.5 L benchtop reactors were then performed to compare 3-HP production in medium with pure glucose, glucose:xylose at a 2:1 mixture, and glucose with additional NaCl as a salt stress (FIGS. 23A-23C). This data indicated that salt stress has the potential to lower bioproduct formation while maintaining similar 3-HP titers. Interestingly, there was a correlation of glucose concentration to 3-HP production rates over the course of 20 different 0.5 L fermentation runs. At ˜50 g/L of sugar, the 3-HP production tended to decrease compared to initial rates (FIG. 24). Once parameter space for bioreactor cultivations had been established, various conditions were screened to increase production and sugar consumption.

### Example 11: Shaking Flasks Optimization

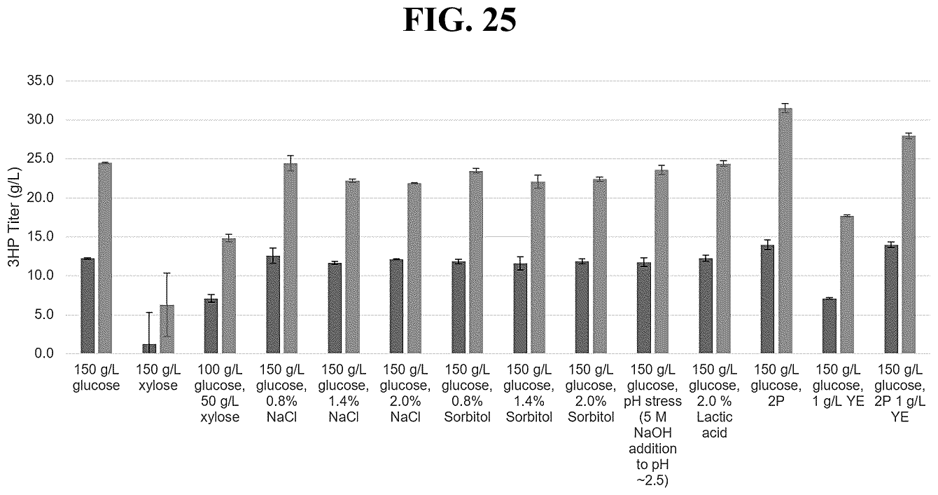

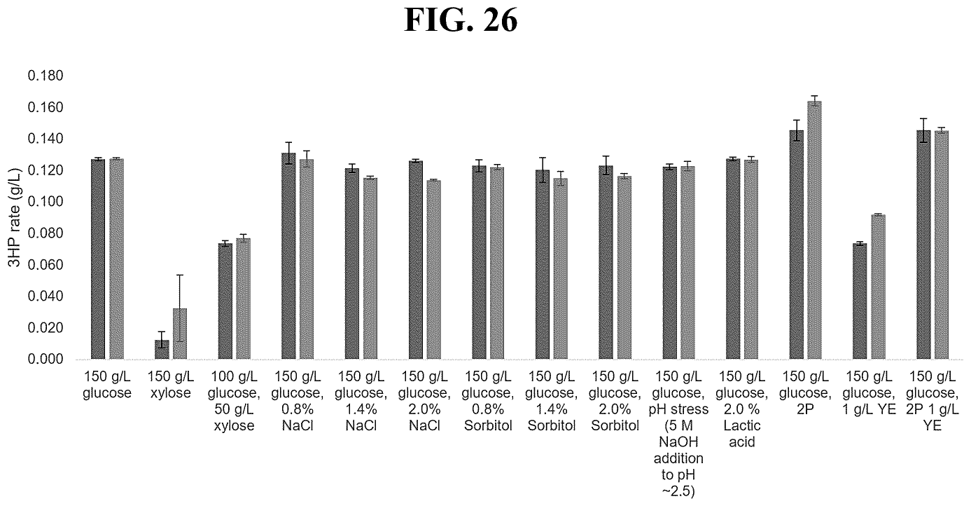

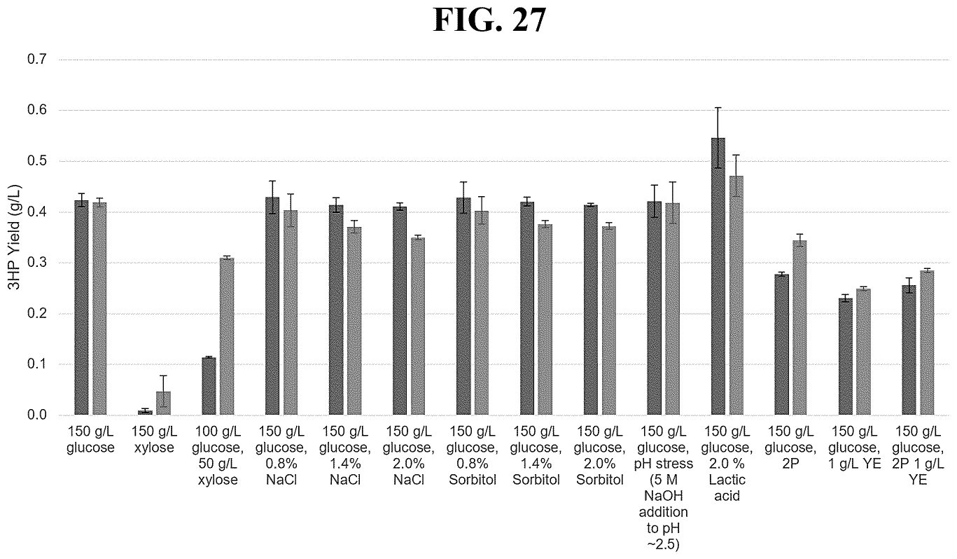

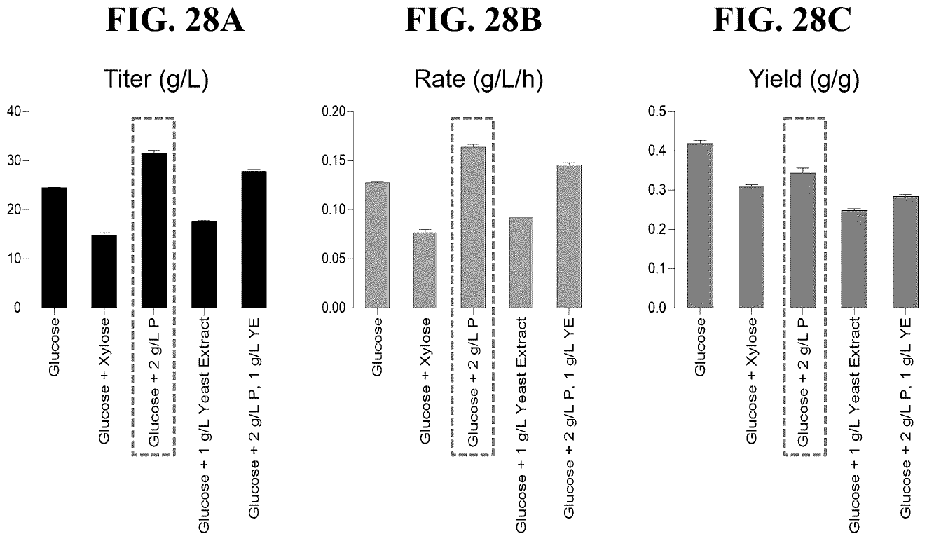

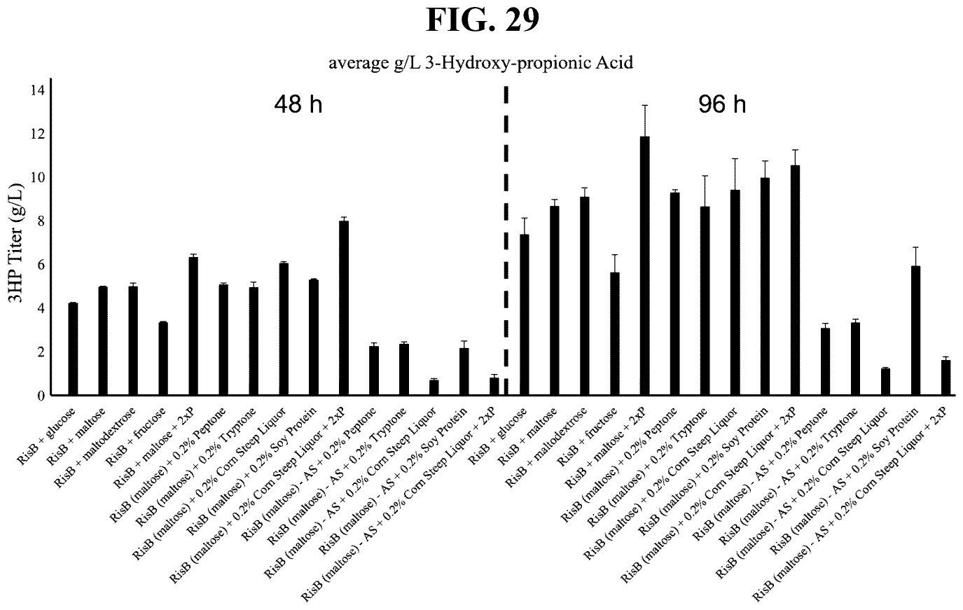

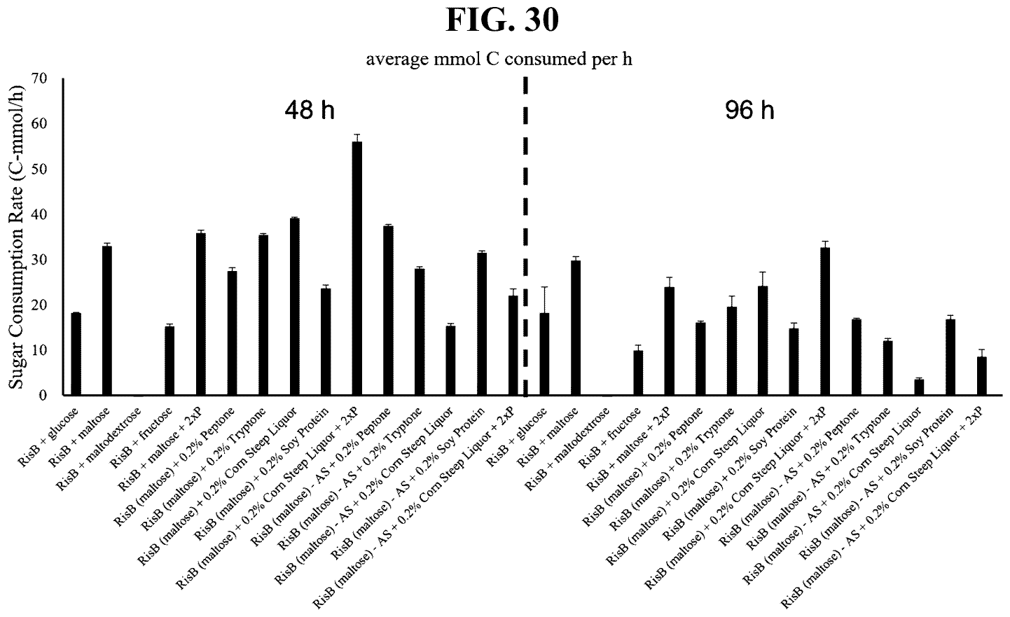

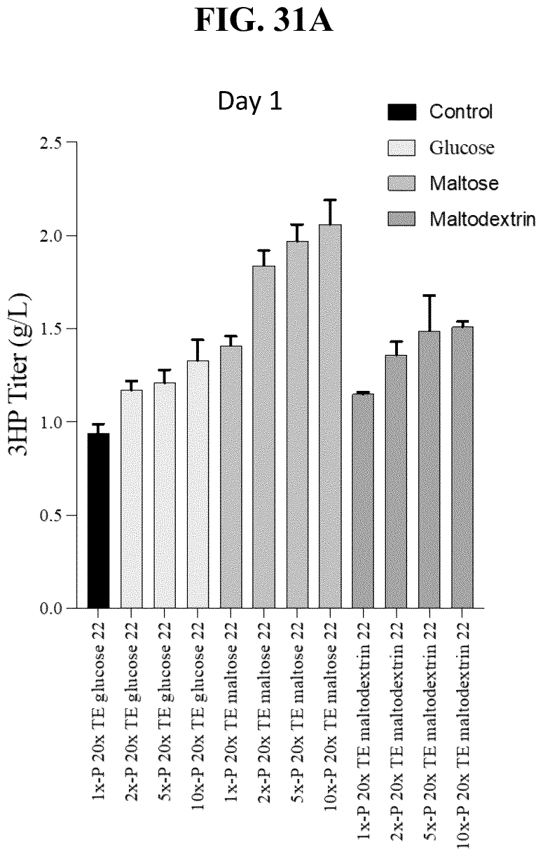

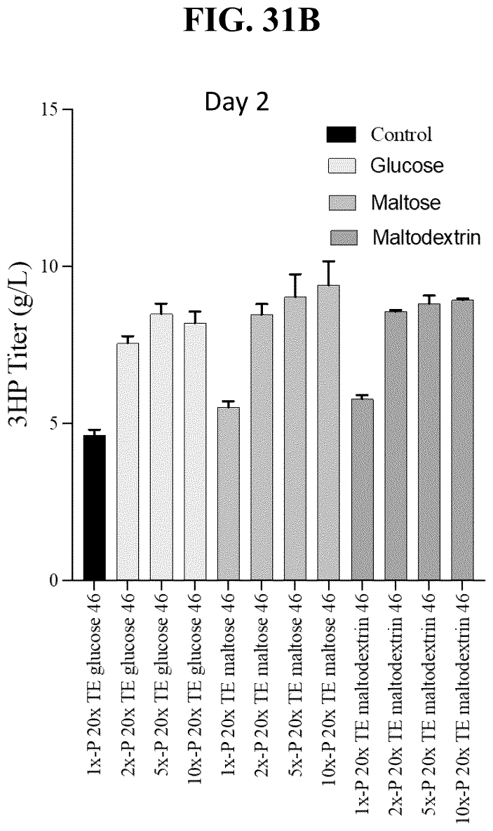

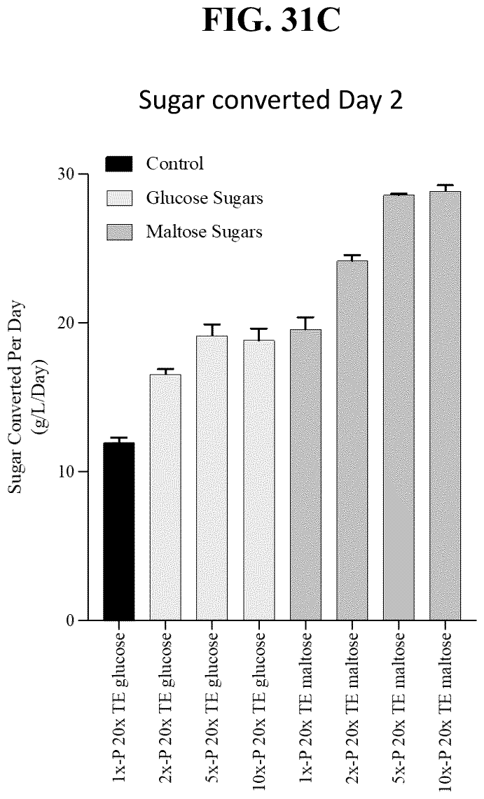

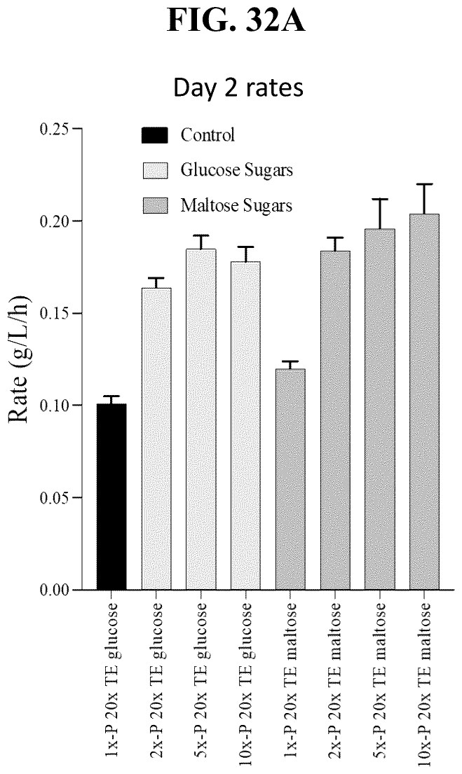

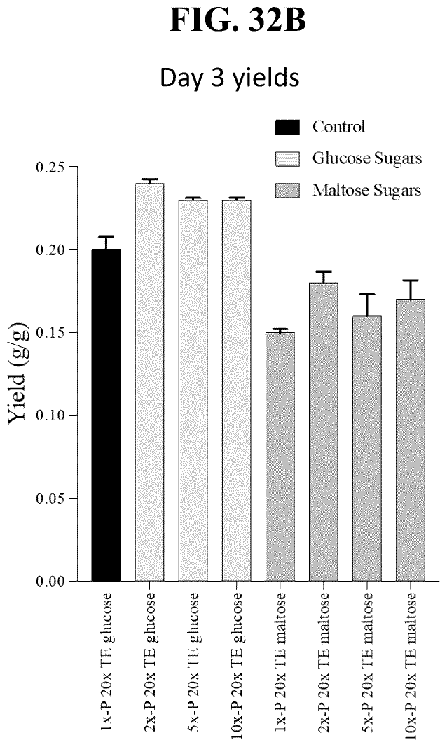

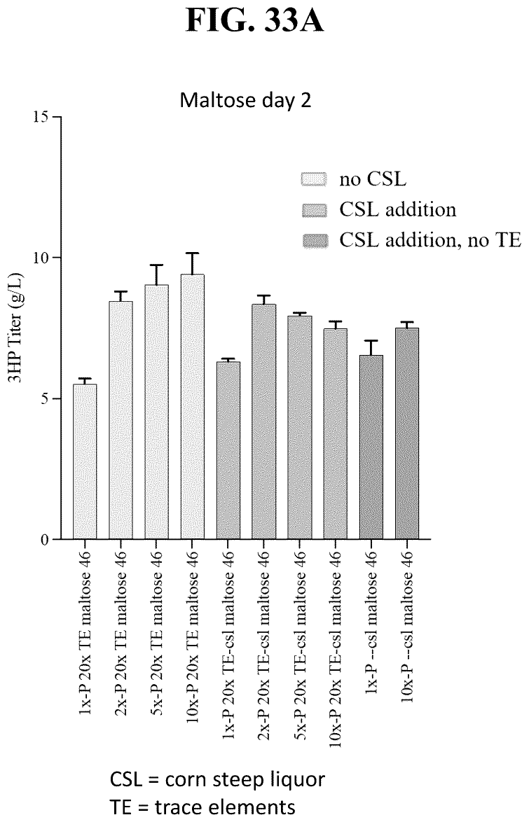

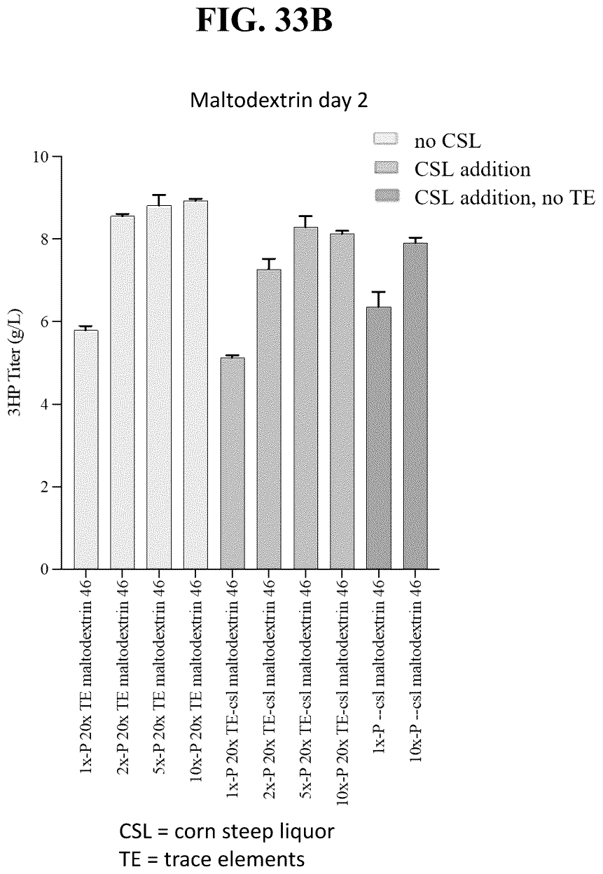

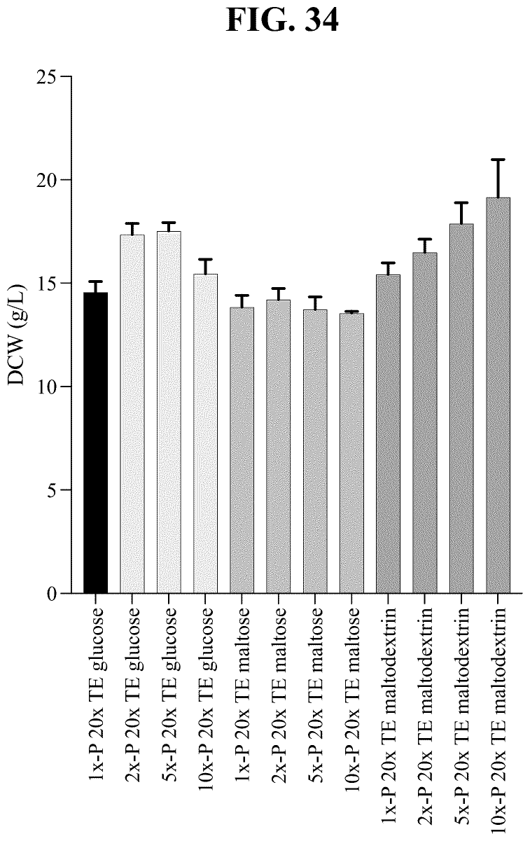

The initial shaking flask experiment investigated further osmotic stressor effects on production (using NaCl or Sorbitol) or addition of further phosphate and/or yeast extract (rich component). Titers, rates, and yields are shown in FIG. 25, FIG. 26, and FIG. 27, respectively. Compared to the control condition (pure glucose), supplying additional phosphate led to enhanced rates of 3-HP production (FIGS. 28A-28C). Unexpectedly, this did not appear to be due to increased cell mass, as the yeast extract (with rich components for growth) had detrimental effects on 3-HP production. Next, other rich components, such as peptone, tryptone, corn steep liquor and soy protein, were examined. Again, the results indicated that the addition of phosphate, in combination with corn steep liquor, led to the initial highest production of 3-HP (FIG. 29). Interesting, maltose uptake was also at a significantly faster rate (3-5×) than the control condition (FIG. 30). Maltose is a disaccharide and contains two glucose units. Lastly, a screen of culture conditions using maltose, maltodextrin, and glucose as the carbon source across a wide variety of phosphate conditions was performed, as well as the addition of corn steep liquor to the different cultures. The results indicated that maltose led to the fastest initial rates of 3HP production and sugar consumption (note: maltodextrin was not detectable via HPLC and thus, no data is available for sugar consumption in those conditions). Under these experimental conditions, the sugar conversion rate increased from 13.4±0.4 to 30.1±0.1 g/L/day (FIGS. 31A-31C). There was only minimal impact on the yield compared to the control condition (FIGS. 32A-32B). Phosphorus levels was found to have a similar effect on 3-HP production, with limited improvement in production past 5× phosphorus concentrations. Corn steep liquor was found to not significantly enhance production with maltose or maltodextrin as the sole carbon source under the tested conditions (FIGS. 33A-33B). Thus, there is some unexpected phenomena that is unrelated to the biomass production that leads to enhanced 3HP production. Perhaps the most surprising result was that the increased phosphorus concentration did not seem to lead to increased biomass under maltose conditions (FIG. 34).

Based on these findings, maltose was the best carbon source with an additional phosphorus concentration of 5×. It was unexpected to find that conditions with increased nutrients did not lead to enhanced rates of production. Specifically, the lack of increased biomass when further phosphorus was added into the maltose conditions, but the significant increase of production was unexpected. Additionally, maltose is simply two glucose monomers. However, there seems to be a different response to using pure glucose versus maltose for production.

It will be apparent that the precise details of the methods or compositions described may be varied or modified without departing from the spirit of this disclosure. We claim all such modifications and variations that fall within the scope and spirit of the claims below.

We claim:

1. A recombinant Aspergillus niger capable of producing 3-hydroxypropionic acid (3-HP), comprising an exogenous nucleic acid molecule encoding aspartate 1-decarboxylase (PAND), an exogenous nucleic acid molecule encoding β-alanine-pyruvate aminotransferase (BAPAT), and an exogenous nucleic acid molecule encoding 3-hydroxypropionate dehydrogenase (HPDH).

2. The recombinant Aspergillus niger of claim 1, wherein the copy number of each of PAND, BAPAT, and HPDH is independently about 1 to about 80 in the recombinant Aspergillus niger.

3. The recombinant Aspergillus niger of claim 1, wherein the copy number of each of PAND, BAPAT, and HPDH is independently about 10 to about 30 in the recombinant Aspergillus niger.

4. The recombinant Aspergillus niger of claim 1, wherein:
i) an ald6a, ald6b, ald3, and/or oahA gene is not present or does not produce a functional product in the recombinant Aspergillus niger; and/or
ii) pyc, aat1, and/or mct1 is overexpressed in the recombinant Aspergillus niger.

5. The recombinant Aspergillus niger of claim 1, wherein ald6a is not present or does not produce a functional product in the recombinant Aspergillus niger, and wherein pyc is overexpressed in the recombinant Aspergillus niger.

6. The recombinant Aspergillus niger of claim 1, wherein:
i) the PAND comprises at least 95% sequence identity to SEQ ID NO: 1; and/or
ii) the BAPAT comprises at least 95% sequence identity to SEQ ID NO: 2; and/or
iii) the HPDH comprises at least 95% sequence identity to SEQ ID NO: 3.

7. The recombinant Aspergillus niger of claim 1, wherein:
i) the PAND comprises or consists of SEQ ID NO: 1; and/or
ii) the BAPAT comprises or consists of SEQ ID NO: 2; and/or
iii) the HPDH comprises or consists of SEQ ID NO: 3.

8. The recombinant Aspergillus niger of claim 1, wherein the exogenous nucleic acid molecules encoding PAND, BAPAT, and HPDH are operably linked to a promoter.

9. The recombinant Aspergillus niger of claim 1, wherein the exogenous nucleic acid molecules encoding PAND, BAPAT, and HPDH are encoded by a nucleic acid molecule comprising at least 95% sequence identity to SEQ ID NO: 4 and/or SEQ ID NO: 142, or a nucleic acid molecule comprising or consisting of SEQ ID NO: 4 and/or SEQ ID NO: 142.

10. The recombinant Aspergillus niger of claim 1, comprising a nucleic acid molecule comprising SEQ ID NO: 4 and/or SEQ ID NO: 142.

11. The recombinant Aspergillus niger of claim 1, wherein the exogenous nucleic acid molecules encoding PAND, BAPAT, and HPDH are part of a vector, optionally wherein the vector is a plasmid.

12. A method of producing 3-hydroxypropionic acid (3-HP), comprising:
culturing the recombinant Aspergillus niger of claim 1 under conditions that permit the production of 3-HP, thereby making 3-HP.

13. A method of producing 3-HP, comprising:
inoculating a media with Aspergillus niger comprising a 3-HP β-alanine pathway, thereby generating a cultured media, and
fermenting the cultured media at a temperature of 30° C. to 37° C., under acidic and microaerobic conditions.

14. The method of claim 13, wherein the temperature is 33° C. to 35° C., optionally wherein the temperature is about 34° C.

15. The method of claim 13, wherein the acidic conditions are a pH of 1 to 4, optionally wherein the acidic conditions are about pH 2.

16. The method of claim 13, wherein the microaerobic conditions comprise a dissolved oxygen content of less than 15%, optionally wherein the dissolved oxygen content is about 0% to about 10%.

17. The method of claim 13, wherein the media comprises Riscaldati B medium (RisB).

18. The method of claim 13, wherein the media further comprises trace elements, corn steep liquor, peptone, tryptone, soy protein, sodium chloride, yeast extract, sorbitol, phosphorous, glucose, maltose, fructose, maltodextrin, and/or a glucose:xylose mixture.

19. The method of claim 13, wherein inoculating the media comprises inoculating the media with:
i) 1×10⁵ to 1×10⁷ spores/mL of the Aspergillus niger;
ii) about 1×10⁶ spores/mL of the Aspergillus niger;
iii) 5% to 20% per volume (vol/vol) of a seed culture of the Aspergillus niger; or
iv) about 10% (vol/vol) of a seed culture of the Aspergillus niger.

20. The method of claim 13, wherein the fermenting is performed in a bioreactor.

21. A kit, comprising:
the isolated recombinant Aspergillus niger of claim 1, and
a media, carbon source, nutrient additive, antifoam, antibiotic, filter, or combinations thereof.


SEQUENCE LISTING (143 sequences)

SEQ ID NO: 1 - protein, 540 aa, Tribolium castaneum
MPATGEDQDLVQDLIEEPATFSDAVLSSDEELFHQKCPKPAPIYSPVSKPVSFESLPNRRLHEEFLRSSVDVLLQEAVFEGTNRKNRVLQWREPEELRRLMDFGVRSAPSTHEELLEVLKKVVTYSVKTGHPYFVNQLFSAVDPYGLVAQWATDALNPSVYTYEVSPVFVLMEEVVLREMRAIVGFEGGKGDGIFCPGGSIANGYAISCARYRFMPDIKKKGLHSLPRLVLFTSEDAHYSIKKLASFQGIGTDNVYLIRTDARGRMDVSHLVEEIERSLREGAAPFMVSATAGTTVIGAFDPIEKIADVCQKYKLWLHVDAAWGGGALVSAKHRHLLKGIERADSVTWNPHKLLTAPQQCSTLLLRHEGVLAEAHSTNAAYLFQKDKFYDTKYDTGDKHIQCGRRADVLKFWFMWKAKGTSGLEKHVDKVFENARFFTDCIKNREGFEMVIAEPEYTNICFWYVPKSLRGRKDEADYKDKLHKVAPRIKERMMKEGSMMVTYQAQKGHPNFFRIVFQNSGLDKADMVHLVEEIERLGSDL

SEQ ID NO: 2 - protein, 451 aa, Bacillus cereus
MELMIVQVTEQTQSLKKTDEKYLWHAMRGAAPSPTNLIITKAEGAWVTDIDGNRYLDGMSGLWCVNVGYGRKELARAAFEQLEEMPYFPLTQSHVPAIKLAEKLNEWLDDEYVIFFSNSGSEANETAFKIARQYHQQKGDHGRYKFISRYRAYHGNSMGALAATGQAQRKYKYEPLGQGFLHVAPPDTYRNPEDVHTLASAEEIDRVMTWELSQTVAGVIMEPIITGGGILMPPDGYMEKVKEICEKHGALLICDEVICGFGRTGKPFGFMNYGVKPDIITMAKGITSAYLPLSATAVRREVYEAFVGSDDYDRFRHVNTFGGNPAACALALKNLEIMENEKLIERSKELGERLLYELEDVKEHPNVGDVRGKGLLLGIELVEDKQTKEPASIEKMNKVINACKEKGLIIGKNGDTVAGYNNILQLAPPLSITEEDFTFIVKTMKECLAQL

SEQ ID NO: 3 - protein, 248 aa, Escherichia coli
MIVLVTGATAGFGECITRRFIQQGHKVIATGRRQERLQELKDELGDNLYIAQLDVRNRAAIEEMLASLPAEWCNIDILVNNAGLALGMEPAHKASVEDWETMIDTNNKGLVYMTRAVLPGMVERNHGHIINIGSTAGSWPYAGGNVYGATKAFVRQFSLNLRTDLHGTAVRVTDIEPGLVGGTEFSNVRFKGDDGKAEKTYQNTVALTPEDVSEAVWWVSTLPAHVNINTLEMMPVTQSYAGLNVHRQ

SEQ ID NO: 4 - DNA, 11528 bp, synthetic construct
  source 1..11528: mol_type = other DNA
ggttgtagcagcgtaaacacatggatagttaaataatcggatgtacacccactgttggaaatgacgggggcctacaacacgagattatctgatccaatttctgttcgttggcattctatcattcgcagcgaaaattgtcctattaaattgaccatgaccaaacaatctgcggacagcaacgcaaagtcaggagttacgtccgaaatatgtcattgggcatccaacctggccactgacgacatcccttcggacgtattagaaagagcaaaataccttattctcgacggtattgcatgtgcctgggttggtgcaagagtgccttggtcagagaagtatgttcaggcaacgatgagctttgagccgccgggggcctgcagggtgattggatatggacaggtaaattttattcactctagacggtccacaaagtatactgacgatccttcgtatagaaactggggcctgttgcagcagccatgaccaattccgctttcatacaggctacggagcttgacgactaccacagcgaagcccccctacactctgcaagcattgtccttcctgcggtctttgcagcaagtgaggtcttagccgagcagggcaaaacaatttccggtatagatgttattctagccgccattgtggggtttgaatctggcccacggatcggcaaagcaatctacggatcggacctcttgaacaacggctggcattgtggagctgtgtatggcgctccagccggtgcgctggccacaggaaagctcctcggtctaactccagactccatggaagatgctctcggaattgcgtgcacgcaagcctgtggtttaatgtcggcgcaatacggaggcatggtaaagcgtgtgcaacacggattcgcagcgcgtaatggtcttcttgggggactgttggcccatggtgggtacgaggcaatgaaaggtgtcctggagagatcttacggcggtttcctcaagatgttcaccaaggctactatgaaagaccgcgatgggccgatagtagtagttacttccattacatcatctcatccgcccggttcctcgcctccgcggcagtctacgggtaggatcgtagcaaaaacccgggggatagacccgtcgtcccgagctggagttccgtataacctaggtagaaggtatcaattgaacccgaacaactggcaaaacattctcgagatcgtaggagtgagtacccggcgtgatggagggggagcacgctcattggtccgtacggcagctgccgagggggagcaggagatccaaatatcgtgagtctcctgctttgcccggtgtatgaaaccggaaaggactgctggggaactggggagcggcgcaagccgggaatcccagctgacaattgacccatcctcatgccgtggcagagcttgaggtagcttttgccccgtctgtctccccggtgtgcgcattcgactgggcgcggcatctgtgcctcctccaggagcggaggacccagtagtaagtaggcctgacctggtcgttgcgtcagtccagaggttccctcccctaccctttttctacttcccctcccccgccgctcaacttttctttcccttttactttctctctctcttcctcttcatccatcctctcttcatcacttccctcttcccttcatccaattcatcttccaagtgagtcttcctccccatctgtccctccatctttcccatcatcatctcccttcccagctcctcccctcctctcgtctcctcacgaagcttgactaaccattaccccgccacatagacacatctaaacaatgcccgccaccggcgaggaccaggacctggtgcaggacctgatcgaggaacccgccaccttctccgacgccgtcctgtcctccgacgaggaactgttccaccagaagtgccccaagccggctccgatctacagccccgtcagcaagcccgtcagcttcgagtccctgccgaaccgccgcctgcacgaagagttcctccgctcctccgtcgacgtcctgctgcaagaggccgtgttcgagggcaccaaccgcaagaaccgcgtcctgcagtggcgcgagcccgaagaactgcgccgcctgatggacttcggcgtccgcagcgccccgtccacgcatgaggaactgctcgaggtcctgaagaaggtcgtcacctactccgtcaagaccggccatccgtacttcgtcaaccagctgttctccgccgtcgatccctacggcctggtcgcccagtgggccaccgacgcgctgaacccctccgtctacacctacgaggtcagccccgtgttcgtcctgatggaagaggtcgtcctgcgcgagatgcgcgccatcgtcggcttcgaaggcggcaaaggcgacggcatcttctgccctggcggctcgatcgccaacggctacgccatcagctgcgcccgctaccgcttcatgcccgacatcaagaagaagggcctgcactccctgccgcgcctggtcctgttcacctccgaggacgcccactactcgatcaagaagctggcctcgttccaaggcatcggcaccgacaacgtctacctgatccgcaccgacgctcgcggtcgcatggacgtcagccacctggtcgaagagatcgagcgctccctccgcgagggcgctgccccgttcatggtcagcgccaccgccggcaccaccgtcatcggcgccttcgatcccatcgagaagatcgccgacgtctgccagaagtacaagctctggctgcacgtcgacgccgcctggggcggaggcgctctggtgtccgccaagcaccgccatctgctgaagggcatcgagcgcgccgactccgtcacctggaatccccacaagctgctgaccgctccgcagcagtgcagcaccctgctgctgcgccacgagggcgtcctggccgaggcgcactccaccaacgccgcctacctgttccagaaggacaagttctacgacaccaagtacgacaccggcgacaagcacatccagtgcggccgtcgcgccgacgtgctgaagttctggttcatgtggaaggccaagggcacctccggcctcgagaagcacgtggacaaggtgttcgagaacgcccgcttcttcaccgactgcatcaagaaccgtgagggcttcgagatggtgatcgccgagcctgagtacaccaacatctgtttctggtacgtccccaagagcctgcgcggacgcaaggacgaggccgactacaaggacaagctgcacaaggtcgcccctcgcatcaaagaacgcatgatgaaggaaggctccatgatggtcacctaccaggcgcagaagggccatccgaatttcttccgcatcgtctttcagaactccggcctggacaaggccgacatggtccatctggtcgaggaaatcgaacgcctgggctccgacctctgatgggttggatgacgatgacttcatgtgattttgttatttagaatattttatatttccttttcttcttctcaccaccgatccccttaacactcttgcttcatttgcttcagatttctcggtttcttcttttttcttctccccagttatccactatatctttgctagaccggcctgcgccctggcatgcatcataaaatcatgtccgttggtcatcatctgttttgtatatccgtcatataaagtattcttttattccctcccccctcggtcgtctttcgctgtcccgcttcctacctccggtttatagagcatggttcatctcttccgtacatttccgttggtactagcatttatgtcttcagctagtatagaagctgccgcagttgttcgcttactacctgcctaagtccttaactttttaaagtgtttaacctatacgtagtgttaaacgagtactgggaggtggtgaggtagaaaatgttctgcacgggcagtgggtatttggtagtgtgtaaggcggttatttatcaggctgacgctaaagacttctatgggagcagtatgggatcgcggctcatagaagtacacaaaatctaagagtcgtttgataattaattgattcccggcagggtcttcttgggattgagagaactggttactttgatttgagatattgtaaagcttaaggctcttaacacgtacgagcgaaacagcaggggggaaatcgggaaaaggggcgtggggtgaataaaaaagttgaaataagacactgtatcttgctgggggtgaataaagagagaataaaagagaggtaaattccactcagccccttttcttcgctctccaaacatcaaactccgccggccgacccacaggatcccgaacaagtggaagatatgtgccggtccagacccttcgcacagctaaaagcagaccttcataagcgtttccgggtagtattcgcacacctgaactggcacgtcggggacacaactgtttttgatacacaagaacacacaccacccatctaggactcagagctgggccagacattccttcatagtcttgacgatgaaggtgaagtcctcttcggtgatggacagcggaggggcgagctgcaggatgttgttgtagccggccacggtgtcgccgttcttgccgatgatcagacccttctctttgcaggcgttgatgaccttgttcatcttttcgatggaggcgggctctttggtctgcttatcctcgacgagttcgatacccagcaggaggcccttgccgcggacgtccccgacgttggggtgctctttgacgtcctccaactcgtacagcaggcgctcgcccagttctttggaccgctcgatgagcttctcgttttccatgatctcgaggttcttcagggccagcgcgcaggcggcagggttgccgccgaaggtgttgacatggcggaagcggtcgtagtcgtcggagccgacgaaggcctcgtagacctcgcggcggaccgcggtggcagacagcggcaggtaggccgaggtgatacccttggccatggtgataatgtcgggcttgacgccgtagttcatgaagccgaagggcttgccggtgcgaccgaagccgcagatgacctcgtcgcagatcagcagggcgccgtgcttttcgcagatctctttgaccttttccatgtagccgtccggcggcatcaggatgccaccaccggtgatgatgggttccatgatgacgccggcgacggtctgggacagctcccaggtcatgacgcggtcgatttcttcggcggaggccagggtgtgcacgtcctcggggttgcgataggtgtccggaggggccacgtgcaggaagccctgaccgaggggctcgtacttgtacttgcgctgggcctgaccggtcgcggccagggcacccatggagttgccgtggtaggcgcggtagcgagagatgaacttgtagcggccgtggtcacccttctgctggtggtactggcgggcgatcttgaaggcggtttcgttggcctccgagccggagttggagaagaagatgacgtactcgtcgtccagccactcgttcagcttctcggccagcttgatggcggggacgtgcgactgcgtcagcgggaagtacggcatctcttccagctgctcgaaggcagcgcgagccagctctttgcggccgtagccgacgttgacgcaccacaggccggacatgccgtccaggtagcggttgccgtcgatgtcggtgacccacgcgccttcggccttggtgatgatcaggttggtcggactcggagcggcaccgcgcatggcgtgccacaggtacttctcgtcggttttcttcaggctctgggtctgctcggtgacctggacgatcatcagttccatgttgatggactggagggggatgagttatggatcagtgaaactgggagaaaacaaagatggcaaagggagaacatggcccagatataggaaaaaacggaggaggcaaaaatgtaagcgctccggacttgctgtttcggtgtgcactagcagcagcgggggggaaggtggtgagtgttcaccgaggacccaaaaagaatgagcggatggcggatgagtgacggagaagggaaggacggggggggaattagaggtggagaggtccgatccatcaaatagaccaggctcggcacagccaagtttcccaaatgatcaactaatcaatgggacttggtgctaaatccggagatgccagatcattgatagacagacaggatggagtgatggcatatagacaggaggatggatggatggatagatggaggggtcaagcacaacatggtgggatgatggcggggtcatgactagcagctaagaggaagaagaggaggatgaaatggacagagaaagatgggaggggtgataaaatgagtatatgggacaagtcatacttacaggaccttgaagatggtggttgtactatctaagaaaggctttttttgagagtactcttaacacaagaggaggagggaggagggggaagtagtagataaataataaacacgaccacagacttgctacaggctacttcttgtaagctcgagtttctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgatcgtgctggtcacgggcgcgaccgccggtttcggcgagtgcatcacccgccgcttcatccagcagggccacaaggtgatcgctaccggacgccgccaagagcgcctccaagagctgaaggatgagctgggcgacaacctgtacattgcccagctggacgtgcgcaaccgggctgccatcgaagaaatgctcgcctcgctgcccgccgagtggtgcaacatcgacatcctggtcaacaacgccggtctggccctcggcatggaaccggcgcacaaggccagcgtcgaggactgggaaaccatgatcgacaccaacaacaagggactcgtctacatgacccgcgctgtgctgcccggcatggtcgagcgcaaccacggccacatcatcaacatcggctccaccgctggcagctggccctacgctggcggcaacgtctatggcgcgaccaaggcgttcgtccgccagttctccctgaacctgcgcaccgacctgcacggcaccgccgtccgcgtgaccgacattgagcccggtctggtcggcggcaccgagttcagcaacgtccgcttcaagggcgacgacggcaaggccgagaaaacctaccagaacaccgtcgctctgacccctgaggatgtcagcgaggccgtctggtgggtcagcactctgcccgcgcacgtcaacatcaacaccctcgagatgatgcccgtcacgcagtcctacgccggcctgaacgtccaccgccaataggaccgatggctgtgtagaagtactcgccgatagtggaaaccgacgccccagcactcgtccgagggcaaaggaatagagtagatgccgaccgggatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgtttcgggtttacctcttccagatacagctcatctgcaatgcattaatgcattggacctcgcaaccctagtacgcccttcaggctccggcgaagcagaagaatagcttagcagagtctattttcattttcgggagacgagatcaagcagatcaacggtcgtcaagagacctacgagactgaggaatccgctctctgacagacgggcaattgattacgggatcccattggtaacgaaatgtaaaagctaggagatcgtccgccgatgtcaggatgatttcacttgtttcttgtccggctcaccggtcaaagctaaagaggagcaaaaggaacggatagaatcgggtgccgctgatctatacggtatagtgcccttatcacgttgactcaacccatgctatttaactcaacccctccttctgaaccccaccatcttcttccttttcctctcatcccacacaattctctatctcagatttgaattccaaaagtcctcggacgaaactgaacaagtcttcctcccttcgataaacctttggtgattggaataactgaccatcttctatagttcccaaaccaaccgacaatgtaaatacactcctcgattagccctctagagggcatacgatggaagtcatggaatacttttggctggactctcacaatgatcaaggtatcttaggtaacgtctttggcgtgggccggtgttcgttcccagtcatcgatgcattcacatgccctccctaagctgggccctagactctaggatcctagtctagaaggacatggcatcgatggactgggttcgttctgagattatacggctaaaacttgatctggataataccagcgaaaagggtcatgccttctctcgttcttcctgttgatggaatggctaacagatgatagtcattgcaacttgaaacatgtctcctccagctgccatctacgaacccactgtggccgctaccggcctcaagggtaaggtcgtggtttctgagaccgtccccgttgagggagcttctcagaccaagctgttggaccatttcggtggcaagtgggacgagttcaagttcgcccctatccgcgaaagccaggtctctcgtgccatgaccagacgttactttgaggacctggacaagtacgctgaaagtgacgttgtcattgttggtgctggttcctgcggtctgagcactgcgtacgtcttggccaaggctcgtccggacctgaagattgctatcgtcgaggccagcgtctctcctggtcagtagtccatgatggattgccttgcactcagctttccggaactaacgtgcaataggtggcggtgcctggttgggtggccaactcttttctgctatggtcatgcgccgtcccgcggaagtcttcctgaacgagctgggtgttccttacgaagaggacgcaaaccccaactacgttgtcgtcaagcacgcctccctgtttacctcgacactcatgtcgaaggttctctccttccccaatgtcaagctcttcaatgctaccgctgttgaggacttgatcacccgtccgaccgagaacggcaacccccagattgctggtgttgtcgtcaactggacgctggtcacccttcaccacgatgatcactcctgcatggaccccaacactatcaacgctcctgtcatcatcagtaccactggtcacgatgggccattcggcgccttctgtgcgaagcgcttggtgtccatgggcagcgtcgacaagctaggtggcatgcgtggtctcgacatgagctcggccgaggatgccatcgtcaagaacacccgcgaggttactaagggcttgataatcggcggtatggagctgtctgaaattgatggctttaaccgcatgggccctaccttcggtgccatggttctcagtggtgtcaaggctgccgaggaggcattgaaggtgttcgacgagcgtcagcgcgagtgtgctgagtaaatgactcactacccgaatgggttcagtgcatgaaccggatttgtcttacggtctttgacgataggggaatgatgattatgtgatagttctgagatttgaatgaactcgttagctcgtaatccacatgcatatgtaaatggctgtgtcccgtatgtaacggtggggcattctagaataattatgtgtaacaagaaagacagtataatacaaacaaagatgcaagagcggctcctcagcaacattcgccatgttcatgtacagctttcaacggcctcgaacagtcactgtggatggataccagaggagagacccatcagttcaatcgcagggcagatgagtgtcgcatacattctcgccgtccagctggtcgaccagcaatgtcttttgtcccagttttctgagtttgatgacaacctggagaggccagaagtttgggatctggccaggaaggttacttcatctcaaagcgaagagtttgatcaagacggcaactgtctcagtgcgggtcgcgtgaggattgagttcaacgatggttcttctattacggaaagtgtcgagaagcctcttggtgtcaaagagcccatgccaaacgaacggattctccacaaataccgaacccttgctggtagcgtgacggacgaatcccgggtgaaagagattgaggatcttgtcctcggcctggacaggctcaccgacattagcccattgctggagctgctgaattgccccgtgaaatcgccactggtataaatgggaagcgatatggaaacatttcatgtcacgggcacaaattctaggtcatatcgtacctggatggtgaaaccaccagcggtttagcagatagaagatagactccttctgctctgcgttgcgtcttgaatttagttcgttcactggcttaagaacttagaatgcaatacagtctctcttatttcttattaaaatcacgtattcccacattcggcgactggaggatacgaaagcagtgttggtggtgctccccgtaatggatatgattttgctgactggactattctatgaccattccctccaacggagatcctttctcgacactttagatgttgacgctgtctggaggaactacttttgcgctgcaaagactatgagcagtggagctg

SEQ ID NO: 5 - protein, 1192 aa, Aspergillus niger
MAAPRQPEEAVDDTEFIDDHHDQHRDSVHTRLRANSAIMQFQKILVANRGEIPIRIFRTAHELSLQTVAVYSHEDRLSMHRQKADEAYMIGKRGQYTPVGAYLAIDEIVKIALEHGVHLIHPGYGFLSENAEFARKVEQSGMVFVGPTPQTIESLGDKVSARQLAIRCDVPVVPGTPGPVERYEEVKAFTDTYGFPIIIKAAFGGGGRGMRVVRDQAELRDSFERATSEARSAFGNGTVFVERFLDRPKHIEVQLLGDNHGNVVHLFERDCSVQRRHQKVVEIAPAKDLPADVRDRILADAVKLAKSVNYRNAGTAEFLVDQQNRYYFIEINPRIQVEHTITEEITGIDIVAAQIQIAAGATLEQLGLTQDRISTRGFAIQCRITTEDPSKGFSPDTGKIEVYRSAGGNGVRLDGGNGFAGAIITPHYDSMLVKCTCRGSTYEIARRKVVRALVEFRIRGVKTNIPFLTSLLSHPVFVDGTCWTTFIDDTPELFALVGSQNRAQKLLAYLGDVAVNGSSIKGQIGEPKLKGDIIKPVLHDAAGKPLDVSVPATKGWKQILDSEGPEAFARAVRANKGCLIMDTTWRDAHQSLLATRVRTIDLLNIAHETSHALANAYSLECWGGATFDVAMRFLYEDPWDRLRKLRKAVPNIPFQMLLRGANGVAYSSLPDNAIYHFCKQAKKCGVDIFRVFDALNDVDQLEVGIKAVHAAEGVVEATICYSGDMLNPSKKYNLPYYLDLVDKVVQFKPHVLGIKDMAGVLKPQAARLLIGSIRERYPDLPIHVHTHDSAGTGVASMIACAQAGADAVDAATDSLSGMTSQPSIGAILASLEGTEHDPGLNSAQVRALDTYWAQLRLLYSPFEAGLTGPDPEVYEHEIPGGQLTNLIFQASQLGLGQQWAETKKAYESANDLLGDVVKVTPTSKVVGDLAQFMVSNKLTAEDVIARAGELDFPGSVLEFLEGLMGQPYGGFPEPLRSRALRDRRKLDKRPGLYLEPLDLAKIKSQIRENYGAATEYDVASYAMYPKVFEDYKKFVAKFGDLSVLPTRYFLAKPEIGEEFHVELEKGKVLILKLLAIGPLSEQTGQREVFYEVNGEVRQVSVDDKKASVENTARPKAELGDSSQVGAPMSGVVVEIRVHDGLEVKKGDPIAVLSAMKMEMVISAPHSGKVSSLLVKEGDSVDGQDLVCKIVKA

SEQ ID NO: 6 - DNA, 1955 bp, Aspergillus niger
  source 1..1955: mol_type = genomic DNA
atggccgcccgtcggtctattgtccgttccatccccgctctccgcgccgcctctccccgcacggccgtggccacctccagatctgtagctgcagcttcggcctcgaggcgcctttctactgttataatgccctcttcttcttcgacaacttcttctgcaacatctaagctctcctcatcctcacctgccaccatgtcagcaacacgtcgactccacgctactgcccagcagttcacccccgcaactacctccgcggctactaccgccaccgagtacccgacggaccatagccccatcgccaaccccatcgacaccgccaatttcctggacaaccaatttgtggcttctaaggctaccacctggatcgatctgcacgatcctgctaccaacaacctcgttactcgggtgccgcaaagcaccgatgaggaactccgtgccgccgtcgagtcggccgagaaggccttcccagcctggcgcgcaaccagcgttattgccaagcagcagatcatgttcaagttcgtgagcttgattcgtgccaactgggaccgacttgctgcatccatcactctcgagcagggcaagacctttgccgatgccaagggcgatgttctccgcggtcttcaagttgccgaaacagcatgtggtattaccacccagctaacgggcgaggtgctcgaagtggccaaggatatggagactaggagctacagagagcccttgggtgtggttgctgccatttgtcctttcagtaagtgattctgcatccgtcctgcaagctaggtgacattgtccgacgctaaccgcaccccctcgccttctcggcagacttccccgcaatgattcccctgtggtgcatccccatcgctaccgtgacgggcaactgtctcatcctcaagccatctgagcgggaccccggagctgcaatgatcctggccgagctggcccgggaagcaggcttcccccccggtgtcatcaacattgtccacggctccgccaagacagtcgatttcatcctcgacgaacccgccatcaaggccatcagcttcgtcggcagcaaccgcgccggagagtacatctacacccgcggctctgccaacggcaagcgtgtccaggccaatctgggcgccaagaaccatgccgccgtagtcccagactgcaacaagaaccagaccctgaacgccctcgtcggagcggccttcggtgctgccggccaacgctgcatggccctcagtacggtcgtcatggtcggcgagactcaggactggctccccgagatggccgaacgggccaaggccctgaacgtcaacggaggcttcgaagaaggcgctgacctcggccccgtcatcagccccgaaagcaagaagcgcatcgaagacctgatcgccagcgccgaagaagagggtgcgaccatcctcctcgacggcagaggctacaagccagagaagtaccccaacggcaactttgtaagcttccacccaacccccttccccgtctcctatccagctacatcttctctctaaccagcaccaaaaccacaggttggccccaccatcatcaccaacgtaacccccgaaatgaaatgctacaaggaagaaatcttcggccccgtcctcgtctgcttgtccgtgcccacactagacgacgccattgagctcatcaacaagaacgagtacggaaacggagctgccatcttcactcgctccggttccacggcctctcgcttccagaaggacatcgaggccggccaggtcggtgttaacgtgcccattcctgtgccgttgcccatgttctctttcaccggtaacaagaagagtattgccggcggtggcgctaatactttctatggtaagcccgggttgcaattttacacgcagcagaagacggtgactagtttgtggaggagtgaggatgctgttagcactaaggctcatacggttatgccgactcattcgtga

SEQ ID NO: 7 - DNA, 1713 bp, Aspergillus niger
  source 1..1713: mol_type = genomic DNA
atgtcaagatcgcagaattggacgaatgctttggatccggtatttggtttgatcaacctcagattattgcgattagtgctaaagtgaaggacaggtttcacaacgtctactttgcagggtcccggggagtacgcttcaggaagtaaagcgggctgttgatgctgctgaagatgcccagcctggatgggctgctctggggttccaggtgaggcgtgaacatctgttgcggctggtggatgtattgagacagatgtctcctgagattgtaagctgccgggacagctgacaggcaaaggagtggaactgacgatcgtaggtgacctgcctgtctcgagaagttggaaagacattagccgatgcagacgcggaggttttccgtggtctggactgcatacatgccgcatgttcgatcgggcctgaaatggctggcatgtttttaggaggcgatgcaacattgctgcaaactttctacgagccagttgtgagtctagatgccggttaactacagatatgctactaacagaccgtggcagggcgtctgtgtctcaatcactccatttagctttcccttcatgattcccctgtggtcgcttccgtacgcgcttatcactggcaacaccgtcatcctgaaaccatccgagaaaacaccaactacgtcttcgttattagcgcaggcattcatcaagactggcttccctcccggggtcttcaatgtgctccacggcggcccatccacagtgcagatgcttgtgacccagccgacggttcaagcggtgagtttcgttgggtccgagtcagcagctaggcaggtgcatgacctggcaagggccgcagggaagcgaatacaagccgagtgtggtggtaagaatcatggggttgtcttggaggatgctaatatgtcctcgacgctttttgcgatcgctggaagtgcctttggggccgccggtcagcgttgtatggccttgagcgtagcggtgtttgtaggtgcgacgagagactggatacctaggctggtggagctggcacagtcgatggtagtaggatgtggtggtgatcaggaatctaagattgggccgttgattgacaaaacagccaaagacaaagtcagcgagatgatccagcgagcagttgaggagagagcaaccgttcttttggacggtcgagatattgaggtccccggctatccggatggcaacttcatgggcccaactattctcggggatgtgcagacttacatggaatgctatcaggcggaaatatttgggcctgtgctcatctgcatggaggtggacactcttgaggaagcgatcgatctgataagccaaaataagtgtcagtctcccacaacccgggcttgaaggggatggattgctgacctgatcgcagatggtaacggatgctctattttcaccaccagcggcaagcatgcaaatacgttccaacgctgtgtcaatgttggtcagattggtgtcaacatcccgttgatcggtatggcggtccccatttccgtagcgcaaccaacccctgacagtacaacagcgccgtatggaaccgcggtccgaacgagcaacaaagattctttcctgggtggtgagtgcccagacatctccggtggaaggaaagctgacctctgcagatcgacattcccctgggaagacatattggccatttttcacaacaactaaaacggtctcatctcgatgggatcagtga

SEQ ID NO: 8 - DNA, 1758 bp, Aspergillus niger
  source 1..1758: mol_type = genomic DNA
atgcccttctcctccttccccgctccctttcgctgtctttccttctcctctctgcggctgcgtggtgtgtcattctcccgtctgccagttgttcctttctcccctcatcgtcatcacttccatagctcgtccatcacaatgagcgatctgtccgtgcagctgacagccccgaatgggcgcacatacgcccagccggttggtttgttcatcaacaatgaatttgtcgcttccaagtccggcgagaagttcgccaccatcaacccttcgtacgtgacctcccaatcctagtatctttcttggatctgctgctgaccagtgctagaaatgaagcggaaatcacctcggtctatgctgcgggtgaagaagacgtggatattgctgtgaaggctgcccggaaagccctcaaccacccgtcatggaagttgttgcctccgactgaacggggcaccctgatgttgaagctggccgacttgatcgagcagcacaaggagacgcttgccaccattgaaacgtgggacaatggtagggagcctcgcattgtcctcaaaggcgatcaccatgtactgattctctccaggcaagccatactcagtttcgctgaacgatgacctgggagaggttgttggctgcctccggtactatgctggatacgccgacaaggtccatggtcagactattagcaccaccccggccaagtttgcctatactcttcgtcagccaattggtgtcgtgggtcagattatcccctggaacttccctctcgcgatggctgcctggaagttgggtcctgccttggcctgcggaaacaccgtggttctgaaaccggctgagcagactccgctcagtgttctctatcttgccaatctcatcaaggaagccggtttcccgccaggtgttgtcaacatcctgaacggctttggacgcgtagctggaagtgcgctcgtgacccaccccggggtggacaaggttgccttcacaggttcgacgttgactggccgcgaaattatgaagttggctgcaggcaccctgaagaacattacgctcgagacaggtggcaaatctcctctggtcgtcttcagtgatgccgatatcgaccaggccgctaagtgggcgcatgcgggtatcatgtacaaccaaggacaggtttgcaccgccacctctcgtatcctggtgcacgagtccgtctatgacaagttcgtggcgctcttcaaggaagccgtggccaacaccagcaaggtgggcgatccctttgccgacgacaccttccagggtccccaggtcaccaaggcccaatacgaccgcgtgctctcctatatcgaagctggaaagtccgaaggagcgaccctggtggccggtggcgagccgttcaagaacgtcggcgacggcaagggtttctttatcgcgcccaccatcttcaccaacgtcaaggataacatgcggatctaccgggaggaggttttcggaccatttgtcgtgatttccagcttctcagaggaggatgaggctgtgagacgggccaacgatacgacctatggactgggcgctgccctgttcaccaaggacattgagcgtgcacaccgggttgcgtcggagatcgaagcgggcatggtctggatcaacagcagcaacgacagcgatatccgtgtgcctttcgggggtgtgaagcagagcggaattgggcgcgaacttggcgaggccggtctggaggcctactcacagatcaaagcggtccatgtcaacttgggaacgaagctgtag

SEQ ID NO: 9 - DNA, 1232 bp, Aspergillus niger
  source 1..1232: mol_type = genomic DNA
atgaaagttgatacccccgattctgcttccaccatcagcatgaccaacactatcaccatcaccgtagagcaggacggtatctatgagatcaacggtgcccgtcaagagcccgtggtcaacctgaacatggtcaccggtgcgagcaaactgcgcaagcagcttcgcgagaccaatgagttgctcgtgtgtcctggtgtgtacgacggtctgtccgcccgtattgccatcaacctgggcttcaagggcatgtacatggtatgttggattcctcacactacctttccccacagtcaacacttctccgcttccgcgatggagaaaaaagatcatactaacggaagggtcagaccggcgccggtactaccgcgtctagactgggcatggccgatctgggtctagcccacatctacgacatgaagaccaacgcggagatgatcgcaaacctggacccctacggtcctcccctgatcgcagacatggacactggctacggaggtgagaatcccccatctccactgtctgccaagacataatgatctacccgcgccaaaaagcaaaacggcaatatagacccagttccccactaacacaaaaaaaaaacaaaaataggccccctgatggtcgcccgttccgttcaacaatacatccaagccggagtcgcgggattccacatcgaagatcagatccaaaacaagcgatgcggacacctggcaggcaagcgcgtcgtcaccatggacgaatacttgactcgcatccgcgccgccaagctcaccaaggaccgcctccgcagcgacatcgtgctgattgcccgcaccgacgccctccagcagcacggctacgacgagtgcattcgccgccttaaggccgcccgcgatcttggcgccgatgttggtctcctcgagggcttcaccagtaaggagatggcgaggcggtgtgtccaggaccttgcgccttggccgcttcttctcaacatggtggagaacggtgctgggccggttatttccgtcgatgaggctagggaaatgggcttccgcattatgatcttctcgttcgcttgcattactcctgcctatatggggattacggctgctctggagaggctcaagaaggatggtgtggttgggttgcccgaggggatggggccgaagaagctgtttgaggtttgcggattgatggactcggtgagggttgataccgaggctggtggagatgggtttgctaatggtgtttaa

SEQ ID NO: 10 - DNA, 50 bp, synthetic construct
  source 1..50: mol_type = other DNA
ccctcgaggtcgacggtatcgatagatatcggttgtagcagcgtaaacac

SEQ ID NO: 11 - DNA, 33 bp, synthetic construct
  source 1..33: mol_type = other DNA
tctttcatagtagccttggtgaacatcttgagg

SEQ ID NO: 12 - DNA, 34 bp, synthetic construct
  source 1..34: mol_type = other DNA
atgttcaccaaggctactatgaaagaccgcgatg

SEQ ID NO: 13 - DNA, 36 bp, synthetic construct
  source 1..36: mol_type = other DNA
cgccggtggcgggcattgtttagatgtgtctatgtg

SEQ ID NO: 14 - DNA, 33 bp, synthetic construct
  source 1..33: mol_type = other DNA
catctaaacaatgcccgccaccggcgaggacca

SEQ ID NO: 15 - DNA, 35 bp, synthetic construct
  source 1..35: mol_type = other DNA
atccaacccatcagaggtcggagcccaggcgttcg

SEQ ID NO: 16 - DNA, 33 bp, synthetic construct
  source 1..33: mol_type = other DNA
gggctccgacctctgatgggttggatgacgatg

SEQ ID NO: 17 - DNA, 33 bp, synthetic construct
  source 1..33: mol_type = other DNA
tctggcccagctctgagtcctagatgggtggtg

SEQ ID NO: 18 - DNA, 35 bp, synthetic construct
  source 1..35: mol_type = other DNA
catctaggactcagagctgggccagacattccttc

SEQ ID NO: 19 - DNA, 36 bp, synthetic construct
  source 1..36: mol_type = other DNA
gtccatcaacatggaactgatgatcgtccaggtcac

SEQ ID NO: 20 - DNA, 33 bp, synthetic construct
  source 1..33: mol_type = other DNA
cgatcatcagttccatgttgatggactggaggg

SEQ ID NO: 21 - DNA, 102 bp, synthetic construct
  source 1..102: mol_type = other DNA
gaactagtggatcccccgggctgcgttaactcgagcttacaagaagtagccggctacttcttgtaagctcgagttaacgcagcccgggggatccactagttc

SEQ ID NO: 22 - DNA, 99 bp, synthetic construct
  source 1..99: mol_type = other DNA
tgaccagcacgatcatggtgatgtctgctcaagcttgagcagacatcaccatgatcgtgctggtcacttgagcagacatcaccatgatcgtgctggtca

SEQ ID NO: 23 - DNA, 33 bp, synthetic construct
  source 1..33: mol_type = other DNA
agacatcaccatgatcgtgctggtcacgggcgc

SEQ ID NO: 24 - DNA, 66 bp, synthetic construct
  source 1..66: mol_type = other DNA
gccatcggtcctattggcggtggacgttcaggcgcctgaacgtccaccgccaataggaccgatggc

SEQ ID NO: 25 - DNA, 33 bp, synthetic construct
  source 1..33: mol_type = other DNA
cgtccaccgccaataggaccgatggctgtgtag

SEQ ID NO: 26 - DNA, 66 bp, synthetic construct
  source 1..66: mol_type = other DNA
cccgtctgtcagagagcggattcctcagtctcgcgagactgaggaatccgctctctgacagacggg

SEQ ID NO: 27 - DNA, 37 bp, synthetic construct
  source 1..37: mol_type = other DNA
gaggaatccgctctctgacagacgggcaattgattac

SEQ ID NO: 28 - DNA, 66 bp, synthetic construct
  source 1..66: mol_type = other DNA
gaatgttgctgaggagccgctcttgcatctttgcaaagatgcaagagcggctcctcagcaacattc

SEQ ID NO: 29 - DNA, 35 bp, synthetic construct
  source 1..35: mol_type = other DNA
gcaagagcggctcctcagcaacattcgccatgttc

SEQ ID NO: 30 - DNA, 49 bp, synthetic construct
  source 1..49: mol_type = other DNA
actaaagggaacaaaagctggagctcagctccactgctcatagtctttg

SEQ ID NO: 31 - DNA, 48 bp, synthetic construct
  source 1..48: mol_type = other DNA
acacaattctctatctcagatttgctactatgaaagaccgcgatgggc

SEQ ID NO: 32 - DNA, 50 bp, synthetic construct
  source 1..50: mol_type = other DNA
ttcagtttcgtccgaggacttttggaattcaaatctgagatagagaattg

SEQ ID NO: 33 - DNA, 43 bp, synthetic construct
  source 1..43: mol_type = other DNA
cgaggtcgacggtatcgataacggatcggcaaagcaatctacg

SEQ ID NO: 34 - DNA, 30 bp, synthetic construct
  source 1..30: mol_type = other DNA
atccaacccatcagaggtcggagcccaggc

SEQ ID NO: 35 - DNA, 32 bp, synthetic construct
  source 1..32: mol_type = other DNA
cgacctctgatgggttggatgacgatgacttc

SEQ ID NO: 36 - DNA, 44 bp, synthetic construct
  source 1..44: mol_type = other DNA
agtggatcccccgggctgcactcgagcttacaagaagtagcctg

SEQ ID NO: 37 - DNA, 42 bp, synthetic construct
  source 1..42: mol_type = other DNA
gcccgggggatccactagttgctggagctagtggaggtcaac

SEQ ID NO: 38 - DNA, 31 bp, synthetic construct
  source 1..31: mol_type = other DNA
ctgtacagagcggtcggcatctactctattc

SEQ ID NO: 39 - DNA, 31 bp, synthetic construct
  source 1..31: mol_type = other DNA
atgccgaccgctctgtacagtgaccggtgac

SEQ ID NO: 40 - DNA, 43 bp, synthetic construct
  source 1..43: mol_type = other DNA
agggaacaaaagctggagcttctagagaattgtgtgggatgag

SEQ ID NO: 41 - DNA, 40 bp, synthetic construct
  source 1..40: mol_type = other DNA
ctctcatcccacacaattctgagcatcatcccatgatagc

SEQ ID NO: 42 - DNA, 34 bp, synthetic construct
  source 1..34: mol_type = other DNA
aaaggggcgaactgttgtagaagatatccgttag

SEQ ID NO: 43 - DNA, 30 bp, synthetic construct
  source 1..30: mol_type = other DNA
ctacaacagttcgcccctttcctcctcttc

SEQ ID NO: 44 - DNA, 48 bp, synthetic construct
  source 1..48: mol_type = other DNA
gggaacaaaagctggagcttctagacgagcaatacggaagcgaatatc

SEQ ID NO: 45 - DNA, 56 bp, synthetic construct
  source 1..56: mol_type = other DNA
ctcgaggtcgacggtatcgatagtttaaactgaggtgcagtggatgattattaatc

SEQ ID NO: 46 - DNA, 30 bp, synthetic construct
  source 1..30: mol_type = other DNA
gaggtcaacgttgatctgcttgatctcgtc

SEQ ID NO: 47 - DNA, 31 bp, synthetic construct
  source 1..31: mol_type = other DNA
agcagatcaacgttgacctcacagggatttc

SEQ ID NO: 48 - DNA, 37 bp, synthetic construct
  source 1..37: mol_type = other DNA
aaaggggcgacttactgttgtagaagatatccgttag

SEQ ID NO: 49 - DNA, 30 bp, synthetic construct
  source 1..30: mol_type = other DNA
caacagtaagtcgcccctttcctcctcttc

SEQ ID NO: 50 - DNA, 33 bp, synthetic construct
  source 1..33: mol_type = other DNA
gacagggcagttatgaagtctcccgaactacgc

SEQ ID NO: 51 - DNA, 35 bp, synthetic construct
  source 1..35: mol_type = other DNA
gacttcataactgccctgtcgagtaagtaaatttg

SEQ ID NO: 52 - DNA, 48 bp, synthetic construct
  source 1..48: mol_type = other DNA
actagtggatcccccgggctgcaagcttactacagagaggagctgaag

SEQ ID NO: 53 - DNA, 32 bp, synthetic construct
  source 1..32: mol_type = other DNA
atggccactgttgatctgcttgatctcgtctc

SEQ ID NO: 54 - DNA, 35 bp, synthetic construct
  source 1..35: mol_type = other DNA
caaggcctagctgccctgtcgagtaagtaaatttg

SEQ ID NO: 55 - DNA, 55 bp, synthetic construct
  source 1..55: mol_type = other DNA
tcttcagctcctctctgtagtactaggccttgacgatcttgcagacaagatcctg

SEQ ID NO: 56 - DNA, 30 bp, synthetic construct
  source 1..30: mol_type = other DNA
cttcaaaatggctgctccccgccagcccga

SEQ ID NO: 57 - DNA, 34 bp, synthetic construct
  source 1..34: mol_type = other DNA
ggggagcagccattttgaagatggatgagaagtc

SEQ ID NO: 58 - DNA, 50 bp, synthetic construct
  source 1..50: mol_type = other DNA
ctccaccgcggtggcggccgctgtttaaacagtggccatgaaatccaatc

SEQ ID NO: 59 - DNA, 42 bp, synthetic construct
  source 1..42: mol_type = other DNA
cgaggtcgacggtatcgatacagaagatgatattgaaggagc

SEQ ID NO: 60 - DNA, 31 bp, synthetic construct
  source 1..31: mol_type = other DNA
aagtagtcatttggatgcttgggtagaatag

SEQ ID NO: 61 - DNA, 32 bp, synthetic construct
  source 1..32: mol_type = other DNA
aagcatccaaatgactactttggatgacactg

SEQ ID NO: 62 - DNA, 32 bp, synthetic construct
  source 1..32: mol_type = other DNA
aaagctagagttatggacatggcatggacatg

SEQ ID NO: 63 - DNA, 39 bp, synthetic construct
  source 1..39: mol_type = other DNA
atgtccataactctagctttgtattgtctttaaatttac

SEQ ID NO: 64 - DNA, 40 bp, synthetic construct
  source 1..40: mol_type = other DNA
agtggatcccccgggctgcagtaagtagaaagctttgggg

SEQ ID NO: 65 - DNA, 43 bp, synthetic construct
  source 1..43: mol_type = other DNA
cgaggtcgacggtatcgataacagaagatgatattgaaggagc

SEQ ID NO: 66 - DNA, 31 bp, synthetic construct
  source 1..31: mol_type = other DNA
aacgaagaggcagtaagtagaaagctttggg

SEQ ID NO: 67 - DNA, 30 bp, synthetic construct
  source 1..30: mol_type = other DNA
ctacttactgcctcttcgtttctgtgatgc

SEQ ID NO: 68 - DNA, 34 bp, synthetic construct
  source 1..34: mol_type = other DNA
cggtcgtatgcattttgaagatggatgagaagtc

SEQ ID NO: 69 - DNA, 30 bp, synthetic construct
  source 1..30: mol_type = other DNA
cttcaaaatgcatacgaccgagaagatacc

SEQ ID NO: 70 - DNA, 41 bp, synthetic construct
  source 1..41: mol_type = other DNA
agtggatcccccgggctgcaggttctctccttgcagcaaag

SEQ ID NO: 71 - DNA, 45 bp, synthetic construct
  source 1..45: mol_type = other DNA
gaggtcgacggtatcgataagcttctcagctgggtgaagaacaac

SEQ ID NO: 72 - DNA, 31 bp, synthetic construct
  source 1..31: mol_type = other DNA
tagctccagcgtgatagtgttggtcatgctg

SEQ ID NO: 73 - DNA, 32 bp, synthetic construct
  source 1..32: mol_type = other DNA
acactatcacgctggagctagtggaggtcaac

SEQ ID NO: 74 - DNA, 32 bp, synthetic construct
  source 1..32: mol_type = other DNA
cgttagtatgcggtcggcatctactctattcc

SEQ ID NO: 75 - DNA, 30 bp, synthetic construct
  source 1..30: mol_type = other DNA
atgccgaccgcatactaacggaagggtcag

SEQ ID NO: 76 - DNA, 42 bp, synthetic construct
  source 1..42: mol_type = other DNA
agtggatcccccgggctgcagtaccacgcaagcttcgatatg

SEQ ID NO: 77 - DNA, 44 bp, synthetic construct
  source 1..44: mol_type = other DNA
gaggtcgacggtatcgatatctagagtaaactggtgcagctatc

SEQ ID NO: 78 - DNA, 33 bp, synthetic construct
  source 1..33: mol_type = other DNA
tagctccagcaggtgaggatgaggagagcttag

SEQ ID NO: 79 - DNA, 32 bp, synthetic construct
  source 1..32: mol_type = other DNA
atcctcacctgctggagctagtggaggtcaac

SEQ ID NO: 80 - DNA, 32 bp, synthetic construct
  source 1..32: mol_type = other DNA
gactgtcttgcggtcggcatctactctattcc

SEQ ID NO: 81 - DNA, 32 bp, synthetic construct
  source 1..32: mol_type = other DNA
atgccgaccgcaagacagtcgatttcatcctc

SEQ ID NO: 82 - DNA, 41 bp, synthetic construct
  source 1..41: mol_type = other DNA
agtggatcccccgggctgcacacttgtccagaccgaggtac

SEQ ID NO: 83 - DNA, 44 bp, synthetic construct
  source 1..44: mol_type = other DNA
gaggtcgacggtatcgatagaggatatgatctcttcaactatac

SEQ ID NO: 84 - DNA, 30 bp, synthetic construct
  source 1..30: mol_type = other DNA
tagctccagctctcaggagacatctgtctc

SEQ ID NO: 85 - DNA, 32 bp, synthetic construct
  source 1..32: mol_type = other DNA
tctcctgagagctggagctagtggaggtcaac

SEQ ID NO: 86 - DNA, 32 bp, synthetic construct
  source 1..32: mol_type = other DNA
ctaccatcgacggtcggcatctactctattcc

SEQ ID NO: 87 - DNA, 30 bp, synthetic construct
  source 1..30: mol_type = other DNA
atgccgaccgtcgatggtagtaggatgtgg

SEQ ID NO: 88 - DNA, 40 bp, synthetic construct
  source 1..40: mol_type = other DNA
agtggatcccccgggctgcacagagaaggtgcagattgtg

SEQ ID NO: 89 - DNA, 41 bp, synthetic construct
  source 1..41: mol_type = other DNA
cgaggtcgacggtatcgatagctggcggtagaaggattttc

SEQ ID NO: 90 - DNA, 32 bp, synthetic construct
  source 1..32: mol_type = other DNA
tagctccagcaattggctgacgaagagtatag

SEQ ID NO: 91 - DNA, 29 bp, synthetic construct
  source 1..29: mol_type = other DNA
tcagccaattgctggagctagtggaggtc

SEQ ID NO: 92 - DNA, 31 bp, synthetic construct
  source 1..31: mol_type = other DNA
actgaagacgcggtcggcatctactctattc

SEQ ID NO: 93 - DNA, 32 bp, synthetic construct
  source 1..32: mol_type = other DNA
atgccgaccgcgtcttcagtgatgccgatatc

SEQ ID NO: 94 - DNA, 41 bp, synthetic construct
  source 1..41: mol_type = other DNA
agtggatcccccgggctgcacgtttgggctttgtcctttag

SEQ ID NO: 95 - DNA, 41 bp, synthetic construct
  source 1..41: mol_type = other DNA
cgaggtcgacggtatcgataccaactggcgctgtatagatc

SEQ ID NO: 96 - DNA, 30 bp, synthetic construct
  source 1..30: mol_type = other DNA
tagctccagcggtctgaaagtagtcctgtg

SEQ ID NO: 97 - DNA, 29 bp, synthetic construct
  source 1..29: mol_type = other DNA
ctttcagaccgctggagctagtggaggtc

SEQ ID NO: 98 - DNA, 31 bp, synthetic construct
  source 1..31: mol_type = other DNA
acgaggattgcggtcggcatctactctattc

SEQ ID NO: 99 - DNA, 34 bp, synthetic construct
  source 1..34: mol_type = other DNA
atgccgaccgcaatcctcgtcgaccatcaaaaag

SEQ ID NO: 100 - DNA, 39 bp, synthetic construct
  source 1..39: mol_type = other DNA
agtggatcccccgggctgcaagagggtatgaaggaggag

SEQ ID NO: 101 - DNA, 43 bp, synthetic construct
  source 1..43: mol_type = other DNA
aggtcgacggtatcgatatcacagaagatgatattgaaggagc

SEQ ID NO: 102 - DNA, 41 bp, synthetic construct
  source 1..41: mol_type = other DNA
gatagggtggttgtctggatcagtaagtagaaagctttggg

SEQ ID NO: 103 - DNA, 41 bp, synthetic construct
  source 1..41: mol_type = other DNA
cgaggtcgacggtatcgatatccagacaaccaccctatctc

SEQ ID NO: 104 - DNA, 30 bp, synthetic construct
  source 1..30: mol_type = other DNA
gtctggacatcttgatgaaggtctgggttg

SEQ ID NO: 105 - DNA, 30 bp, synthetic construct
  source 1..30: mol_type = other DNA
cttcatcaagatgtccagactcgataagtc

SEQ ID NO: 106 - DNA, 33 bp, synthetic construct
  source 1..33: mol_type = other DNA
cgtgatacgctccatgattcatgacgtatattc

SEQ ID NO: 107 - DNA, 30 bp, synthetic construct
  source 1..30: mol_type = other DNA
gaatcatggagcgtatcacgaggccctttc

SEQ ID NO: 108 - DNA, 31 bp, synthetic construct
  source 1..31: mol_type = other DNA
gccgaagaccgatcctcaatcaccatcctcc

SEQ ID NO: 109 - DNA, 37 bp, synthetic construct
  source 1..37: mol_type = other DNA
attgaggatcggtcttcggctatagttcatttttatc

SEQ ID NO: 110 - DNA, 42 bp, synthetic construct
  source 1..42: mol_type = other DNA
agtggatcccccgggctgcagttgcgatcaggtgtgtaattg

SEQ ID NO: 111 - DNA, 33 bp, synthetic construct
  source 1..33: mol_type = other DNA
tcatcctcaggaacgactccagaagtgactaag

SEQ ID NO: 112 - DNA, 31 bp, synthetic construct
  source 1..31: mol_type = other DNA
gttcaatcatggtgaaatttgggattgtgac

SEQ ID NO: 113 - DNA, 32 bp, synthetic construct
  source 1..32: mol_type = other DNA
aaatttcaccatgattgaacaagatggattgc

SEQ ID NO: 114 - DNA, 41 bp, synthetic construct
  source 1..41: mol_type = other DNA
agtggatcccccgggctgcagactctgctaagctattcttc

SEQ ID NO: 115 - DNA, 70 bp, synthetic construct
  source 1..70: mol_type = other DNA
ggtcgacggtatcgataataacttcgtatagcatacattatacgaagttatgaacgactccagaagtgac

SEQ ID NO: 116 - DNA, 70 bp, synthetic construct
  source 1..70: mol_type = other DNA
atcccccgggctgcaataacttcgtataatgtatgctatacgaagttatgactctgctaagctattcttc

SEQ ID NO: 117 - DNA, 52 bp, synthetic construct
  source 1..52: mol_type = other DNA
attgggtaccgggcccccccgtttaaacttcggagtagcaacgagtattttc

SEQ ID NO: 118 - DNA, 30 bp, synthetic construct
  source 1..30: mol_type = other DNA
attgtttagaagcgcagttaatggtgtatg

SEQ ID NO: 119 - DNA, 30 bp, synthetic construct
  source 1..30: mol_type = other DNA
taactgcgcttctaaacaatgcccgccacc

SEQ ID NO: 120 - DNA, 22 bp, synthetic construct
  source 1..22: mol_type = other DNA
tcccatactgctcccatagaag

SEQ ID NO: 121 - DNA, 22 bp, synthetic construct
  source 1..22: mol_type = other DNA
tctatgggagcagtatgggatc

SEQ ID NO: 122 - DNA, 31 bp, synthetic construct
  source 1..31: mol_type = other DNA
cttcatcaagatggaactgatgatcgtccag

SEQ ID NO: 123 - DNA, 30 bp, synthetic construct
  source 1..30: mol_type = other DNA
tcagttccatcttgatgaaggtctgggttg

SEQ ID NO: 124 - DNA, 41 bp, synthetic construct
  source 1..41: mol_type = other DNA
tattatcgataccgtcgacctccagacaaccaccctatctc

SEQ ID NO: 125 - DNA, 43 bp, synthetic construct
  source 1..43: mol_type = other DNA
atacgaagttattgcagcccgggacatgctggaagggattttc

SEQ ID NO: 126 - DNA, 33 bp, synthetic construct
  source 1..33: mol_type = other DNA
gcacgatcattttgaagatggatgagaagtcgg

SEQ ID NO: 127 - DNA, 30 bp, synthetic construct
  source 1..30: mol_type = other DNA
catcttcaaaatgatcgtgctggtcacggg

SEQ ID NO: 128 - DNA, 51 bp, synthetic construct
  source 1..51: mol_type = other DNA
ctagaactagtggatcccccgtttaaaccaatgggatcccgtaatcaattg

SEQ ID NO: 129 - DNA, 1272 bp, Aspergillus niger
  source 1..1272: mol_type = genomic DNA
atgcatacgaccgagaagatacccagcttcctatctcgccggaaacccagcaatgattcagaacacgtacccccaccacccgatggaggagtccaagcctggacccaagtcgtctgcatgcacttcgtattcttcaacacctgggggatcaccactagcttctccgtctttgagcaattatacaccaaaaccctaccacagtcagcatcctctatctcctggatcggcagcgtccaggtatctctcctgttcttcctgagcgcccttgccggacgcgcaacagacgccggcttcttcagagtcatgtacccactcggggtcctcctccagctcgtgggaatcttcatgctctctctgtgcaagacctactggcaaatcttcctcgctcaggcggtatgcatgggtctcggcaatggacttaccttcagccccggactttcagtcatgtccgcctacttcatgaaaaaccgagcctttgcagtgggcttggctgcgtctggtgcagcaacagggggtttgatataccccgtactcatcaaccaattgctctataatcaccagataggcttcgcatggacaatacgcgcagcaggacttctcatgctaattacccatatctttggcctggttttcttcagaccccgtcttccaccccggaccaccggccctctcatcgaactcggcgcatttaccgagccgcctttcgtcttcttcaccttatgctacttcttcaccttctggggcctgtacttcgcctggttctatcttggcacctttgcccgagaccgtctcggcatcgcggatacacagaacctgctccttgtgttgaatggagtcggcatcgtcggacgtatcgggccgagtataattggtgatcgatggacggggcgattaaacatcttgatccccatcactctttcatgtgcgattctgatgttttgctggatagccgtcagtactgttgcagggctgtatgcgtttgttgttgtgtatgggctcgtgggtggcgccgcgcaatcggtgatccctgcgacggcgacgaccatgacgccggatatcaatcggacgggtacaaggctgggaatgatcatgagtattgtgggatttgctacgctgactggaccggctattgaaggggcgttgatcgctactgacggggggaggtatgtggcggctcagatttttgcgggtgtgagtattctgctgggcgtgtgtgcggttgcggcggcgcgggtggctaaggctgggtgggggttggatgtgaaggtgtga

SEQ ID NO: 130 - DNA, 1623 bp, synthetic construct
  source 1..1623: mol_type = other DNA
atgcccgccaccggcgaggaccaggacctggtgcaggacctgatcgaggaacccgccaccttctccgacgccgtcctgtcctccgacgaggaactgttccaccagaagtgccccaagccggctccgatctacagccccgtcagcaagcccgtcagcttcgagtccctgccgaaccgccgcctgcacgaagagttcctccgctcctccgtcgacgtcctgctgcaagaggccgtgttcgagggcaccaaccgcaagaaccgcgtcctgcagtggcgcgagcccgaagaactgcgccgcctgatggacttcggcgtccgcagcgccccgtccacgcatgaggaactgctcgaggtcctgaagaaggtcgtcacctactccgtcaagaccggccatccgtacttcgtcaaccagctgttctccgccgtcgatccctacggcctggtcgcccagtgggccaccgacgcgctgaacccctccgtctacacctacgaggtcagccccgtgttcgtcctgatggaagaggtcgtcctgcgcgagatgcgcgccatcgtcggcttcgaaggcggcaaaggcgacggcatcttctgccctggcggctcgatcgccaacggctacgccatcagctgcgcccgctaccgcttcatgcccgacatcaagaagaagggcctgcactccctgccgcgcctggtcctgttcacctccgaggacgcccactactcgatcaagaagctggcctcgttccaaggcatcggcaccgacaacgtctacctgatccgcaccgacgctcgcggtcgcatggacgtcagccacctggtcgaagagatcgagcgctccctccgcgagggcgctgccccgttcatggtcagcgccaccgccggcaccaccgtcatcggcgccttcgatcccatcgagaagatcgccgacgtctgccagaagtacaagctctggctgcacgtcgacgccgcctggggcggaggcgctctggtgtccgccaagcaccgccatctgctgaagggcatcgagcgcgccgactccgtcacctggaatccccacaagctgctgaccgctccgcagcagtgcagcaccctgctgctgcgccacgagggcgtcctggccgaggcgcactccaccaacgccgcctacctgttccagaaggacaagttctacgacaccaagtacgacaccggcgacaagcacatccagtgcggccgtcgcgccgacgtgctgaagttctggttcatgtggaaggccaagggcacctccggcctcgagaagcacgtggacaaggtgttcgagaacgcccgcttcttcaccgactgcatcaagaaccgtgagggcttcgagatggtgatcgccgagcctgagtacaccaacatctgtttctggtacgtccccaagagcctgcgcggacgcaaggacgaggccgactacaaggacaagctgcacaaggtcgcccctcgcatcaaagaacgcatgatgaaggaaggctccatgatggtcacctaccaggcgcagaagggccatccgaatttcttccgcatcgtctttcagaactccggcctggacaaggccgacatggtccatctggtcgaggaaatcgaacgcctgggctccgacctctga

SEQ ID NO: 131 - DNA, 1356 bp, synthetic construct
  source 1..1356: mol_type = other DNA
atggaactgatgatcgtccaggtcaccgagcagacccagagcctgaagaaaaccgacgagaagtacctgtggcacgccatgcgcggtgccgctccgagtccgaccaacctgatcatcaccaaggccgaaggcgcgtgggtcaccgacatcgacggcaaccgctacctggacggcatgtccggcctgtggtgcgtcaacgtcggctacggccgcaaagagctggctcgcgctgccttcgagcagctggaagagatgccgtacttcccgctgacgcagtcgcacgtccccgccatcaagctggccgagaagctgaacgagtggctggacgacgagtacgtcatcttcttctccaactccggctcggaggccaacgaaaccgccttcaagatcgcccgccagtaccaccagcagaagggtgaccacggccgctacaagttcatctctcgctaccgcgcctaccacggcaactccatgggtgccctggccgcgaccggtcaggcccagcgcaagtacaagtacgagcccctcggtcagggcttcctgcacgtggcccctccggacacctatcgcaaccccgaggacgtgcacaccctggcctccgccgaagaaatcgaccgcgtcatgacctgggagctgtcccagaccgtcgccggcgtcatcatggaacccatcatcaccggtggtggcatcctgatgccgccggacggctacatggaaaaggtcaaagagatctgcgaaaagcacggcgccctgctgatctgcgacgaggtcatctgcggcttcggtcgcaccggcaagcccttcggcttcatgaactacggcgtcaagcccgacattatcaccatggccaagggtatcacctcggcctacctgccgctgtctgccaccgcggtccgccgcgaggtctacgaggccttcgtcggctccgacgactacgaccgcttccgccatgtcaacaccttcggcggcaaccctgccgcctgcgcgctggccctgaagaacctcgagatcatggaaaacgagaagctcatcgagcggtccaaagaactgggcgagcgcctgctgtacgagttggaggacgtcaaagagcaccccaacgtcggggacgtccgcggcaagggcctcctgctgggtatcgaactcgtcgaggataagcagaccaaagagcccgcctccatcgaaaagatgaacaaggtcatcaacgcctgcaaagagaagggtctgatcatcggcaagaacggcgacaccgtggccggctacaacaacatcctgcagctcgcccctccgctgtccatcaccgaagaggacttcaccttcatcgtcaagactatgaaggaatgtctggcccagctctga

SEQ ID NO: 132 - DNA, 747 bp, synthetic construct
  source 1..747: mol_type = other DNA
atgatcgtgctggtcacgggcgcgaccgccggtttcggcgagtgcatcacccgccgcttcatccagcagggccacaaggtgatcgctaccggacgccgccaagagcgcctccaagagctgaaggatgagctgggcgacaacctgtacattgcccagctggacgtgcgcaaccgggctgccatcgaagaaatgctcgcctcgctgcccgccgagtggtgcaacatcgacatcctggtcaacaacgccggtctggccctcggcatggaaccggcgcacaaggccagcgtcgaggactgggaaaccatgatcgacaccaacaacaagggactcgtctacatgacccgcgctgtgctgcccggcatggtcgagcgcaaccacggccacatcatcaacatcggctccaccgctggcagctggccctacgctggcggcaacgtctatggcgcgaccaaggcgttcgtccgccagttctccctgaacctgcgcaccgacctgcacggcaccgccgtccgcgtgaccgacattgagcccggtctggtcggcggcaccgagttcagcaacgtccgcttcaagggcgacgacggcaaggccgagaaaacctaccagaacaccgtcgctctgacccctgaggatgtcagcgaggccgtctggtgggtcagcactctgcccgcgcacgtcaacatcaacaccctcgagatgatgcccgtcacgcagtcctacgccggcctgaacgtccaccgccaatag

SEQ ID NO: 133 - DNA, 3579 bp, Aspergillus niger
  source 1..3579: mol_type = genomic DNA
atggctgctccccgccagcccgaggaggcggtcgatgacaccgagttcatcgatgaccaccatgaccagcaccgggactctgtccacacccgtctgcgtgccaattcggctatcatgcagttccaaaagatccttgttgccaaccgtggtgaaatccccattcgtatcttccggacggctcacgagctgtccctgcagaccgtcgccgtctactcccatgaggaccgtctctccatgcaccgtcagaaggccgacgaggcctacatgattggcaagcgcggtcaatatacaccggttggggcctacttggccattgacgagatcgtcaagattgctctggagcatggtgtacacctgatccacccgggttacggtttcctgtccgagaacgccgagtttgcccgcaaggtggagcagtccggcatggttttcgtcggccctaccccccagaccatcgagagcctcggtgacaaggtctccgcccgtcagctggctatccgctgcgacgtgcccgtcgtgcccggtaccccgggccctgtcgagcgctacgaggaggtcaaggccttcaccgacacctacggcttccccatcatcatcaaggccgcctttggtggtggtggtcgtggtatgcgtgtcgttcgcgaccaggccgaactgcgtgactccttcgagcgtgccacctccgaggcccgctctgcctttggcaacggcaccgtcttcgtcgagcgcttcctcgaccgccccaagcacatcgaagtccagctgctgggtgacaaccacggcaacgtcgtccacctgttcgagcgtgactgctccgtccagcgtcgccaccagaaggtcgttgaaattgccccggccaaggacctgcctgccgatgtccgtgaccgcatcctggctgatgctgtcaagctggccaagtcggtcaactaccgcaacgccggtactgcagagttcctggttgaccagcagaaccgttactacttcattgagatcaacccccgtatccaggtcgagcacactatcaccgaagagatcacgggtatcgatatcgttgctgctcagatccagattgcggccggtgctaccctggaacagctgggtctgacccaggaccgcatctccactcgtggattcgccattcagtgccgtatcaccaccgaggacccctccaagggcttctcccccgacaccggtaagatcgaggtctaccgctccgccggtggtaacggtgtccgtctggatggtggaaacggtttcgccggtgccatcatcacccctcactacgactccatgttggtcaagtgtacctgccgtggttccacctatgagatcgctcgccgcaaggtcgttcgtgctttggtcgagttccgtatccgtggtgtcaagaccaacattcctttcctcacctctcttctgagtcaccctgtgttcgtggatggtacctgctggaccacgttcattgatgacactcccgagctgttcgcccttgtcggcagtcagaaccgtgcccagaagctgctggcttacctgggtgatgtggctgtcaacggcagcagcatcaagggccagatcggcgagcccaagctcaagggcgatatcatcaagcccgttctgcatgacgctgccggcaagcccctcgacgtctctgtccccgccaccaagggatggaagcagatcctggacagtgagggtcccgaggcttttgcccgcgctgtgcgtgccaacaagggctgcttgatcatggatactacctggcgtgatgcccaccagtcgctgctggccactcgtgtgcgtaccattgacctcctgaacatcgcccacgagacgagccacgccctcgccaacgcctacagtttggaatgctggggtggtgccaccttcgatgtcgccatgcgcttcctgtacgaggacccctgggaccgtcttcgcaagctgcgcaaggccgttcccaacatccccttccagatgttgctccgtggtgccaacggtgttgcttactcctccctccctgacaacgccatctaccacttctgtaagcaggccaagaagtgcggtgtcgacatcttccgtgtcttcgatgctctcaacgacgttgaccagctcgaggtcggtatcaaggctgtccacgctgccgagggtgttgttgaggctactatttgctacagtggtgatatgctcaaccccagcaagaagtacaacctgccttactacctcgaccttgttgataaggtggtccaattcaagccccacgtcttgggtatcaaggacatggctggtgtgctgaagccccaggctgctcgtctgctgatcggttccatccgcgagcgctaccccgacctccccatccacgtgcacacccacgattctgctggtaccggtgtggcttccatgattgcttgcgctcaggccggtgctgatgccgttgatgctgccacggacagcctttccggtatgacctcccagcccagcattggtgctatcctcgcttccctcgagggaactgagcacgaccccggcctcaactcggcccaggttcgcgcccttgacacctactgggcgcagctgcgtctcctctactcgcccttcgaggcaggtctgactggtcccgaccccgaggtttacgagcatgagatccctggtggtcaattgaccaacctgatcttccaggccagccagcttggtctgggccagcagtgggcggagacgaagaaggcttacgagtctgccaacgatcttttgggcgatgtcgtcaaggtcacccccacttccaaggtggtcggtgatttggctcagttcatggtctccaacaagttgactgctgaagatgtgattgctcgcgccggcgagcttgacttccccggttccgttctggagttcctcgagggtctcatgggccagccctacggtggattccccgagcctctgcgctctcgcgctctgcgtgaccgtcgcaagctcgacaagcgccctggtctgtacctggagccccttgacctggccaagatcaagagccagatccgggagaactatggcgcagctaccgagtacgatgtggccagctatgccatgtaccccaaggtcttcgaggattacaagaagtttgtcgccaagttcggtgatttgtccgtcctgcccacgcgttacttcttggccaagcccgagatcggcgaggaattccacgtcgagctggagaagggtaaggtgctgatcctgaagttgttggccattggtcccctctccgagcagaccggccagcgtgaggtcttctacgaagtcaacggtgaggttcgccaggttagtgtggacgacaagaaggcgtccgtcgagaacaccgcccgccccaaggccgagctgggtgacagcagccaggttggtgctcctatgagcggtgtggttgtcgagatccgcgtccacgacggcctggaagtcaagaagggtgaccccattgccgtcctgagcgccatgaagatggaaatggttatctctgctccccacagtggcaaggtctccagcttgctggtcaaggaaggtgactcggtggatggccaggatcttgtctgcaagatcgtcaaggcctag

SEQ ID NO: 134 - DNA, 827 bp, Aspergillus niger
  source 1..827: mol_type = genomic DNA
cttcggagtagcaacgagtattttcaccgggagtttcaacgggttctatttcaggaacacggctgcggtctggattgggtcgggctgagataccgactggtggcgtcagtggcgggtacggacggagtcgtcctggccgctcgtagacactcccccggactgatatcaggccccggcaactggcttcgtctcactccagggcatcaggagtgcctaccacatgggttcaggctttgccccgtcgtctaagtttgcaggacaaaattttcgtatgcgttaccactctttcctttcagcaaccattccgtagtgaaaacccaataataggtggctgccgtgggagcctgagtcaacccaaccagaacctttctagtagattctcccccaagcgcttcagcaacgaagcgtattggagaaccaaatgacgcagaccaagcggattccggtgcaatagccggatggcaagggaatcccccaggaggtgccagaagcgtcgcccgaaaggtacttcgtctgacaggctaacaccgctcgggctaaggtccctgctgctcttttccctttattgcgacttaacctctaagccattcccttgcatcacgttatctcactgaccgacctctgactaaggcgcttcgcctccgccgcctcccctcattcacctcctctcctgactacttaagccttctcttccttcctctcactaccaaccctccttcatccctcatacctctcatcctaccactcacctttcgcgcatcgccatctgcgatcctccccacaacactccacctagatacatacaccattaactgcgcttctacaac

SEQ ID NO: 135 - DNA, 826 bp, Aspergillus niger
  source 1..826: mol_type = genomic DNA
cattatcgatctatccacattgcctcttataactgacgggaggaggcgacctgtggcctgtgagggtttgaaacattatttcacccttcctctgcaatagccgatttaataattgacgcctatcccgcatgctattcaatcaataattagcaacagcgagccgagggatgagagggcggcaaatgatgacgatgacgaagttgacgtcgatgctgagaggctggattgaccacttcgtctagttggttttaggcctagtcgtactttctgcctggcttttccgggccgtgcctatgcctggcgatgcaggcaccaaactgacgtatccaagaaaccgttaaagtcagcaaatcccgtgacctcagccgcagaccatcacatgactgaggacccggagggctgattatccgagtgtccacccgcactacgcctgactcggtaatctggacaccacgtgaccgcacaaccgattttaacgaagcccaccaccaccgaggatattttcctaccacgatcgccgaaggtttaggaagacaagtcttgtcgaggtgagttgatgaccctatacatttactatgtgacaaggaaaaggattcaatattatttggaacatctcatcttcatttctttccacttatcccagaaaatacctcctgcgcacactttagcgcgcgagataccaaaattccgggggagtaatatgttgaacaagaaacgaagatgagagctgcgacgcagataatattgaactttatccttgctcacgcaccagtatcaagtaaaacaatgaactaacatcctttttccaacccagaccttcatcaag

SEQ ID NO: 136 - DNA, 1198 bp, Aspergillus niger
  source 1..1198: mol_type = genomic DNA
ggacatgctggaagggattttctggctcaataccacgtctgtatttgaccctttccagacagttaatccgctgcaggagggcgaactgtagctcctcgttctccttgtagcgcttgatccagtctttttggatgttgcacttgcttggcctatgcttctcatataatcttgccctgtcatagagacgacgtctgagattgtagcgttcgtctttgatcacccggagccagataggcctgagtatatctgacattagatcaaagggtctgtggatagtctccttcagcatcagcgacgcatgtgactcgcatgtcggagagagcttgtgggtggtcatctttgatggcgtcctctgctttcccttgattttcgttgattgtttttcgaaagttaagtctggaagtcaagagaatccttctgccagacattatatttacgtatactgacgtagtagaaacagcgtcaggatgaggacatggtgtgtgctggaccacggaatcatagttcatcagtatattgggttggacaaataacgctgagcatgtatatgtctttacacactataaaagccagcgaacgccaataaaatagggcatattgatgtgaaaatatgacaccagttaaaagcagtgtattgattttatctctcttcacctcggacctatactaccgtatacaagactcaacttacttccagatatagtaatatacaccctatggacgaaccagcacaataattacagccaaacaacaccacccaaatggcatattcctaatcagcactaagcacaaataccactgtcatcacagcataatcaataagaatcccagacaaccgactcactctgactcaccttacacaaacccccaagcaaagcgcagcccagaacctcagccaacaatcgggcaacgtacggggaaagattggccgatccatgatgtcagcagccctaacccaaagcggactagcgcataccgcccctctgactccgccatcccagggctcgagaagcttccgtggcgtcgatataaattcagcgggccttgaacatccctccttacgacacacctcacgcgatcgattttgacactcacacaccgccaccctcacatcctccacccacaccacaccccttaatcaacccaccatcaccgctagaacgtctatctcatcaccgacttctcatccatcttcaaa

SEQ ID NO: 137 - protein, 423 aa, Aspergillus niger
MHTTEKIPSFLSRRKPSNDSEHVPPPPDGGVQAWTQVVCMHFVFFNTWGITTSFSVFEQLYTKTLPQSASSISWIGSVQVSLLFFLSALAGRATDAGFFRVMYPLGVLLQLVGIFMLSLCKTYWQIFLAQAVCMGLGNGLTFSPGLSVMSAYFMKNRAFAVGLAASGAATGGLIYPVLINQLLYNHQIGFAWTIRAAGLLMLITHIFGLVFFRPRLPPRTTGPLIELGAFTEPPFVFFTLCYFFTFWGLYFAWFYLGTFARDRLGIADTQNLLLVLNGVGIVGRIGPSIIGDRWTGRLNILIPITLSCAILMFCWIAVSTVAGLYAFVVVYGLVGGAAQSVIPATATTMTPDINRTGTRLGMIMSIVGFATLTGPAIEGALIATDGGRYVAAQIFAGVSILLGVCAVAAARVAKAGWGLDVKV

SEQ ID NO: 138 - protein, 418 aa, Aspergillus niger
MGSTTPSVFSTAVVPAAPEDALFGLAQAFRQDSSDKKVDLVIGAYRDDNAKPWILPVVKKADELVRNDPALNHEYLPIKGLADYTTAAQKLIIGADSPAIRENRVCTFQTISGTGAVHLGALFLSKFHPSNPKPTVYLSNPTWANHNQIFTNVNLSLANYPYFDPQTKGLNFSGMLSALRDAPTGSIILLHVCAHNPTGVDLTQSQWKDVAVVMRERNHFPFFDCAYQGFASGDLIRDSWAVRYFVEQGFELCVAQSFAKNFGLYGQRTGAFHFVSAPGAEASQANAHVASQLAILQRSEISNPPAYGARIASRVLNDEGLFKEWEEDLKTMSGRIAEMRQGLRERLEKKGTPGTWNHITDQIGMFSFTGLTESQVKVLKEKWHVYMTKNGRISMAGLNTHNLDYFAEAVDSVVRETS

SEQ ID NO: 139 - DNA, 1404 bp, synthetic construct
  source 1..1404: mol_type = other DNA
atgtcgcccctttcctcctcttcttcttctccatcttcttctctctccctcacttcttcttcatcctctccatccgcctcctcctccatcgcttctacttctacttctaccgctcgttcccgtctcgcttctctctcctcccacatcatgggctctaccaccccctccgtcttttccaccgccgtcgtgcccgccgcgccagaagatgctctcttcggtctggctcaggcgtttcgccaggattcctccgacaagaaggtcgatctagtcattggcgcttatcgcgacgacaatgccaaaccctggatcctgcccgtggtcaagaaggctgacgagctcgttcgcaatgaccccgccctcaatcacgaatacctccccatcaaaggtctcgccgactacaccactgccgcccagaaattgatcattggcgctgatagccctgccattcgcgagaaccgcgtatgcaccttccaaaccatctccggcaccggtgccgtgcacctgggcgctctcttcctctccaaattccacccctccaaccctaaacccaccgtctacctctccaacccaacctgggccaaccacaaccaaatcttcaccaacgtcaacctctccctcgccaactacccctacttcgacccgcaaaccaaaggcctcaacttctccggcatgctctccgccctgcgcgatgctcccaccggctccatcatcctcctgcacgtctgcgcccacaaccccaccggtgtcgatctgacccagtcccaatggaaggacgtcgccgtcgtcatgcgcgagcgcaaccatttccccttctttgactgcgcctaccagggtttcgcctctggtgatctcattcgcgactcctgggccgtgcgctacttcgtcgagcagggcttcgagctgtgcgtggcccaatccttcgctaagaactttggtctgtatggccagcgcacgggcgcattccatttcgtttccgcacccggtgcggaggccagccaggctaatgcgcatgtcgcgtcgcagctggctattctgcagcgcagtgagatcagtaacccgccggcgtatggcgcgcgtatcgctagccgggtgttgaatgatgagggactgttcaaggagtgggaggaggatctgaagacgatgagtggtcgcattgcggagatgcgccagggcctgagggagcgcttggagaagaagggcacgccgggcacttggaaccacattacggatcagattggcatgttcagtttcacgggtttgaccgagtcgcaagttaaggtgttgaaggagaagtggcatgtttacatgaccaagaacggccgcatctccatggcgggtctaaacacacataacctggactacttcgccgaagcagtggacagcgtagttcgggagacttcataa

SEQ ID NO: 140 - DNA, 810 bp, Aspergillus niger
  source 1..810: mol_type = genomic DNA
ctactatgaaagaccgcgatgggccgatagtagtagttacttccattacatcatctcatccgcccggttcctcgcctccgcggcagtctacgggtaggatcgtagcaaaaacccgggggatagacccgtcgtcccgagctggagttccgtataacctaggtagaaggtatcaattgaacccgaacaactggcaaaacattctcgagatcgtaggagtgagtacccggcgtgatggagggggagcacgctcattggtccgtacggcagctgccgagggggagcaggagatccaaatatcgtgagtctcctgctttgcccggtgtatgaaaccggaaaggactgctggggaactggggagcggcgcaagccgggaatcccagctgacaattgacccatcctcatgccgtggcagagcttgaggtagcttttgccccgtctgtctccccggtgtgcgcattcgactgggcgcggcatctgtgcctcctccaggagcggaggacccagtagtaagtaggcctgacctggtcgttgcgtcagtccagaggttccctcccctaccctttttctacttcccctcccccgccgctcaacttttctttcccttttactttctctctctcttcctcttcatccatcctctcttcatcacttccctcttcccttcatccaattcatcttccaagtgagtcttcctccccatctgtccctccatctttcccatcatcatctcccttcccagctcctcccctcctctcgtctcctcacgaagcttgactaaccattaccccgccacatagacacatctaaaca

SEQ ID NO: 141 - DNA, 704 bp, Aspergillus niger
  source 1..704: mol_type = genomic DNA
ctcgagcttacaagaagtagcctgtagcaagtctgtggtcgtgtttattatttatctactacttccccctcctccctcctcctcttgtgttaagagtactctcaaaaaaagcctttcttagatagtacaaccaccatcttcaaggtcctgtaagtatgacttgtcccatatactcattttatcacccctcccatctttctctgtccatttcatcctcctcttcttcctcttagctgctagtcatgaccccgccatcatcccaccatgttgtgcttgacccctccatctatccatccatccatcctcctgtctatatgccatcactccatcctgtctgtctatcaatgatctggcatctccggatttagcaccaagtcccattgattagttgatcatttgggaaacttggctgtgccgagcctggtctatttgatggatcggacctctccacctctaattccccccccgtccttcccttctccgtcactcatccgccatccgctcattctttttgggtcctcggtgaacactcaccaccttcccccccgctgctgctagtgcacaccgaaacagcaagtccggagcgcttacatttttgcctcctccgttttttcctatatctgggccatgttctccctttgccatctttgttttctcccagtttcactgatccataactcatccccctccagtccatcaacatg

SEQ ID NO: 142 - DNA, 10353 bp, synthetic construct
  source 1..10353: mol_type = other DNA
gtttaaacttcggagtagcaacgagtattttcaccgggagtttcaacgggttctatttcaggaacacggctgcggtctggattgggtcgggctgagataccgactggtggcgtcagtggcgggtacggacggagtcgtcctggccgctcgtagacactcccccggactgatatcaggccccggcaactggcttcgtctcactccagggcatcaggagtgcctaccacatgggttcaggctttgccccgtcgtctaagtttgcaggacaaaattttcgtatgcgttaccactctttcctttcagcaaccattccgtagtgaaaacccaataataggtggctgccgtgggagcctgagtcaacccaaccagaacctttctagtagattctcccccaagcgcttcagcaacgaagcgtattggagaaccaaatgacgcagaccaagcggattccggtgcaatagccggatggcaagggaatcccccaggaggtgccagaagcgtcgcccgaaaggtacttcgtctgacaggctaacaccgctcgggctaaggtccctgctgctcttttccctttattgcgacttaacctctaagccattcccttgcatcacgttatctcactgaccgacctctgactaaggcgcttcgcctccgccgcctcccctcattcacctcctctcctgactacttaagccttctcttccttcctctcactaccaaccctccttcatccctcatacctctcatcctaccactcacctttcgcgcatcgccatctgcgatcctccccacaacactccacctagatacatacaccattaactgcgcttctaaacaatgcccgccaccggcgaggaccaggacctggtgcaggacctgatcgaggaacccgccaccttctccgacgccgtcctgtcctccgacgaggaactgttccaccagaagtgccccaagccggctccgatctacagccccgtcagcaagcccgtcagcttcgagtccctgccgaaccgccgcctgcacgaagagttcctccgctcctccgtcgacgtcctgctgcaagaggccgtgttcgagggcaccaaccgcaagaaccgcgtcctgcagtggcgcgagcccgaagaactgcgccgcctgatggacttcggcgtccgcagcgccccgtccacgcatgaggaactgctcgaggtcctgaagaaggtcgtcacctactccgtcaagaccggccatccgtacttcgtcaaccagctgttctccgccgtcgatccctacggcctggtcgcccagtgggccaccgacgcgctgaacccctccgtctacacctacgaggtcagccccgtgttcgtcctgatggaagaggtcgtcctgcgcgagatgcgcgccatcgtcggcttcgaaggcggcaaaggcgacggcatcttctgccctggcggctcgatcgccaacggctacgccatcagctgcgcccgctaccgcttcatgcccgacatcaagaagaagggcctgcactccctgccgcgcctggtcctgttcacctccgaggacgcccactactcgatcaagaagctggcctcgttccaaggcatcggcaccgacaacgtctacctgatccgcaccgacgctcgcggtcgcatggacgtcagccacctggtcgaagagatcgagcgctccctccgcgagggcgctgccccgttcatggtcagcgccaccgccggcaccaccgtcatcggcgccttcgatcccatcgagaagatcgccgacgtctgccagaagtacaagctctggctgcacgtcgacgccgcctggggcggaggcgctctggtgtccgccaagcaccgccatctgctgaagggcatcgagcgcgccgactccgtcacctggaatccccacaagctgctgaccgctccgcagcagtgcagcaccctgctgctgcgccacgagggcgtcctggccgaggcgcactccaccaacgccgcctacctgttccagaaggacaagttctacgacaccaagtacgacaccggcgacaagcacatccagtgcggccgtcgcgccgacgtgctgaagttctggttcatgtggaaggccaagggcacctccggcctcgagaagcacgtggacaaggtgttcgagaacgcccgcttcttcaccgactgcatcaagaaccgtgagggcttcgagatggtgatcgccgagcctgagtacaccaacatctgtttctggtacgtccccaagagcctgcgcggacgcaaggacgaggccgactacaaggacaagctgcacaaggtcgcccctcgcatcaaagaacgcatgatgaaggaaggctccatgatggtcacctaccaggcgcagaagggccatccgaatttcttccgcatcgtctttcagaactccggcctggacaaggccgacatggtccatctggtcgaggaaatcgaacgcctgggctccgacctctgatgggttggatgacgatgacttcatgtgattttgttatttagaatattttatatttccttttcttcttctcaccaccgatccccttaacactcttgcttcatttgcttcagatttctcggtttcttcttttttcttctccccagttatccactatatctttgctagaccggcctgcgccctggcatgcatcataaaatcatgtccgttggtcatcatctgttttgtatatccgtcatataaagtattcttttattccctcccccctcggtcgtctttcgctgtcccgcttcctacctccggtttatagagcatggttcatctcttccgtacatttccgttggtactagcatttatgtcttcagctagtatagaagctgccgcagttgttcgcttactacctgcctaagtccttaactttttaaagtgtttaacctatacgtagtgttaaacgagtactgggaggtggtgaggtagaaaatgttctgcacgggcagtgggtatttggtagtgtgtaaggcggttatttatcaggctgacgctaaagacttctatgggagcagtatgggatcgcggctcatagaagtacacaaaatctaagagtcgtttgataattaattgattcccggcagggtcttcttgggattgagagaactggttactttgatttgagatattgtaaagcttaaggctcttaacacgtacgagcgaaacagcaggggggaaatcgggaaaaggggcgtggggtgaataaaaaagttgaaataagacactgtatcttgctgggggtgaataaagagagaataaaagagaggtaaattccactcagccccttttcttcgctctccaaacatcaaactccgccggccgacccacaggatcccgaacaagtggaagatatgtgccggtccagacccttcgcacagctaaaagcagaccttcataagcgtttccgggtagtattcgcacacctgaactggcacgtcggggacacaactgtttttgatacacaagaacacacaccacccatctaggactcagagctgggccagacattccttcatagtcttgacgatgaaggtgaagtcctcttcggtgatggacagcggaggggcgagctgcaggatgttgttgtagccggccacggtgtcgccgttcttgccgatgatcagacccttctctttgcaggcgttgatgaccttgttcatcttttcgatggaggcgggctctttggtctgcttatcctcgacgagttcgatacccagcaggaggcccttgccgcggacgtccccgacgttggggtgctctttgacgtcctccaactcgtacagcaggcgctcgcccagttctttggaccgctcgatgagcttctcgttttccatgatctcgaggttcttcagggccagcgcgcaggcggcagggttgccgccgaaggtgttgacatggcggaagcggtcgtagtcgtcggagccgacgaaggcctcgtagacctcgcggcggaccgcggtggcagacagcggcaggtaggccgaggtgatacccttggccatggtgataatgtcgggcttgacgccgtagttcatgaagccgaagggcttgccggtgcgaccgaagccgcagatgacctcgtcgcagatcagcagggcgccgtgcttttcgcagatctctttgaccttttccatgtagccgtccggcggcatcaggatgccaccaccggtgatgatgggttccatgatgacgccggcgacggtctgggacagctcccaggtcatgacgcggtcgatttcttcggcggaggccagggtgtgcacgtcctcggggttgcgataggtgtccggaggggccacgtgcaggaagccctgaccgaggggctcgtacttgtacttgcgctgggcctgaccggtcgcggccagggcacccatggagttgccgtggtaggcgcggtagcgagagatgaacttgtagcggccgtggtcacccttctgctggtggtactggcgggcgatcttgaaggcggtttcgttggcctccgagccggagttggagaagaagatgacgtactcgtcgtccagccactcgttcagcttctcggccagcttgatggcggggacgtgcgactgcgtcagcgggaagtacggcatctcttccagctgctcgaaggcagcgcgagccagctctttgcggccgtagccgacgttgacgcaccacaggccggacatgccgtccaggtagcggttgccgtcgatgtcggtgacccacgcgccttcggccttggtgatgatcaggttggtcggactcggagcggcaccgcgcatggcgtgccacaggtacttctcgtcggttttcttcaggctctgggtctgctcggtgacctggacgatcatcagttccatcttgatgaaggtctgggttggaaaaaggatgttagttcattgttttacttgatactggtgcgtgagcaaggataaagttcaatattatctgcgtcgcagctctcatcttcgtttcttgttcaacatattactcccccggaattttggtatctcgcgcgctaaagtgtgcgcaggaggtattttctgggataagtggaaagaaatgaagatgagatgttccaaataatattgaatccttttccttgtcacatagtaaatgtatagggtcatcaactcacctcgacaagacttgtcttcctaaaccttcggcgatcgtggtaggaaaatatcctcggtggtggtgggcttcgttaaaatcggttgtgcggtcacgtggtgtccagattaccgagtcaggcgtagtgcgggtggacactcggataatcagccctccgggtcctcagtcatgtgatggtctgcggctgaggtcacgggatttgctgactttaacggtttcttggatacgtcagtttggtgcctgcatcgccaggcataggcacggcccggaaaagccaggcagaaagtacgactaggcctaaaaccaactagacgaagtggtcaatccagcctctcagcatcgacgtcaacttcgtcatcgtcatcatttgccgccctctcatccctcggctcgctgttgctaattattgattgaatagcatgcgggataggcgtcaattattaaatcggctattgcagaggaagggtgaaataatgtttcaaaccctcacaggccacaggtcgcctcctcccgtcagttataagaggcaatgtggatagatcgataatggagatagggtggttgtctggaggtcgacggtatcgataataacttcgtatagcatacattatacgaagttatgaacgactccagaagtgactaagcaaccgtaaaccggaagcaacaatccggctctcatggcaaagatggcatgtccggattgcggaggaaagatttggcgtgtgcctgacggttttgttccacaaggaccctgtaaacttggtgatggtaatgaggtgctctggccttgattgagcaaaagtcagctcgcggggactacgtgagttgatggtggaggatcatgtactaaatagagcatacctaggggatggtgaacggacgtccagggttccgtagaattgtaacaactctactcgacttgctgcccactggggtatgtaaatacatgccttttcaaagttgccgagtgtactaaatggagtgtatgtttggccaatgtcatttgggaggggccgggaaccctttgggacacccgcggatgggctggatctcggtcactggagacatagcctagtggtctagtagtctagcagcaggacagcttcatggttaaccccacatgtgatcctccgtccggtttccgtagtatataatggacctgaagcttctccctctctcgatgcggttgaaacataaatttgctcataacgtatgtccatctaaccctttcattgtttcttgaccactgtctcctttctggaaaaaaaaagactctggtaagtatgggcctaggatcgagctactgatcccccaatccttcctaatccctctgccacttgccccgacctgttcaccgtgaccgtaccaccaccaccccgccccctcctcaactaaacccctcccttcccctcccccctgatgttttctcttttctttaatctgactgtcacctccctctctacttcttttccctcaattttcttctcctcttcttactctctgtcatctgtttcgatacccatactaatctcgcgtgctttttcttcaccttcgtgtatagatttgtcacaatcccaaatttcaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgtttcgggtttacctcttccagatacagctcatctgcaatgcattaatgcattggacctcgcaaccctagtacgcccttcaggctccggcgaagcagaagaatagcttagcagagtcataacttcgtatagcatacattatacgaagttattgcagcccgggacatgctggaagggattttctggctcaataccacgtctgtatttgaccctttccagacagttaatccgctgcaggagggcgaactgtagctcctcgttctccttgtagcgcttgatccagtctttttggatgttgcacttgcttggcctatgcttctcatataatcttgccctgtcatagagacgacgtctgagattgtagcgttcgtctttgatcacccggagccagataggcctgagtatatctgacattagatcaaagggtctgtggatagtctccttcagcatcagcgacgcatgtgactcgcatgtcggagagagcttgtgggtggtcatctttgatggcgtcctctgctttcccttgattttcgttgattgtttttcgaaagttaagtctggaagtcaagagaatccttctgccagacattatatttacgtatactgacgtagtagaaacagcgtcaggatgaggacatggtgtgtgctggaccacggaatcatagttcatcagtatattgggttggacaaataacgctgagcatgtatatgtctttacacactataaaagccagcgaacgccaataaaatagggcatattgatgtgaaaatatgacaccagttaaaagcagtgtattgattttatctctcttcacctcggacctatactaccgtatacaagactcaacttacttccagatatagtaatatacaccctatggacgaaccagcacaataattacagccaaacaacaccacccaaatggcatattcctaatcagcactaagcacaaataccactgtcatcacagcataatcaataagaatcccagacaaccgactcactctgactcaccttacacaaacccccaagcaaagcgcagcccagaacctcagccaacaatcgggcaacgtacggggaaagattggccgatccatgatgtcagcagccctaacccaaagcggactagcgcataccgcccctctgactccgccatcccagggctcgagaagcttccgtggcgtcgatataaattcagcgggccttgaacatccctccttacgacacacctcacgcgatcgattttgacactcacacaccgccaccctcacatcctccacccacaccacaccccttaatcaacccaccatcaccgctagaacgtctatctcatcaccgacttctcatccatcttcaaaatgatcgtgctggtcacgggcgcgaccgccggtttcggcgagtgcatcacccgccgcttcatccagcagggccacaaggtgatcgctaccggacgccgccaagagcgcctccaagagctgaaggatgagctgggcgacaacctgtacattgcccagctggacgtgcgcaaccgggctgccatcgaagaaatgctcgcctcgctgcccgccgagtggtgcaacatcgacatcctggtcaacaacgccggtctggccctcggcatggaaccggcgcacaaggccagcgtcgaggactgggaaaccatgatcgacaccaacaacaagggactcgtctacatgacccgcgctgtgctgcccggcatggtcgagcgcaaccacggccacatcatcaacatcggctccaccgctggcagctggccctacgctggcggcaacgtctatggcgcgaccaaggcgttcgtccgccagttctccctgaacctgcgcaccgacctgcacggcaccgccgtccgcgtgaccgacattgagcccggtctggtcggcggcaccgagttcagcaacgtccgcttcaagggcgacgacggcaaggccgagaaaacctaccagaacaccgtcgctctgacccctgaggatgtcagcgaggccgtctggtgggtcagcactctgcccgcgcacgtcaacatcaacaccctcgagatgatgcccgtcacgcagtcctacgccggcctgaacgtccaccgccaataggaccgatggctgtgtagaagtactcgccgatagtggaaaccgacgccccagcactcgtccgagggcaaaggaatagagtagatgccgaccgggatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgtttcgggtttacctcttccagatacagctcatctgcaatgcattaatgcattggacctcgcaaccctagtacgcccttcaggctccggcgaagcagaagaatagcttagcagagtctattttcattttcgggagacgagatcaagcagatcaacggtcgtcaagagacctacgagactgaggaatccgctctctgacagacgggcaattgattacgggatcccattggtttaaac

SEQ ID NO: 143 - DNA, 47 bp, synthetic construct
  source 1..47: mol_type = other DNA
acaggctacttcttgtaagctcgagtttctgtacagtgaccggtgac